(19) United States
(12) Patent Application Publication
(10) Pub. No.: US 2018/0194861 A1
(43) Pub. Date: Jul. 12, 2018

(54) IgM- or IgE-Modified Binding Proteins and Uses Thereof

(71) Applicant: AbbVie Inc., North Chicago, IL (US)

(72) Inventors: Feng Dong, Lansdale, PA (US); Jijie Gu, Shrewsbury, MA (US); Tariq Ghayur, Holliston, MA (US)

(21) Appl. No.: 15/742,331

(22) PCT Filed: Jul. 8, 2016
(86) PCT No.: PCT/US16/41618

§ 371 Date: Jan. 5, 2018

Related U.S. Application Data
(60) Provisional application No. 62/191,038, filed on Jul. 10, 2015.
(60) Provisional application No. 62/316,951, filed on Apr. 1, 2016.

(51) Int. Cl. C07K 16/46

(52) U.S. Cl. CPC C07K 16/468; C07K 2317/31; C07K 2317/35; C07K 2319/30; C07K 2317/526; C07K 2317/64; C07K 2317/524

(57) ABSTRACT

Engineered binding proteins comprising a modified constant region, such as an IgG constant region modified to contain a CH2 domain from an IgM, a CH2 domain from an IgE, or a variant thereof, are disclosed. The binding proteins can be multispecific, including bi-, tri-, and tetra-specific constructs. Also disclosed are uses of the binding proteins in the diagnosis, prevention, and/or treatment of disease.

This is a national stage application under 35 U.S.C. § 371 of international application number PCT/US2016/041618, filed Jul. 8, 2016, which designated the U.S. and which claims priority to U.S. Provisional Application Ser. No. 62/191,038, filed Jul. 10, 2015, and U.S. Provisional Application Ser. No. 62/316,951 filed Apr. 1, 2016, all of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Sep. 13, 2016, is named 12252_0204-00304_SL.txt and is 902,459 bytes in size.

### FIELD

Disclosed herein are engineered binding proteins comprising a modified constant region, such as an IgG constant region modified to contain a CH2 domain from an IgM, a CH2 domain from an IgE, or a variant thereof, as well as their uses in the diagnosis, prevention, and/or treatment of disease.

### BACKGROUND

Engineered proteins, such as multispecific binding proteins capable of binding two or more antigens, are known in the art. Such multispecific binding proteins can be generated using cell fusion, chemical conjugation, or recombinant DNA techniques.

Production of multispecific binding proteins by co-expression of light and heavy chains, e.g., from different antibodies, in a single host cell can result in low yield of the desired bispecific due to mispairing of heterologous heavy and light chain sequences. For instance, where a bispecific antibody is intended to have heterologous binding domains on the two antibody arms (i.e., a binding site for antigen A on the first arm and for antigen B on the second arm), various mispairings can occur during co-expression of the light and heavy chains in a single cell. These include a heavy chain heterodimer with light chain mispairings, and heavy chain homodimers with or without light chain mispairing. When co-expressing two different antibody heavy and light chains in one cell line, assuming random chain association, a total of 16 combinations are possible. Of those, six are identical. Thus, a purely statistical association leads to 6 tetramers that occur twice (each 12.5% yield) and 4 tetramers that occur once (each 6.25%). The desired bispecific binding protein makes up statistically 12.5% of the total yield. Purification by removing the nine closely-related mispaired structures that occur during single cell synthesis is often difficult and inefficient.

Homo-dimerization of two heavy chains, rather than the desired heterodimerization, during formation of a binding protein such as an IgG is largely mediated by interaction between the CH3 domains. One option to ensure correct hetero-dimerization in a bispecific antibody format has been to engineer modified heavy chain CH3 domains that only interact in a heterodimeric format. Several IgG CH3 hetero-dimerization strategies are known in the art.

An alternative option for overcoming the heavy chain-pairing problem in bispecific antibodies is to use a common heavy chain. For example, κλ-bodies contain a common heavy chain plus κ and λ light chains to confer the two different antigen specificities. Two sequential affinity purification steps are used to purify κλ-bodies with their κ and λ light chains away from monospecific antibodies that contain a single type of light chain.

While the structures described above may address the issue of random heavy chain association, they do not ensure correct light chain association. Thus, even with complete heavy chain hetero-dimerization, a mixture of the desired multispecific construct and unwanted contaminants may result from random light chain association. As such, a method of eliminating light chain mispairing would be beneficial to improve multispecific binding protein yield.

### SUMMARY OF THE DISCLOSURE

Disclosed herein are engineered binding proteins comprising a modified constant region to improve pairing of the correct heavy and light chain sequences, as well as ensuring heterodimeric heavy chain pairing. In various embodiments, the binding proteins, such as heterodimeric binding proteins containing an IgG constant region, are modified to contain a CH2 domain from an IgM or IgE in place of a wild-type CH1 domain, as well as further modifications to ensure correct heavy-light chain pairing and heterodimeric heavy chain pairing. Also disclosed, in various embodiments, are bispecific, trispecific, tetraspecific and other multispecific molecules containing the modified heavy and light chains, as well as their uses in the diagnosis, prevention, and/or treatment of various disease.

In various embodiments, a binding protein is disclosed, comprising a first heavy chain and a first light chain forming an antigen binding region and a modified constant region comprising a modified CH1 domain (CH1*) and a modified CL domain (CL*), wherein the CH1* domain comprises an IgM CH2 domain, an IgE CH2 domain, or a variant thereof; and the CL* domain comprises an IgM CH2 domain, an IgE CH2 domain, or a variant thereof, and wherein the heavy chain and light chain interact at one or more interface between the CH1* and CL*. In some embodiments, the CH1* domain is an IgM CH2 domain, an IgE CH2 domain, or a variant thereof; and the CL* domain is an IgM CH2 domain, an IgE CH2 domain, or a variant thereof. In some embodiments, the CH1* and CL* comprise variants of an IgM or IgE CH2 domain that have been modified to increase electrostatic or hydrophobic interactions at the one or more interface. In some embodiments, the IgM or IgE CH2 domain variants promote heavy chain and light chain heterodimer pairing, and inhibit homodimer pairing of two heavy chains or two light chains. In certain embodiments, the constant region comprises an IgG hinge region, and wherein the hinge region is further modified to remove at least one cysteine residue found in a wild-type IgG hinge region, which may reduce the number of disulfide bonds formed with the IgM or IgE CH2 domain. In some embodiments, the IgM or IgE CH2 domain variant comprises a CH1, C kappa, or C lambda DE loop in place of a wild-type IgM or IgE CH2 DE loop.

In some embodiments, the binding protein comprises, prior to modification, a wild-type human IgG constant region. In some embodiments, the IgG constant region prior to modification is a human wild-type IgG1, IgG2a, IgG2b, IgG3, or IgG4 subtype. In certain embodiments, the modified constant region comprises a fragment of a wild-type IgG, e.g., one which lacks all or a part of an IgG CH3 domain.

In various embodiments, the binding protein is a bispecific or multispecific binding protein, e.g., a bispecific antibody, a multispecific antibody, or a dual variable domain immunoglobulin (DVD-Ig) binding protein.

In some embodiments, the antigen binding region in a binding protein disclosed herein comprises one, two, three, four, or more antigen binding sites that bind the same or different antigen targets. In some embodiments, the antigen binding sites are derived from parental antibody variable domains and/or T-cell receptor binding regions.

In various embodiments, the binding protein comprises a second heavy chain and a second light chain, wherein the second heavy chain and second light chain interact at one or more interface. In some embodiments, the second heavy chain comprises a wild-type IgG heavy chain constant region and a wild-type IgG light chain constant region. In some embodiments, the first heavy chain comprises a modified CH3 domain, and the second heavy chain comprises a modified CH3 domain, and wherein the modified CH3 domains are preferably modified IgG CH3 domains, wherein the modifications promote pairing of the first and second heavy chains at one or more interface in the CH3 domains on the first and second heavy chains, and inhibit homodimer formation of two first heavy chains or two second heavy chains (e.g., using knobs-into holes or electrostatic modifications).

In various embodiments, a binding protein disclosed herein can be conjugated to another agent, e.g., an immunoadhesion molecule, an imaging agent, a therapeutic agent, or a cytotoxic agent. In various embodiments, a pharmaceutical composition is disclosed, comprising a binding protein disclosed herein and a pharmaceutically acceptable carrier, and optionally a further therapeutic agent.

Also disclosed herein are nucleic acid(s) encoding the binding proteins, as well as vectors and host cells containing the nucleic acid(s). Also disclosed, in various embodiments, are methods of treating a subject for a disease or a disorder by administering a binding protein disclosed herein. Also disclosed are methods of detecting the presence, amount, or concentration of at least one target or fragment thereof in a test sample by an immunoassay using a binding protein disclosed herein, and kits for use in detecting the presence, amount, or concentration of at least one target or fragment thereof comprising a binding protein disclosed herein.

### BRIEF DESCRIPTION OF THE DRAWINGS

FIG. 1 shows exemplary IgM CH2 and IgE CH2 sequences and structures. FIG. 1A is a schematic illustration of IgG and IgM immunoglobulins. FIG. 1B shows sequences of the human MH2 and EH2 (SEQ ID NOS 323-324, respectively, in order of appearance). Beta sheets are annotated as A, B, C, D, E, F and G. The loops between each beta sheet are annotated as AB, BC, CD, DE, EF, and FG. Dimer interface residues are underlined, which include residues within 5 Å of paired chain in modeled human MH2 dimer and 2Y7Q.pdb for human EH2 dimer. The potential N-glycosylation sites, 120NAS on MH2 and 38NIT on EH2, are italicized.

FIG. 2 shows exemplary MH2 hetero-dimerization engineering through electrostatic interactions. FIG. 2A shows an alignment of MH2, MH2p, and MH2n amino acid sequences (SEQ ID NOS 325-327, respectively, in order of appearance). The mutations are underlined.

FIG. 2B shows multiple electrostatic interactions on the MH2 dimer interface through 2 sets of 3 major inter-chain pairs: D12-Q119, K20-Q24, and D81-K85.1. FIG. 2C shows the MH2p is created by 2 positive mutations Q24K and D81K while MH2n is created by 3 negative mutations K20E, Q24E, and K85.1D. Attractive electrostatic interactions form between MH2p and MH2n.

FIG. 3 shows exemplary MH2 heterodimer engineering through hydrophobic interactions. FIG. 3A shows an alignment of MH2, MH2k, and MH2h amino acid sequences (SEQ ID NOS 328-330, respectively, in order of appearance). The mutations are underlined. FIG. 3B shows that residue I22, Q24, and T86 are on the MH2 dimer interface and close to each other. FIG. 3C shows that MH2k is created by introducing mutation I22W on one MH2 while MH2h is created by introducing mutation I22A, Q24S, and T86A on the other MH2. Attractive hydrophobic interactions form between MH2k and MH2h.

FIG. 4A shows an expression vector used to produce the wild type or engineered MH2 domains as described in Examples 2.1 and 2.2. FIG. 4A discloses SEQ ID NO: 331. FIG. 4B shows the expression profiles of the wild type and engineered MH2 domains under a non-reducing or reducing condition. FIG. 4B discloses “6His” as SEQ ID NO: 331.

FIG. 5 compares an MH2 or EH2 homodimer with an IgG CH1/C_{κ}(λ) heterodimer. FIG. 5A shows an alignment of IgG CH1, C_{κ}, Cλ, MH2 and EH2 amino acid sequences (SEQ ID NOS 332-336, respectively, in order of appearance). The beta sheets are annotated as A, B, C, D, E, F and G. The loops between each beta sheet are annotated as AB, BC, CD, DE, EF, and FG. The dimerization interface residues are underlined (residues within 5 Å of the paired chain in 1N8Z.pdb for CH1 and Cκ, modeled human MH2 dimer for MH2, and 2Y7Q.pdb for EH2). FIG. 5B shows the modeled human MH2 dimer based on 4JVU.pdb (left image) and a modeled MH2 dimer superimposed with CH1/Cκ hetero-dimer from 1N8Z.pdb (right image). Inter-chain and intra-chain disulfide bonds are shown as sticks. The glycosylation sites on the MH2 dimer are shown as spheres.

FIG. 6 shows the use of an MH2 or EH2 homodimer to replace CH1/κ(λ) in the IgG molecule. In addition to the desired molecule A, alternate structures result from MH2 or EH2 homo-dimerization, shown in box B.

FIG. 7 shows DE Loop engineering on MH2 to improve VH/MH2 and/or VL/MH2 interface. DE loops in IgG CH1, Cκ, and MH2 are underlined. The DE loop of IgG CH1 is grafted onto MH2 when MH2 is used to replace the IgG CH1 to create MH2mH. The DE loop of IgG Cκ is grafted onto MH2 when MH2 is used to replace the IgG Cκ to create MH2 mL. Figure discloses SEQ ID NOS 337-341, respectively, in order of appearance.

FIG. 8 shows use of MH2 (MH2a/MH2b) or EH2 (EH2a/EH2b) heterodimers to replace one arm CH1/κ(λ) in knobs-into-holes format to overcome light chain and heavy chain mispairing simultaneously in bispecific IgG generation.

FIG. 9 shows sequence alignments for TCR Cα, Cβ, IgG CH1, Cκ, Cλ, MH2, and EH2 (SEQ ID NOS 342-348, respectively, in order of appearance). The beta sheets are annotated as A, B, C, D, E, F, and G. The loops between each beta sheet are annotated as AB, BC, CD, DE, EF, and FG.

FIG. 10 shows the building of an Ig-like molecule with Vα/Vβ to obtain binding to peptide presented by antigen presenting cells. MH2a/MH2b or EH2a/EH2b stands for engineered MH2 or EH2 hetero-dimer.

FIG. 11 shows MH2 or EH2 hetero-dimer use to stabilize outer or inner variable domains in DVD-Ig molecules. In format A, B, G, and H, both the heavy and light chains are connected. In format B, C, I, and J, only the heavy chain is connected. In format D, E, K, and L, only the light chain is connected. The linker in the heavy and/or light chain may be cleavable. MH2a/MH2b may be replaced by EH2a/EH2b. Both MH2a/MH2b and EH2a/EH2b are engineered hetero-dimers. VD1 pairs with VD2 to form an antigen binding domain. VD3 pairs with VD4 to form another antigen binding domain.

FIG. 12 shows exemplary MH2 or EH2 homo- and/or hetero-dimers used as dimerization building blocks to build bi/multi-specific IgG like molecules with antibody variable domains (VH/VL) and/or TCR (Vα/Vβ). MH2WT is an MH2 homodimer. MH2pn and MH2hk are MH2 heterodimers engineered by the approaches described in Example 2.

FIG. 13 shows exemplary MH2 or EH2 used as dimerization building blocks to build bi/multi-specific fragment molecules with antibody variable domains VH/VL and/or TCR. Vα/Vβ MH2WT is MH2 homodimer. MH2pn and MH2hk are MH2 heterodimers engineered by the approaches described in Example 2.

FIG. 14 shows the binding of three exemplary bivalent monospecific MH2n/p molecules (AB596-MH2n/p, D2E7-MH2n/p, and Herceptin-MH2n/p) to three different cell lines expressing their target antigens. The first column shows the binding of AB596-MH2n/p molecule to Jurkat cells. The middle column shows the binding of D2E7-MH2n/p molecule to L929 cells. The last column shows the binding of Herceptin-MH2n/p molecule to N87 cells. The ability of each molecule to bind to its target was confirmed by a FACS binding assay.

FIG. 15 shows the molecular profiles of exemplary bispecific MH2 molecules analyzed by SEC assay. The SEC profiles of molecules KIH2, MMH3, BMH6, BMH7, BMH8, BMH9 and BMH10 are shown from top to bottom, respectively.

FIG. 16 shows the binding of bispecific BMH molecules (BMH6-10) to A431 cells expressing hEGFR, as confirmed by a FACS binding assay. A knobs-into-hole bispecific molecule KIH2 and a monovalent Cetuximab in Half-DVD-Cetux-CD3 were also tested for comparison.

FIG. 17 shows the binding of bispecific BMH molecules (BMH6-10) to N87 cells expressing hHER2, as confirmed by a FACS binding assay. A knobs-into-hole bispecific molecule KIH2 and a monovalent herceptin in half Herceptin molecule were tested for comparison.

FIG. 18 shows the binding of binding protein PLY11 (olive), TS2/18 halfbody (magenta), DVD860 halfbody (yellow), and binding protein TMH1 (green) to three different cell lines (CD3 negative Jurkat cells, regular Jurkat cells, and A431 cells), as measured by a FACS binding assay.

FIG. 19A shows a schematic structure of exemplary trispecific molecules, TMH16-18, used in a FACS binding assay. FIG. 19B shows the binding of those three trispecific molecules to three different cell lines expressing their target antigens (A431 cells, JKT CD3 positive cells, and CHO-PDL1 cells), as measured by a FACS binding assay. A KIH6 knobs-into-holes binding protein, an anti-PDL1 YW243 halfbody, and an anti-EGFR/CD3 DVD860.2 halfbody were also tested in this binding assay. A control DVD-Ig binding protein (DVD889 [hu IgG1/k]) was used as a negative control.

FIG. 20A depicts a schematic structure of the trispecific molecules, TMH21-23 used in a FACS binding assay. FIG. 20B shows the binding of those trispecific molecules to three different cell lines expressing their target antigens (A431 cells, JKT CD3 positive cells, and 293G-PD1 cells). A KIH7 knobs-into-holes binding protein, an anti-PD1 AB426 halfbody, and an anti-EGFR/CD3 DVD860.2 halfbody were also tested for comparison. The binding was measured by a FACS binding assay. DVD889 [hu IgG1/k] was used as a negative control.

FIG. 21A-C show the binding of the teteraspecific molecules PLY13-20 to three cell lines expressing their target antigens (A431 cells, Jurkat CD3 positive cells, and Jurkat CD3 negative cells, respectively), as measured by a FACS binding assay. The anti-CD2 TS2/18 halfbody, anti-4-1-BB AB430 halfbody, and anti-EGFR/CD3 DVD860 halfbody were also tested for comparison. FIG. 21D shows a schematic structure of the tetraspecific molecules used in this study.

FIG. 22A shows schematic structures of a DuoFab Ig MH2n/p molecule and each heavy and light chain used in the complete structure. FIG. 22B shows the binding of DuoFab Ig MH2n/p molecules, NBD001-003, to three cell lines expressing their target antigens (293/PSMA cells, 293/STEAP1 cells, and LnCap cells), as measured by a FACS binding assay. The parental anti-STEAP1 antibody TPP3956 and anti-PSMA hPSMA17.1 were also tested for comparison. DVD889 [hu IgG1/k] was used as a negative control.

### DETAILED DESCRIPTION

In order to reduce or eliminate the unwanted mispairing of heavy and light chains in multispecific binding proteins, a strategy to overcome both heavy chain and light chain mispairing is needed. Heavy and light chains pair through a dimerization symmetry of the CH1/Cκ or CH1/Cλ (referred to collectively herein as CH1/Cκ(λ)). On a binding protein such as an IgG antibody, this heavy and light chain pairing occurs independently on both arms of the construct. While a common light chain could be used to eliminate possible mispairing, many bispecific constructs require the use of different light chains for the two antigen binding sites.

Disclosed herein are binding proteins that overcome the light chain mispairing problem in multispecific constructs. In various embodiments, IgM CH2 (“MH2”) or IgE CH2 (“EH2”) domains are used to address the problem. For instance, in a bispecific antibody or other heterologous two-arm construct (such as a DVD-Ig binding protein having different binding domains on the first arm and second arm), an MH2 or EH2 domain or variant thereof can be used on one arm in place of a CH1/κ(λ) (e.g., to replace a CH1/κ(λ) in an IgG constant region), and normal CH1/κ(λ) can be used on the other arm to ensure correct heavy-light chain pairing on both arms while preserving the structural and functional integrity of the variable domains. In various embodiments, other modifications can also be used to ensure correct heterodimeric heavy chain pairing, such as any of the modifications mentioned in Table 1 below (e.g., knobs-into-holes or duobody techniques).

In various embodiments, mutations to wild type MH2 or EH2 that support hetero-dimerization (MH2a paired with MH2b, or EH2a paired with EH2b) can be identified by molecular modeling-based rational design, or by library-based molecular evolution including, but not limited to, phage display, yeast display, bacterial display, DNA display, mRNA display, and ribosomal display technologies. The mutations identified on MH2a or EH2a and their complementary MH2b or EH2b, respectively, that enable MH2a/MH2b or EH2a/EH2b hetero-dimerization can be based on complementary hydrophobic interaction, or electrostatic interaction, or a combination of the two, via changes introduced between MH2a and MH2b or between EH2a and EH2b. In some embodiments, changes are introduced into interface regions of MH2a and MH2b, or of EH2a and EH2b (e.g., those amino acid positions on MH2a that are within 5 angstroms of an amino acid on the counterpart MH2b, or those amino acid positions on EH2a that are within 5 angstroms of an amino acid on the counterpart EH2b). In some embodiments, the modifications alter electrostatic or hydrophobic interactions (e.g., “knobs-into-holes”) at the interface.

In various embodiments, the engineered MH2a/MH2b or EH2a/EH2b can replace CH1/Cκ(λ) dimer (e.g., in an IgG such as an IgG1 constant region) and function properly (e.g., in an IgG format by supporting the formation of functional variable domains capable of binding their antigen targets). In some embodiments, the engineered MH2a/MH2b or EH2a/EH2b replaces CH1/κ(λ) on one arm of a binding protein, while a wild-type CH1/κ(λ) remains on the other arm. In various embodiments, the “wild-type” sequences are those of human wild-type sequences.

In some embodiments, further modifications to the CH3 regions (e.g., modifications to the CH3 regions in an IgG binding protein) ensure proper pairing of the arm containing the engineered MH2a/MH2b or EH2a/EH2b with the arm that contains a wild-type CH1/Cκ(λ). For instance, modifications in the CH3 domains to alter electrostatic or hydrophobic interactions at the interface can be introduced (e.g., “knobs-into-holes” such as those described in U.S. Pat. No. 8,216,805).

In various embodiments, one or more N-glycosylation site on MH2 or EH2 could be added or removed to match the glycosylation pattern of a wild-type construct, such as an IgG, or to alter other desired properties such as pharmacokinetic properties or manufacturability. In some embodiments, the MH2 or EH2 domains can be modified to remove their DE Loop domains and replace them with IgG CH1 loop domains to mimic the interactions between CH1 and VH or the interactions between Cκ(λ) and Vκ (λ), respectively. In some embodiments, one or more cysteine residues can be removed from an MH2 or EH2 to mimic the disulfide bond interaction between an IgG CH1 domain and the hinge region.

In some embodiments, one or more of the antigen binding domains in an MH2 or EH2-modified binding protein comprises sequences from a T-cell receptor (TCR), such as the Vα and Vβ sequences. In some embodiments, the binding protein comprises a mixture of (1) antibody variable domains forming functional binding sites, and (λ) TCR binding domains.

In various embodiments, the MH2 or EH2-modified binding proteins (e.g., modified IgG binding proteins) can provide heterodimeric building blocks for constructing multi-specific binding protein formats (e.g., bi-, tri- or tetra-specific) molecules with improved functional and biophysical properties, and/or improved manufacture efficiency. In some embodiments, the binding protein is a bispecific antibody. In some embodiments, the binding protein is a DVD-Ig binding protein. In some embodiments, the DVD-Ig binding protein is further modified. In some embodiments, the modified DVD-Ig binding protein is referred as a Duo-Fab Ig binding protein. In some embodiments, the MH2a/MH2b or EH2a/EH2b heterodimer can stabilize an outer or inner binding domain of a DVD-Ig binding protein. In some embodiments, the MH2a/MH2b or EH2a/EH2b is connected to one or more DVD-Ig variable domains directly or via a linker (including a cleavable linker).

### Binding Proteins

In various embodiments, the modified binding protein disclosed herein can be an antibody or antigen-binding fragment thereof. In an embodiment, the binding protein is an antibody, a murine antibody, a CDR-grafted antibody, a human antibody, a humanized antibody, a bispecific antibody, a chimeric antibody, a Fab, a Fab′, a F(ab′)₂, an scFv, an SMIP, an affibody, an avimer, a versabody, a nanobody, a fynomab, a domain antibody, or an antigen binding fragment of any of the foregoing. In an embodiment, the binding protein is capable of binding one or more of its antigen targets with high affinity and/or potency. In an embodiment, the binding protein is a neutralizing binding protein.

In various embodiments, the binding protein is a multispecific binding protein. In an embodiment, the binding protein is a bispecific antibody. In certain embodiments, the bispecific antibody is produced by quadroma technology (Milstein and Cuello (1983) Nature 305(5934): 537-40), by chemical conjugation of two different monoclonal antibodies (Staerz et al. (1985) Nature 314(6012): 628-31), or by knob-into-hole or similar approaches which introduces mutations in the Fc region (e.g., U.S. Pat. No. 8,216,805 and Holliger et al. (1993) Proc. Natl. Acad. Sci. USA 90(14): 6444-6448).

In some embodiments, the multispecific binding protein is a dual variable domain immunoglobulin (DVD-Ig), e.g., as disclosed in U.S. Pat. No. 7,612,181 (incorporated herein by reference in their entirety). In some embodiments, the DVD-Ig binding protein comprises first and second polypeptide chains, each independently comprising VD1-(X1)n-VD2-C-X2, wherein: VD1 is a first variable domain; VD2 is a second variable domain; C is a constant domain; X1 is a linker; X2 is an Fc region that is either present or absent; n is independently 0 or 1 on the first and second chains, and wherein the VD1 domains on the first and second polypeptide chains form a first functional target binding site and the VD2 domains on the first and second polypeptide chains form a second functional target binding site. In some embodiments, the binding protein is a tri-variable domain binding protein, similar to a DVD-Ig with an additional antigen binding site attached to the N-terminus of the DVD-Ig either directly or via a linker, such that three antigen binding domains are present in parallel in the construct. In some embodiments, the MH2 or EH2 hetero-dimer is used to stabilize outer or inner variable domains in a DVD-Ig binding protein. In some embodiments, the MH2 or EH2 hetero-dimer is placed between the first and second functional target binding site. In some embodiments, the MH2 or EH2 hetero-dimer is placed at the C-terminus of the second functional target binding site.

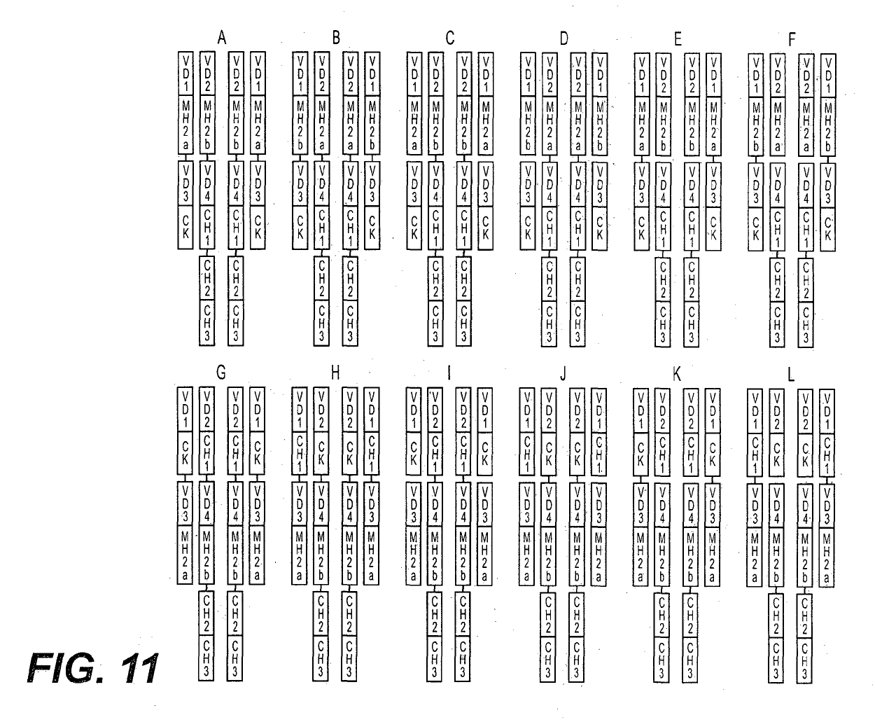

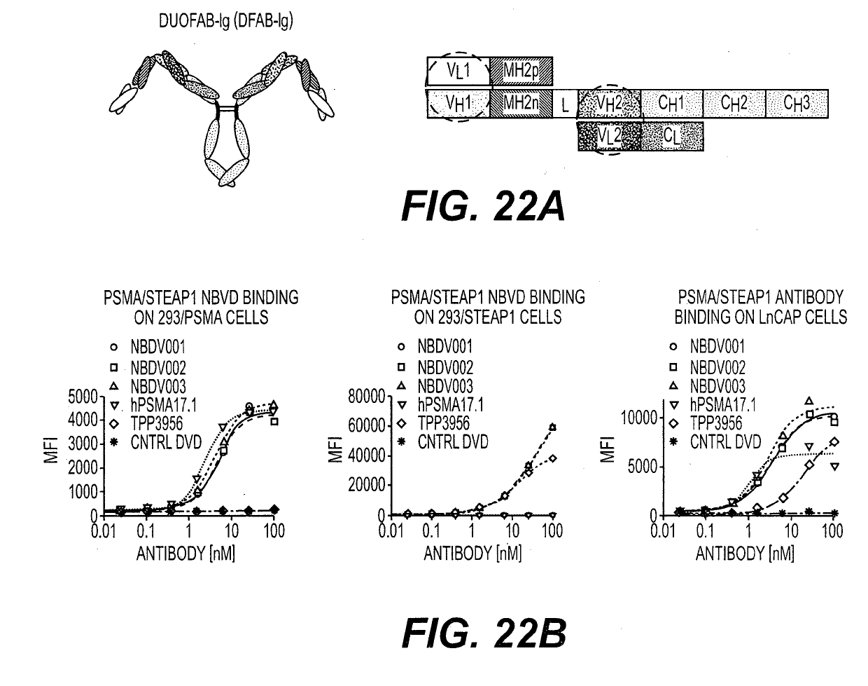

In some embodiments, the DVD-Ig binding protein is further modified. In some embodiments, the modified DVD-Ig binding protein is referred as a Duo-Fab Ig binding protein. For instance, the modified DVD-Ig binding protein may comprise first, second, and third polypeptide chains, wherein the first polypeptide chain comprises two variable domains while the second and third polypeptide chains independently comprise one variable domain. The two variable domains of the first polypeptide chain form two functional target binding sites by independently interacting with each variable domain in the second and third polypeptide chains. In some embodiments, the MH2 or EH2 hetero-dimer is used to stabilize outer or inner variable domains in the modified DVD-Ig binding protein. In some embodiments, the MH2 or EH2 hetero-dimer is placed between the first and second functional target binding site. In some embodiments, the MH2 or EH2 hetero-dimer is placed at the C-terminus of the second functional target binding site. In some embodiments, the modified DVD-Ig binding protein comprises two first polypeptide chains, two second polypeptide chains, and two third polypeptide chains, forming four functional target binding sites. Various exemplary structures of the modified DVD-Ig binding protein are depicted in FIG. 11. An exemplary structure of a Duo-Fab Ig binding protein and exemplary first, second, and third polypeptide chains used to construct the complete Duo-Fab Ig binding protein are shown in FIG. 22A.

In some embodiments, a binding protein described herein comprises multiple antigen binding sites on each arm of the construct (e.g., a DVD-Ig comprising a first binding site linked to a second binding site directly or through intervening linkers). For instance, the binding protein can be a DVD-Ig binding protein and comprise an X1 linker on each of the first and second polypeptide chain and an X2 Fc region on one of the two chains. The X1 linkers on the first and second polypeptide chains, if present, can have the same or different sequences. In one embodiment, the X1 on the first and second polypeptide chains are short (“S”) (e.g., 6 amino acid or shorter) linkers. In another embodiment, the X1 on the first and second polypeptide chains are long (“L”) (e.g., greater than 6 amino acid) linkers. In another embodiment, the X1 on the first chain is a short linker and the X1 on the second chain is a long linker. In another embodiment, the X1 on the first chain is a long linker and the X1 on the second chain is a short linker.

In some embodiments, at least one linker between variable domains in a binding protein comprises AKTTPKLEEGEFSEAR (SEQ ID NO: 1); AKTTPKLEEGEFSEARV (SEQ ID NO: 2); AKTTPKLGG (SEQ ID NO: 3); SAKTTPKLGG (SEQ ID NO: 4); SAKTTP (SEQ ID NO: 5); RADAAP (SEQ ID NO: 6); RADAAPTVS (SEQ ID NO: 7); RADAAAAGGPGS (SEQ ID NO: 8); RADAAAA(G₄S)₄ (SEQ ID NO: 9), SAKTTPKLEEGEFSEARV (SEQ ID NO: 10); ADAAP (SEQ ID NO: 11); ADAAPTVSIFPP (SEQ ID NO: 12); TVAAP (SEQ ID NO: 13); TVAAPSVFIFPP (SEQ ID NO: 14); QPKAAP (SEQ ID NO: 15); QPKAAPSVTLFPP (SEQ ID NO: 16); AKTTPP (SEQ ID NO: 17); AKTTPPSVTPLAP (SEQ ID NO: 18); AKTTAP (SEQ ID NO: 19); AKTTAPSVYPLAP (SEQ ID NO: 20); ASTKGP (SEQ ID NO: 21); ASTKGPSVFPLAP (SEQ ID NO: 22), GGGGSGGGGSGGGGS (SEQ ID NO: 23); GENKVEYAPALMALS (SEQ ID NO: 24); GPAKELTPLKEAKVS (SEQ ID NO: 25); or GHEAAAVMQVQYPAS (SEQ ID NO: 26); TVAAPSVFIFPPTVAAPSVFIFPP (SEQ ID NO: 27); ASTKGPSVFPLAPASTKGPSVFPLAP (SEQ ID NO: 28); GGGGSGGGGS (SEQ ID NO: 29); GGSGGGGSG (SEQ ID NO: 30); or G/S based sequences (e.g., G4S and G4S repeats; SEQ ID NO: 31).

In an embodiment, the linker is a cleavable linker. In an embodiment, the linker is cleavable by one or more enzyme or agent selected from the group consisting of a zinc-dependent endopeptidase, Matrix Metalloproteinase (MMP), a serralysin, an astacin, an adamalysin, MMP-1; MMP-2; MMP-3; MMP-7; MMP-8; MMP-9; MMP-10; MMP-11; MMP-12; MMP-13; MMP-14; MMP-15; MMP-16; MMP-17; MMP-18; MMP-19; MMP-20; MMP-21; MMP-22; MMP-23A; MMP-23B; MMP-24; MMP-25; MMP-26; MMP-27; MMP-28; a Disintegrin and Metalloproteinase (ADAM); ADAM17; ADAMTS1; ADAM1; ADAM10; ADAM8; ADAMTS4; ADAMTS13; ADAM12; ADAM15; ADAM9; ADAMTS5; ADAM33; ADAM11; ADAM2; ADAMTS2; ADAMTS9; ADAMTS3; ADAMTS7; ADAM22; ADAM28; ADAMTS12; ADAM19; ADAMTS8; ADAM29; ADAM23; ADAM3A; ADAM18; ADAMTS6; ADAM7; ADAMDES1; ADAM20; ADAM6; ADAM21; ADAM3B; ADAMTSL3; ADAMTSL4; ADAM30; ADAMTS20; ADAMTSL2; a Caspase; Caspases 1-12, Caspase 14; a Cathepsin; Cathepsin G; Cathepsin B; Cathepsin D; Cathepsin L1; Cathepsin C; Cathepsin K; Cathepsin S; Cathepsin H; Cathepsin A; Cathepsin E; Cathepsin L; Cathepsin Z; Cathepsin F; Cathepsin G-like 2; Cathepsin L-like 1; Cathepsin W; Cathepsin L-like 2; Cathepsin L-like 3; Cathepsin L-like 4; Cathepsin L-like 5; Cathepsin L-like 6; Cathepsin L-like 7; Cathepsin O; a Calpain; Calpain 3; Calpain 10; Calpain 1 (mu/l) large subunit; Calpain, small subunit 1; Calpain 2, (mu/l); large subunit; Calpain 9; Calpain 11; Calpain 5; Calpain 6; Calpain 13; Calpain 8; Calpain, small subunit 2; Calpain 15; Calpain 12; Calpain 7; and Calpain 8.

In an embodiment, a binding protein disclosed herein has an on rate constant (Kₒₙ) to one or more targets of at least about 10²M⁻¹s⁻¹; at least about 10³M⁻¹s⁻¹; at least about 10⁴M⁻¹s⁻¹; at least about 10⁵M⁻¹s⁻¹; or at least about 10⁶M⁻¹s⁻¹, as measured by surface plasmon resonance. In an embodiment, the binding protein has an on rate constant (Kₒₙ) to one or more targets from about 10²M⁻¹s⁻¹ to about 10³M⁻¹s⁻¹; from about 10³M⁻¹s⁻¹ to about 10⁴M⁻¹s⁻¹; from about 10⁴M⁻¹s⁻¹ to about 10⁵M⁻¹s⁻¹; or from about 10⁵M⁻¹s⁻¹ to about 10⁶M⁻¹s⁻¹, as measured by surface plasmon resonance.

In another embodiment, the binding protein has an off rate constant (K_{off}) for one or more targets of at most about 10⁻³s⁻¹; at most about 10⁻⁴s⁻¹; at most about 10⁻⁵s⁻¹; or at most about 10⁻⁶s⁻¹, as measured by surface plasmon resonance. In an embodiment, the binding protein has an off rate constant (K_{off}) to one or more targets of about 10⁻³s⁻¹ to about 10⁻⁴s⁻¹; of about 10⁻⁴s⁻¹ to about 10⁻⁵s⁻¹; or of about 10⁻⁵s⁻¹ to about 10⁻⁶s⁻¹, as measured by surface plasmon resonance.

In another embodiment, the binding protein has a dissociation constant (K_{d}) to one or more targets of at most about 10⁻⁷M; at most about 10⁻⁸M; at most about 10⁻⁹M; at most about 10⁻¹⁰M; at most about 10⁻¹¹M; at most about 10⁻¹²M; or at most 10⁻¹³M. In an embodiment, the binding protein has a dissociation constant (K_{d}) to its targets of about 10⁻⁷M to about 10⁻⁸M; of about 10⁻⁸M to about 10⁻⁹M; of about 10⁻⁹M to about 10⁻¹⁰M; of about 10⁻¹⁰M to about 10⁻¹¹M; of about 10⁻¹¹M to about 10⁻¹²M; or of about 10⁻¹² to M about 10⁻¹³M.

In an embodiment, a binding protein disclosed herein is conjugated to an agent. In an embodiment, the agent is an immunoadhesion molecule, an imaging agent, a therapeutic agent, or a cytotoxic agent. In an embodiment, the imaging agent is a radiolabel, an enzyme, a fluorescent label, a luminescent label, a bioluminescent label, a magnetic label, or biotin. In another embodiment, the radiolabel is ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm. In yet another embodiment, the therapeutic or cytotoxic agent is an anti-metabolite, an alkylating agent, an antibiotic, a growth factor, a cytokine, an anti-angiogenic agent, an anti-mitotic agent, an anthracycline, toxin, or an apoptotic agent, or an immunosuppressive agent.

In an embodiment, the binding protein is a crystallized binding protein and exists as a crystal. In an embodiment, the crystal is a carrier-free pharmaceutical controlled release crystal. In another embodiment, the crystallized binding protein has a greater half-life in vivo than the soluble counterpart of the binding protein. In yet another embodiment, the crystallized binding protein retains biological activity.

In an embodiment, a composition is provided for the release of a binding protein, wherein the composition comprises a crystallized binding protein, an ingredient, and at least one polymeric carrier. In an embodiment, the polymeric carrier is poly (acrylic acid), a poly (cyanoacrylate), a poly (amino acid), a poly (anhydride), a poly (depsipeptide), a poly (ester), poly (lactic acid), poly (lactic-co-glycolic acid) or PLGA, poly (b-hydroxybutryate), poly (caprolactone), poly (dioxanone), poly (ethylene glycol), poly ((hydroxypropyl) methacrylamide, poly [(organo)phosphazene], a poly (ortho ester), poly (vinyl alcohol), poly (vinylpyrrolidone), a maleic anhydride-alkyl vinyl ether copolymer, a pluronic polyol, albumin, alginate, cellulose, a cellulose derivative, collagen, fibrin, gelatin, hyaluronic acid, an oligosaccharide, a glycaminoglycan, a sulfated polysaccharide, or blends and copolymers thereof. In an embodiment, the ingredient is albumin, sucrose, trehalose, lactitol, gelatin, hydroxypropyl-β-cyclodextrin, methoxypolyethylene glycol, or polyethylene glycol.

In an embodiment, the binding protein described herein is glycosylated. For example, the glycosylation pattern is a human glycosylation pattern.

Also disclosed herein is a pharmaceutical composition comprising a binding protein and a pharmaceutically acceptable carrier. In an embodiment, the pharmaceutical composition also comprises at least one additional therapeutic agent for treating a disorder. For example, the additional agent may be a therapeutic agent, an imaging agent, a cytotoxic agent, an angiogenesis inhibitor (including but not limited to an anti-VEGF antibody or a VEGF-trap), a kinase inhibitor (including but not limited to a KDR and a TIE-2 inhibitor), a co-stimulation molecule blocker (including but not limited to anti-B7.1, anti-B7.2, CTLA4-Ig, anti-CD20), an adhesion molecule blocker (including but not limited to an anti-LFA-1 antibody, an anti-E/L selectin antibody, a small molecule inhibitor), an anti-cytokine antibody or functional fragment thereof (including but not limited to an anti-IL-18, an anti-TNF, and an anti-IL-6/cytokine receptor antibody), methotrexate, cyclosporin, rapamycin, FK506, a detectable label or reporter, a TNF antagonist, an antirheumatic, a muscle relaxant, a narcotic, a non-steroid anti-inflammatory drug (NSAID), an analgesic, an anesthetic, a sedative, a local anesthetic, a neuromuscular blocker, an antimicrobial, an antipsoriatic, a corticosteriod, an anabolic steroid, an erythropoietin, an immunization, an immunoglobulin, an immunosuppressive, a growth hormone, a hormone replacement drug, a radiopharmaceutical, an antidepressant, an antipsychotic, a stimulant, an asthma medication, a beta agonist, an inhaled steroid, an epinephrine or analog, a cytokine, or a cytokine antagonist.

### Preparation of Binding Proteins

In another aspect, the disclosure provides a method of making the binding proteins disclosed herein. In an embodiment, the method of making a binding protein comprises the steps of a) obtaining a binding protein comprising an IgG constant region and determining the nucleic acid sequence encoding the heavy and light chains of the binding protein; b) inserting a sequence encoding an MH2 or EH2, or a modified MH2 or EH2 sequence, in place of an IgG CH1 and C kappa or C lambda domain in the nucleic acids encoding the heavy and light chains; c) preparing construct(s) containing the modified nucleic acid sequences and the original nucleic acid sequences, and inserting them in a host cell; and d) expressing the nucleic acids such that a binding protein is generated.

One or more isolated nucleic acids encoding any one of the binding proteins disclosed herein is also provided. A further embodiment provides a vector or vectors comprising the isolated nucleic acid disclosed herein. In an embodiment, the vector(s) is/are one or more of pcDNA; pTT; pTT3 (pTT with additional multiple cloning site); pEFBOS; pBV; pJV; pcDNA3.1 TOPO; pEF6 TOPO; pBOS; pHybE; and pBJ. In an embodiment, the vector is a vector disclosed in U.S. Pat. No. 7,612,181.

In an embodiment, a host cell is disclosed, wherein the host cell is transformed with a vector or vectors disclosed herein. In an embodiment, the host cell is a prokaryotic cell, for example, E. coli. In another embodiment, the host cell is a eukaryotic cell, for example, a protist cell, an animal cell, a plant cell, or a fungal cell. In an embodiment, the host cell is a mammalian cell including, but not limited to, CHO, COS, NS0, SP2, PER.C6, or a fungal cell, such as Saccharomyces cerevisiae, or an insect cell, such as Sf9. In an embodiment, two or more binding proteins, e.g., with different specificities, are produced in a single recombinant host cell. For example, the expression of a mixture of antibodies has been called Oligoclonics™ (Merus B. V., The Netherlands) U.S. Pat. Nos. 7,262,028 and 7,429,486.

A method of producing a binding protein is disclosed herein, comprising culturing any one of the host cells disclosed herein in a culture medium under conditions sufficient to produce the binding protein. In an embodiment, 50%-100% of the binding protein produced by this method exhibits the correct multispecific pairing of a binding protein disclosed herein (e.g., 50-100%, 50-90%, 75%-90%, 75-100%, 80-100%, 90-100%, 91-100%, 92-100%, 93-100%, 94-100%, 95-100%, 96-100%, 97-100%, 98-100%, 99-100%, or any percentage in between.

### Use of Disclosed Binding Proteins in Treating Various Diseases

In various embodiments, the binding proteins provided herein may be used as therapeutic molecules to treat various diseases, e.g., wherein the targets that are recognized by the binding proteins are detrimental. Such binding proteins may bind one or more targets involved in a specific disease. In an embodiment, the method comprises administering a binding protein disclosed herein to a subject in need thereof.

In an embodiment, a method for treating a mammal is provided, comprising the step of administering to the mammal an effective amount of a composition disclosed herein (e.g., a binding protein or a pharmaceutical composition comprising the binding protein.

The binding proteins provided herein can be used to treat humans suffering from autoimmune diseases such as, for example, those associated with inflammation. In an embodiment, the binding proteins provided herein are used to treat asthma, allergies, allergic lung disease, allergic rhinitis, atopic dermatitis, inflammatory pustular skin disease, Behcet's disease, Systemic Juvenile Idiopathic Arthritis, Familial Mediterranean Fever, Neonatal Onset Multisystem Inflammatory disease, acute heart failure, post-infarction remodeling, pulmonary hypertension, type 1 diabetes, proliferative Diabetic Retinopathy, Congenital Hyperinsulinism, Schnitzler Syndrome, gout flares, pyoderma gangrenosum, chronic obstructive pulmonary disease (COPD), fibrosis, cystic fibrosis (CF), fibrotic lung disease, idiopathic pulmonary fibrosis, liver fibrosis, lupus, hepatitis B-related liver diseases and fibrosis, sepsis, systemic lupus erythematosus (SLE), glomerulonephritis, inflammatory skin diseases, psoriasis, diabetes, insulin dependent diabetes mellitus, infectious diseases caused by HIV, inflammatory bowel disease (IBD), ulcerative colitis (UC), Crohn's disease (CD), rheumatoid arthritis (RA), osteoarthritis (OA), multiple sclerosis (MS), graft-versus-host disease (GVHD), transplant rejection, ischemic heart disease (IHD), celiac disease, contact hypersensitivity, alcoholic liver disease, Behcet's disease, atherosclerotic vascular disease, ocular surface inflammatory diseases, or Lyme disease.

In another embodiment, the disorder or condition to be treated comprises a viral infection and/or the symptoms caused by viral infection in a human, for example, HIV, the human rhinovirus, an enterovirus, a coronavirus, a herpes virus, an influenza virus, a parainfluenza virus, a respiratory syncytial virus or an adenovirus.

The binding proteins provided herein can be used to treat neurological disorders. In an embodiment, the binding proteins provided herein are used to treat neurodegenerative diseases and conditions involving neuronal regeneration and spinal cord injury.

In an embodiment, diseases that can be treated or diagnosed with the compositions and methods disclosed herein include, but are not limited to, primary and metastatic cancers, including carcinomas of breast, colon, rectum, lung, oropharynx, hypopharynx, esophagus, stomach, pancreas, liver, gallbladder and bile ducts, small intestine, urinary tract (including kidney, bladder and urothelium), female genital tract (including cervix, uterus, and ovaries as well as choriocarcinoma and gestational trophoblastic disease), male genital tract (including prostate, seminal vesicles, testes and germ cell tumors), endocrine glands (including the thyroid, adrenal, and pituitary glands), and skin, as well as hemangiomas, melanomas, sarcomas (including those arising from bone and soft tissues as well as Kaposi's sarcoma), tumors of the brain, nerves, eyes, and meninges (including astrocytomas, gliomas, glioblastomas, retinoblastomas, neuromas, neuroblastomas, Schwannomas, and meningiomas), solid tumors arising from hematopoietic malignancies such as leukemias, and lymphomas (both Hodgkin's and non-Hodgkin's lymphomas).

Another embodiment provides for the use of the binding protein in the diagnosis or treatment of a disease or disorder, wherein the disease or disorder is rheumatoid arthritis, osteoarthritis, juvenile chronic arthritis, septic arthritis, Lyme arthritis, psoriatic arthritis, reactive arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, graft versus host disease, organ transplant rejection, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, Addison's disease, sporadic, polyglandular deficiency type I and polyglandular deficiency type II, Schmidt's syndrome, adult (acute) respiratory distress syndrome, alopecia, alopecia areata, arthropathy, Reiter's disease, psoriatic arthropathy, ulcerative colitic arthropathy, enteropathic synovitis, chlamydia, Yersinia and salmonella associated arthropathy, athermanous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, acquired immunodeficiency related diseases, hepatitis B, hepatitis C, common varied immunodeficiency (common variable hypogammaglobulinaemia), dilated cardiomyopathy, female infertility, ovarian failure, premature ovarian failure, fibrotic lung disease, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjögren's disease associated lung disease, ankylosing spondylitis associated lung disease, vasculitic diffuse lung disease, haemosiderosis associated lung disease, drug-induced interstitial lung disease, fibrosis, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopaenia, autoimmune neutropaenia, renal disease NOS, glomerulonephritides, microscopic vasculitis of the kidneys, lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Sjögren's syndrome, Takayasu's disease/arteritis, autoimmune thrombocytopaenia, idiopathic thrombocytopaenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo acute liver disease, chronic liver diseases, alcoholic cirrhosis, alcohol-induced liver injury, cholestatis, idiosyncratic liver disease, drug-induced hepatitis, non-alcoholic steatohepatitis, allergy and asthma, group B streptococci (GBS) infection, mental disorders, depression, schizophrenia, Th2 Type and Th1 Type mediated diseases, acute and chronic pain, different forms of pain, cancers, lung cancer, breast cancer, stomach cancer, bladder cancer, colon cancer, pancreatic cancer, ovarian cancer, prostate cancer, rectal cancer, hematopoietic malignancies, leukemia, lymphoma, Abetalipoprotemia, acrocyanosis, acute and chronic parasitic or infectious processes, acute leukemia, acute lymphoblastic leukemia (ALL), acute myeloid leukemia (AML), acute or chronic bacterial infection, acute pancreatitis, acute renal failure, adenocarcinomas, aerial ectopic beats, AIDS dementia complex, alcohol-induced hepatitis, allergic conjunctivitis, allergic contact dermatitis, allergic rhinitis, allograft rejection, alpha-1-antitrypsin deficiency, amyotrophic lateral sclerosis, anemia, angina pectoris, anterior horn cell degeneration, anti cd3 therapy, antiphospholipid syndrome, anti-receptor hypersensitivity reactions, aortic and peripheral aneuryisms, aortic dissection, arterial hypertension, arteriosclerosis, arteriovenous fistula, ataxia, atrial fibrillation (sustained or paroxysmal), atrial flutter, atrioventricular block, B cell lymphoma, bone graft rejection, bone marrow transplant (BMT) rejection, bundle branch block, Burkitt's lymphoma, burns, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy associated disorders, chronic myelocytic leukemia (CML), chronic alcoholism, chronic inflammatory pathologies, chronic lymphocytic leukemia (CLL), chronic obstructive pulmonary disease (COPD), chronic salicylate intoxication, colorectal carcinoma, congestive heart failure, conjunctivitis, contact dermatitis, cor pulmonale, coronary artery disease, Creutzfeldt-Jakob disease, culture negative sepsis, cystic fibrosis, cytokine therapy associated disorders, dementia pugilistica, demyelinating diseases, dengue hemorrhagic fever, dermatitis, dermatologic conditions, diabetes, diabetes mellitus, diabetic ateriosclerotic disease, diffuse Lewy body disease, dilated congestive cardiomyopathy, disorders of the basal ganglia, Down's syndrome in middle age, drug-induced movement disorders induced by drugs which block CNS dopamine receptors, drug sensitivity, eczema, encephalomyelitis, endocarditis, endocrinopathy, epiglottitis, Epstein-Barr virus infection, erythromelalgia, extrapyramidal and cerebellar disorders, familial hematophagocytic lymphohistiocytosis, fetal thymus implant rejection, Friedreich's ataxia, functional peripheral arterial disorders, fungal sepsis, gas gangrene, gastric ulcer, glomerular nephritis, graft rejection of any organ or tissue, gram negative sepsis, gram positive sepsis, granulomas due to intracellular organisms, hairy cell leukemia, Hallervorden-Spatz disease, Hashimoto's thyroiditis, hay fever, heart transplant rejection, hemachromatosis, hemodialysis, hemolytic uremic syndrome/thrombolytic thrombocytopenic purpura, hemorrhage, hepatitis A, His bundle arrythmias, HIV infection/HIV neuropathy, Hodgkin's disease, hyperkinetic movement disorders, hypersensitivity reactions, hypersensitivity pneumonitis, hypertension, hypokinetic movement disorders, hypothalamic-pituitary-adrenal axis evaluation, idiopathic Addison's disease, idiopathic pulmonary fibrosis, antibody mediated cytotoxicity, Asthenia, infantile spinal muscular atrophy, inflammation of the aorta, influenza a, ionizing radiation exposure, iridocyclitis/uveitis/optic neuritis, ischemia-reperfusion injury, ischemic stroke, juvenile rheumatoid arthritis, juvenile spinal muscular atrophy, Kaposi's sarcoma, kidney transplant rejection, legionella, leishmaniasis, leprosy, lesions of the corticospinal system, lipedema, liver transplant rejection, lymphederma, malaria, malignant lymphoma, malignant histiocytosis, malignant melanoma, meningitis, meningococcemia, metabolic/idiopathic, migraine headache, mitochondrial multisystem disorder, mixed connective tissue disease, monoclonal gammopathy, multiple myeloma, multiple systems degenerations (Mencel Dejerine-Thomas Shi-Drager and Machado-Joseph), mycobacterium avium intracellulare, mycobacterium tuberculosis, my elodyplastic syndrome, myocardial infarction, myocardial ischemic disorders, nasopharyngeal carcinoma, neonatal chronic lung disease, nephritis, nephrosis, neurodegenerative diseases, neurogenic muscular atrophies, neutropenic fever, non-Hodgkins lymphoma, occlusion of the abdominal aorta and its branches, occlusive arterial disorders, okt3 therapy, orchitis/epidydimitis, orchitis/vasectomy reversal procedures, organomegaly, osteoporosis, pancreas transplant rejection, pancreatic carcinoma, paraneoplastic syndrome/hypercalcemia of malignancy, parathyroid transplant rejection, pelvic inflammatory disease, perennial rhinitis, pericardial disease, peripheral atherlosclerotic disease, peripheral vascular disorders, peritonitis, pernicious anemia, pneumocystis carinii pneumonia, pneumonia, POEMS syndrome (polyneuropathy, organomegaly, endocrinopathy, monoclonal gammopathy, and skin changes syndrome), post perfusion syndrome, post pump syndrome, post-MI cardiotomy syndrome, preeclampsia, progressive supranucleo palsy, primary pulmonary hypertension, radiation therapy, Raynaud's phenomenon and disease, Raynoud's disease, Refsum's disease, regular narrow QRS tachycardia, renovascular hypertension, reperfusion injury, restrictive cardiomyopathy, sarcomas, scleroderma, senile chorea, senile dementia of Lewy body type, seronegative arthropathies, shock, sickle cell anemia, skin allograft rejection, skin changes syndrome, small bowel transplant rejection, solid tumors, specific arrhythmias, spinal ataxia, spinocerebellar degenerations, streptococcal myositis, structural lesions of the cerebellum, subacute sclerosing panencephalitis, syncope, syphilis of the cardiovascular system, systemic anaphalaxis, systemic inflammatory response syndrome, systemic onset juvenile rheumatoid arthritis, T-cell or FAB ALL telangiectasia, thromboangitis obliterans, thrombocytopenia, toxicity, transplants, trauma/hemorrhage, type III hypersensitivity reactions, type IV hypersensitivity, unstable angina, uremia, urosepsis, valvular heart diseases, varicose veins, vasculitis, venous diseases, venous thrombosis, ventricular fibrillation, viral and fungal infections, vital encephalitis/aseptic meningitis, vital-associated hemaphagocytic syndrome, Wernicke-Korsakoff syndrome, Wilson's disease, xenograft rejection of any organ or tissue, acute coronary syndromes, acute idiopathic polyneuritis, acute inflammatory demyelinating polyradiculoneuropathy, acute ischemia, adult Still's disease, anaphylaxis, anti-phospholipid antibody syndrome, aplastic anemia, atopic eczema, atopic dermatitis, autoimmune dermatitis, autoimmune disorder associated with streptococcus infection, autoimmune enteropathy, autoimmune hearing loss, autoimmune lymphoproliferative syndrome (ALPS), autoimmune myocarditis, autoimmune premature ovarian failure, blepharitis, bronchiectasis, bullous pemphigoid, cardiovascular disease, catastrophic antiphospholipid syndrome, celiac disease, cervical spondylosis, chronic ischemia, cicatricial pemphigoid, clinically isolated syndrome (cis) with risk for multiple sclerosis, childhood onset psychiatric disorder, dacryocystitis, dermatomyositis, diabetic retinopathy, disk herniation, disk prolaps, drug induced immune hemolytic anemia, endometriosis, endophthalmitis, episcleritis, erythema multiforme, erythema multiforme major, gestational pemphigoid, Guillain-Barré syndrome (GBS), Hughes syndrome, idiopathic Parkinson's disease, idiopathic interstitial pneumonia, IgE-mediated allergy, immune hemolytic anemia, inclusion body myositis, infectious ocular inflammatory disease, inflammatory demyelinating disease, inflammatory heart disease, inflammatory kidney disease, IPF/UIP, iritis, keratitis, keratojuntivitis sicca, Kussmaul disease or Kussmaul-Meier disease, Landry's paralysis, Langerhan's cell histiocytosis, livedo reticularis, macular degeneration, microscopic polyangiitis, morbus bechterev, motor neuron disorders, mucous membrane pemphigoid, multiple organ failure, myasthenia gravis, myelodysplastic syndrome, myocarditis, nerve root disorders, neuropathy, non-A non-B hepatitis, optic neuritis, osteolysis, pauciarticular JRA, peripheral artery occlusive disease (PAOD), peripheral vascular disease (PVD), peripheral artery, disease (PAD), phlebitis, polyarteritis nodosa (or periarteritis nodosa), polychondritis, poliosis, polyarticular JRA, polyendocrine deficiency syndrome, polymyositis, polymyalgia rheumatica (PMR), primary Parkinsonism, prostatitis, pure red cell aplasia, primary adrenal insufficiency, recurrent neuromyelitis optica, restenosis, rheumatic heart disease, sapho (synovitis, acne, pustulosis, hyperostosis, and osteitis), secondary amyloidosis, shock lung, scleritis, sciatica, secondary adrenal insufficiency, silicone associated connective tissue disease, Sneddon-Wilkinson dermatosis, spondilitis ankylosans, Stevens-Johnson syndrome (SJS), temporal arteritis, toxoplasmic retinitis, toxic epidermal necrolysis, transverse myelitis, TRAPS (tumor necrosis factor receptor, type 1 allergic reaction, type II diabetes, urticaria, usual interstitial pneumonia (UIP), vasculitis, vernal conjunctivitis, viral retinitis, Vogt-Koyanagi-Harada syndrome (VKH syndrome), wet macular degeneration, or wound healing. In some embodiments, any one or a combination of the binding proteins disclosed herein can be used to diagnose or treat a disorder listed above.

Also disclosed herein are methods of determining the presence, amount or concentration of one or more antigen targets, or fragment thereof, in a test sample. In some embodiments, the method comprises assaying the test sample for the antigen, or fragment thereof, by an immunoassay. The immunoassay (i) employs at least one binding protein and at least one detectable label and (ii) comprises comparing a signal generated by the detectable label as a direct or indirect indication of the presence, amount or concentration of the antigen, or fragment thereof, in the test sample to a signal generated as a direct or indirect indication of the presence, amount or concentration of the antigen, or fragment thereof, in a control or a calibrator. The calibrator is optionally part of a series of calibrators in which each of the calibrators differs from the other calibrators in the series by the concentration of the antigen, or fragment thereof. The method can comprise (i) contacting the test sample with at least one capture agent, which binds to an epitope on the antigen, or fragment thereof, so as to form a capture agent/antigen, or fragment thereof, complex, (ii) contacting the capture agent/antigen, or fragment thereof, complex with at least one detection agent, which comprises a detectable label and binds to an epitope on the antigen, or fragment thereof, that is not bound by the capture agent, to form a capture agent/antigen, or fragment thereof/detection agent complex, and (iii) determining the presence, amount or concentration of the antigen, or fragment thereof, in the test sample based on the signal generated by the detectable label in the capture agent/antigen, or fragment thereof/detection agent complex formed in (ii), wherein at least one capture agent and/or at least one detection agent is the at least one binding protein.

Alternatively, the method can comprise (i) contacting the test sample with at least one capture agent, which binds to an epitope on the antigen, or fragment thereof, so as to form a capture agent/antigen, or fragment thereof, complex, and simultaneously or sequentially, in either order, contacting the test sample with detectably labeled antigen, or fragment thereof, which can compete with any antigen, or fragment thereof, in the test sample for binding to the at least one capture agent, wherein any antigen, or fragment thereof, present in the test sample and the detectably labeled antigen compete with each other to form a capture agent/antigen, or fragment thereof, complex and a capture agent/detectably labeled antigen, or fragment thereof, complex, respectively, and (ii) determining the presence, amount or concentration of the antigen, or fragment thereof, in the test sample based on the signal generated by the detectable label in the capture agent/detectably labeled antigen, or fragment thereof, complex formed in (ii), wherein at least one capture agent is the at least one binding protein and wherein the signal generated by the detectable label in the capture agent/detectably labeled antigen, or fragment thereof, complex is inversely proportional to the amount or concentration of antigen, or fragment thereof, in the test sample.

The test sample can be from a patient, in which case the method can further comprise diagnosing, prognosticating, or assessing the efficacy of therapeutic/prophylactic treatment of the patient. If the method further comprises assessing the efficacy of therapeutic/prophylactic treatment of the patient, the method optionally further comprises modifying the therapeutic/prophylactic treatment of the patient as needed to improve efficacy. The method can be adapted for use in an automated system or a semi-automated system. Accordingly, the methods described herein also can be used to determine whether or not a subject has or is at risk of developing a given disease, disorder or condition. Specifically, such a method can comprise the steps of:
(a) determining the concentration or amount in a test sample from a subject of analyte, or fragment thereof, (e.g., using the methods described herein, or methods known in the art); and
(b) comparing the concentration or amount of analyte, or fragment thereof, determined in step (a) with a predetermined level, wherein, if the concentration or amount of analyte determined in step (a) is favorable with respect to a predetermined level, then the subject is determined not to have or be at risk for a given disease, disorder or condition.
   However, if the concentration or amount of analyte determined in step (a) is unfavorable with respect to the predetermined level, then the subject is determined to have or be at risk for a given disease, disorder or condition.

Additionally, provided herein is method of monitoring the progression of disease in a subject. Optimally the method comprising the steps of: (a) determining the concentration or amount in a test sample from a subject of analyte; (b) determining the concentration or amount in a later test sample from the subject of analyte; and (c) comparing the concentration or amount of analyte as determined in step (b) with the concentration or amount of analyte determined in step (a), wherein if the concentration or amount determined in step (b) is unchanged or is unfavorable when compared to the concentration or amount of analyte determined in step (a), then the disease in the subject is determined to have continued, progressed or worsened. By comparison, if the concentration or amount of analyte as determined in step (b) is favorable when compared to the concentration or amount of analyte as determined in step (a), then the disease in the subject is determined to have discontinued, regressed or improved.

Optionally, the method further comprises comparing the concentration or amount of analyte as determined in step (b), for example, with a predetermined level. Further, optionally the method comprises treating the subject with one or more pharmaceutical compositions for a period of time if the comparison shows that the concentration or amount of analyte as determined in step (b), for example, is unfavorably altered with respect to the predetermined level.

Also provided is a kit for assaying a test sample for one or more antigen targets, or fragments thereof. The kit comprises at least one component for assaying the test sample for an antigen, or fragment thereof, and instructions for assaying the test sample for an antigen, or fragment thereof, wherein the at least one component includes at least one composition comprising the binding protein disclosed herein, wherein the binding protein is optionally detectably labeled.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” are not limiting. Any range disclosed herein is intended to encompass the endpoints of that range unless stated otherwise.

Generally, nomenclatures used in connection with cell and tissue culture, molecular biology, immunology, microbiology, genetics and protein and nucleic acid chemistry and hybridization described herein are those known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The term “antibody” refers to an immunoglobulin (Ig) molecule, which is may comprise four polypeptide chains, two heavy (H) chains and two light (L) chains, or it may comprise a functional fragment (such as a half body), mutant, variant, or derivative thereof, that retains the epitope binding features of an Ig molecule. Such fragment, mutant, variant, or derivative antibody formats are known in the art. In an embodiment of a full-length antibody, each heavy chain is comprised of a heavy chain variable region (VH) and a heavy chain constant region (CH). In the case of an IgG molecule, the CH comprises three domains, CH1, CH2 and CH3 (prior to the modifications disclosed herein). Each light chain is comprised of a light chain variable region (VL) and a light chain constant region (CL). The CL is comprised of a single CL domain. The VH and VL can be further subdivided into regions of hypervariability, termed complementarity determining regions (CDRs), interspersed with regions that are more conserved, termed framework regions (FRs). Generally, each VH and VL is composed of three CDRs and four FRs, arranged from amino-terminus to carboxy-terminus in the following order: FR1, CDR1, FR2, CDR2, FR3, CDR3, and FR4. CDR regions may be determined by standard methods, e.g., those of Kabat et al. Immunoglobulin molecules can be of any type (e.g., IgG, IgE, IgM, IgD, IgA and IgY), class (e.g., IgG1, IgG2, IgG3, IgG4, IgA1 and IgA2), or subclass. An antibody is a type of binding protein.

The term “multispecific” binding protein refer to binding proteins that have binding specificities for at least two different antigens. Traditionally, the recombinant production of multispecific antibodies is based on the co-expression of two immunoglobulin heavy chain-light chain pairs, where the two heavy chains have different specificities (Milstein et al. (1983) Nature 305: 537). Similar procedures are disclosed, e.g., in PCT Publication Nos. WO 93/08829, WO 91/00360, and WO 92/00373; U.S. Pat. Nos. 6,210,668; 6,193,967; 6,132,992; 6,106,833; 6,060,285; 6,037,453; 6,010,902; 5,989,530; 5,959,084; 5,959,083; 5,932,448; 5,833,985; 5,821,333; 5,807,706; 5,643,759, 5,601,819; 5,582,996, 5,496,549, and 4,676,980; Traunecker et al. (1991) EMBO J. 10: 3655; and Suresh et al. (1986) Methods in Enzymol. 121: 210; incorporated herein by reference.

The term “bispecific” antibody or binding protein refers to an antibody or binding protein that binds one antigen (or epitope) on one of its two binding arms (one pair of HC/LC), and binds a different antigen (or epitope) on its second binding arm (a different pair of HC/LC). A bispecific antibody is a type of bispecific binding protein. A bispecific antibody may have two distinct antigen binding arms (in both specificity and CDR sequences), and may be monovalent for each antigen to which it binds. Bispecific antibodies include those generated by quadroma technology (Milstein and Cuello (1983) Nature 305(5934): 537-40), by chemical conjugation of two different monoclonal antibodies (Staerz et al. (1985) Nature 314(6012): 628-31), or by knob-into-hole or similar approaches which introduces mutations in the Fc region (Holliger et al. (1993) Proc. Natl. Acad. Sci. USA 90(14): 6444-6448).

The term “affinity matured” refers to an antibody or binding protein with one or more alterations in one or more CDR or framework (FR) regions thereof, which may result in an improvement in the affinity for an antigen, compared to a parent antibody or binding protein which does not possess those alteration(s). Exemplary affinity matured antibodies or binding protein will have nanomolar or even picomolar affinities for the target antigen. Affinity matured antibodies or binding protein may be produced by procedures known in the art, e.g., Marks et al. (1992) BioTechnology 10: 779-783 describes affinity maturation by VH and VL domain shuffling. Random mutagenesis of CDR and/or framework residues is described by Barbas et al. (1994) Proc. Nat. Acad. Sci. USA 91:3809-3813; Schier et al. (1995) Gene 169: 147-155; Yelton et al. (1995) J. Immunol. 155: 1994-2004; Jackson et al. (1995) J. Immunol. 154(7): 3310-9; Hawkins et al. (1992) J. Mol. Biol. 226: 889-896 and mutation at selective mutagenesis positions, contact or hypermutation positions with an activity enhancing amino acid residue as described in U.S. Pat. No. 6,914,128.

The term “CDR-grafted” refers to an antibody or binding protein that comprises heavy and light chain variable region sequences in which the sequences of one or more of the CDR regions of the VH and/or VL domains are replaced with CDR sequences of another antibody or binding protein. For example, the two antibodies or binding protein can be from different species, such as antibodies or binding protein having murine heavy and light chain variable regions in which one or more of the murine CDRs has been replaced with human CDR sequences.

The term “humanized” refers to an antibody or binding protein from a non-human species that has been altered to be more “human-like”, i.e., more similar to human germline sequences. One type of humanized antibody or binding protein is a CDR-grafted antibody or binding protein, in which non-human CDR sequences are introduced into human VH and VL sequences to replace the corresponding human CDR sequences. A humanized antibody or binding protein also encompasses a variant, derivative, analog or fragment of an antibody or binding protein that comprises framework region (FR) sequences having substantially (e.g., at least 80%, at least 85%, at least 90%, at least 95%, at least 98% or at least 99% identity to) the amino acid sequence of a human antibody and at least one CDR having substantially the amino acid sequence of a non-human antibody. A humanized antibody or binding protein may comprise substantially all of at least one variable domain (Fab, Fab′, F(ab′)₂, Fv) in which the sequence of all or substantially all of the CDR regions correspond to those of a non-human immunoglobulin (i.e., donor antibody) and the sequence of all or substantially all of the FR regions are those of a human immunoglobulin. The humanized antibody or binding protein also may include the CH1, hinge, CH2, CH3, and/or CH4 regions of the heavy chain. In an embodiment, a humanized antibody or binding protein may also comprise at least a portion of a human immunoglobulin Fc region. In some embodiments, a humanized antibody or binding protein only contains a humanized light chain. In some embodiments, a humanized antibody or binding protein only contains a humanized heavy chain. In some embodiments, a humanized antibody or binding protein only contains a humanized variable domain of a light chain and/or humanized variable domain of a heavy chain. In some embodiments, a humanized antibody or binding protein contains a humanized light chain as well as at least a variable domain of a heavy chain. In some embodiments, a humanized antibody or binding protein contains a humanized heavy chain as well as at least a variable domain of a light chain.

The term “protuberance” in some embodiments refers to one or more amino acid modifications to increase the bulk (e.g., the total volume) taken up by the amino acids. For instance, smaller amino acids can be modified or replaced by those having larger side chains which projects from the interface of the first polypeptide chain (heavy or light chain) and can therefore be positioned in a related cavity in the adjacent second polypeptide chain (light or heavy) so as to stabilize the heterodimer, and thereby favor heterodimer formation over homodimer formation. The protuberance may exist in the original interface or may be introduced synthetically (e.g., by altering one or more nucleic acid encoding the amino acid(s) at the interface). In some embodiments, a protuberance is introduced by modifying the nucleic acid encoding at least one “original” amino acid residue in the interface of the first polypeptide with a nucleic acid encoding at least one “engineered” amino acid residue which has a larger side chain volume than the original amino acid residue. It will be appreciated that there can be more than one original and corresponding engineered residue. The upper limit for the number of original residues which are replaced is the total number of residues in the interface of the first polypeptide. In some embodiments, a protuberance is referred to as a “knob.”

A “cavity” refers to at least one amino acid side chain which is recessed from the interface of the first or second polypeptide chain (heavy or light chain) and therefore accommodates a corresponding protuberance on the adjacent second polypeptide chain (light or heavy). The cavity may exist in the original interface or may be introduced synthetically (e.g., by altering one or more nucleic acid encoding the amino acid(s) at the interface). In some embodiments, a protuberance is introduced by modifying the nucleic acid encoding at least one “original” amino acid residue in the interface of the first polypeptide with a nucleic acid encoding at least one “engineered” amino acid residue which has a smaller side chain volume than the original amino acid residue. It will be appreciated that there can be more than one original and corresponding engineered residue. The upper limit for the number of original residues which are replaced is the total number of residues in the interface of the first polypeptide. In some embodiments, a cavity is referred to as a “hole.”

The “interface” between a first and second polypeptide chain can comprise those amino acid residues (or other non-amino acid groups such as carbohydrate groups, NADH, biotin, FAD or haem group) in contact and/or which interact between the first polypeptide chain (heavy or light chain) and the counterpart second polypeptide chain (light or heavy chain). The interaction can be covalent, non-covalent (e.g., ionic) or other interaction. In some embodiments, amino acids on the first and second polypeptide chains that are within 5 Angstroms of each other are considered part of the interface.

The term “biological activity” refers to any one or more biological properties of a molecule (whether present naturally as found in vivo, or provided or enabled by recombinant means). Biological properties include, but are not limited to, binding a receptor, inducing cell proliferation, inhibiting cell growth, inducing other cytokines, inducing apoptosis, and enzymatic activity.

The term “neutralizing” refers to counteracting the biological activity of an antigen when a binding protein specifically binds to the antigen. In an embodiment, a neutralizing binding protein binds to an antigen and reduces the antigen's biological activity by at least about 20%, about 40%, about 60%, about 80%, about 85%, about 90%, about 95%, or about 100% (or any percentage in between).

The term “specificity” refers to the ability of a binding protein to selectively bind an antigen.

The term “affinity” refers to the strength of the interaction between a binding protein and an antigen, and is determined by the sequence of the CDRs of the binding protein as well as by the nature of the antigen, such as its size, shape, and/or charge. Binding proteins may be selected for affinities that provide desired therapeutic end-points while minimizing negative side-effects. Affinity may be measured using methods known to one skilled in the art (see, e.g., U.S. Pat. No. 7,612,181).

The term “potency” refers to the ability of a binding protein to achieve a desired effect, and is a measurement of its therapeutic efficacy. Potency may be assessed using methods known to one skilled in the art (see, e.g., U.S. Pat. No. 7,612,181).

The term “cross-reactivity” refers to the ability of a binding protein to bind a target other than that against which it was raised. Generally, a binding protein will bind its target tissue(s)/antigen(s) with an appropriately high affinity, but will display an appropriately low affinity for non-target normal tissues. Methods of assessing cross-reactivity are known to one skilled in the art (see, e.g., U.S. Pat. No. 7,612,181).

The term “competitive binding” refers to the ability of a binding protein to compete for binding to a target with a reference binding protein and therefore reduce the binding of the reference binding protein to the target. In certain embodiments, competitive binding can be evaluated using routine cross-blocking assays, such as the assay described in ANTIBODIES, A LABORATORY MANUAL, Cold Spring Harbor Laboratory, Ed Harlow and David Lane (1st edition 1988, 2nd edition 2014). In some embodiments, competitive binding is identified when a test antibody or binding protein reduces binding of a reference antibody or binding protein disclosed herein by at least about 50% in the cross-blocking assay (e.g., 50%, 60%, 70%, 80%, 90%, 95%, 99%, 99.5%, or more, or any percentage in between), and/or vice versa. In some embodiments, competitive binding can be due to shared or similar (e.g., partially overlapping) epitopes, or due to steric hindrance where antibodies or binding proteins bind at nearby epitopes. See, e.g., Tzartos, Methods in Molecular Biology, vol. 66, Epitope Mapping Protocols, pages 55-66, Humana Press Inc. (1998) (“only marked mutual crosscompetition should be taken as unequivocal evidence of overlapping epitopes, since weak or one-way inhibition may simply reflect a decrease in affinity owing to steric or allosteric effects. Therefore, we completely ignored cases of weak inhibition (<25%) and essentially only considered inhibition of >50%”). In some embodiments, competitive binding can be used to sort groups of binding proteins that share similar epitopes, e.g., those that compete for binding can be “binned” as a group of binding proteins that have overlapping or nearby epitopes, while those that do not compete are placed in a separate group of binding proteins that do not have overlapping or nearby epitopes.

The term “biological function” refers the specific in vitro or in vivo actions of a binding protein. Binding proteins may target several classes of antigens and achieve desired therapeutic outcomes through multiple mechanisms of action. Binding proteins may target soluble proteins, cell surface antigens, as well as extracellular protein deposits. Binding proteins may agonize, antagonize, or neutralize the activity of their targets. Binding proteins may assist in the clearance of the targets to which they bind, or may result in cytotoxicity when bound to cells.

Portions of two or more antibodies may be incorporated into a multivalent format to achieve distinct functions in a single binding protein molecule. The in vitro assays and in vivo models used to assess biological function are known to one skilled in the art (see, e.g., U.S. Pat. No. 7,612,181).

A “stable” binding protein refers to one in which the binding protein retains some level of its physical stability, chemical stability and/or biological activity upon storage. Methods of stabilizing binding proteins and assessing their stability at various temperatures are known to one skilled in the art (see, e.g., U.S. Pat. No. 7,612,181).

The term “solubility” refers to the ability of a protein to remain dispersed within an aqueous solution. The solubility of a protein in an aqueous formulation depends upon the proper distribution of hydrophobic and hydrophilic amino acid residues, and therefore, solubility can correlate with the production of correctly folded proteins. A person skilled in the art will be able to detect an increase or decrease in solubility of a binding protein using routine HPLC techniques and methods known to one skilled in the art (see, e.g., U.S. Pat. No. 7,612,181).

Binding proteins may be produced using a variety of host cells or may be produced in vitro, and the relative yield per effort determines the “production efficiency.” Factors influencing production efficiency include, but are not limited to, host cell type (prokaryotic or eukaryotic), choice of expression vector, choice of nucleotide sequence, and methods employed. The materials and methods used in binding protein production, as well as the measurement of production efficiency, are known to one skilled in the art (see, e.g., U.S. Pat. No. 7,612,181).

The term “immunogenicity” means the ability of a substance to induce an immune response. Administration of a therapeutic binding protein may result in a certain incidence of an immune response. Potential elements that might induce immunogenicity in a multivalent format may be analyzed during selection of the parental antibodies, and steps to reduce such risk can be taken to optimize the parental antibodies prior to incorporating their sequences into a multivalent binding protein format. Methods of reducing the immunogenicity of antibodies and binding proteins are known to one skilled in the art (U.S. Pat. No. 7,612,181).

The terms “label” and “detectable label” refer to a moiety attached to a member of a specific binding pair, such as an antibody/binding protein or its analyte to render a reaction (e.g., binding) between the members of the specific binding pair, detectable. The labeled member of the specific binding pair is referred to as “detectably labeled.” Thus, the term “labeled binding protein” refers to a protein with a label incorporated that provides for the identification of the binding protein. In an embodiment, the label is a detectable marker that can produce a signal that is detectable by visual or instrumental means, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). Examples of labels for polypeptides include, but are not limited to, the following: radioisotopes or radionuclides (e.g., ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm); chromogens, fluorescent labels (e.g., FITC, rhodamine, lanthanide phosphors), enzymatic labels (e.g., horseradish peroxidase, luciferase, alkaline phosphatase); chemiluminescent markers; biotinyl groups; predetermined polypeptide epitopes recognized by a secondary reporter (e.g., leucine zipper pair sequences, binding sites for secondary antibodies, metal binding domains, epitope tags); and magnetic agents, such as gadolinium chelates. Representative examples of labels commonly employed for immunoassays include moieties that produce light, e.g., acridinium compounds, and moieties that produce fluorescence, e.g., fluorescein. In this regard, the moiety itself may not be detectably labeled but may become detectable upon reaction with yet another moiety.

The term “conjugate” refers to a binding protein that is chemically linked to a second chemical moiety, such as a therapeutic or cytotoxic agent. The term “agent” includes a chemical compound, a mixture of chemical compounds, a biological macromolecule, or an extract made from biological materials. In an embodiment, the therapeutic or cytotoxic agents include, but are not limited to, pertussis toxin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. When employed in the context of an immunoassay, the conjugate antibody may be a detectably labeled antibody used as the detection antibody.

The terms “crystal” and “crystallized” refer to a binding protein (e.g., an antibody), or antigen binding portion thereof, that exists in the form of a crystal. Crystals are one form of the solid state of matter, which is distinct from other forms such as the amorphous solid state or the liquid crystalline state. Crystals are composed of regular, repeating, three-dimensional arrays of atoms, ions, molecules (e.g., proteins such as antibodies), or molecular assemblies (e.g., antigen/antibody complexes). These three-dimensional arrays are arranged according to specific mathematical relationships that are well-understood in the field. The fundamental unit, or building block, that is repeated in a crystal is called the asymmetric unit. Repetition of the asymmetric unit in an arrangement that conforms to a given, well-defined crystallographic symmetry provides the “unit cell” of the crystal. Repetition of the unit cell by regular translations in all three dimensions provides the crystal. (See Giege and Ducruix (1999) CRYSTALLIZATION OF NUCLEIC ACIDS AND PROTEINS, A PRACTICAL APPROACH, 2nd ed., pp. 20 1-16, Oxford University Press, NY, NY).

The term “vector” refers to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Other vectors include RNA vectors. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, other forms of expression vectors are also included, such as viral vectors (e.g., replication defective retroviruses, adenoviruses and adeno-associated viruses), which serve equivalent functions. A group of pHybE vectors (e.g., U.S. Pat. No. 8,187,836) may be used for parental antibody and DVD-binding protein cloning. V1, derived from pJP183; pHybE-hCgl,z,non-a V2, may be used for cloning of antibody and DVD heavy chains with a wild type constant region. V2, derived from pJP191; pHybE-hCk V3, may be used for cloning of antibody and DVD light chains with a kappa constant region. V3, derived from pJP192; pHybE-hCl V2, may be used for cloning of antibody and DVD light chains with a lambda constant region. V4, built with a lambda signal peptide and a kappa constant region, may be used for cloning of DVD light chains with a lambda-kappa hybrid V domain. V5, built with a kappa signal peptide and a lambda constant region, may be used for cloning of DVD light chains with a kappa-lambda hybrid V domain. V7, derived from pJP183; pHybE-hCgl,z,non-a V2, may be used for cloning of antibody and DVD heavy chains with a (234,235 AA) mutant constant region.

The terms “recombinant host cell” or “host cell” refer to a cell into which exogenous, e.g., recombinant, DNA has been introduced. Such terms refer not only to the particular subject cell, but to the progeny of such a cell. Because certain modifications may occur in succeeding generations due to either mutation or environmental influences, such progeny may not, in fact, be identical to the parent cell, but are still included within the scope of the term “host cell” as used herein. In an embodiment, host cells include prokaryotic and eukaryotic cells. In an embodiment, eukaryotic cells include protist, fungal, plant and animal cells. In another embodiment, host cells include but are not limited to the prokaryotic cell line E. coli; mammalian cell lines CHO, HEK 293, COS, NS0, SP2 and PER.C6; the insect cell line Sf9; and the fungal cell Saccharomyces cerevisiae.

The term “transfection” encompasses a variety of techniques commonly used for the introduction of exogenous nucleic acid (e.g., DNA) into a host cell, e.g., electroporation, calcium-phosphate precipitation, DEAE-dextran transfection and the like.

The term “cytokine” refers to a protein released by one cell population that acts on another cell population as an intercellular mediator. The term “cytokine” includes proteins from natural sources or from recombinant cell culture and biologically active equivalents of the native sequence cytokines.

The term “biological sample” refers to a quantity of a substance from a living thing or formerly living thing. Such substances include, but are not limited to, blood, plasma, serum, urine, amniotic fluid, synovial fluid, endothelial cells, leukocytes, monocytes, other cells, organs, tissues, bone marrow, lymph nodes and spleen.

The term “component” refers to an element of a composition. In relation to a diagnostic kit, for example, a component may be a capture antibody, a detection or conjugate antibody, a control, a calibrator, a series of calibrators, a sensitivity panel, a container, a buffer, a diluent, a salt, an enzyme, a co-factor for an enzyme, a detection reagent, a pretreatment reagent/solution, a substrate (e.g., as a solution), a stop solution, and the like that can be included in a kit for assay of a test sample. Thus, a “component” can include a polypeptide or other analyte as above, that is immobilized on a solid support, such as by binding to an anti-analyte (e.g., anti-polypeptide) antibody. Some components can be in solution or lyophilized for reconstitution for use in an assay.

The term “control” refers to a composition known to not analyte (“negative control”) or to contain analyte (“positive control”). A positive control can comprise a known concentration of analyte. A “positive control” can be used to establish assay performance characteristics and is a useful indicator of the integrity of reagents (e.g., analytes).

The term “predetermined level” refers generally to an assay cutoff value that is used to assess diagnostic/prognostic/therapeutic efficacy results by comparing the assay results against the predetermined cutoff/level, where the predetermined cutoff/level already has been linked or associated with various clinical parameters (e.g., severity of disease, progression/nonprogression/improvement, etc.). While the present disclosure may provide exemplary predetermined levels, it is well-known that cutoff values may vary depending on the nature of the immunoassay (e.g., antibodies employed, etc.). It further is well within the ordinary skill of one in the art to adapt the disclosure herein for other immunoassays to obtain immunoassay-specific cutoff values for those other immunoassays based on this disclosure. Whereas the precise value of the predetermined cutoff/level may vary between assays, correlations as described herein (if any) may be generally applicable.

The term “specific binding partner” refers to a member of a specific binding pair. A specific binding pair comprises two different molecules that specifically bind to each other through chemical or physical means. Therefore, in addition to antigen and antibody specific binding, other specific binding pairs can include biotin and avidin (or streptavidin), carbohydrates and lectins, complementary nucleotide sequences, effector and receptor molecules, cofactors and enzymes, enzyme inhibitors and enzymes, and the like. Furthermore, specific binding pairs can include members that are analogs of the original specific binding members, for example, an analyte-analog Immunoreactive specific binding members include antigens, antigen fragments, and antibodies, including monoclonal and polyclonal antibodies as well as complexes, fragments, and variants (including fragments of variants) thereof, whether isolated or recombinantly produced.

The term “Fc region” refers to the C-terminal region of an immunoglobulin heavy chain, which in some instances may be generated by papain digestion of an intact antibody or binding protein. The Fc region may be a native sequence Fc region or a variant Fc region. The Fc region of an immunoglobulin generally comprises two constant domains, a CH2 domain and a CH3 domain, and optionally comprises a CH4 domain. Replacement of amino acid residues in the Fc portion is contemplated by the disclosure. The Fc region mediates several effector functions, e.g., cytokine induction, antibody dependent cell mediated cytotoxicity (ADCC), phagocytosis, complement dependent cytotoxicity (CDC), and half-life/clearance rate of antibody or binding protein and antigen-antibody or antigen-binding protein complexes. In some cases these effector functions are desirable for a therapeutic immunoglobulin but in other cases might be unnecessary or even deleterious, depending on the therapeutic objectives.

The term “antigen-binding portion” of a binding protein refers to one or more fragments of a binding protein that retain the ability to specifically bind to an antigen. The antigen-binding function of a binding protein may be performed by fragments of a full-length binding protein, including bispecific, dual specific, or multi-specific formats; for instance, binding to two or more different antigens. Examples of binding fragments encompassed within the term “antigen-binding portion” of an binding protein include (i) an Fab fragment, a monovalent fragment consisting of the VL, VH, CL and CH1 domains; (ii) an F(ab′)₂ fragment, a bivalent fragment comprising two Fab fragments linked by a disulfide bridge at the hinge region; (iii) an Fd fragment consisting of the VH and CH1 domains; (iv) an Fv fragment consisting of the VL and VH domains of a single arm of an antibody or binding protein, (v) a dAb fragment, which comprises a single variable domain; and (vi) an isolated complementarity determining region (CDR). Furthermore, although the two domains of the Fv fragment, VL and VH, are encoded by separate genes, they may be joined, e.g., using recombinant methods, by a synthetic linker that enables them to be made as a single protein chain in which the VL and VH regions pair to form monovalent molecules (known as single chain Fv (scFv). Such single chain antibodies or binding proteins are also intended to be encompassed within the term “antigen-binding portion” of an antibody or binding protein. Other forms of single chain antibodies, such as diabodies are also encompassed. In addition, single chain antibodies or binding protein also include “linear” antibodies or binding protein comprising a pair of tandem Fv segments (VH-CH1-VH-CH1) which, together with complementary light chain polypeptides, form a pair of antigen binding regions.

The terms “antigen binding site” and “binding site for an antigen” are used interchangeably, and refer to a region formed by the association between three CDRs from a heavy chain variable domain and three CDRs from a light chain variable domain. Thus, the term also encompasses a region formed by the association between a heavy chain variable domain and a light chain variable domain. An antigen binding site as described herein is capable of specifically binding to an antigen. The term “antigen binding region” refers to a portion of a binding protein that comprises one, two, three, four, or more antigen binding sites. An antigen binding region of a binding protein as described herein therefore is capable of binding one, two, three, four, or more antigens that are the same or different.

The term “multivalent binding protein” refers to a binding protein comprising two or more antigen binding sites. In an embodiment, the multivalent binding protein is engineered to have three or more antigen binding sites, and may not be a naturally occurring antibody. The term “multispecific binding protein” refers to a binding protein capable of binding two or more related or unrelated targets. In an embodiment, the dual variable domain (DVD) binding proteins provided herein may comprise two or more antigen binding sites and are tetravalent or multivalent binding proteins.

A “bivalent” binding protein described herein comprises two antigen binding sites that bind to the same or different antigens (or epitopes). For instance, a bivalent binding protein described herein may be monospecific or bispecific depending on whether two antigen binding sites of the bivalent binding protein bind to the same or different antigens. If the two antigen binding sites bind to the same antigen, the bivalent binding protein is monospecific. Otherwise, the bivalent binding protein binds to two different antigens and therefore is bispecific.

The term “linker” refers to an amino acid residue or a polypeptide comprising two or more amino acid residues joined by peptide bonds that are used to link two polypeptides (e.g., two VH or two VL domains) Such linker polypeptides are well known in the art (see, e.g., Holliger et al. (1993) Proc. Natl. Acad. Sci. USA 90:6444-6448; Poljak et al. (1994) Structure 2:1121-1123).

The terms “Kabat numbering”, “Kabat definitions” and “Kabat labeling” are used interchangeably herein. These terms, which are recognized in the art, refer to a system of numbering amino acid residues which are more variable (i.e., hypervariable) than other amino acid residues in the heavy and light chain variable regions of an antibody or binding protein, or an antigen binding portion thereof (Kabat et al. (1971) Ann. NY Acad. Sci. 190:382-391 and

Kabat et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3. In some embodiments, the CDR sequences, framework sequences, and or constant region sequences are identified using Kabat numbering.

The term “CDR” refers to a complementarity determining region within an immunoglobulin variable region sequence. There are three CDRs in each of the variable regions of the heavy chain and the light chain, which are designated CDR1, CDR2 and CDR3, for each of the heavy and light chain variable regions. The term “CDR set” refers to a group of three CDRs that occur in a single variable region capable of binding the antigen. The exact boundaries of these CDRs have been defined differently according to different systems. The system described by Kabat (Kabat et al. (1987) and (1991)) not only provides an unambiguous residue numbering system applicable to any variable region of an antibody or binding protein, but also provides precise residue boundaries defining the three CDRs in each heavy or light chain sequence. These CDRs may be referred to as Kabat CDRs. Chothia and coworkers (Chothia and Lesk (1987) J. Mol. Biol. 196:901-917; Chothia et al. (1989) Nature 342:877-883) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chain regions, respectively. These regions may be referred to as Chothia CDRs, which have boundaries that overlap with Kabat CDRs. Other boundaries defining CDRs overlapping with the Kabat CDRs have been described by Padlan (1995) FASEB J. 9:133-139 and MacCallum (1996) J. Mol. Biol. 262(5):732-45). Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat or Chothia defined CDRs.

The term “epitope” refers to a region of an antigen that is bound by a binding protein. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. In an embodiment, an epitope comprises the amino acid residues of a region of an antigen (or fragment thereof) that are recognized by and/or bound by the complementary site on the specific binding partner. An antigenic fragment can contain more than one epitope. In certain embodiments, a binding protein specifically binds an antigen when it recognizes its target antigen in a complex mixture of proteins and/or macromolecules. Binding proteins “bind to the same epitope” if the antibodies or binding proteins cross-compete (one prevents the binding or modulating effect of the other). Methods of visualizing and modeling epitope recognition are known to one skilled in the art (see, e.g., U.S. Pat. No. 7,612,181).

The term “pharmacokinetic(s)” refers to the process by which a drug is absorbed, distributed, metabolized, and excreted by an organism. To generate a multivalent binding protein molecule with a desired pharmacokinetic profile, parent monoclonal antibodies with similarly desired pharmacokinetic profiles are selected. The PK profiles of the selected parental monoclonal antibodies can be easily determined in rodents using methods known to one skilled in the art (see, e.g., U.S. Pat. No. 7,612,181).

The term “bioavailability” refers to the degree and rate at which a drug is absorbed into a living system or is made available at the site of physiological activity. Bioavailability can be a function of several of the previously described properties, including stability, solubility, immunogenicity and pharmacokinetics, and can be assessed using methods known to one skilled in the art (see, e.g., U.S. Pat. No. 7,612,181).

The term “surface plasmon resonance” refers to an optical phenomenon that allows for the analysis of real-time biospecific interactions by detection of alterations in protein concentrations within a biosensor matrix, for example using the BIAcore® system (BIAcore International AB, a GE Healthcare company, Uppsala, Sweden and Piscataway, N.J.). For further descriptions, see Jonsson et al. (1993) Ann. Biol. Clin. 51:19-26. The term “Kₒₙ” refers to the on rate constant for association of a binding protein (e.g., an antibody or DVD-Ig) to the antigen to form, e.g., a DVD-Ig/antigen complex. The term “Kₒₙ” also refers to “association rate constant”, or “ka”, as is used interchangeably herein. This value indicating the binding rate of a binding protein to its target antigen or the rate of complex formation between a binding protein, e.g., an antibody, and antigen also is shown by the equation below:

Antibody(“Ab”)+Antigen(“Ag”)→Ab−Ag

The term “K_{off}” refers to the off rate constant for dissociation, or “dissociation rate constant”, of a binding protein (e.g., an antibody or DVD-Ig) from the, e.g., DVD-Ig/antigen complex as is known in the art. This value indicates the dissociation rate of a binding protein, e.g., an antibody, from its target antigen or separation of Ab−Ag complex over time into free antibody and antigen as shown by the equation below:

Ab+Ag←Ab−Ag

The terms “K_{d}” and “equilibrium dissociation constant” may refer to the value obtained in a titration measurement at equilibrium, or by dividing the dissociation rate constant (K_{off}) by the association rate constant (Kₒₙ). The association rate constant, the dissociation rate constant and the equilibrium dissociation constant, are used to represent the binding affinity of a binding protein (e.g., an antibody or DVD-Ig) to an antigen. Methods for determining association and dissociation rate constants are well known in the art. Using fluorescence-based techniques offers high sensitivity and the ability to examine samples in physiological buffers at equilibrium. Other experimental approaches and instruments such as a BIAcore® (biomolecular interaction analysis) assay, can be used (e.g., instrument available from BIAcore International AB, a GE Healthcare company, Uppsala, Sweden). Additionally, a KinExA® (Kinetic Exclusion Assay) assay, available from Sapidyne Instruments (Boise, Id.), can also be used.

The term “variant” refers to a polypeptide that differs from a given polypeptide in amino acid sequence by the addition (e.g., insertion), deletion, or conservative substitution of amino acids, but that retains the biological activity of the given polypeptide. A conservative substitution of an amino acid, i.e., replacing an amino acid with a different amino acid of similar properties (e.g., hydrophilicity and degree and distribution of charged regions) is recognized in the art as typically involving a minor change. These minor changes can be identified, in part, by considering the hydropathic index of amino acids, as understood in the art (see, e.g., Kyte et al. (1982) J. Mol. Biol. 157: 105-132). The hydropathic index of an amino acid is based on a consideration of its hydrophobicity and charge. It is known in the art that amino acids of similar hydropathic indexes in a protein can be substituted and the protein still retains protein function. In one aspect, amino acids having hydropathic indexes of ±2 are substituted. The hydrophilicity of amino acids also can be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity (see, e.g., U.S. Pat. No. 4,554,101). Substitution of amino acids having similar hydrophilicity values can result in peptides retaining biological activity, for example immunogenicity, as is understood in the art. In one aspect, substitutions are performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. The term “variant” also includes polypeptide or fragment thereof that has been differentially processed, such as by proteolysis, phosphorylation, or other post-translational modification, yet retains its biological activity or antigen reactivity, e.g., the ability to bind to VEGF. The term “variant” encompasses fragments of a variant unless otherwise defined. A variant may be about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, or 75% identical to the wild type sequence.

It will be readily apparent to those skilled in the art that other suitable modifications and adaptations of the methods described herein are obvious and may be made using suitable equivalents without departing from the scope of the embodiments disclosed herein. Having now described certain embodiments in detail, the same will be more clearly understood by reference to the following examples, which are included for purposes of illustration only and are not intended to be limiting.

### EXAMPLES

### Example 1: IgM CH2 and IgE CH2 Structure and Sequence

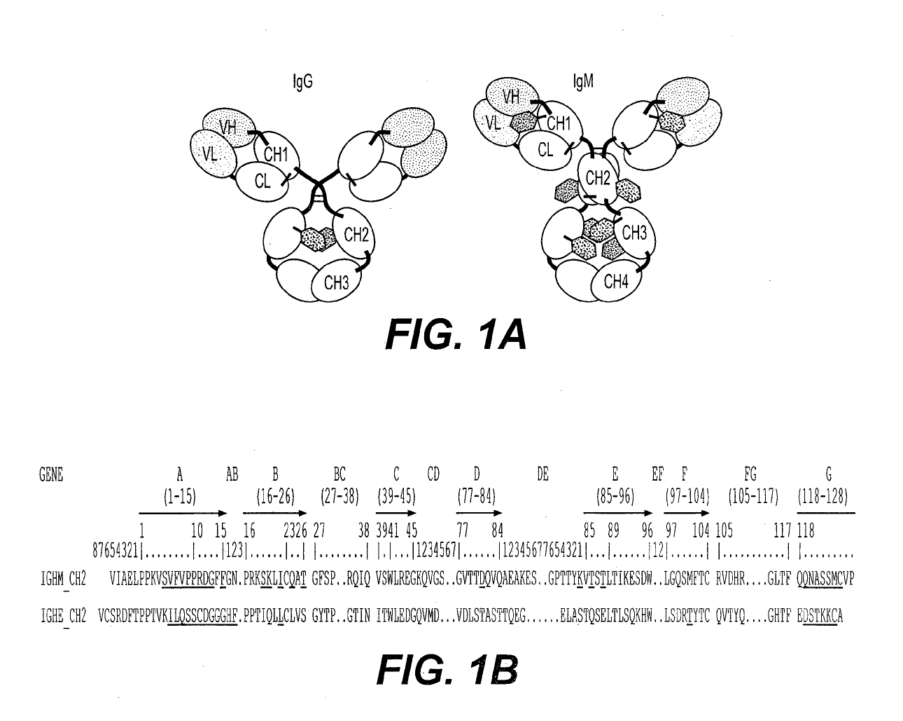

The heavy chains of IgM and IgE molecules contain an additional domain (CH2, Cm2 and C12) in place of the hinge region seen in an IgG molecule (Perkins et al. (1991) J. Mol. Biol. 221: 1345-1366; Beavil et al. (1995) Biochemistry 34: 14449-14461; Wan et al. (2002) Nature Immunol. 3: 681-686), as shown in FIG. 1A. The IgM CH2 domain (MH2) consists of 111 amino acid residues (12.2 kDa) forming a homodimer covalently held together by a disulfide bond formed between cysteine residue 125 on each CH2 domain (Davis et al. (1989) EMBO J. 8: 2519-2526; Davis et al. (1989) Immunol. Today 10: 118-122 and 127-118). Each domain is further stabilized by an internal disulfide bond between cysteine residues 23 and 104 (Putnam et al. (1973) Science 182: 287-291). MH2 has an N-glycosylation site at residue 120. MH2 alone is capable of forming covalently linked dimers. A melting point of 55° C. was determined by dynamic light scattering. All MH2 variants with a mutation to remove disulfide bond(s) or N-glycosylation exhibit a reduced thermal stability when compared with the wild-type MH2, indicating that the interdomain disulfide bond, as well as N-glycans on the molecule, contribute to MH2 stability. The N-glycosylation may be altered by glyco-engineering to modulate the pharmacokinetic properties of MH2 or MH2 variant-containing molecules.

The IgE CH2 domain (EH2) consists of 107 amino acid residues forming a homodimer covalently held together by two inter-chain disulfide bonds, which are formed between cysteine residue 11 and 124 of two domains. Each domain is further stabilized by an intra-chain disulfide bond between cysteine residue 23 and 104. EH2 has one N-glycosylation site at residue 38. The N-glycosylation may be altered by glyco-engineering to modulate the pharmacokinetic properties of EH2 or EH2 variant-containing molecules.

The MH2 and EH2 may be used as a covalently linked dimerization building block to build bispecific or multispecific molecules by fusing other domains at the N and/or C-terminus of MH2 or EH2. In particular, the central location of the MH2 and EH2 within their respective heavy chains, containing further heavy chain sequences at both ends, as well as their contribution to segmental flexibility, suggest they may be suitable for dimerization in multispecific molecules.

### Example 2: Engineering MH2 or EH2 Hetero-Dimerization Domains

MH2 or EH2 hetero-dimerization may occur when different domains are fused with MH2 or EH2 to form a heterodimer. For example, when IgG VH and VL are fused at the MH2 or EH2 N-terminal, MH2 or EH2 hetero-dimerization will help to form a VH-MH2a/VL-MH2b or VH-EH2a/VL-EH2b heterodimer to obtain an antigen binding domain, while eliminating the formation of non-functional VH-MH2/VH-MH2, VL-MH2/VL-MH2, VH-EH2/VH-EH2, or VL-EH2/VL-EH2 homodimers.

MH2 or EH2 heterodimers may be engineered by modifying the MH2 or EH2 homodimer interface through electrostatic interactions and/or hydrophobic interactions. When incorporated with other domains to form bispecific or multispecific molecules, the engineering approach also needs to avoid increasing the possibility of forming a dimer between the MH2 and non-MH2 domains or the EH2 and non-EH2 domains. The dimer interface residues may be defined as the residues within 5 Å of paired chain in modeled human MH2 dimer structure or 2Y7Q.pdb for human EH2 dimer. The human MH2 dimer structure can be modeled on a mouse MH2 dimer x-ray structure (4JVU.pdb). The interface residues are underlined in FIG. 1B, which represent potential hetero-dimerization engineering sites.

MH2 domain hetero-dimerization strategies are discussed below, which could also be applied using the EH2 domain.

### Example 2.1: Engineering MH2 Hetero-Dimerization Through Electrostatic Interactions

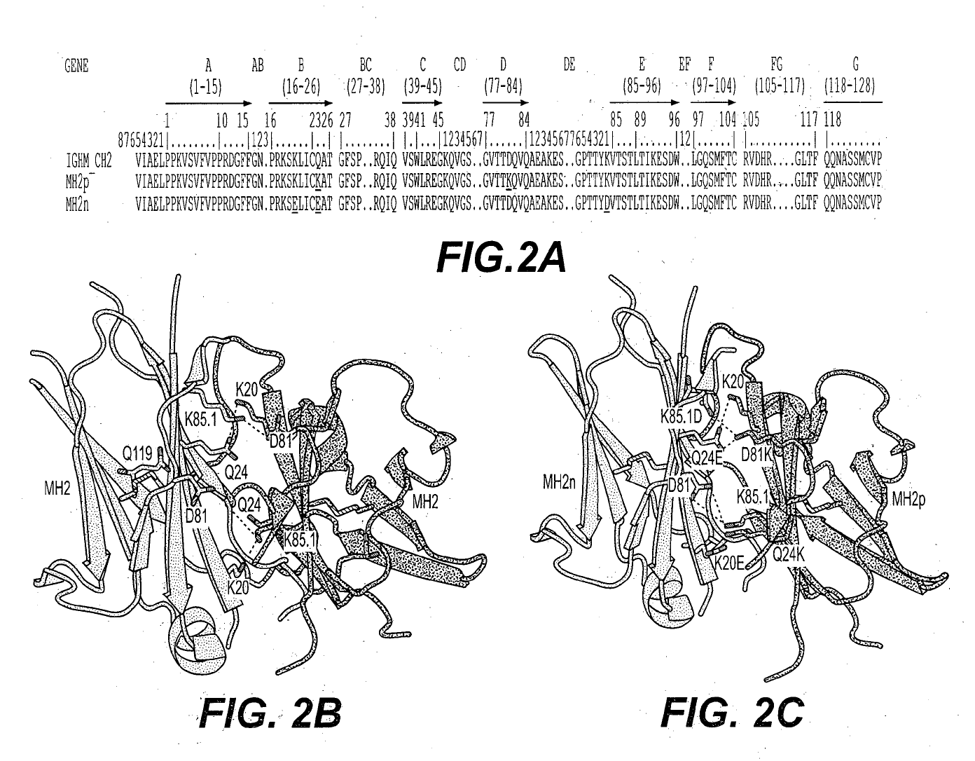

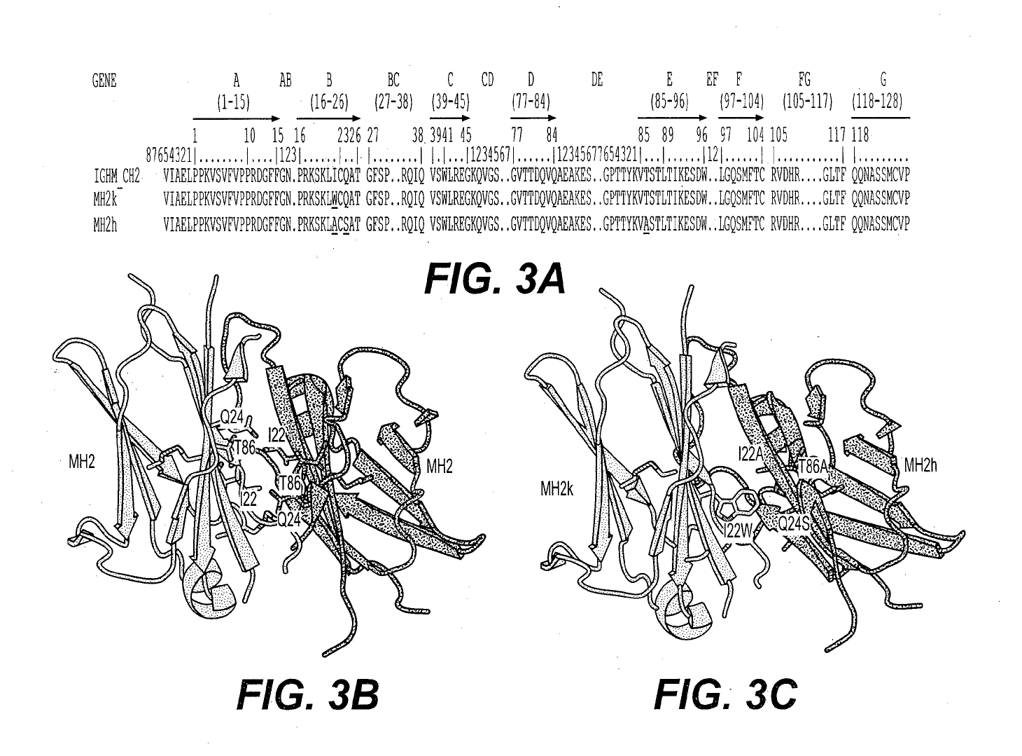

Residue D12, K20, Q24, D81, K85.1, and Q119 on both MH2 domains at the MH2 dimer interface form multiple electrostatic interactions through 2 sets of 3 inter-chain pairs: D12-Q119, K20-Q24, and D81-K85.1, as shown in FIG. 2B. A MH2 heterodimer can be constructed by creating a MH2 positive chain (MH2p) by introducing positive residues on one MH2, and negative residues on the other MH2 (MH2n) to pair with positive residues introduced on MH2p. MH2p prefers to pair with MH2n due to attractive electrostatic interactions, and it does not prefer to pair with MH2p due to repulsive electrostatic interactions. MH2n prefers to pair with MH2p rather than with another MH2n for the same reason. One way to create MH2p is to introduce Q24K and D81K on one MH2 and one way to create MH2n is to introduce mutations K20E, Q24E, and K85.1D on the other MH2. The attractive electrostatic interactions formed between MH2p and MH2n are shown in FIG. 2C. The new mutations can be identified and the current set of mutations can be further optimized among the interface residues underlined in FIG. 1B and their surrounding residues through computational and/or experimental approaches for improving hetero-dimerization and thermal stability.

### Example 2.2: Engineering MH2 Heterodimers Through Hydrophobic Interactions

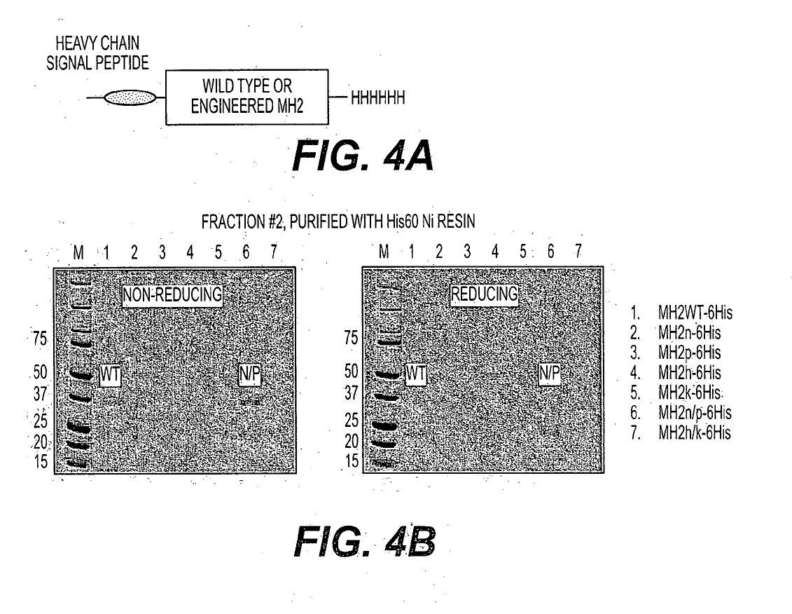

MH2 heterodimerization may also be achieved by engineering hydrophobic interactions on the MH2:MH2 dimer interface. One way to engineer heterodimers through hydrophobic interactions is to introduce one or more bulky residues on one MH2 to create MH2 ‘knobs’ (MH2k) and to introduce one or more small residues on the other MH2 to create MH2 ‘holes’ (MH2h) to compensate for the bulky residues introduced on MH2k. For example, residues I22, Q24 and T86 on the MH2 dimer interface, as shown in FIG. 3B, can be modified. MH2k is created by introducing mutation I22W on one MH2 and MH2h is created by introducing mutations I22A, Q24S, and T86A on the other MH2 to compensate for the bulky residue W22 on MH2k, as shown in FIG. 3C. An attractive hydrophobic interaction forms between MH2k and MH2h. MH2k prefers to pair with MH2h instead of forming a homodimer with itself, and vice versa. In addition to these mutations, further modifications at interface residues underlined in FIG. 1B and their surrounding residues can be introduced through computational and/or experimental approaches for improving heterodimerization and thermal stability.

### Example 2.3: Engineering MH2 Heterodimers Through Library-Based Molecular Evolution

Synthetic libraries, which include all potential mutations at MH2 interface residues and surrounding residues, can be used to optimize MH2 hetero-dimerization.

### Example 2.4: Engineered MH2 Domain Only Forming a Heterodimer

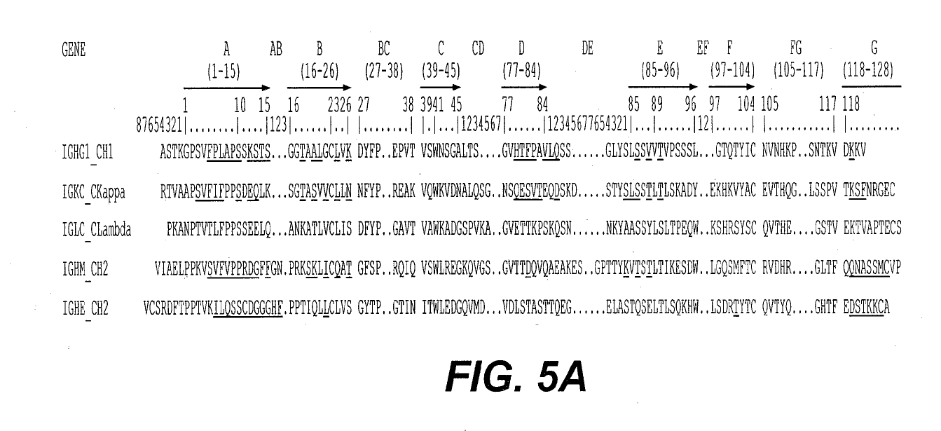

The wild type and engineered MH2 domains as described in Examples 2.1 and 2.2 (sequences are listed in Table 2) are cloned into expression vector as shown in FIG. 4A. The wild type and engineered MH2 domains (MH2n, MH2p, MH2h, and MH2k) were expressed alone in HEK293 cells. Co-expression of MH2n/MH2p and MH2h/MH2k was also carried out. All proteins were purified by Ni-NTA beads. As shown in FIG. 4B, in the Non-reducing SDS-PAGE, MH2 formed homodimers with wild type MH2 expressed alone. There was no homodimer formation with MH2n, MH2p, or MH2h expressed alone. There was slight dimer formation with MH2k expressed alone. Dimers formed with the co-expression of MH2n/MH2p and MH2h/MH2k. Dimerization improved protein expression as MH2n/MH2p dimer had better expression levels than wild type MH2 dimer and MH2h/MH2k dimer. Since there was no homodimer formation with MH2n or MH2p expressed alone, the dimer formed with MH2n/MH2p co-expression was heterodimeric. In the reducing SDS gel, the inter-chain disulfide bond between MH2 domains was reduced, and only monomer was presented.

### Example 3: Using MH2 or EH2 Homodimer or Engineered Heterodimer to Replace CH1/Cκ(λ) Heterodimer in IgG Molecule

### Example 3.1: Comparing MH2 or EH2 Homodimer with IgG CH1/Cκ(λ) Heterodimer

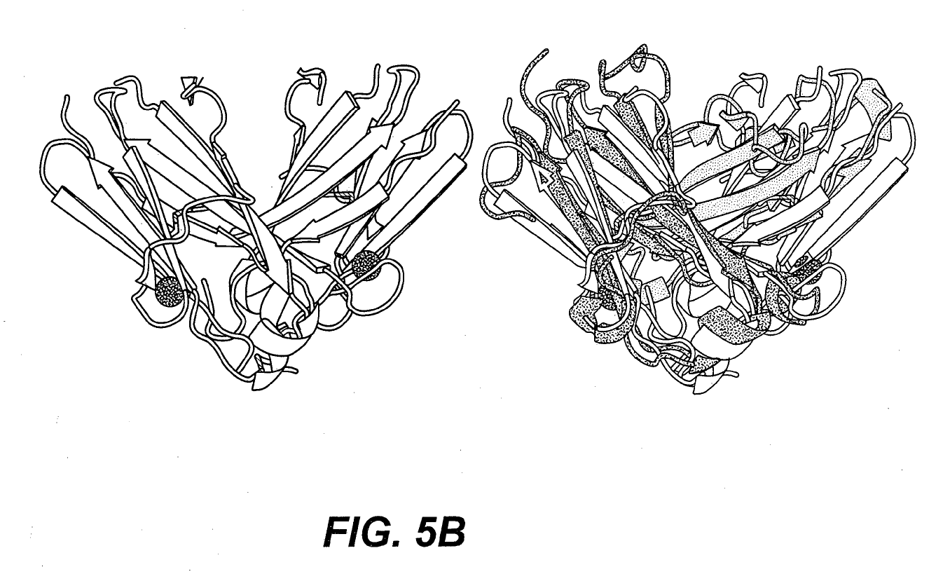

MH2 or EH2 includes an anti-parallel beta-sheet Ig fold structure, which is very similar to IgG CH1, Cκ and Cλ, as shown in FIG. 5A. Both the MH2 and EH2 homodimer are covalently linked by a disulfide bond, as is the CH1/Cκ heterodimer. The overall MH2 or EH2 homodimer holds a conformation that is very similar to the CH1/Cκ heterodimer, as shown in FIG. 5B. The dimerization interface residues include the residues within 5 Å of the paired chain, which are underlined in FIG. 5A. The MH2 or EH2 dimer interface sequence diverges enough from the IgG CH1 and Cκ(λ) to avoid MH2 or EH2 pairing with IgG CH1 or Cκ(λ).

### Example 3.2: Replacing CH1/Cκ(λ) Heterodimer by MH2 or EH2 Homodimer in IgG Molecule

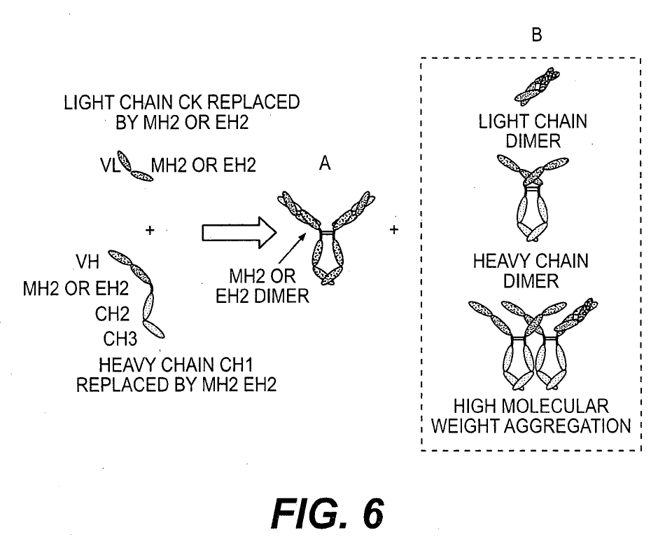

As described in Example 3.1, when compared with CH1/Cκ(λ), the MH2 homodimer or EH2 homodimer has a similar structure, conformation, and stability. The MH2 homodimer or EH2 homodimer provides similar support to VH/VL in an IgG format, preserving the structural and functional integrity of the IgG variable domain. The MH2 or EH2 domain is covalently linked by disulfide bond(s) to form a dimer and will not pair with another domain in the IgG molecule, such as VH, VL, CH2, or CH3. As shown in FIG. 6, using an MH2 or EH2 homodimer to replace CH1/κ(λ) in the IgG molecule may still produce a mixture of the desired molecule and others caused by MH2 or EH2 homodimerization, as shown in FIG. 6, box B. These alternate structures listed in FIG. 6, box B are easily separated from the desired molecule by standard protein purification methods. However, their presence impacts the yield of the desired molecules.

### Example 3.3: Using MH2 or EH2 Heterodimer to Replace CH1/Cκ(λ) Heterodimer in IgG Molecule

The MH2 or EH2 heterodimers engineered through the methods described in Example 2 have the same or improved stability, with similar structure conformation, as the original MH2 or EH2 homodimer and CH1/κ(λ) heterodimer. The engineered MH2 or EH2 heterodimer domain is covalently linked by disulfide bond(s) and will not pair with itself or other domains in IgG molecules, such as VH, VL, CH2, or CH3. Further, using the MH2 (MH2a/MH2b) or EH2 (EH2a/EH2b) heterodimer instead of MH2 or EH2 homodimer to replace CH1/κ(λ) in an IgG molecule eliminates the contaminants listed in FIG. 6, box B and preserves the structural and functional integrity of the IgG variable domain.

### Example 3.4: DE Loop Engineering on MH2 to Improve the Interface Between VII and MH2 or the Interface Between VL and MH2

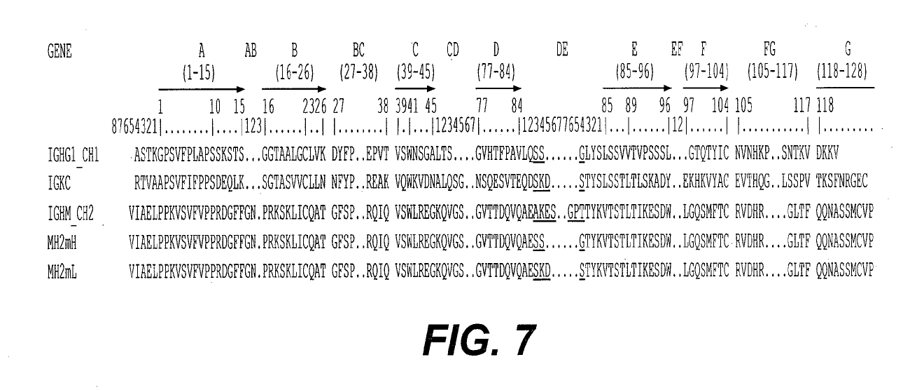

In an IgG molecule, the DE loop of IgG CH1 contacts a heavy chain variable domain (VH) and the DE loop of Cκ contacts a light chain variable domain (VL). When CH1/Cκ is replaced by a MH2 homo- or hetero-dimer, the DE loop of MH2 will contact VH or VL, respectively. As shown in FIG. 7, the DE loop of MH2 has a different length and sequence from the DE loop of CH1 and Cκ. In order to mimic the interface between CH1 and VH, the MH2 DE loop underlined in FIG. 7, which includes residue 84.3A, 84.4K, 84.5E, 84.65, 85.6G, 85.5P, and 85.4T, can be replaced by an IgG CH1 DE loop including residue 84.3S, 84.4S and 85.4G when MH2 is used to replace CH1. The mutated MH2 domain is named MH2mH. The same DE loop of MH2 can also be replaced by the DE loop of IgG Cκ, including residue 84.3S, 84.4K, 84.5D and 85.4S, to mimic VL/Cκ interface at VL/MH2 interface. The mutated MH2 domain is named MH2 mL.

### Example 3.5: Linker Between Variable Domains and MH2 or EH2 Domain

When an IgG VH or VL is fused at the N-terminal of MH2 or EH2, a short linker may help to optimize the interface between the variable domain and the MH2 or EH2 domain. The linker may be, for example, a natural extension of an IgG variable domain, a GS linker, and/or any other short peptide.

### Example 3.6: Modification of IgG Hinge Region when MH2 or EH2 Domains are Fused at the N-Terminal

The hinge region of human IgG1, EPKSCDKTHTCPPCP (SEQ ID NO:32), has three cysteine residues. The first cysteine residue in the hinge region forms an inter-chain disulfide bond with the last cysteine residue in the light chain constant domain. The other two cysteine residues form two inter-heavy chain disulfide bonds to stabilize heavy chain dimerization. When CH1/Cκ(λ) is replaced by MH2 homo- or hetero-dimer or EH2 homo- or hetero-dimer in an IgG molecule, the first cysteine residue in the hinge region may form an extra inter-heavy chain disulfide bond. If two inter-heavy chain disulfide bonds are preferred, this may be achieved by mutating out the first cysteine or shortening the hinge region by 5 residues at the N-terminal to DKTHTCPPCP (SEQ ID NO:33).

### Example 4: Modulating Glycosylation Site on MH or EH2 Domain to Introduce 0-4 Glycosylation Site in IgG Molecules when Replacing CH1/Cκ(λ) by MH2 or EH2 Homo- or Engineered Hetero-Dimer

As described in Examples 1 and 3, there is one N-glycosylation site in the MH2 domain and one in the EH2 domain. Replacing CH1/κ(λ) by a MH2 or EH2 homo- or engineered hetero-dimer will introduce 4 additional glycosylation sites in IgG molecules. The N-glycosylation site on MH2 or EH2 may be eliminated by mutation at positions 120 or 122 in MH2 or at positions 38 or 40 in EH2 respectively to reduce molecular heterogeneity. Alternative glyco-engineering to modulate the pharmacokinetic properties of the molecules can also be used.

### Example 5: Replacing One Arm CH1/Cκ(λ) by MH2 or EH2 Homo- or Engineered Hetero-Dimer in Heavy Chain Hetero-Dimerization Based Bispecific Molecule

Efficient production of bispecific IgG in a single host cell requires simultaneously overcoming both light chain and heavy chain pairing problems. Table 1 below lists exemplary currently available heavy chain heterodimerization strategies through IgG CH3 engineering. The symmetry of CH1/κ(λ) dimerization on both arms of IgG is the main reason to cause light chain mispairing in bispecific IgG generation. As described in Example 3, the MH2 or EH2 homo- or engineered hetero-dimer is structurally similar to CH1/Cκ(λ) and may be used to replace CH1/κ(λ) in an IgG molecule to support VH/VL pairing. The sequence divergence among the interface residues eliminates the pairing between MH2 and non-MH2 domains, or EH2 and non-EH2 domains, such as VH, VL, CH1, Cκ(λ), CH2, or CH3. Replacing one arm CH1/κ(λ) by a MH2 or EH2 dimer overcomes both light chain and heavy chain mispairing for one arm in bispecific IgG generation. The knobs-into-holes format is used as an example of the heavy chain hetero-dimerization approach for generating heterodimers of the two arms of an IgG, but any other heavy chain hetero-dimerization approach could also be used.

TABLE 1
Strategies And Mutations To Overcome The Bispecific IgG Heavy Chain-Pairing Problem

| | | Mutations In First | Mutations In Second | |
|---|---|---|---|---|
| Company | Technology Name | Heavy Chain | Heavy Chain | Reference |
| Genentech | Knobs-into-holes | T366W | T366S, L368A, Y407V | Ridgway et al. (1996) |
| | | | | Protein Engineering 9: |
| | | | | 617-621; Atwell et al. |
| | | | | (1997) J. Mol. Biol. 270: |
| | | | | 26-35 |
| Genmab | DuoBody | F405L | K409R | Labrijn et al. (2013) Proc. |
| | | | | Natl. Acad. Sci. USA 110: |
| | | | | 5145-5150 |
| Zymeworks | Azymetric | T350V, L351Y, F405A, | T350V, T366L, K392L, | Von Kreudenstein et al. |
| | | Y407A | T394W | (2013) mAbs 5: 646-654 |
| Amgen | Charge pair | K409D, K392D | D399K, E356K | Gunasekaran et al. (2010) |
| | | | | J. Biol. Chem. 285: 19637- |
| | | | | 19646 |
| Rinat-Pfizer | Charge pair | D221E, P228E, L368E | D221R, P228R, K409R | Strop et al. (2012) J. Mol. |
| | | | | Biol. 420: 204-219 |
| Xencor | HA-TF | S364H, F405A | Y349T, T394F | Moore et al. (2011) mAbs |
| | | | | 3: 546-557 |
| EMD Serono | SEEDbody | IgG/A chimera | IgA/G chimera | Davis et al. (2010) Protein |
| | | | | Engineering, Design & |
| | | | | Selection: PEDS 23: 195-202 |
| Regeneron | Differential protein | H435R | None | Davis et al. (2013) Regeneron |
| | A affinity | | | Pharmaceuticals, Ed. |

### Example 5.1: Replacing One Arm CH1/Cκ(λ) by MH2 or EH2 Dimer in Knobs-into-Holes Format

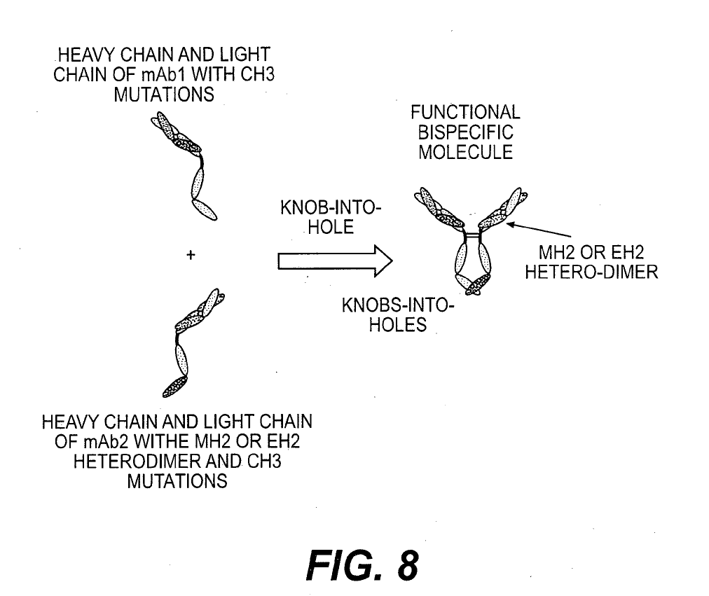

Replacing a CH1/κ(λ) heterodimer on one arm of an IgG with an MH2 or EH2 homodimer in knobs-into-holes format eliminates MH2 or EH2 pairing with IgG CH or Cκ(λ). However, MH2 or EH2 homo-dimerization might still cause potential contaminants, as discussed above. Using an MH2 or EH2 heterodimer engineered by the strategies presented in Example 2, such as MH2p/MH2n or MH2k/MH2h, eliminates the contaminants, as shown in FIG. 8.

### Example 5.2: Modification of IgG Hinge Region when One Arm CH1/Cκ(λ) is Replaced by MH2 or EH2 Dimer in Knobs-into-Holes Format

The first cysteine residue in the IgG1 hinge region, EPKSCDKTHTCPPCP (SEQ ID NO:32) forms an inter-chain disulfide bond with the last cysteine residue in the light chain constant domain. When one arm CH1/κ(λ) in knobs-into-holes format is replaced by a MH2 or EH2 dimer, the hinge region after the MH2 or EH2 domain may be reduced by 5 amino acid residues at the N-terminal to DKTHTCPPCP (SEQ ID NO:33). The hinge region after CH1 in the other heavy chain keeps the original length.

### Example 6: Using CH1/Cλ, CH/Cλ, MH2(MH2a/MH2b), or EH2(EH2a/EH2b) Dimer to Replace Cα/Cβ Heterodimer to Build TCR-Ig

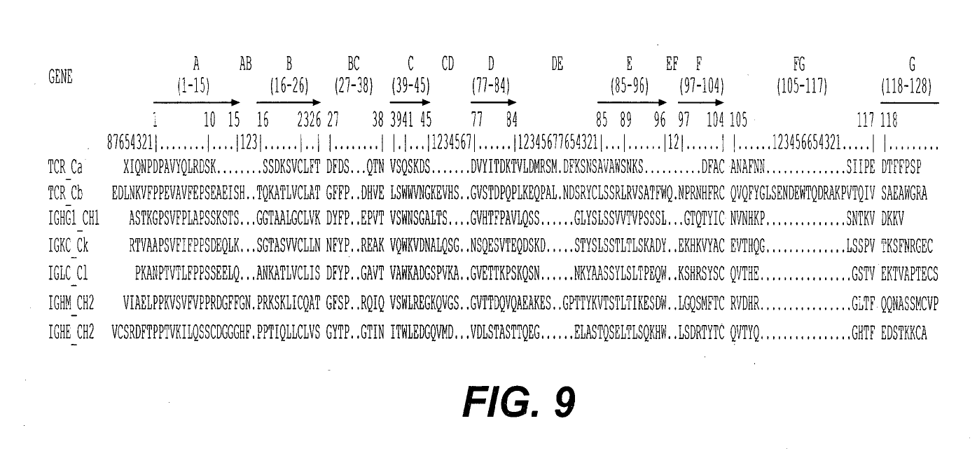

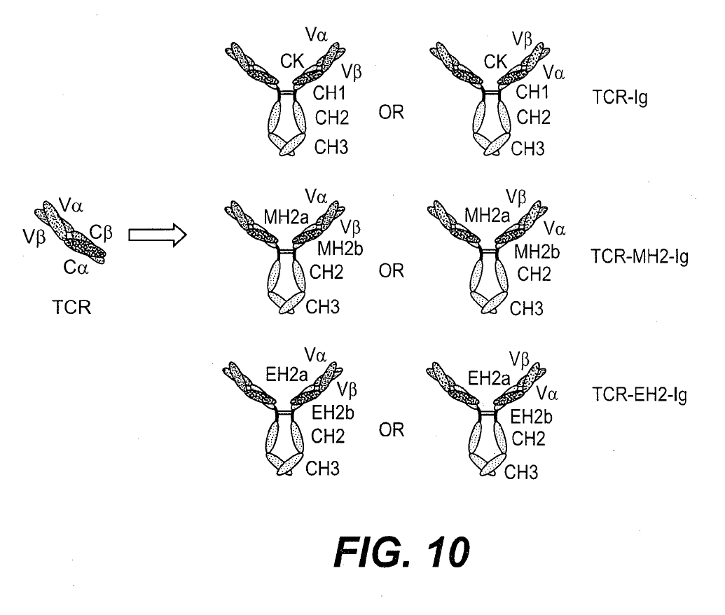

T cell receptor (TCR) Cα and Cβ have similar anti-parallel β sheet Ig fold structure to IgG CH1, Cκ, Cλ, MH2, and EH2, as shown in FIG. 9. Cα and Cβ domains are stabilized by an intra-chain disulfide bond between cysteine residues 23 and 104. There is no inter-chain disulfide bond between TCR Cα and Cβ. Replacing a Cα/Cβ dimer by a CH1/κ(λ) dimer, an MH2 homo- or engineered hetero-dimer, or an EH2 homo- or engineered hetero-dimer to support Vα/Vβ brings in one or two extra inter-chain disulfide bond(s) to stabilize the pairing between Vα and Vβ. As shown in FIG. 10, IgG-like molecules with Vα/Vβ domains combine the TCR binding ability with the effector function and half-life of regular IgG.

### Example 7: Using MH2 or EH2 Dimer to Stabilize DVD-Ig Outer or Inner Variable Domain

As shown in FIG. 11, an MH2 or EH2 hetero-dimer can be used to stabilize the outer or inner variable domains in a DVD-Ig format with a linker on either chain, or on both the heavy and light chain. The linker is a regular or cleavable linker. The inner and outer variable domains are antibody and/or T cell receptor variable domains.

### Example 8: MH2 or EH2 Dimer is Used as a Dimerization Building Block to Build Bi-/Multi-Specific Molecules with Antibody Variable Domains and/or T Cell Receptor Domains

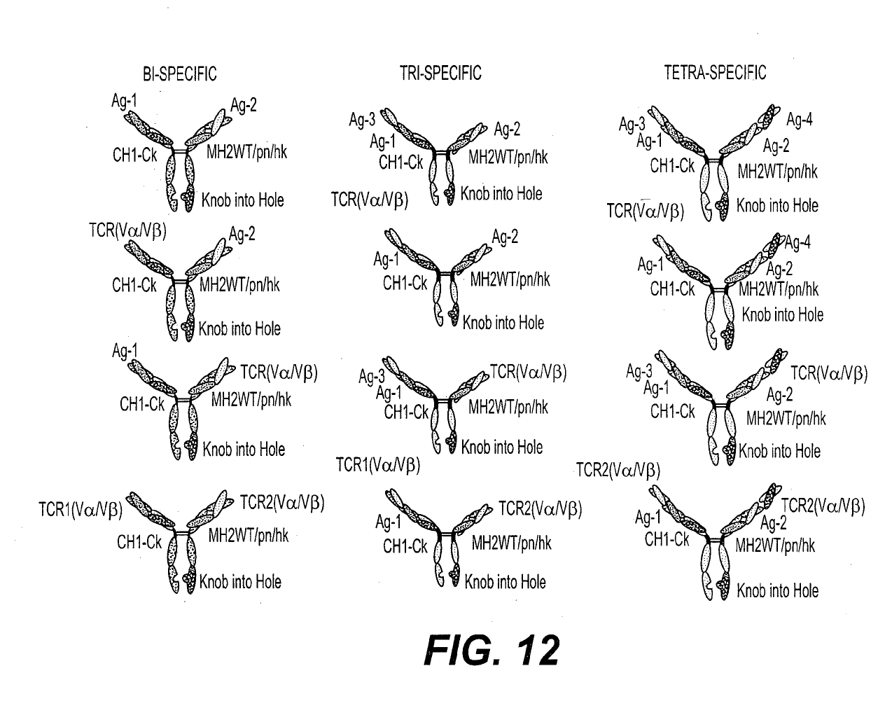

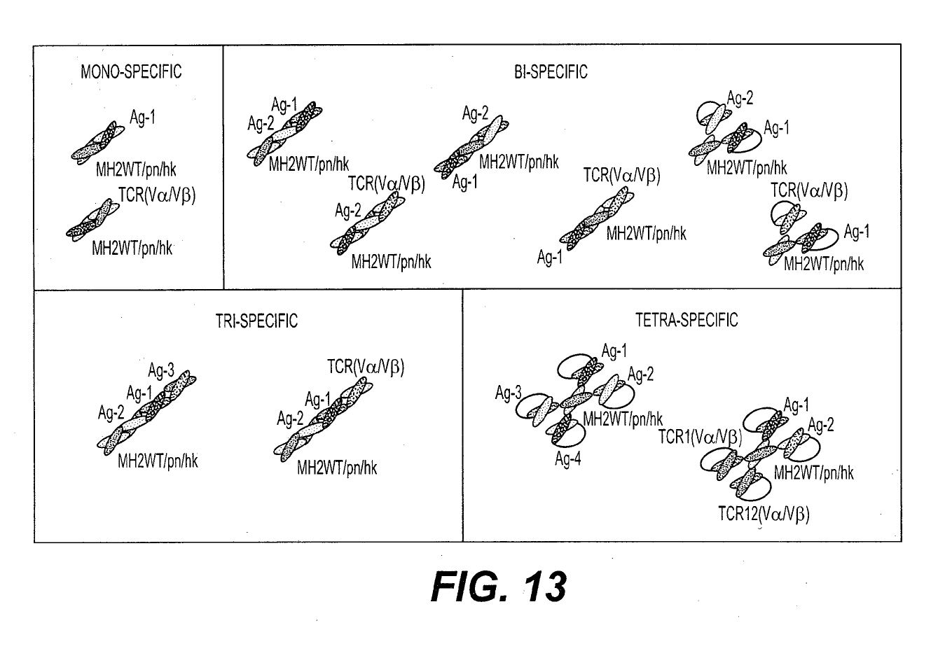

As shown in FIG. 12, an MH2 or EH2 homo- or engineered hetero-dimer is used as a dimerization building block in conjunction with heavy chain hetero-dimerization approaches to build bi-, tri-, and tetra-specific IgG-like molecules with antibody variable domains VH/VL and/or T cell receptor Vα/Vβ domains. Knobs-into-holes techniques are used here as an example of a heavy chain hetero-dimerization approach. FIG. 13 lists mono-, bi-, tri-, and tetra-specific fragment molecules using an MH2/EH2 homo- or engineered hetero-dimer as a building block. Molecular valency and specificity may be modulated by using the same or different antigen binding domains.

### Example 9: Generation of IgG-Like Molecules with MH2 Domains

Table 2 summarizes exemplary sequences of building blocks that may be used to build IgG-like molecules containing wild type or modified IgG and/or IgM domains. Knobs-into-holes technology or other methods listed in Table 1 can be utilized to enhance heavy chain hetero-dimerization.

TABLE 2
Sequences of Building Blocks To Build IgG-Like Molecules Containing Wild
Type Or Engineered Hetero-Dimerization MH2 Domains

| | | SEQ |
|---|---|---|
| | Sequence | ID |
| Domain | 12345678901234567890123456789012345678901234567890 | NO: |
| MH2 | ELPPKVSVFVPPRDGFFGNPRKSKLICQATGFSPRQIQVSWLREGKQVGS | 34 |
| | GVTTDQVQAEAKESGPTTYKVTSTLTIKESDWLGQSMFTCRVDHRGLTFQ | |
| | QNASSMC | |
| MH2p | ELPPKVSVFVPPRDGFFGNPRKSKLICKATGFSPRQIQVSWLREGKQVGS | 35 |
| | GVTTKQVQAEAKESGPTTYKVTSTLTIKESDWLGQSMFTCRVDHRGLTFQ | |
| | QNASSMC | |
| MH2n | ELPPKVSVFVPPRDGFFGNPRKSELICEATGFSPRQIQVSWLREGKQVGS | 36 |
| | GVTTDQVQAEAKESGPTTYDVTSTLTIKESDWLGQSMFTCRVDHRGLTFQ | |
| | QNASSMC | |
| MH2k | ELPPKVSVFVPPRDGFFGNPRKSKLWCQATGFSPRQIQVSWLREGKQVGS | 37 |
| | GVTTDQVQAEAKESGPTTYKVTSTLTIKESDWLGQSMFTCRVDHRGLTFQ | |
| | QNASSMC | |
| MH2h | ELPPKVSVFVPPRDGFFGNPRKSKLACSATGFSPRQIQVSWLREGKQVGS | 38 |
| | GVTTDQVQAEAKESGPTTYKVASTLTIKESDWLGQSMFTCRVDHRGLTFQ | |
| | QNASSMC | |
| MH2.S | ELPPKVSVFVPPRDGFFGNPRKSKLICQATGFSPRQIQVSWLREGKQVGS | 39 |
| | GVTTDQVQAEAKESGPTTYKVTSTLTIKESDWLGQSMFTCRVDHRGLTFQ | |
| | QSASSMC | |
| MH2p.S | ELPPKVSVFVPPRDGFFGNPRKSKLICKATGFSPRQIQVSWLREGKQVGS | 40 |
| | GVTTKQVQAEAKESGPTTYKVTSTLTIKESDWLGQSMFTCRVDHRGLTFQ | |
| | QSASSMC | |
| MH2n.S | ELPPKVSVFVPPRDGFFGNPRKSELICEATGFSPRQIQVSWLREGKQVGS | 41 |
| | GVTTDQVQAEAKESGPTTYDVTSTLTIKESDWLGQSMFTCRVDHRGLTFQ | |
| | QSASSMC | |
| MH2k.S | ELPPKVSVFVPPRDGFFGNPRKSKLWCQATGFSPRQIQVSWLREGKQVGS | 42 |
| | GVTTDQVQAEAKESGPTTYKVTSTLTIKESDWLGQSMFTCRVDHRGLTFQ | |
| | QSASSMC | |
| MH2h.S | ELPPKVSVFVPPRDGFFGNPRKSKLACSATGFSPRQIQVSWLREGKQVGS | 43 |
| | GVTTDQVQAEAKESGPTTYKVASTLTIKESDWLGQSMFTCRVDHRGLTFQ | |
| | QSASSMC | |
| CH1 | ASTKGPSVFPLAPSSKSTSGGTAALGCLVKDYFPEPVTVSWNSGALTSGV | 44 |
| | HTFPAVLQSSGLYSLSSVVTVPSSSLGTQTYICNVNHKPSNTKVDKKV | |
| Cκ | RTVAAPSVFIFPPSDEQLKSGTASVVCLLNNFYPREAKVQWKVDNALQSG | 45 |
| | NSQESVTEQDSKDSTYSLSSTLTLSKADYEKHKVYACEVTHQGLSSPVTK | |
| | SFNRGEC | |
| hinge after | EPKSCDKTHTCPPCP | 32 |
| CH1 | | |
| hinge after | DKTHTCPPCP | 33 |
| MH2/p/n/k/h | | |
| CH2 | APELLGGPSVFLFPPKPKDTLMISRTPEVTCVVVDVSHEDPEVKFNWYVD | 46 |
| | GVEVHNAKTKPREEQYNSTYRVVSVLTVLHQDWLNGKEYKCKVSNKALPA | |
| | PIEKTISKAK | |
| CH2(LALA) | APEAAGGPSVFLFPPKPKDTLMISRTPEVTCVVVDVSHEDPEVKFNWYVD | 47 |
| | GVEVHNAKTKPREEQYNSTYRVVSVLTVLHQDWLNGKEYKCKVSNKALPA | |
| | PIEKTISKAK | |
| CH3 | GQPREPQVYTLPPSREEMTKNQVSLTCLVKGFYPSDIAVEWESNGQPENN | 48 |
| | YKTTPPVLDSDGSFFLYSKLTVDKSRWQQGNVFSCSVMHEALHNHYTQKS | |
| | LSLSPGK | |
| CH3(knobs) | GQPREPQVYTLPPSREEMTKNQVSLWCLVKGFYPSDIAVEWESNGQPENN | 49 |
| | YKTTPPVLDSDGSFFLYSKLTVDKSRWQQGNVFSCSVMHEALHNHYTQKS | |
| | LSLSPGK | |
| CH3(holes) | GQPREPQVYTLPPSREEMTKNQVSLSCAVKGFYPSDIAVEWESNGQPENN | 50 |
| | YKTTPPVLDSDGSFFLVSKLTVDKSRWQQGNVFSCSVMHEALHNHYTQKS | |
| | LSLSPGK | |
| CH3(halfbody) | GQPREPQVYTLPPSREEMTKNQVSLTCLVKGFYPSDIAVEWESNGQPENN | 51 |
| | YKTTPPVLDSDGSFRLYSKLTVDKSRWQQGNVFSCSVMHEALHNHYTQKS | |
| | LSLSPGK | |
| linker | AST | |
| between VH | | |
| and | | |
| MH2/p/n/k/h | | |
| linker | RTA | |
| between VL | | |
| and | | |
| MH2/p/n/k/h | | |

### Example 9.1: Generation of Monospecific Molecules with Wild Type MH2 Homodimer or Engineered MH2a/MH2b Heterodimer

As described in Example 3.2 and Example 6, a monospecific IgG-like molecule with CH1/Cκ replaced by wild type MH2 homodimer or MH2a/MH2b heterodimer may be generated by two chain transfection. Variable domains may be VH or VL domains from an antibody, or Vα or Vβ from a TCR. VH and VL are paired to bind to specific antigens. Vα and Vβ are paired to bind to specific peptides. Table 3 summarizes 5 exemplary combinations to build bivalent mono-specific molecules using wild type or engineered MH2 dimers with variable domains from antibodies or TCRs.

TABLE 3
Generation Of Monospecific IgG-Molecules Containing
A Wild Type Or Engineered MH2 Dimer With Variable
Domains From Antibody Or TCR

Monospecific IgG -Like Molecule

| | Chain 1 | Chain 2 |
|---|---|---|
| 1 | VH-linker-MH2-hinge-CH2—CH3 | VL-linker-MH2 |
| 2 | VH-linker-MH2p-hinge-CH2—CH3 | VL-linker-MH2n |
| 3 | VH-linker-MH2n-hinge-CH2—CH3 | VL-linker-MH2p |
| 4 | VH-linker-MH2h-hinge-CH2—CH3 | VL-linker-MH2k |
| 5 | VH-linker-MH2k-hinge-CH2—CH3 | VL-linker-MH2h |

Three bivalent mono-specific molecules with CH1/Cκ replaced by MH2n/MH2p were generated. The variable domains used to generate these molecules are summarized in Table 4, which are from an anti-CD3 antibody (AB596), an anti-TNFa antibody (D2E7), and an anti-HER2 antibody (Herceptin).

TABLE 4
Antibody Variable Domains Used To Build Monospecific Molecules Containing
MH2 Domains

| | | | SEQ |
|---|---|---|---|
| Antibody | | Variable Domain Sequence | ID |
| Target | Domain | 12345678901234567890123456789012345678901234567890 | NO: |
| CD3 | AB596VH | EVTLKESGPVLVKPTETLTLTCTVSGFSLTSFGVSWVRQPPGKGLEWIAA | 52 |
| (AB596) | | IWNSGRMDYNSALKSRLTISRDTSKSQVVLTMTNMDPVDTAMYFCARSGG | |
| | | SHWGQGTLVTVSS | |
| | AB596VK | DIQMTQSPSSLSASVGDRVTITCKPSQNIDKYLNWYQQKPGKAPKRLIYN | 53 |
| | | TNNLQTGIPSRFSGSGSGTEYTLTISSLQPEDFATYFCLQHRSGWTFGGG | |
| | | TKVEIK | |
| TNFa | D2E7VH | EVQLVESGGGLVQPGRSLRLSCAASGFTFDDYAMHWVRQAPGKGLEWVSA | 54 |
| (D2E7) | | ITWNSGHIDYADSVEGRFTISRDNAKNSLYLQMNSLRAEDTAVYYCAKVS | |
| | | YLSTASSLDYWGQGTLVTVSS | |
| | D2E7VK | DIQMTQSPSSLSASVGDRVTITCRASQGIRNYLAWYQQKPGKAPKLLIYA | 55 |
| | | ASTLQSGVPSRFSGSGSGTDFTLTISSLQPEDVATYYCQRYNRAPYTFGQ | |
| | | GTKVEIK | |
| HER2 | Herceptin | EVQLVESGGGLVQPGGSLRLSCAASGFNIKDTYIHWVRQAPGKGLEWVAR | 56 |
| (Herceptin) | VH | IYPTNGYTRYADSVKGRFTISADTSKNTAYLQMNSLRAEDTAVYYCSRWG | |
| | | GDGFYAMDYWGQGTLVTVSS | |
| | Herceptin | DIQMTQSPSSLSASVGDRVTITCRASQDVNTAVAWYQQKPGKAPKLLIYS | 57 |
| | VK | ASFLYSGVPSRFSGSRSGTDFTLTISSLQPEDFATYYCQQHYTTPPTFGQ | |
| | | GTKVEIK | |

The MH2n and MH2p domains were synthesized by Integrated DNA Technologies. MH2n was incorporated into a heavy chain to replace CH1 while MH2p was incorporated into a light chain to replace Cκ. A Sal I restriction site was introduced to the 5′ end of the MH2 for constructing the MH2 heavy chain vector, and a BsiW I site for the MH2 light chain vector. Two plasmid vectors were used for the transfection of each bivalent mono-specific MH2 molecule. The sequence of each molecule is summarized in the Table 5.

TABLE 5
Sequences of Generated Bivalent Monospecific Molecules Containing MH2
Domains

| Mono- | | | SEQ |
|---|---|---|---|
| specific | | Sequence | ID |
| molecules | Chain | 12345678901234567890123456789012345678901234567890 | NO |
| AB596- | AB596VH- | EVTLKESGPVLVKPTETLTLTCTVSGFSLTSFGVSWVRQPPGKGLEWIAA | 58 |
| MH2n/p | linker- | IWNSGRMDYNSALKSRLTISRDTSKSQVVLTMTNMDPVDTAMYFCARSGG | |
| | MH2n- | SHWGQGTLVTVSSASTELPPKVSVFVPPRDGFFGNPRKSELICEATGFSP | |
| | Hinge- | RQIQVSWLREGKQVGSGVTTDQVQAEAKESGPTTYDVTSTLTIKESDWLG | |
| | CH2-CH3 | QSMFTCRVDHRGLTFQQNASSMCDKTHTCPPCPAPEAAGGPSVFLFPPKP | |
| | | KDTLMISRTPEVTCVVVDVSHEDPEVKFNWYVDGVEVHNAKTKPREEQYN | |
| | | STYRVVSVLTVLHQDWLNGKEYKCKVSNKALPAPIEKTISKAKGQPREPQ | |
| | | VYTLPPSREEMTKNQVSLWCLVKGFYPSDIAVEWESNGQPENNYKTTPPV | |
| | | LDSDGSFFLYSKLTVDKSRWQQGNVFSCSVMHEALHNHYTQKSLSLSPGK | |
| | AB596VL- | DIQMTQSPSSLSASVGDRVTITCKPSQNIDKYLNWYQQKPGKAPKRLIYN | 59 |
| | linker- | TNNLQTGIPSRFSGSGSGTEYTLTISSLQPEDFATYFCLQHRSGWTFGGG | |
| | MH2p | TKVEIKRTAELPPKVSVFVPPRDGFFGNPRKSKLICKATGFSPRQIQVSW | |
| | | LREGKQVGSGVTTKQVQAEAKESGPTTYKVTSTLTIKESDWLGQSMFTCR | |
| | | VDHRGLTFQQNASSMC | |
| D2E7- | D2E7VH- | EVQLVESGGGLVQPGRSLRLSCAASGFTFDDYAMHWVRQAPGKGLEWVSA | 60 |
| MH2n/p | linker- | ITWNSGHIDYADSVEGRFTISRDNAKNSLYLQMNSLRAEDTAVYYCAKVS | |
| | MH2n- | YLSTASSLDYWGQGTLVTVSSASTELPPKVSVFVPPRDGFFGNPRKSELI | |
| | Hinge- | CEATGFSPRQIQVSWLREGKQVGSGVTTDQVQAEAKESGPTTYDVTSTLT | |
| | CH2-CH3 | IKESDWLGQSMFTCRVDHRGLTFQQNASSMCDKTHTCPPCPAPEAAGGPS | |
| | | VFLFPPKPKDTLMISRTPEVTCVVVDVSHEDPEVKFNWYVDGVEVHNAKT | |
| | | KPREEQYNSTYRVVSVLTVLHQDWLNGKEYKCKVSNKALPAPIEKTISKA | |
| | | KGQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQK | |
| | | SLSLSPGK | |
| | D2E7VL- | DIQMTQSPSSLSASVGDRVTITCRASQGIRNYLAWYQQKPGKAPKLLIYA | 61 |
| | linker- | ASTLQSGVPSRFSGSGSGTDFTLTISSLQPEDVATYYCQRYNRAPYTFGQ | |
| | MH2p | GTKVEIKRTAELPPKVSVFVPPRDGFFGNPRKSKLICKATGFSPRQIQVS | |
| | | WLREGKQVGSGVTTKQVQAEAKESGPTTYKVTSTLTIKESDWLGQSMFTC | |
| | | RVDHRGLTFQQNASSMC | |
| Herceptin- | Herceptin | EVQLVESGGGLVQPGGSLRLSCAASGFNIKDTYIHWVRQAPGKGLEWVAR | 62 |
| MH2n/p | VH- | IYPTNGYTRYADSVKGRFTISADTSKNTAYLQMNSLRAEDTAVYYCSRWG | |
| | linker- | GDGFYAMDYWGQGTLVTVSSASTELPPKVSVFVPPRDGFFGNPRKSELIC | |
| | MH2n- | EATGFSPRQIQVSWLREGKQVGSGVTTDQVQAEAKESGPTTYDVTSTLTI | |
| | Hinge- | KESDWLGQSMFTCRVDHRGLTFQQNASSMCDKTHTCPPCPAPEAAGGPSV | |
| | CH2-CH3 | FLFPPKPKDTLMISRTPEVTCVVVDVSHEDPEVKFNWYVDGVEVHNAKTK | |
| | | PREEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPENN | |
| | | YKTTPPVLDSDGSFFLYSKLTVDKSRWQQGNVFSCSVMHEALHNHYTQKS | |
| | | LSLSPGK | |
| | Herceptin | DIQMTQSPSSLSASVGDRVTITCRASQDVNTAVAWYQQKPGKAPKLLIYS | 63 |
| | VL- | ASFLYSGVPSRFSGSRSGTDFTLTISSLQPEDFATYYCQQHYTTPPTFGQ | |
| | linker- | GTKVEIKRTAELPPKVSVFVPPRDGFFGNPRKSKLICKATGFSPRQIQVS | |
| | MH2p | WLREGKQVGSGVTTKQVQAEAKESGPTTYKVTSTLTIKESDWLGQSMFTC | |
| | | RVDHRGLTFQQNASSMC | |

All molecules were expressed in HEK293 cells and purified with MabSelect SuRe beads. Their molecular profiles were analyzed by SEC as shown in Table 6.

TABLE 6
Expression Of Bivalent Monospecific
Molecules Containing MH2 Domains

| | Octet Titer | Yield | SEC |
|---|---|---|---|
| Antibody ID | (mg/L) | (mg/L) | Monomer % |
| AB596-MH2n/p | 6.2 | 5.8 | 84.35 |
| D2E7-MH2n/p | 10.1 | 7.3 | 91.1 |
| Herceptin-MH2n/p | 7.4 | 8.5 | 80.8 |

### Example 9.2: Bivalent Monospecific Molecules Containing MH2 Domains Maintain the Binding Specificity and Affinity of their Parental Antibodies

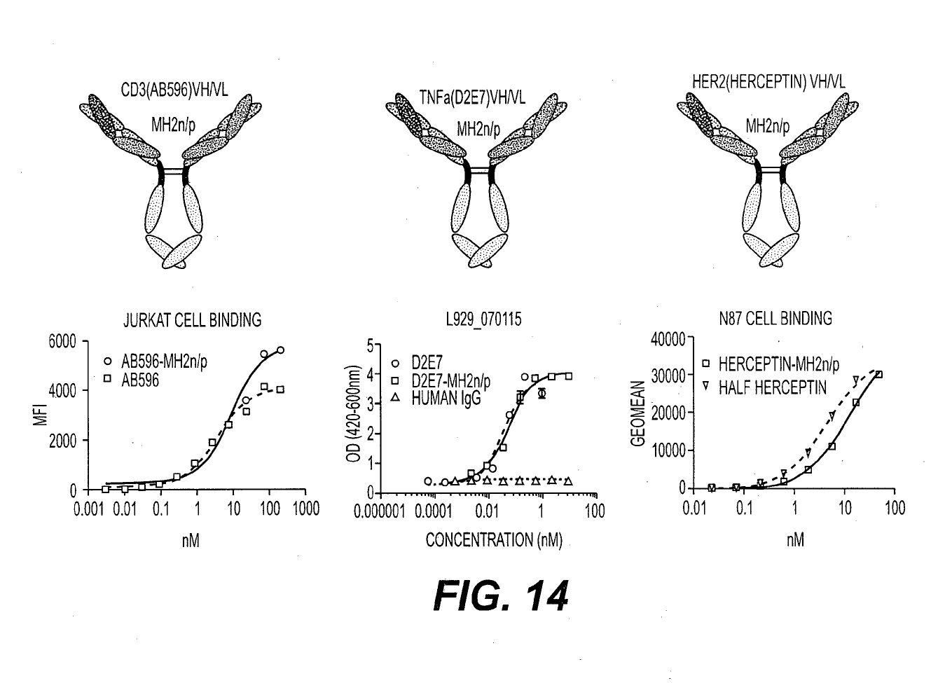

The bivalent mono-specific molecules listed in Table 5 were tested in a FACS binding assay. Jurkat cells were used for testing CD3 binding. L929 cells were used for testing TNFα binding. N87 cells were used for testing HER2 binding. Cells were incubated with different concentrations of antibodies for 30 minutes and then incubated with fluorescence-conjugated secondary antibodies for another 30 minutes. Cells were then analyzed by flow cytometry and data was analyzed using FlowJo software. As shown in FIG. 14, bivalent mono-specific MH2n/p molecules retained binding affinity to the specific target comparable to the parental antibody.

### Example 9.3: Generation of Monospecific Molecules with Non-Glycosylated Wild Type or Engineered MH2 Domains

As described in Example 4, there is a glycosylation site at position 120 on wild type and engineered MH2 domains. Multiple mutations to remove this glycosylation site have been evaluated on the Herceptin-MH2n/p molecule. The expression levels of the mutated molecules are comparable with the wild type molecules as shown in Table 7. In addition to mutating Asparagine at position 120, the non-Serine or Threonine mutation at position 122 also can eliminate a glycosylation site in the MH2 domain Alanine mutation was evaluated on a D2E7-MH2n/p molecule. The glycosylation sites were removed without impact on binding properties. Using a non-glycosylated MH2 domain to replace a glycosylated MH2 domain modulates the number of additional glycosylation sites (0-4) introduced by a MH2 domain.

TABLE 7
Expression Titer of the Non-Glycosylated Bivalent
Monospecific Molecules Containing MH2 Domains

| μg/ml | LC WT | LC N-A | LC N-G | LC N-S | LC N-Q |
|---|---|---|---|---|---|
| HC WT | 0.165 | 0.176 | 0.165 | 0.188 | 0.165 |
| HC N-A | 0.259 | 0.188 | 0.165 | 0.165 | 0.165 |
| HC N-G | 0.282 | 0.165 | 0.165 | 0.165 | 0.176 |
| HC N-S | 1.000 | 0.224 | 0.165 | 0.188 | 0.165 |
| HC N-Q | 0.965 | 0.294 | 0.165 | 0.212 | 0.165 |

### Example 10: Generation of Bispecific Molecules with Wild Type MH2 Homodimer or Engineered MH2a/MH2b Heterodimer

Table 8 summarizes 10 possible combinations to build bispecific molecules using MH2/MH2, MH2p/MH2n, or MH2k/MH2h domains in a knobs-into-holes format. In each, VH1 and VL1 are from one antibody, while VH2 and VL2 are from another antibody. Each bispecific molecule is generated with four chains: 2 heavy chains (chain 1 and chain 3) and 2 light chains (chain 2 and chain 4).

TABLE 8
Combinations of 4 Chains To Generate Bispecific
Molecules Using MH2/MH2, MH2p/MH2n, Or MH2k/MH2h
Dimer In Knobs-Into-Holes Format

| | Bispecific IgG Chain Combination | | | |
|---|---|---|---|---|
| | Chain 1 | Chain 2 | Chain 3 | Chain 4 |
| 1 | VH1-linker- | VL1-linker- | VH2-CH1-hinge- | VL2-Cκ (λ) |
| | MH2-hinge- | MH2 | CH2—CH3 | |
| | CH2—CH3 | | (holes) | |
| | (knobs) | | | |
| 2 | VH1-linker- | VL1-linker- | VH2-CH1-hinge- | VL2- Cκ (λ) |
| | MH2p-hinge- | MH2n | CH2—CH3 | |
| | CH2—CH3 | | (holes) | |
| | (knobs) | | | |
| 3 | VH1-linker- | VL1-linker- | VH2-CH1-hinge- | VL2- Cκ (λ) |
| | MH2n-hinge- | MH2p | CH2—CH3 | |
| | CH2—CH3 | | (holes) | |
| | (knobs) | | | |
| 4 | VH1-linker- | VL1-linker- | VH2-CH1-hinge- | VL2- Cκ (λ) |
| | MH2k-hinge- | MH2h | CH2—CH3 | |
| | CH2—CH3 | | (holes) | |
| | (knobs) | | | |
| 5 | VH1-linker- | VL1-linker- | VH2-CH1-hinge- | VL2- Cκ (λ) |
| | MH2h-hinge- | MH2k | CH2—CH3 | |
| | CH2—CH3 | | (holes) | |
| | (knobs) | | | |
| 6 | VH1-linker- | VL1-linker- | VH2-CH1-hinge- | VL2- Cκ (λ) |
| | MH2-hinge- | MH2 | CH2—CH3 | |
| | CH2—CH3 | | (knobs) | |
| | (holes) | | | |
| 7 | VH1-linker- | VL1-linker- | VH2-CH1-hinge- | VL2- Cκ (λ) |
| | MH2p-hinge- | MH2n | CH2—CH3 | |
| | CH2—CH3 | | (knobs) | |
| | (holes) | | | |
| 8 | VH1-linker- | VL1-linker- | VH2-CH1-hinge- | VL2- Cκ (λ) |
| | MH2n-hinge- | MH2p | CH2—CH3 | |
| | CH2—CH3 | | (knobs) | |
| | (holes) | | | |
| 9 | VH1-linker- | VL1-linker- | VH2-CH1-hinge- | VL2- Cκ (λ) |
| | MH2k-hinge- | MH2h | CH2—CH3 | |
| | CH2—CH3 | | (knobs) | |
| | (holes) | | | |
| 10 | VH1-linker- | VL1-linker- | VH2-CH1-hinge- | VL2- Cκ (λ) |
| | MH2h-hinge- | MH2k | CH2—CH3 | |
| | CH2—CH3 | | (knobs) | |
| | (holes) | | | |

### Example 10.1: Generation of Bispecific Molecules with Wild Type MH2 Homodimer or Engineered MH2a/MH2b Heterodimer

Five bispecific molecules were generated based on the chain combinations 1-5 listed in Table 8, where VH1 and VL1 are from one anti-HER2 antibody Herceptin (Herceptin VH and Herceptin VK), and VH2 and VL2 are from an anti-EGFR antibody Cetuximab (Cetuximab VH and Cetuximab VL), listed in Table 9. One original knobs-into-holes bispecific antibody, and one anti-Her2 Herceptin halfbody were also generated for comparison. Table 10 summarizes the sequence of 4 chains for each of the bispecific molecules and halfbodies that have been generated.

TABLE 9
Antibody Variable Domains Used To Build Bispecific Molecules Containing MH2
Domains

| | | | SEQ |
|---|---|---|---|
| Antibody | | Variable Domain Sequence | ID |
| Target | Domain | 12345678901234567890123456789012345678901234567890 | NO: |
| HER2 | Herceptin | EVQLVESGGGLVQPGGSLRLSCAASGFNIKDTYIHWVRQAPGKGLEWVAR | 64 |
| (Herceptin) | VH | IYPTNGYTRYADSVKGRFTISADTSKNTAYLQMNSLRAEDTAVYYCSRWG | |
| | | GDGFYAMDYWGQGTLVTVSS | |
| | Herceptin | DIQMTQSPSSLSASVGDRVTITCRASQDVNTAVAWYQQKPGKAPKLLIYS | 65 |
| | VK | ASFLYSGVPSRFSGSRSGTDFTLTISSLQPEDFATYYCQQHYTTPPTFGQ | |
| | | GTKVEIK | |
| EGFR | Cetuximab | QVQLKQSGPGLVQPSQSLSITCTVSGFSLTNYGVHWVRQSPGKGLEWLGV | 66 |
| (Cetuximab) | VH | IWSGGNTDYNTPFTSRLSINKDNSKSQVFFKMNSLQSNDTAIYYCARALT | |
| | | YYDYEFAYWGQGTLVTVSA | |
| | Cetuximab | DILLTQSPVILSVSPGERVSFSCRASQSIGTNIHWYQQRTNGSPRLLIKY | 67 |
| | VK | ASESISGIPSRFSGSGSGTDFTLSINSVESEDIADYYCQQNNNWPTTFGA | |
| | | GTKLELK | |

TABLE 10
Sequences Of Generated Bispecific Molecules containing MH2 domains And Halfbodies

| | | | SEQ |
|---|---|---|---|
| Bispecific | | Sequence | ID |
| Molecules | Chain | 12345678901234567890123456789012345678901234567890 | NO: |
| BMH6 | Herceptin | EVQLVESGGGLVQPGGSLRLSCAASGFNIKDTYIHWVRQAPGKGLEWVAR | 68 |
| | VH-linker- | IYPTNGYTRYADSVKGRFTISADTSKNTAYLQMNSLRAEDTAVYYCSRWG | |
| | MH2-hinge- | GDGFYAMDYWGQGTLVTVSSASTELPPKVSVFVPPRDGFFGNPRKSKLIC | |
| | CH2- | QATGFSPRQIQVSWLREGKQVGSGVTTDQVQAEAKESGPTTYKVTSTLTI | |
| | CH3(knobs) | KESDWLGQSMFTCRVDHRGLTFQQNASSMCDKTHTCPPCPAPELLGGPSV | |
| | | FLFPPKPKDTLMISRTPEVTCVVVDVSHEDPEVKFNWYVDGVEVHNAKTK | |
| | | PREEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPENN | |
| | | YKTTPPVLDSDGSFFLYSKLTVDKSRWQQGNVFSCSVMHEALHNHYTQKS | |
| | | LSLSPGK | |
| | Herceptin | DIQMTQSPSSLSASVGDRVTITCRASQDVNTAVAWYQQKPGKAPKLLIYS | 69 |
| | VK-linker- | ASFLYSGVPSRFSGSRSGTDFTLTISSLQPEDFATYYCQQHYTTPPTFGQ | |
| | MH2 | GTKVEIKRTAELPPKVSVFVPPRDGFFGNPRKSKLICQATGFSPRQIQVS | |
| | | WLREGKQVGSGVTTDQVQAEAKESGPTTYKVTSTLTIKESDWLGQSMFTC | |
| | | RVDHRGLTFQQNASSMC | |
| | Cetuximab | QVQLKQSGPGLVQPSQSLSITCTVSGFSLTNYGVHWVRQSPGKGLEWLGV | 70 |
| | VH-CH1- | IWSGGNTDYNTPFTSRLSINKDNSKSQVFFKMNSLQSNDTAIYYCARALT | |
| | hinge-CH2- | YYDYEFAYWGQGTLVTVSAASTKGPSVFPLAPSSKSTSGGTAALGCLVKD | |
| | CH3(holes) | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSREEMTKNQVSLSCAVKGFYPSDIAVEWESNGQPENNYKTTPPVL | |
| | | DSDGSFFLVSKLTVDKSRWQQGNVFSCSVMHEALHNHYTQKSLSLSPGK | |
| | Cetuximab | DILLTQSPVILSVSPGERVSFSCRASQSIGTNIHWYQQRTNGSPRLLIKY | 71 |
| | VK-Cκ | ASESISGIPSRFSGSGSGTDFTLSINSVESEDIADYYCQQNNNWPTTFGA | |
| | | GTKLELKRTVAAPSVFIFPPSDEQLKSGTASVVCLLNNFYPREAKVQWKV | |
| | | DNALQSGNSQESVTEQDSKDSTYSLSSTLTLSKADYEKHKVYACEVTHQG | |
| | | LSSPVTKSFNRGEC | |
| BMH7 | HerceptinVH- | EVQLVESGGGLVQPGGSLRLSCAASGFNIKDTYIHWVRQAPGKGLEWVAR | 72 |
| | linker-MH2n- | IYPTNGYTRYADSVKGRFTISADTSKNTAYLQMNSLRAEDTAVYYCSRWG | |
| | hinge-CH2- | GDGFYAMDYWGQGTLVTVSSASTELPPKVSVFVPPRDGFFGNPRKSELIC | |
| | CH3(knobs) | EATGFSPRQIQVSWLREGKQVGSGVTTDQVQAEAKESGPTTYDVTSTLTI | |
| | | KESDWLGQSMFTCRVDHRGLTFQQNASSMCDKTHTCPPCPAPELLGGPSV | |
| | | FLFPPKPKDTLMISRTPEVTCVVVDVSHEDPEVKFNWYVDGVEVHNAKTK | |
| | | PREEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPENN | |
| | | YKTTPPVLDSDGSFFLYSKLTVDKSRWQQGNVFSCSVMHEALHNHYTQKS | |
| | | LSLSPGK | |
| | Herceptin | DIQMTQSPSSLSASVGDRVTITCRASQDVNTAVAWYQQKPGKAPKLLIYS | 73 |
| | VK-linker- | ASFLYSGVPSRFSGSRSGTDFTLTISSLQPEDFATYYCQQHYTTPPTFGQ | |
| | MH2p | GTKVEIKRTAELPPKVSVFVPPRDGFFGNPRKSKLICKATGFSPRQIQVS | |
| | | WLREGKQVGSGVTTKQVQAEAKESGPTTYKVTSTLTIKESDWLGQSMFTC | |
| | | RVDHRGLTFQQNASSMC | |
| | Cetuximab | QVQLKQSGPGLVQPSQSLSITCTVSGFSLTNYGVHWVRQSPGKGLEWLGV | 74 |
| | VH-CH1- | IWSGGNTDYNTPFTSRLSINKDNSKSQVFFKMNSLQSNDTAIYYCARALT | |
| | hinge-CH2- | YYDYEFAYWGQGTLVTVSAASTKGPSVFPLAPSSKSTSGGTAALGCLVKD | |
| | CH3(holes) | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSREEMTKNQVSLSCAVKGFYPSDIAVEWESNGQPENNYKTTPPVL | |
| | | DSDGSFFLVSKLTVDKSRWQQGNVFSCSVMHEALHNHYTQKSLSLSPGK | |
| | Cetuximab | DILLTQSPVILSVSPGERVSFSCRASQSIGTNIHWYQQRTNGSPRLLIKY | 75 |
| | VK-Cκ | ASESISGIPSRFSGSGSGTDFTLSINSVESEDIADYYCQQNNNWPTTFGA | |
| | | GTKLELKRTVAAPSVFIFPPSDEQLKSGTASVVCLLNNFYPREAKVQWKV | |
| | | DNALQSGNSQESVTEQDSKDSTYSLSSTLTLSKADYEKHKVYACEVTHQG | |
| | | LSSPVTKSFNRGEC | |
| BMH8 | Herceptin | EVQLVESGGGLVQPGGSLRLSCAASGFNIKDTYIHWVRQAPGKGLEWVAR | 76 |
| | VH-linker- | IYPTNGYTRYADSVKGRFTISADTSKNTAYLQMNSLRAEDTAVYYCSRWG | |
| | MH2p-hinge- | GDGFYAMDYWGQGTLVTVSSASTELPPKVSVFVPPRDGFFGNPRKSKLIC | |
| | CH2- | KATGFSPRQIQVSWLREGKQVGSGVTTKQVQAEAKESGPTTYKVTSTLTI | |
| | CH3(knobs) | KESDWLGQSMFTCRVDHRGLTFQQNASSMCDKTHTCPPCPAPELLGGPSV | |
| | | FLFPPKPKDTLMISRTPEVTCVVVDVSHEDPEVKFNWYVDGVEVHNAKTK | |
| | | PREEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPENN | |
| | | YKTTPPVLDSDGSFFLYSKLTVDKSRWQQGNVFSCSVMHEALHNHYTQKS | |
| | | LSLSPGK | |
| | Herceptin | DIQMTQSPSSLSASVGDRVTITCRASQDVNTAVAWYQQKPGKAPKLLIYS | 77 |
| | VK-linker- | ASFLYSGVPSRFSGSRSGTDFTLTISSLQPEDFATYYCQQHYTTPPTFGQ | |
| | MH2n | GTKVEIKRTAELPPKVSVFVPPRDGFFGNPRKSELICEATGFSPRQIQVS | |
| | | WLREGKQVGSGVTTDQVQAEAKESGPTTYDVTSTLTIKESDWLGQSMFTC | |
| | | RVDHRGLTFQQNASSMC | |
| | Cetuximab | QVQLKQSGPGLVQPSQSLSITCTVSGFSLTNYGVHWVRQSPGKGLEWLGV | 78 |
| | VH-CH1- | IWSGGNTDYNTPFTSRLSINKDNSKSQVFFKMNSLQSNDTAIYYCARALT | |
| | hinge-CH2- | YYDYEFAYWGQGTLVTVSAASTKGPSVFPLAPSSKSTSGGTAALGCLVKD | |
| | CH3(holes) | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSREEMTKNQVSLSCAVKGFYPSDIAVEWESNGQPENNYKTTPPVL | |
| | | DSDGSFFLVSKLTVDKSRWQQGNVFSCSVMHEALHNHYTQKSLSLSPGK | |
| | Cetuximab | DILLTQSPVILSVSPGERVSFSCRASQSIGTNIHWYQQRTNGSPRLLIKY | 79 |
| | VK-Cκ | ASESISGIPSRFSGSGSGTDFTLSINSVESEDIADYYCQQNNNWPTTFGA | |
| | | GTKLELKRTVAAPSVFIFPPSDEQLKSGTASVVCLLNNFYPREAKVQWKV | |
| | | DNALQSGNSQESVTEQDSKDSTYSLSSTLTLSKADYEKHKVYACEVTHQG | |
| | | LSSPVTKSFNRGEC | |
| BMH9 | Herceptin | EVQLVESGGGLVQPGGSLRLSCAASGFNIKDTYIHWVRQAPGKGLEWVAR | 80 |
| | VH-linker- | IYPTNGYTRYADSVKGRFTISADTSKNTAYLQMNSLRAEDTAVYYCSRWG | |
| | MH2h-hinge- | GDGFYAMDYWGQGTLVTVSSASTELPPKVSVFVPPRDGFFGNPRKSKLAC | |
| | CH2- | SATGFSPRQIQVSWLREGKQVGSGVTTDQVQAEAKESGPTTYKVASTLTI | |
| | CH3(knobs) | KESDWLGQSMFTCRVDHRGLTFQQNASSMCDKTHTCPPCPAPELLGGPSV | |
| | | FLFPPKPKDTLMISRTPEVTCVVVDVSHEDPEVKFNWYVDGVEVHNAKTK | |
| | | PREEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPENN | |
| | | YKTTPPVLDSDGSFFLYSKLTVDKSRWQQGNVFSCSVMHEALHNHYTQKS | |
| | | LSLSPGK | |
| | Herceptin | DIQMTQSPSSLSASVGDRVTITCRASQDVNTAVAWYQQKPGKAPKLLIYS | 81 |
| | VK-linker- | ASFLYSGVPSRFSGSRSGTDFTLTISSLQPEDFATYYCQQHYTTPPTFGQ | |
| | MH2k | GTKVEIKRTAELPPKVSVFVPPRDGFFGNPRKSKLWCQATGFSPRQIQVS | |
| | | WLREGKQVGSGVTTDQVQAEAKESGPTTYKVTSTLTIKESDWLGQSMFTC | |
| | | RVDHRGLTFQQNASSMC | |
| | Cetuximab | QVQLKQSGPGLVQPSQSLSITCTVSGFSLTNYGVHWVRQSPGKGLEWLGV | 82 |
| | VH-CH1- | IWSGGNTDYNTPFTSRLSINKDNSKSQVFFKMNSLQSNDTAIYYCARALT | |
| | hinge-CH2- | YYDYEFAYWGQGTLVTVSAASTKGPSVFPLAPSSKSTSGGTAALGCLVKD | |
| | CH3(holes) | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSREEMTKNQVSLSCAVKGFYPSDIAVEWESNGQPENNYKTTPPVL | |
| | | DSDGSFFLVSKLTVDKSRWQQGNVFSCSVMHEALHNHYTQKSLSLSPGK | |
| | Cetuximab | DILLTQSPVILSVSPGERVSFSCRASQSIGTNIHWYQQRTNGSPRLLIKY | 83 |
| | VK-Cκ | ASESISGIPSRFSGSGSGTDFTLSINSVESEDIADYYCQQNNNWPTTFGA | |
| | | GTKLELKRTVAAPSVFIFPPSDEQLKSGTASVVCLLNNFYPREAKVQWKV | |
| | | DNALQSGNSQESVTEQDSKDSTYSLSSTLTLSKADYEKHKVYACEVTHQG | |
| | | LSSPVTKSFNRGEC | |
| BMH10 | Herceptin | EVQLVESGGGLVQPGGSLRLSCAASGFNIKDTYIHWVRQAPGKGLEWVAR | 84 |
| | VH-linker- | IYPTNGYTRYADSVKGRFTISADTSKNTAYLQMNSLRAEDTAVYYCSRWG | |
| | MH2k-hinge- | GDGFYAMDYWGQGTLVTVSSASTELPPKVSVFVPPRDGFFGNPRKSKLWC | |
| | CH2- | QATGFSPRQIQVSWLREGKQVGSGVTTDQVQAEAKESGPTTYKVTSTLTI | |
| | CH3(knobs) | KESDWLGQSMFTCRVDHRGLTFQQNASSMCDKTHTCPPCPAPELLGGPSV | |
| | | FLFPPKPKDTLMISRTPEVTCVVVDVSHEDPEVKFNWYVDGVEVHNAKTK | |
| | | PREEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPENN | |
| | | YKTTPPVLDSDGSFFLYSKLTVDKSRWQQGNVFSCSVMHEALHNHYTQKS | |
| | | LSLSPGK | |
| | Herceptin | DIQMTQSPSSLSASVGDRVTITCRASQDVNTAVAWYQQKPGKAPKLLIYS | 85 |
| | VK-linker- | ASFLYSGVPSRFSGSRSGTDFTLTISSLQPEDFATYYCQQHYTTPPTFGQ | |
| | MH2h | GTKVEIKRTAELPPKVSVFVPPRDGFFGNPRKSKLACSATGFSPRQIQVS | |
| | | WLREGKQVGSGVTTDQVQAEAKESGPTTYKVASTLTIKESDWLGQSMFTC | |
| | | RVDHRGLTFQQNASSMC | |
| | Cetuximab | QVQLKQSGPGLVQPSQSLSITCTVSGFSLTNYGVHWVRQSPGKGLEWLGV | 86 |
| | VH-CH1- | IWSGGNTDYNTPFTSRLSINKDNSKSQVFFKMNSLQSNDTAIYYCARALT | |
| | hinge-CH2- | YYDYEFAYWGQGTLVTVSAASTKGPSVFPLAPSSKSTSGGTAALGCLVKD | |
| | CH3(holes) | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSREEMTKNQVSLSCAVKGFYPSDIAVEWESNGQPENNYKTTPPVL | |
| | | DSDGSFFLVSKLTVDKSRWQQGNVFSCSVMHEALHNHYTQKSLSLSPGK | |
| | Cetuximab | DILLTQSPVILSVSPGERVSFSCRASQSIGTNIHWYQQRTNGSPRLLIKY | 87 |
| | VK-Cκ | ASESISGIPSRFSGSGSGTDFTLSINSVESEDIADYYCQQNNNWPTTFGA | |
| | | GTKLELKRTVAAPSVFIFPPSDEQLKSGTASVVCLLNNFYPREAKVQWKV | |
| | | DNALQSGNSQESVTEQDSKDSTYSLSSTLTLSKADYEKHKVYACEVTHQG | |
| | | LSSPVTKSFNRGEC | |
| KIH2 | Herceptin | EVQLVESGGGLVQPGGSLRLSCAASGFNIKDTYIHWVRQAPGKGLEWVAR | 88 |
| | VH-CH1- | IYPTNGYTRYADSVKGRFTISADTSKNTAYLQMNSLRAEDTAVYYCSRWG | |
| | hinge-CH2- | GDGFYAMDYWGQGTLVTVSSASTKGPSVFPLAPSSKSTSGGTAALGCLVK | |
| | CH3(knobs) | DYFPEPVTVSWNSGALTSGVHTFPAVLQSSGLYSLSSVVTVPSSSLGTQT | |
| | | YICNVNHKPSNTKVDKKVEPKSCDKTHTCPPCPAPELLGGPSVFLFPPKP | |
| | | KDTLMISRTPEVTCVVVDVSHEDPEVKFNWYVDGVEVHNAKTKPREEQYN | |
| | | STYRVVSVLTVLHQDWLNGKEYKCKVSNKALPAPIEKTISKAKGQPREPQ | |
| | | VYTLPPSREEMTKNQVSLWCLVKGFYPSDIAVEWESNGQPENNYKTTPPV | |
| | | LDSDGSFFLYSKLTVDKSRWQQGNVFSCSVMHEALHNHYTQKSLSLSPGK | |
| | Herceptin | DIQMTQSPSSLSASVGDRVTITCRASQDVNTAVAWYQQKPGKAPKLLIYS | 89 |
| | VK-CK | ASFLYSGVPSRFSGSRSGTDFTLTISSLQPEDFATYYCQQHYTTPPTFGQ | |
| | | GTKVEIKRTVAAPSVFIFPPSDEQLKSGTASVVCLLNNFYPREAKVQWKV | |
| | | DNALQSGNSQESVTEQDSKDSTYSLSSTLTLSKADYEKHKVYACEVTHQG | |
| | | LSSPVTKSFNRGEC | |
| | Cetuximab | QVQLKQSGPGLVQPSQSLSITCTVSGFSLTNYGVHWVRQSPGKGLEWLGV | 90 |
| | VH-CH1- | IWSGGNTDYNTPFTSRLSINKDNSKSQVFFKMNSLQSNDTAIYYCARALT | |
| | hinge-CH2- | YYDYEFAYWGQGTLVTVSAASTKGPSVFPLAPSSKSTSGGTAALGCLVKD | |
| | CH3(holes) | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSREEMTKNQVSLSCAVKGFYPSDIAVEWESNGQPENNYKTTPPVL | |
| | | DSDGSFFLVSKLTVDKSRWQQGNVFSCSVMHEALHNHYTQKSLSLSPGK | |
| | Cetuximab | DILLTQSPVILSVSPGERVSFSCRASQSIGTNIHWYQQRTNGSPRLLIKY | 91 |
| | VK-Cκ | ASESISGIPSRFSGSGSGTDFTLSINSVESEDIADYYCQQNNNWPTTFGA | |
| | | GTKLELKRTVAAPSVFIFPPSDEQLKSGTASVVCLLNNFYPREAKVQWKV | |
| | | DNALQSGNSQESVTEQDSKDSTYSLSSTLTLSKADYEKHKVYACEVTHQG | |
| | | LSSPVTKSFNRGEC | |
| Half- | Herceptin | EVQLVESGGGLVQPGGSLPISCAASGFNIKDTTIHWVRQAPGKGLEIVAR | 92 |
| Herceptin | VH-CH1- | IYPTNGYTRYADSVRGRFTISADTSKNTAYLQMNSLRAEDTAVYYCSRWG | |
| | hinge-CH2- | GDGFYANDYWGQGTLVTVSSASTKGPSVFPLAPSSKSTSGGTAALGCLVK | |
| | CH3(halfbody) | DYFPEPVTVSWNSGALTSGVHTFPAVLQSSGLYSLSSVVTVPSSSLGTQT | |
| | | YICNVNHKPSNTKVDKKVEPKSCDKTHTSPPSPAPELLGGPSVFLFPPKP | |
| | | KDTLMISRTPEVTCVVVDVSHEDPEVKFNWYVDGVEVHNAKTKPREEQYN | |
| | | STYRVVSVLTVLHQDWLNGKEYKCKVSNKALPAPIEKTISKAKGQPREPQ | |
| | | VYTLPPSREEMTKNQVSLTCLVKGFYPSDIAVEWESNGQPENNYKTTPPV | |
| | | LDSDGSFRLYSKLTVDKSRWQQGNVFSCSVMHEALHNHYTQKSLSLSPGK | |
| | Herceptin | DIQMTQSPSSLSASVGDRVTITCRASQDVNTAVAWYQQKPGKAPKLLIYS | 93 |
| | VK-CK | ASFLYSGVPSRFSGSRSGTDFTLTISSLQPEDFATYYCQQHYTTPPTFGQ | |
| | | GTKVEIKRTVAAPSVFIFPPSDEQLKSGTASVVCLLNNFYPREAKVQWKV | |
| | | DNALQSGNSQESVTEQDSKDSTYSLSSTLTLSKADYEKHKVYACEVTHQG | |
| | | LSSPVTKSFNRGEC | |

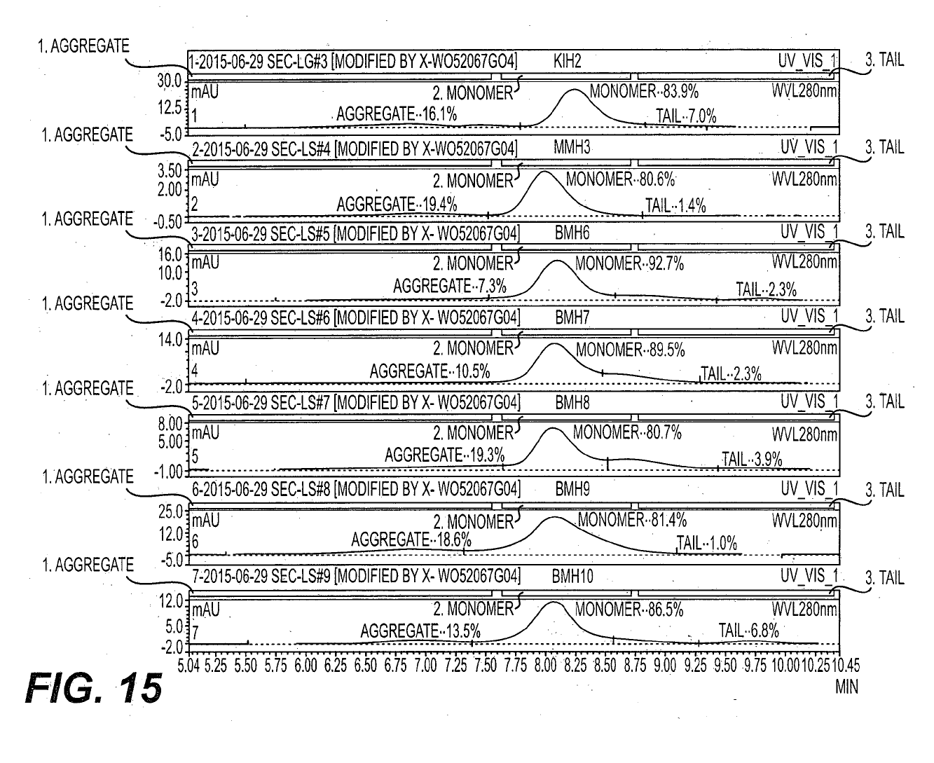

The MH2 domains (MH2, MH2n, MH2p, MH2h, and MH2k) were synthesized by Integrated DNA Technologies. To further stabilize the bispecific molecules, knobs-into-holes heterodimerization technology was utilized by introducing T366W (knobs) or T366S, L368A, and Y407V (holes) mutations into the CH3 domains of the antibody. Four chain vectors were used for each molecule. The anti-HER2 Herceptin VH domain and a selected MH2 domain were integrated into a pHybE huIgG1 vector with the knob mutation to form the knob heavy chain. The anti-HER2 Herceptin Vκ and the pairing MH2 domain were assembled and then introduced to a pHybE huCκ vector to form the corresponding light chain. The anti-EGFR VH and Vκ were incorporated into a pHybE huIgG1 vector with the hole mutations in CH3 domain and a pHybE huCκ vector, respectively. All cloning was completed with homologous recombination and transformation in DH5a cells. All bispecific molecules were expressed in HEK293 cells and purified with MabSelect SuRe beads. Their molecular profiles were analyzed by SEC and mass spectrometry. The SEC profile of each molecule is shown in FIG. 15.

TABLE 11
Expression Of Bispecific Molecules Containing
MH2 Domains and Halfbodies

| | Octet Titer | Yield | SEC |
|---|---|---|---|
| Antibody ID | (mg/L) | (mg/L) | Monomer % |
| BMH6(MH2WT) | 46 | 43.2 | 92.7 |
| BMH7(MH2n/p) | 18.6 | 20.1 | 89.5 |
| BMH8(MH2p/n) | 25.5 | 14.1 | 80.7 |
| BMH9(MH2h/k) | 27.8 | 16 | 81.4 |
| BMH10(MH2k/h) | 27.4 | 22.2 | 86.5 |
| KIH2 | 81.5 | 65.8 | 83.9 |
| Half-Herceptin | 150 | 82.4 | |

### Example 10.2: Bispecific BMH Molecules Eliminating the Mispairing Issue Between Light Chain and Heavy Chain

The molecular weight and identification of bispecific BMH and KIH molecules were determined by mass spec (MS) (Instrument: Agilent HPLC-TOF or HPLC-QTOF; Column: Vydac C4, CN#214MS5115, and CapTrap cartridge; Buffer A: 0.1% FA+0.01% TFA in H2O, buffer B: 0.1% FA+0.01% TFA in CAN; Flow rate: 50 μL/minute; Gradient: 5% buffer B for 5 minutes, 28% to 50% buffer B in 10 minutes, 50% to 95% buffer B in 10 minutes and back to 5% buffer B for 3 minutes for C4 column. 5% buffer B for 7 minutes, 100% buffer B for 7 minutes and back to 5% buffer B for 5 minutes for CapTrap cartridge; MS conditions: For reduced protein: gas temperature 350 C, drying gas 12 L/min, nebulizer 60 psg, fragmentor 350v, skimmer 75v, OCTI RF Vpp 750v, Vcap 5000v. For intact protein: gas temperature 300 C, drying gas 12 L/min, nebulizer 60 psg, fragmentor 350v, skimmer 85v, OCTI RF Vpp 750v, Vcap 5500v).

As shown in Table 12, 48% of paired heavy/light chains were mispaired in KIH2 molecule (33% Herceptin L/Cetuximab H and 15% Cetuximab L/Herceptin H). The % of mispaired heavy/light chain was reduced to 10% in BMH6 and to 0% in BMH7 and BMH8. In addition to eliminating heavy/light chain mispairing, there is enhanced heavy chain hetero-dimerization in BMH molecules. The percentage of heavy chain homo-dimer was reduced from 5% in KIH2 (4% knob-knob dimer and 1% hole-hole dimer) to 0% in BMH6, 2% in BMH7 (2% hole-hole dimer) and 0% in BMH8.

TABLE 12
Semi-Quantitative Analysis of Fab Formation in Bispecific Molecules by Mass Spec

| | Molecule | | | | | | | |
|---|---|---|---|---|---|---|---|---|
| | KIH2 | | BMH6 | | BMH7 | | BMH8 | |
| | Total | % of total | Total | % of total | Total | % of total | Total | % of total |
| Fragment | Abundance | Abundance | Abundance | Abundance | Abundance | Abundance | Abundance | Abundance |
| Herceptin | 1,488,958.90 | 40% | 1,459,845.50 | 51% | 747,249 | 33.40% | 599,491.30 | 30% |
| Fab | | | | | | | | |
| Cetuximab | 450,436.80 | 12% | 1,110,360.20 | 39% | 1,492,578 | 66.60% | 1,391,949.90 | 70% |
| Fab | | | | | | | | |
| Herceptin L/ | 1,216,982.60 | 33% | 179,288.60 | 6% | 0 | 0.00% | 0 | 0% |
| Cetuximab | | | | | | | | |
| H Fab | | | | | | | | |
| Cetuximab L/ | 549,515.30 | 15% | 114,531.70 | 4% | 0 | 0.00% | 0 | 0% |
| Herceptin | | | | | | | | |
| H Fab | | | | | | | | |
| Total of | 3,705,893.50 | 100% | 2,864,026.00 | 100% | 2,239,828 | 100.00% | 1,991,441.20 | 100% |
| Fabs | | | | | | | | |
| Fc knob- | 1,908,688.00 | 95% | 1,633,886.50 | 100% | 1,269,913 | 98.00% | 1,141,235.60 | 100% |
| hole | | | | | | | | |
| Fc knob- | 85,008.20 | 4% | 0 | 0% | 0 | 0.00% | 0 | 0% |
| knob | | | | | | | | |
| Fc hole- | 21,187.40 | 1% | 0 | 0% | 26,283 | 2.00% | 0 | 0% |
| hole | | | | | | | | |
| Total of | 2,014,883.50 | 100% | 1,633,886.50 | 100% | 1,296,196 | 100.00% | 1,141,235.60 | 100% |
| Fc | | | | | | | | |

### Example 10.3: Bispecific BMH Molecules Maintain Binding Specificity and Affinity

The bispecific BMH molecules listed in Table 10 were tested in a FACS binding assay. The KIH, monovalent Herceptin, and monovalent Cetuximab constructs were also tested for comparison. A431 cells were used for testing EGFR binding. N87 cells were used for testing HER2 binding. Cells were incubated with different concentrations of antibodies for 30 minutes and then incubated with fluorescence-conjugated secondary antibodies for another 30 minutes. Cells were then analyzed by flow cytometry and data was analyzed using FlowJo software.

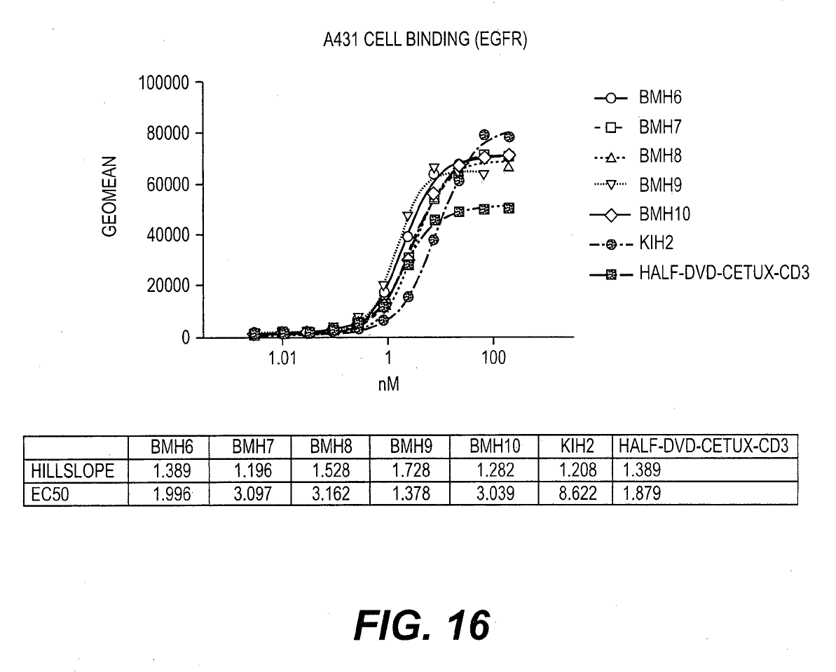

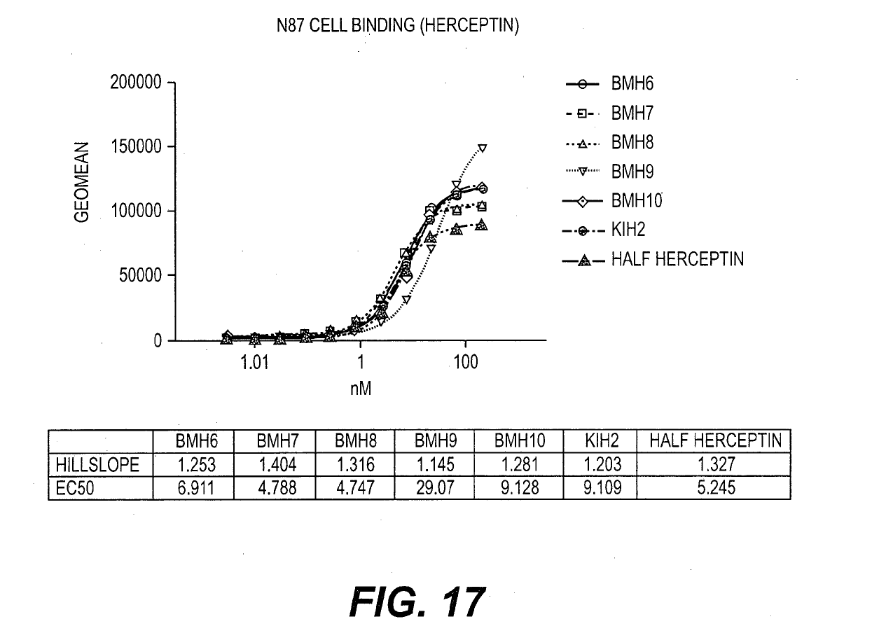

As shown in FIG. 16, all BMH molecules bind to hEGFR on A431 cell with comparable affinity to the original knobs-into-hole bispecific molecule KIH2 and the monovalent Cetuximab in a Half-DVD-Cetux-CD3. As shown in FIG. 17, all BMH molecules bind to hHER2 on N87 cells with comparable affinity to the original knobs-into-holes bispecific molecules KIH2 and the monovalent Herceptin in half Herceptin.

### Example 10.4: Mutation to Knock Out Glycosylation Site on MH2 Domain

As described in Example 4, there is a glycosylation site at position 120 on wild type and engineered hetero-dimerization MH2 domains. In the MH2.S, MH2p.S, MH2n.S, MH2k.S, and MH2h.S domain listed in Table 2, residue Asparagine at position 120 is replaced by a serine residue. Using a non-glycosylated MH2 domain to replace a glycosylated MH2 domain modulates the number of additional glycosylation sites (0-4) introduced by using an MH2 domain. Table 13 summarizes bispecific molecules generated by using those non-glycosylated MH2 domains

TABLE 13
Sequences Of Bispecific Molecules With Non-Glycosylated MH2 Domains

| | | | SEQ |
|---|---|---|---|
| Bispecific | | Sequence | ID |
| Molecules | Chain | 12345678901234567890123456789012345678901234567890 | NO: |
| BMH6.S | Herceptin | EVQLVESGGGLVQPGGSLRLSCAASGFNIKDTYIHWVRQAPGKGLEWVAR | 94 |
| | VH-linker- | IYPTNGYTRYADSVKGRFTISADTSKNTAYLQMNSLRAEDTAVYYCSRWG | |
| | MH2.S-hinge- | GDGFYAMDYWGQGTLVTVSSASTELPPKVSVFVPPRDGFFGNPRKSKLIC | |
| | CH2- | QATGFSPRQIQVSWLREGKQVGSGVTTDQVQAEAKESGPTTYKVTSTLTI | |
| | CH3(knobs) | KESDWLGQSMFTCRVDHRGLTFQQSASSMCDKTHTCPPCPAPELLGGPSV | |
| | | FLFPPKPKDTLMISRTPEVTCVVVDVSHEDPEVKFNWYVDGVEVHNAKTK | |
| | | PREEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPENN | |
| | | YKTTPPVLDSDGSFFLYSKLTVDKSRWQQGNVFSCSVMHEALHNHYTQKS | |
| | | LSLSPGK | |
| | Herceptin | DIQMTQSPSSLSASVGDRVTITCRASQDVNTAVAWYQQKPGKAPKLLIYS | 95 |
| | VK-linker- | ASFLYSGVPSRFSGSRSGTDFTLTISSLQPEDFATYYCQQHYTTPPTFGQ | |
| | MH2.S | GTKVEIKRTAELPPKVSVFVPPRDGFFGNPRKSKLICQATGFSPRQIQVS | |
| | | WLREGKQVGSGVTTDQVQAEAKESGPTTYKVTSTLTIKESDWLGQSMFTC | |
| | | RVDHRGLTFQQSASSMC | |
| | Cetuximab | QVQLKQSGPGLVQPSQSLSITCTVSGFSLTNYGVHWVRQSPGKGLEWLGV | 96 |
| | VH-CH1- | IWSGGNTDYNTPFTSRLSINKDNSKSQVFFKMNSLQSNDTAIYYCARALT | |
| | hinge-CH2- | YYDYEFAYWGQGTLVTVSAASTKGPSVFPLAPSSKSTSGGTAALGCLVKD | |
| | CH3(holes) | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSREEMTKNQVSLSCAVKGFYPSDIAVEWESNGQPENNYKTTPPVL | |
| | | DSDGSFFLVSKLTVDKSRWQQGNVFSCSVMHEALHNHYTQKSLSLSPGK | |
| | Cetuximab | DILLTQSPVILSVSPGERVSFSCRASQSIGTNIHWYQQRTNGSPRLLIKY | 97 |
| | VK-CK | ASESISGIPSRFSGSGSGTDFTLSINSVESEDIADYYCQQNNNWPTTFGA | |
| | | GTKLELKRTVAAPSVFIFPPSDEQLKSGTASVVCLLNNFYPREAKVQWKV | |
| | | DNALQSGNSQESVTEQDSKDSTYSLSSTLTLSKADYEKHKVYACEVTHQG | |
| | | LSSPVTKSFNRGEC | |
| BMH7.S | Herceptin | EVQLVESGGGLVQPGGSLRLSCAASGFNIKDTYIHWVRQAPGKGLEWVAR | 98 |
| | VH-linker- | IYPTNGYTRYADSVKGRFTISADTSKNTAYLQMNSLRAEDTAVYYCSRWG | |
| | MH2n-hinge- | GDGFYAMDYWGQGTLVTVSSASTELPPKVSVFVPPRDGFFGNPRKSELIC | |
| | CH2- | EATGFSPRQIQVSWLREGKQVGSGVTTDQVQAEAKESGPTTYDVTSTLTI | |
| | CH3(knobs) | KESDWLGQSMFTCRVDHRGLTFQQSASSMCDKTHTCPPCPAPELLGGPSV | |
| | | FLFPPKPKDTLMISRTPEVTCVVVDVSHEDPEVKFNWYVDGVEVHNAKTK | |
| | | PREEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPENN | |
| | | YKTTPPVLDSDGSFFLYSKLTVDKSRWQQGNVFSCSVMHEALHNHYTQKS | |
| | | LSLSPGK | |
| | Herceptin | DIQMTQSPSSLSASVGDRVTITCRASQDVNTAVAWYQQKPGKAPKLLIYS | 99 |
| | VK-linker- | ASFLYSGVPSRFSGSRSGTDFTLTISSLQPEDFATYYCQQHYTTPPTFGQ | |
| | MH2p | GTKVEIKRTAELPPKVSVFVPPRDGFFGNPRKSKLICKATGFSPRQIQVS | |
| | | WLREGKQVGSGVTTKQVQAEAKESGPTTYKVTSTLTIKESDWLGQSMFTC | |
| | | RVDHRGLTFQQSASSMC | |
| | Cetuximab | QVQLKQSGPGLVQPSQSLSITCTVSGFSLTNYGVHWVRQSPGKGLEWLGV | 100 |
| | VH-CH1- | IWSGGNTDYNTPFTSRLSINKDNSKSQVFFKMNSLQSNDTAIYYCARALT | |
| | hinge-CH2- | YYDYEFAYWGQGTLVTVSAASTKGPSVFPLAPSSKSTSGGTAALGCLVKD | |
| | CH3(holes) | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSREEMTKNQVSLSCAVKGFYPSDIAVEWESNGQPENNYKTTPPVL | |
| | | DSDGSFFLVSKLTVDKSRWQQGNVFSCSVMHEALHNHYTQKSLSLSPGK | |
| | Cetuximab | DILLTQSPVILSVSPGERVSFSCRASQSIGTNIHWYQQRTNGSPRLLIKY | 101 |
| | VK-CK | ASESISGIPSRFSGSGSGTDFTLSINSVESEDIADYYCQQNNNWPTTFGA | |
| | | GTKLELKRTVAAPSVFIFPPSDEQLKSGTASVVCLLNNFYPREAKVQWKV | |
| | | DNALQSGNSQESVTEQDSKDSTYSLSSTLTLSKADYEKHKVYACEVTHQG | |
| | | LSSPVTKSFNRGEC | |
| BMH8.S | Herceptin | EVQLVESGGGLVQPGGSLRLSCAASGFNIKDTYIHWVRQAPGKGLEWVAR | 102 |
| | VH-linker- | IYPTNGYTRYADSVKGRFTISADTSKNTAYLQMNSLRAEDTAVYYCSRWG | |
| | MH2p.5- | GDGFYAMDYWGQGTLVTVSSASTELPPKVSVFVPPRDGFFGNPRKSKLIC | |
| | hinge-CH2- | KATGFSPRQIQVSWLREGKQVGSGVTTKQVQAEAKESGPTTYKVTSTLTI | |
| | CH3(knobs) | KESDWLGQSMFTCRVDHRGLTFQQSASSMCDKTHTCPPCPAPELLGGPSV | |
| | | FLFPPKPKDTLMISRTPEVTCVVVDVSHEDPEVKFNWYVDGVEVHNAKTK | |
| | | PREEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPENN | |
| | | YKTTPPVLDSDGSFFLYSKLTVDKSRWQQGNVFSCSVMHEALHNHYTQKS | |
| | | LSLSPGK | |
| | Herceptin | DIQMTQSPSSLSASVGDRVTITCRASQDVNTAVAWYQQKPGKAPKLLIYS | 103 |
| | VK-linker- | ASFLYSGVPSRFSGSRSGTDFTLTISSLQPEDFATYYCQQHYTTPPTFGQ | |
| | MH2n.S | GTKVEIKRTAELPPKVSVFVPPRDGFFGNPRKSELICEATGFSPRQIQVS | |
| | | WLREGKQVGSGVTTDQVQAEAKESGPTTYDVTSTLTIKESDWLGQSMFTC | |
| | | RVDHRGLTFQQSASSMC | |
| | Cetuximab | QVQLKQSGPGLVQPSQSLSITCTVSGFSLTNYGVHWVRQSPGKGLEWLGV | 104 |
| | VH-CH1- | IWSGGNTDYNTPFTSRLSINKDNSKSQVFFKMNSLQSNDTAIYYCARALT | |
| | hinge-CH2- | YYDYEFAYWGQGTLVTVSAASTKGPSVFPLAPSSKSTSGGTAALGCLVKD | |
| | CH3(holes) | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSREEMTKNQVSLSCAVKGFYPSDIAVEWESNGQPENNYKTTPPVL | |
| | | DSDGSFFLVSKLTVDKSRWQQGNVFSCSVMHEALHNHYTQKSLSLSPGK | |
| | Cetuximab | DILLTQSPVILSVSPGERVSFSCRASQSIGTNIHWYQQRTNGSPRLLIKY | 105 |
| | VK-CK | ASESISGIPSRFSGSGSGTDFTLSINSVESEDIADYYCQQNNNWPTTFGA | |
| | | GTKLELKRTVAAPSVFIFPPSDEQLKSGTASVVCLLNNFYPREAKVQWKV | |
| | | DNALQSGNSQESVTEQDSKDSTYSLSSTLTLSKADYEKHKVYACEVTHQG | |
| | | LSSPVTKSFNRGEC | |
| BMH9.S | Herceptin | EVQLVESGGGLVQPGGSLRLSCAASGFNIKDTYIHWVRQAPGKGLEWVAR | 106 |
| | VH-linker- | IYPTNGYTRYADSVKGRFTISADTSKNTAYLQMNSLRAEDTAVYYCSRWG | |
| | MH2h.5- | GDGFYAMDYWGQGTLVTVSSASTELPPKVSVFVPPRDGFFGNPRKSKLAC | |
| | hinge-CH2- | SATGFSPRQIQVSWLREGKQVGSGVTTDQVQAEAKESGPTTYKVASTLTI | |
| | CH3(knobs) | KESDWLGQSMFTCRVDHRGLTFQQSASSMCDKTHTCPPCPAPELLGGPSV | |
| | | FLFPPKPKDTLMISRTPEVTCVVVDVSHEDPEVKFNWYVDGVEVHNAKTK | |
| | | PREEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPENN | |
| | | YKTTPPVLDSDGSFFLYSKLTVDKSRWQQGNVFSCSVMHEALHNHYTQKS | |
| | | LSLSPGK | |
| | Herceptin | DIQMTQSPSSLSASVGDRVTITCRASQDVNTAVAWYQQKPGKAPKLLIYS | 107 |
| | VK-linker- | ASFLYSGVPSRFSGSRSGTDFTLTISSLQPEDFATYYCQQHYTTPPTFGQ | |
| | MH2k.S | GTKVEIKRTAELPPKVSVFVPPRDGFFGNPRKSKLWCQATGFSPRQIQVS | |
| | | WLREGKQVGSGVTTDQVQAEAKESGPTTYKVTSTLTIKESDWLGQSMFTC | |
| | | RVDHRGLTFQQSASSMC | |
| | Cetuximab | QVQLKQSGPGLVQPSQSLSITCTVSGFSLTNYGVHWVRQSPGKGLEWLGV | 108 |
| | VH-CH1- | IWSGGNTDYNTPFTSRLSINKDNSKSQVFFKMNSLQSNDTAIYYCARALT | |
| | hinge-CH2- | YYDYEFAYWGQGTLVTVSAASTKGPSVFPLAPSSKSTSGGTAALGCLVKD | |
| | CH3(holes) | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSREEMTKNQVSLSCAVKGFYPSDIAVEWESNGQPENNYKTTPPVL | |
| | | DSDGSFFLVSKLTVDKSRWQQGNVFSCSVMHEALHNHYTQKSLSLSPGK | |
| | Cetuximab | DILLTQSPVILSVSPGERVSFSCRASQSIGTNIHWYQQRTNGSPRLLIKY | 109 |
| | VK-CK | ASESISGIPSRFSGSGSGTDFTLSINSVESEDIADYYCQQNNNWPTTFGA | |
| | | GTKLELKRTVAAPSVFIFPPSDEQLKSGTASVVCLLNNFYPREAKVQWKV | |
| | | DNALQSGNSQESVTEQDSKDSTYSLSSTLTLSKADYEKHKVYACEVTHQG | |
| | | LSSPVTKSFNRGEC | |
| BMH10.S | Herceptin | EVQLVESGGGLVQPGGSLRLSCAASGFNIKDTYIHWVRQAPGKGLEWVAR | 110 |
| | VH-linker- | IYPTNGYTRYADSVKGRFTISADTSKNTAYLQMNSLRAEDTAVYYCSRWG | |
| | MH2k-hinge- | GDGFYAMDYWGQGTLVTVSSASTELPPKVSVFVPPRDGFFGNPRKSKLWC | |
| | CH2- | QATGFSPRQIQVSWLREGKQVGSGVTTDQVQAEAKESGPTTYKVTSTLTI | |
| | CH3(knobs) | KESDWLGQSMFTCRVDHRGLTFQQSASSMCDKTHTCPPCPAPELLGGPSV | |
| | | FLFPPKPKDTLMISRTPEVTCVVVDVSHEDPEVKFNWYVDGVEVHNAKTK | |
| | | PREEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPENN | |
| | | YKTTPPVLDSDGSFFLYSKLTVDKSRWQQGNVFSCSVMHEALHNHYTQKS | |
| | | LSLSPGK | |
| | Herceptin | DIQMTQSPSSLSASVGDRVTITCRASQDVNTAVAWYQQKPGKAPKLLIYS | 111 |
| | VK-linker- | ASFLYSGVPSRFSGSRSGTDFTLTISSLQPEDFATYYCQQHYTTPPTFGQ | |
| | MH2h | GTKVEIKRTAELPPKVSVFVPPRDGFFGNPRKSKLACSATGFSPRQIQVS | |
| | | WLREGKQVGSGVTTDQVQAEAKESGPTTYKVASTLTIKESDWLGQSMFTC | |
| | | RVDHRGLTFQQSASSMC | |
| | Cetuximab | QVQLKQSGPGLVQPSQSLSITCTVSGFSLTNYGVHWVRQSPGKGLEWLGV | 112 |
| | VH-CH1- | IWSGGNTDYNTPFTSRLSINKDNSKSQVFFKMNSLQSNDTAIYYCARALT | |
| | hinge-CH2- | YYDYEFAYWGQGTLVTVSAASTKGPSVFPLAPSSKSTSGGTAALGCLVKD | |
| | CH3(holes) | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSREEMTKNQVSLSCAVKGFYPSDIAVEWESNGQPENNYKTTPPVL | |
| | | DSDGSFFLVSKLTVDKSRWQQGNVFSCSVMHEALHNHYTQKSLSLSPGK | |
| | Cetuximab | DILLTQSPVILSVSPGERVSFSCRASQSIGTNIHWYQQRTNGSPRLLIKY | 113 |
| | VK-Cκ | ASESISGIPSRFSGSGSGTDFTLSINSVESEDIADYYCQQNNNWPTTFGA | |
| | | GTKLELKRTVAAPSVFIFPPSDEQLKSGTASVVCLLNNFYPREAKVQWKV | |
| | | DNALQSGNSQESVTEQDSKDSTYSLSSTLTLSKADYEKHKVYACEVTHQG | |
| | | LSSPVTKSFNRGEC | |

The non-glycosylated MH2 domains were synthesized by Integrated DNA Technologies. To further stabilize the bispecific molecules, knobs-into-holes heterodimerization technology was utilized by introducing T366W (knobs) or T366S, L368A, and Y407V (holes) mutations into the CH3 domains of the antibody. Four chain vectors were used for each molecule. The anti-HER2 Herceptin VH domain and a selected MH2 domain were integrated into a pHybE huIgG1 vector with the knobs mutation to form the knob heavy chain. The anti-HER2 Herceptin Vκ and the pairing MH2 domain were assembled and then introduced to a pHybE huCκ vector to form the corresponding light chain. The anti-EGFR VH and Vκ were incorporated into a pHybE huIgG1 vector with the holes mutations in a CH3 domain to form the hole heavy chain and a pHybE huCκ vector, respectively. All cloning was completed with homologous recombination and transformation in DH5a cells. All bispecific molecules were expressed in HEK293 cells and purified with MabSelect SuRe beads. Their molecular profiles were analyzed by SEC and mass spectrometry.

### Example 11: Generation of Trispecific Molecules with Wild Type MH2 Homodimer or Engineered MH2a/MH2b Heterodimer

Table 14 summarizes ten exemplary combinations that can be used to build trispecific molecules using MH2/MH2, MH2p/MH2n, or MH2k/MH2h domains in a knobs-into-holes format. In each, VH1 and VL1 are the variable domains taken from one parental antibody, VH2 and VL2 are variable domains from another parental antibody, and VH3 and VL3 are variable domains from yet another antibody. Each trispecific molecule is generated with four chains: 2 heavy chains (chain 1 and chain 3) and 2 light chains (chain 2 and chain 4).

TABLE 14
Combinations of Four Chains That Generate Trispecific
Molecules Using MH2/MH2, MH2p/MH2n, Or MH2k/MH2h
Dimers In A Knobs-Into-Holes Format

| | Trispecific IgG Combination | | | |
|---|---|---|---|---|
| | Chain 1 | Chain 2 | Chain 3 | Chain 4 |
| 1 | VH1-linker- | VL1-linker- | VH2-linker- | VL2-linker- |
| | MH2-hinge- | MH2 | VH3-CH1-hinge- | VL3-Cκ (λ) |
| | CH2—CH3 | | CH2—CH3 | |
| | (knobs) | | (holes) | |
| 2 | VH1-linker- | VL1-linker- | VH2-linker- | VL2-linker- |
| | MH2p-hinge- | MH2n | VH3-CH1-hinge- | VL3-Cκ (λ) |
| | CH2—CH3 | | CH2—CH3 | |
| | (knobs) | | (holes) | |
| 3 | VH1-linker- | VL1-linker- | VH2-linker- | VL2-linker- |
| | MH2n-hinge- | MH2p | VH3-CH1-hinge- | VL3-Cκ (λ) |
| | CH2—CH3 | | CH2—CH3 | |
| | (knobs) | | (holes) | |
| 4 | VH1-linker- | VL1-linker- | VH2-linker- | VL2-linker- |
| | MH2k-hinge- | MH2h | VH3-CH1-hinge- | VL3-Cκ (λ) |
| | CH2—CH3 | | CH2—CH3 | |
| | (knobs) | | (holes) | |
| 5 | VH1-linker- | VL1-linker- | VH2-linker- | VL2-linker- |
| | MH2h-hinge- | MH2k | VH3-CH1-hinge- | VL3-Cκ (λ) |
| | CH2—CH3 | | CH2—CH3 | |
| | (knobs) | | (holes) | |
| 6 | VH1-linker- | VL1-linker- | VH2-linker- | VL2-linker- |
| | MH2-hinge- | MH2 | VH3-CH1-hinge- | VL3-Cκ (λ) |
| | CH2—CH3 | | CH2—CH3 | |
| | (holes) | | (knobs) | |
| 7 | VH1-linker- | VL1-linker- | VH2-linker- | VL2-linker- |
| | MH2p-hinge- | MH2n | VH3-CH1-hinge- | VL3-Cκ (λ) |
| | CH2—CH3 | | CH2—CH3 | |
| | (holes) | | (knobs) | |
| 8 | VH1-linker- | VL1-linker- | VH2-linker- | VL2-linker- |
| | MH2n-hinge- | MH2p | VH3-CH1-hinge- | VL3-Cκ (λ) |
| | CH2—CH3 | | CH2—CH3 | |
| | (holes) | | (knobs) | |
| 9 | VH1-linker- | VL1-linker- | VH2-linker- | VL2-linker- |
| | MH2k-hinge- | MH2h | VH3-CH1-hinge- | VL3-Cκ (λ) |
| | CH2—CH3 | | CH2—CH3 | |
| | (holes) | | (knobs) | |
| 10 | VH1-linker- | VL1-linker- | VH2-linker- | VL2-linker- |
| | MH2h-hinge- | MH2k | VH3-CH1-hinge- | VL3-Cκ (λ) |
| | CH2—CH3 | | CH2—CH3 | |
| | (holes) | | (knobs) | |

### Example 11.1: Generation of Trispecific Molecules with Wild Type MH2 Homodimer or Engineered MH2a/MH2b Heterodimer

The variable domain sequences used to generate the tri-specific molecules are listed in Table 15.

TABLE 15
Antibody Variable Domains Used To Build IgG-Like Molecules Containing
MH2 Domains

| Antibody | | Variable Domain Sequence | SEQ ID |
|---|---|---|---|
| Target | Domain | 1234567890123456789012345678901234567890 | NO: |
| EGFR | Cetuximab | QVQLKQSGPGLVQPSQSLSITCTVSGFSLTNYGVHWVRQS | 114 |
| (Cetuximab) | VH | PGKGLEWLGVIWSGGNTDYNTPFTSRLSINKDNSKSQVFF | |
| | | KMNSLQSNDTAIYYCARALTYYDYEFAYWGQGTLVTVSA | |
| | Cetuximab | DILLTQSPVILSVSPGERVSFSCRASQSIGTNIHWYQQRT | 115 |
| | VK | NGSPRLLIKYASESISGIPSRFSGSGSGTDFTLSINSVES | |
| | | EDIADYYCQQNNNWPTTFGAGTKLELK | |
| CD3 | AB002VH | QVQLQQSGAELARPGASVKMSCKASGYTFTRYTMHWVKQR | 116 |
| (AB002) | | PGQGLEWIGYINPSRGYTNYNQKFKDKATLTTDKSSSTAY | |
| | | MQLSSLTSEDSAVYYCARYYDDHYCLDYWGQGTTLTVSS | |
| | AB002VK | QIVLTQSPAIMSASPGEKVTMTCRASSSVSYMNWYQQKSG | 117 |
| | | TSPKRWIYDTSKVASGVPYRFSGSGSGTSYSLTISSMEAE | |
| | | DAATYYCQQWSSNPLTFGSGTKLEIN | |
| EGFR | Cetuximab. | QVQLKQSGPGLVQPSQSLSITCTVSGFSLTNYGVHWVRQS | 118 |
| (Cetuximan. | 2VH | PGKGLEWLGVIWSGGNTDYNTPFTSRLSINKDNSKSQVFF | |
| 2) | | KMNSLQSEDTAIYYCARALTYYDYEFAYWGQGTLVTVSA | |
| | Cetuximab. | DILLTQSPVILSVSPGERVSFSCRASQSIGTNIHWYQQRT | 119 |
| | 2VK | SGSPRLLIKYASESISGIPSRFSGSGSGTDFTLSINSVES | |
| | | EDIADYYCQQNNNWPTTFGAGTKLELK | |
| CD3 | AB002.2VH | QVQLQQSGAELARPGASVKMSCKASGYTFTRYTMHWVKQR | 120 |
| (AB002.2) | | PGQGLEWIGYINPSRGYTNYNQKFKDKATLTTDKSSSTAY | |
| | | MQLSSLTSEDSAVYYCARYYDDHYSLDYWGQGTTLTVSS | |
| | AB002.2VK | QIVLTQSPAIMSASPGEKVTMTCRASSSVSYMNWYQQKSG | 121 |
| | | TSPKRWIYDTSKVASGVPYRFSGSGSGTSYSLTISSMEAE | |
| | | DAATYYCQQWSSNPLTFGSGTKLEIK | |
| CD2 | AB765VH | EVQLVESGGGLVQPGGSLRLSCAASGFAFSSYDMSWVRQA | 122 |
| (AB765) | | PGKGLEWVSYISGGGFTYYPDTVKGRFTISRDNSKNTLYL | |
| | | QMNSLRAEDTAVYYCARQGANWELVYWGQGTLVTVSS | |
| | AB765VK | EIVLTQSPATLSLSPGERATLSCRASQSISDFLHWYQQKP | 123 |
| | | GQAPRLLIKYASQSISGIPARFSGSGSGTDFTLTISSLEP | |
| | | EDFAVYYCQNGHNFPPTFGGGTKVEIK | |
| PD1(AB426) | AB426VH | QVQLVESGGGVVQPGRSLRLDCKASGITFSNSGMHWVRQA | 124 |
| | | PGKGLEWVAVIWYDGSKRYYADSVKGRFTISRDNSKNTLF | |
| | | LQMNSLRAEDTAVYYCATNDDYWGQGTLVTVSS | |
| | AB426VK | EIVLTQSPATLSLSPGERATLSCRASQSVSSYLAWYQQKP | 125 |
| | | GQAPRLLIYDASNRATGIPARFSGSGSGTDFTLTISSLEP | |
| | | EDFAVYYCQQSSNWPRTFGQGTKVEIK | |
| PDL1(YW243) | YW243VH | EVQLVESGGGLVQPGGSLRLSCAASGFTFSDSWIHWVRQA | 126 |
| | | PGKGLEWVAWISPYGGSTYYADSVKGRFTISADTSKNTAY | |
| | | LQMNSLRAEDTAVYYCARRHYPGGFDYWGQGTLVTVSA | |
| | YW243VK | DIQMTQSPSSLSASVGDRVTITCRASQDVSTAVAWYQQKP | 127 |
| | | GKAPKLLIYSASFLYSGVPSRFSGSGSGTDFTLTISSLQP | |
| | | EDFATYYCQQYLYHPATFGQGTKVEIK | |

Five sets of five trispecific molecules were generated based on chain combinations 1-5 listed in Table 14. In the first set of five trispecific molecules TMH1-5, VH1 and VL1 are from an anti-CD2 antibody AB765 (AB765 VH and AB765 VK), VH2 and VL2 are from the anti-EGFR antibody Cetuximab (Cetuximab VH and Cetuximab VK), and VH3 and VL3 are from an anti-CD3 antibody AB002 (AB002 VH and AB002 VK). The anti-CD2 antibody AB765 VH domain and the selected MH2 domain were integrated into a pHybE huIgG1 vector with knobs mutation in the CH3 domain to form knob heavy chain. The anti-CD2 antibody AB765 Vκ domain and the paired MH2 domain were assembled and then introduced to a pHybE huCκ vector to form the corresponding light chain. The anti-EGFR/CD3 Cetuximab VH-linker-AB002 VH and the Cetuximab AB002 Vκ were incorporated into a pHybE huIgG1 vector with holes mutations in the CH3 domain and a pHybE huCκ vector, respectively. Several constructs lacking MH2 or EH2 modifications were also tested for comparison purposes, including: (1) a knobs-into-holes binding protein (PLY11) targeting CD2, CD3, and EGFR with the same variable domains; (2) an anti-CD2 halfbody (TS2/18 half); and (3) an anti-EGFR/CD3 halfbody (DVD860 half).

In the second set of five trispecific molecules TMH6-10, VH1 and VL1 are from anti-PD1 AB426 (AB426 VH and AB426 Vκ), VH2 and VL2 are from the non-glycosylated anti-EGFR antibody Cetuximab.2 (Cetuximab.2 VH and Cetuximab.2 Vκ), and VH3 and VL3 are from the non-glycosylated and non-free Cysteine anti-CD3 antibody AB002.2 (AB002.2 VH and AB002.Vκ). Cetuximab.2 is a mutant of Cetuximab with the glycosylation sites on variable domain removed. AB002.2 is a mutant of AB002 with free Cysteine on the heavy chain CDR3 and the glycosylation site on the light chain removed. The anti-PD1 antibody AB426 VH domain and the selected MH2 domain were integrated into a pHybE huIgG1 vector with knobs mutation in the CH3 domain and LALA mutation in the CH2 domains to reduce Fcγ Receptor binding. The anti-PD1 antibody AB426 Vκ domain and the paired MH2 domain were assembled and then introduced to a pHybE huCκ vector to form the corresponding light chain. The Cetuximab.2 VH-linker-AB002.2 VH was incorporated into a pHybE huIgG1 vector with holes mutations in the CH3 domain and LALA mutations in the CH2 to form a hole heavy chain. The Cetuximab.2 anti-AB002.2 Vκ was incorporated into and a pHybE huCκ vector to form the light chain pairing to the hole heavy chain. Several constructs lacking MH2 or EH2 modifications were also tested for comparison purposes, including: (1) a knobs-into-holes binding protein KIH4 targeting PD1, CD3 and EGFR with the same variable domains used in TMH6-10; (2) an anti-PD1 halfbody (AB426 half); and (3) an anti-EGFR/CD3 halfbody (DVD860.2 half) with variable domains from Cetuximab.2 and AB002.2.

In the third set of five trispecific molecules TMH11-15, VH1 and VL1 are from anti-PDL1 antibody YW243 (YW243 VH and YW243 Vκ), VH2 and VL2 are from the non-glycosylated anti-EGFR antibody Cetuximab.2 (Cetuximab.2 VH and Cetuximab.2 Vκ), and VH3 and VL3 are from the non-glycosylated and non-free Cysteine anti-CD3 antibody AB002.2 (AB002.2 VH and AB002.2 Vκ). Cetuximab.2 is a mutant of Cetuximab with the glycosylation sites on the variable domains removed. AB002.2 is a mutant of AB002 with free Cysteine on the heavy chain CDR3 and the glycosylation site on the light chain removed. The anti-PDL1 antibody YW243 VH domain and selected MH2 domain were integrated into a pHybE huIgG1 vector with LALA mutation in the CH2 domain and knobs mutation in the CH3 domain to form knob heavy chain. The YW243 Vκ domain and the paired MH2 domain were assembled and then introduced to a pHybE huCκ vector to form the corresponding light chain The Cetuximab.2 VH-linker-AB002.2 VH was incorporated into a pHybE huIgG1 vector with LALA mutations in the CH2 domain and holes mutations in the CH3 domain to form a hole heavy chain. The Cetuximab.2 Vκ-linker-AB002.2 Vκ was incorporated into pHybE huCκ vector to form the light chain pairing to the hole heavy chain Several constructs lacking MH2 or EH2 modifications were also tested for comparison purposes, including: (1) a knobs-into-holes binding protein KIH5 targeting PDL1, CD3 and EGFR with the same variable domains used in TMH11-15; (2) an anti-PDL1 halfbody (YW243half); and (3) an anti-EGFR/CD3 halfbody (DVD860.2 half) with variable domains from Cetuximab.2 and AB002.2.

In the fourth set of five trispecific molecules TMH16-20, VH1 and VL1 are from anti-PDL1 antibody YW243(YW243 VH and YW243 VK), VH2 and VL2 are from the anti-EGFR antibody Cetuximab (Cetuximab VH and Cetuximab VK), and VH3 and VL3 are from anti-CD3 antibody AB002 (AB002 VH and AB002Vκ). The anti-PDL1 antibody YW243 VH domain and selected MH2 domain were integrated into a pHybE huIgG1 vector with LALA mutation in the CH2 domain and knobs mutation in the CH3 domain to form a knobs heavy chain. The YW243 Vκ domain and the paired MH2 domain were assembled and then introduced to a pHybE huCκ vector to form the corresponding light chain. The Cetuximab VH-linker-AB002 VH was incorporated into a pHybE huIgG1 vector with LALA mutations in the CH2 domain and holes mutations in the CH3 domain to form a hole heavy chain. The Cetuximab Vκ-linker-AB002 Vκ and a pHybE huCκ vector were used, respectively. Several constructs lacking MH2 or EH2 modifications were also tested for comparison purposes, including: (1) a knobs-into-holes binding protein KIH6 targeting PDL1, CD3 and EGFR with the same variable domains used in TMH16-20; (2) an anti-PDL1 halfbody (YW243 half); and (3) an anti-EGFR/CD3 halfbody (DVD860 half) with variable domains from Cetuximab and AB002.

In the fifth set of five trispecific molecules TMH21-25, VH1 and VL1 are from anti-PD1 antibody AB426 (AB426 VH and AB426 VK), VH2 and VL2 are from the anti-EGFR antibody Cetuximab (Cetuximab VH and Cetuximab VK), and VH3 and VL3 are from anti-CD3 antibody AB002 (AB002 VH and AB002Vκ). The anti-PD1 antibody AB426 VH domain and selected MH2 domain were integrated into a pHybE huIgG1 vector with LALA mutation in the CH2 domain and knobs mutation in the CH3 domain. The AB426 VK domain and the paired MH2 domain were assembled and then introduced into a pHybE huCκ vector to form the corresponding light chain. The Cetuximab VH-linker-AB002 VH was incorporated into a pHybE huIgG1 vector with LALA mutations in the CH2 domain and holes mutations in the CH3 domain to form a hole heavy chain. The Cetuximab Vκ-linker-AB002 Vκ and a pHybE huCκ vector were used, respectively. Several constructs lacking MH2 or EH2 modifications were also tested for comparison purposes, including: (1) a knobs-into-holes binding protein KIH7 targeting PD1, CD3 and EGFR with the same variable domains used in TMH21-25; (2) an anti-PD1 halfbody (AB426 half); and (3) an anti-EGFR/CD3 halfbody (DVD860 half) with variable domains from Cetuximab and AB002.

Table 16 summarizes the sequences of the four chains in each of the trispecific molecules and halfbodies that were generated and tested. Table 16 also shows the sequences of DVD889 [hu IgG1/k] that was used as a negative control. DVD889 [hu IgG1/k] binds to Tetanus toxoid.

TABLE 16
Sequences Of Generated Trispecific Molecules, Halfbodies, and Control DVD-Ig

| Prispecific | | Sequence | |
|---|---|---|---|
| Molecules | Chain | 12345678901234567890123456789012345678901234567890 | SEQ ID NO: |
| TMH1 | AB765 VH-linker- | EVQLVESGGGLVQPGGSLRLSCAASGFAFSSYDMSWVRQAPGKGLEWVSY | 128 |
| | MH2-hinge-CH2- | ISGGGFTYYPDTVKGRFTISRDNSKNTLYLQMNSLRAEDTAVYYCARQGA | |
| | CH3(knobs) | NWELVYWGQGTLVTVSSASTELPPKVSVFVPPRDGFFGNPRKSKLICQAT | |
| | | GFSPRQIQVSWLREGKQVGSGVTTDQVQAEAKESGPTTYKVTSTLTIKES | |
| | | DWLGQSMFTCRVDHRGLTFQQNASSMCDKTHTCPPCPAPELLGGPSVFLF | |
| | | PPKPKDTLMISRTPEVTCVVVDVSHEDPEVKFNWYVDGVEVHNAKTKPRE | |
| | | EQYNSTYRVVSVLTVLHQDWLNGKEYKCKVSNKALPAPIEKTISKAKGQP | |
| | | REPQVYTLPPSREEMTKNQVSLWCLVKGFYPSDIAVEWESNGQPENNYKT | |
| | | TPPVLDSDGSFFLYSKLTVDKSRWQQGNVFSCSVMHEALHNHYTQKSLSL | |
| | | SPGK | |
| | AB765 VK-linker- | EIVLTQSPATLSLSPGERATLSCRASQSISDFLHWYQQKPGQAPRLLIKY | 129 |
| | MH2 | ASQSISGIPARFSGSGSGTDFTLTISSLEPEDFAVYYCQNGHNFPPTFGG | |
| | | GTKVEIKRTAELPPKVSVFVPPRDGFFGNPRKSKLICQATGFSPRQIQVS | |
| | | WLREGKQVGSGVTTDQVQAEAKESGPTTYKVTSTLTIKESDWLGQSMFTC | |
| | | RVDHRGLTFQQNASSMC | |
| | Cetuximab VH- | QVQLKQSGPGLVQPSQSLSITCTVSGFSLTNYGVHWVRQSPGKGLEWLGV | 130 |
| | linker-AB002 VH- | IWSGGNTDYNTPFTSRLSINKDNSKSQVFFKMNSLQSNDTAIYYCARALT | |
| | CH1-hinge-CH2- | YYDYEFAYWGQGTLVTVSAASTKGPSVFPLAPQVQLQQSGAELARPGASV | |
| | CH3(holes) | KMSCKASGYTFTRYTMHWVKQRPGQGLEWIGYINPSRGYTNYNQKFKDKA | |
| | | TLTTDKSSSTAYMQLSSLTSEDSAVYYCARYYDDHYCLDYWGQGTTLTVS | |
| | | SASTKGPSVFPLAPSSKSTSGGTAALGCLVKDYFPEPVTVSWNSGALTSG | |
| | | VHTFPAVLQSSGLYSLSSVVTVPSSSLGTQTYICNVNHKPSNTKVDKKVE | |
| | | PKSCDKTHTCPPCPAPELLGGPSVFLFPPKPKDTLMISRTPEVTCVVVDV | |
| | | SHEDPEVKFNWYVDGVEVHNAKTKPREEQYNSTYRVVSVLTVLHQDWLNG | |
| | | KEYKCKVSNKALPAPIEKTISKAKGQPREPQVYTLPPSREEMTKNQVSLS | |
| | | CAVKGFYPSDIAVEWESNGQPENNYKTTPPVLDSDGSFFLVSKLTVDKSR | |
| | | WQQGNVFSCSVMHEALHNHYTQKSLSLSPGK | |
| | Cetuximab VK- | DILLTQSPVILSVSPGERVSFSCRASQSIGTNIHWYQQRTNGSPRLLIKY | 131 |
| | linker-AB002 Vκ- | ASESISGIPSRFSGSGSGTDFTLSINSVESEDIADYYCQQNNNWPTTFGA | |
| | Cκ | GTKLELKRTVAAPSVFIFPPQIVLTQSPAIMSASPGEKVTMTCRASSSVS | |
| | | YMNWYQQKSGTSPKRWIYDTSKVASGVPYRFSGSGSGTSYSLTISSMEAE | |
| | | DAATYYCQQWSSNPLTFGSGTKLEINRTVAAPSVFIFPPSDEQLKSGTAS | |
| | | VVCLLNNFYPREAKVQWKVDNALQSGNSQESVTEQDSKDSTYSLSSTLTL | |
| | | SKADYEKHKVYACEVTHQGLSSPVTKSFNRGEC | |
| TMH2 | AB765 VH-linker- | EVQLVESGGGLVQPGGSLRLSCAASGFAFSSYDMSWVRQAPGKGLEWVSY | 132 |
| | MH2p-hinge-CH2- | ISGGGFTYYPDTVKGRFTISRDNSKNTLYLQMNSLRAEDTAVYYCARQGA | |
| | CH3(knobs) | NWELVYWGQGTLVTVSSASTELPPKVSVFVPPRDGFFGNPRKSKLICKAT | |
| | | GFSPRQIQVSWLREGKQVGSGVTTKQVQAEAKESGPTTYKVTSTLTIKES | |
| | | DWLGQSMFTCRVDHRGLTFQQNASSMCDKTHTCPPCPAPELLGGPSVFLF | |
| | | PPKPKDTLMISRTPEVTCVVVDVSHEDPEVKFNWYVDGVEVHNAKTKPRE | |
| | | EQYNSTYRVVSVLTVLHQDWLNGKEYKCKVSNKALPAPIEKTISKAKGQP | |
| | | REPQVYTLPPSREEMTKNQVSLWCLVKGFYPSDIAVEWESNGQPENNYKT | |
| | | TPPVLDSDGSFFLYSKLTVDKSRWQQGNVFSCSVMHEALHNHYTQKSLSL | |
| | | SPGK | |
| | AB765 VK-linker- | EIVLTQSPATLSLSPGERATLSCRASQSISDFLHWYQQKPGQAPRLLIKY | 133 |
| | MH2n | ASQSISGIPARFSGSGSGTDFTLTISSLEPEDFAVYYCQNGHNFPPTFGG | |
| | | GTKVEIKRTAELPPKVSVFVPPRDGFFGNPRKSELICEATGFSPRQIQVS | |
| | | WLREGKQVGSGVTTDQVQAEAKESGPTTYDVTSTLTIKESDWLGQSMFTC | |
| | | RVDHRGLTFQQNASSMC | |
| | Cetuximab VH- | QVQLKQSGPGLVQPSQSLSITCTVSGFSLTNYGVHWVRQSPGKGLEWLGV | 134 |
| | linker-AB002 VH- | IWSGGNTDYNTPFTSRLSINKDNSKSQVFFKMNSLQSNDTAIYYCARALT | |
| | CH1-hinge-CH2- | YYDYEFAYWGQGTLVTVSAASTKGPSVFPLAPQVQLQQSGAELARPGASV | |
| | CH3(holes) | KMSCKASGYTFTRYTMHWVKQRPGQGLEWIGYINPSRGYTNYNQKFKDKA | |
| | | TLTTDKSSSTAYMQLSSLTSEDSAVYYCARYYDDHYCLDYWGQGTTLTVS | |
| | | SASTKGPSVFPLAPSSKSTSGGTAALGCLVKDYFPEPVTVSWNSGALTSG | |
| | | VHTFPAVLQSSGLYSLSSVVTVPSSSLGTQTYICNVNHKPSNTKVDKKVE | |
| | | PKSCDKTHTCPPCPAPELLGGPSVFLFPPKPKDTLMISRTPEVTCVVVDV | |
| | | SHEDPEVKFNWYVDGVEVHNAKTKPREEQYNSTYRVVSVLTVLHQDWLNG | |
| | | KEYKCKVSNKALPAPIEKTISKAKGQPREPQVYTLPPSREEMTKNQVSLS | |
| | | CAVKGFYPSDIAVEWESNGQPENNYKTTPPVLDSDGSFFLVSKLTVDKSR | |
| | | WQQGNVFSCSVMHEALHNHYTQKSLSLSPGK | |
| | Cetuximab VK- | DILLTQSPVILSVSPGERVSFSCRASQSIGTNIHWYQQRTNGSPRLLIKY | 135 |
| | linker-AB002 VK- | ASESISGIPSRFSGSGSGTDFTLSINSVESEDIADYYCQQNNNWPTTFGA | |
| | Cκ | GTKLELKRTVAAPSVFIFPPQIVLTQSPAIMSASPGEKVTMTCRASSSVS | |
| | | YMNWYQQKSGTSPKRWIYDTSKVASGVPYRFSGSGSGTSYSLTISSMEAE | |
| | | DAATYYCQQWSSNPLTFGSGTKLEINRTVAAPSVFIFPPSDEQLKSGTAS | |
| | | VVCLLNNFYPREAKVQWKVDNALQSGNSQESVTEQDSKDSTYSLSSTLTL | |
| | | SKADYEKHKVYACEVTHQGLSSPVTKSFNRGEC | |
| TMH3 | AB765 VH-linker- | EVQLVESGGGLVQPGGSLRLSCAASGFAFSSYDMSWVRQAPGKGLEWVSY | 136 |
| | MH2n-hinge-CH2- | ISGGGFTYYPDTVKGRFTISRDNSKNTLYLQMNSLRAEDTAVYYCARQGA | |
| | CH3(knobs) | NWELVYWGQGTLVTVSSASTELPPKVSVFVPPRDGFFGNPRKSELICEAT | |
| | | GFSPRQIQVSWLREGKQVGSGVTTDQVQAEAKESGPTTYDVTSTLTIKES | |
| | | DWLGQSMFTCRVDHRGLTFQQNASSMCDKTHTCPPCPAPELLGGPSVFLF | |
| | | PPKPKDTLMISRTPEVTCVVVDVSHEDPEVKFNWYVDGVEVHNAKTKPRE | |
| | | EQYNSTYRVVSVLTVLHQDWLNGKEYKCKVSNKALPAPIEKTISKAKGQP | |
| | | REPQVYTLPPSREEMTKNQVSLWCLVKGFYPSDIAVEWESNGQPENNYKT | |
| | | TPPVLDSDGSFFLYSKLTVDKSRWQQGNVFSCSVMHEALHNHYTQKSLSL | |
| | | SPGK | |
| | AB765 VK-linker- | EIVLTQSPATLSLSPGERATLSCRASQSISDFLHWYQQKPGQAPRLLIKY | 137 |
| | MH2p | ASQSISGIPARFSGSGSGTDFTLTISSLEPEDFAVYYCQNGHNFPPTFGG | |
| | | GTKVEIKRTAELPPKVSVFVPPRDGFFGNPRKSKLICKATGFSPRQIQVS | |
| | | WLREGKQVGSGVTTKQVQAEAKESGPTTYKVTSTLTIKESDWLGQSMFTC | |
| | | RVDHRGLTFQQNASSMC | |
| | Cetuximab VH- | QVQLKQSGPGLVQPSQSLSITCTVSGFSLTNYGVHWVRQSPGKGLEWLGV | 138 |
| | linker-AB002 VH- | IWSGGNTDYNTPFTSRLSINKDNSKSQVFFKMNSLQSNDTAIYYCARALT | |
| | CH1-hinge-CH2- | YYDYEFAYWGQGTLVTVSAASTKGPSVFPLAPQVQLQQSGAELARPGASV | |
| | CH3(holes) | KMSCKASGYTFTRYTMHWVKQRPGQGLEWIGYINPSRGYTNYNQKFKDKA | |
| | | TLTTDKSSSTAYMQLSSLTSEDSAVYYCARYYDDHYCLDYWGQGTTLTVS | |
| | | SASTKGPSVFPLAPSSKSTSGGTAALGCLVKDYFPEPVTVSWNSGALTSG | |
| | | VHTFPAVLQSSGLYSLSSVVTVPSSSLGTQTYICNVNHKPSNTKVDKKVE | |
| | | PKSCDKTHTCPPCPAPELLGGPSVFLFPPKPKDTLMISRTPEVTCVVVDV | |
| | | SHEDPEVKFNWYVDGVEVHNAKTKPREEQYNSTYRVVSVLTVLHQDWLNG | |
| | | KEYKCKVSNKALPAPIEKTISKAKGQPREPQVYTLPPSREEMTKNQVSLS | |
| | | CAVKGFYPSDIAVEWESNGQPENNYKTTPPVLDSDGSFFLVSKLTVDKSR | |
| | | WQQGNVFSCSVMHEALHNHYTQKSLSLSPGK | |
| | Cetuximab VK- | DILLTQSPVILSVSPGERVSFSCRASQSIGTNIHWYQQRTNGSPRLLIKY | 139 |
| | linker-AB002 VK- | ASESISGIPSRFSGSGSGTDFTLSINSVESEDIADYYCQQNNNWPTTFGA | |
| | Cκ | GTKLELKRTVAAPSVFIFPPQIVLTQSPAIMSASPGEKVTMTCRASSSVS | |
| | | YMNWYQQKSGTSPKRWIYDTSKVASGVPYRFSGSGSGTSYSLTISSMEAE | |
| | | DAATYYCQQWSSNPLTFGSGTKLEINRTVAAPSVFIFPPSDEQLKSGTAS | |
| | | VVCLLNNFYPREAKVQWKVDNALQSGNSQESVTEQDSKDSTYSLSSTLTL | |
| | | SKADYEKHKVYACEVTHQGLSSPVTKSFNRGEC | |
| TMH4 | AB765 VH-linker- | EVQLVESGGGLVQPGGSLRLSCAASGFAFSSYDMSWVRQAPGKGLEWVSY | 140 |
| | MH2h-hinge-CH2- | ISGGGFTYYPDTVKGRFTISRDNSKNTLYLQMNSLRAEDTAVYYCARQGA | |
| | CH3(knobs) | NWELVYWGQGTLVTVSSASTELPPKVSVFVPPRDGFFGNPRKSKLACSAT | |
| | | GFSPRQIQVSWLREGKQVGSGVTTDQVQAEAKESGPTTYKVASTLTIKES | |
| | | DWLGQSMFTCRVDHRGLTFQQNASSMCDKTHTCPPCPAPELLGGPSVFLF | |
| | | PPKPKDTLMISRTPEVTCVVVDVSHEDPEVKFNWYVDGVEVHNAKTKPRE | |
| | | EQYNSTYRVVSVLTVLHQDWLNGKEYKCKVSNKALPAPIEKTISKAKGQP | |
| | | REPQVYTLPPSREEMTKNQVSLWCLVKGFYPSDIAVEWESNGQPENNYKT | |
| | | TPPVLDSDGSFFLYSKLTVDKSRWQQGNVFSCSVMHEALHNHYTQKSLSL | |
| | | SPGK | |
| | AB765 VK-linker- | EIVLTQSPATLSLSPGERATLSCRASQSISDFLHWYQQKPGQAPRLLIKY | 141 |
| | MH2k | ASQSISGIPARFSGSGSGTDFTLTISSLEPEDFAVYYCQNGHNFPPTFGG | |
| | | GTKVEIKRTAELPPKVSVFVPPRDGFFGNPRKSKLWCQATGFSPRQIQVS | |
| | | WLREGKQVGSGVTTDQVQAEAKESGPTTYKVTSTLTIKESDWLGQSMFTC | |
| | | RVDHRGLTFQQNASSMC | |
| | Cetuximab VH- | QVQLKQSGPGLVQPSQSLSITCTVSGFSLTNYGVHWVRQSPGKGLEWLGV | 142 |
| | linker-AB002 VH- | IWSGGNTDYNTPFTSRLSINKDNSKSQVFFKMNSLQSNDTAIYYCARALT | |
| | CH1-hinge-CH2- | YYDYEFAYWGQGTLVTVSAASTKGPSVFPLAPQVQLQQSGAELARPGASV | |
| | CH3(holes) | KMSCKASGYTFTRYTMHWVKQRPGQGLEWIGYINPSRGYTNYNQKFKDKA | |
| | | TLTTDKSSSTAYMQLSSLTSEDSAVYYCARYYDDHYCLDYWGQGTTLTVS | |
| | | SASTKGPSVFPLAPSSKSTSGGTAALGCLVKDYFPEPVTVSWNSGALTSG | |
| | | VHTFPAVLQSSGLYSLSSVVTVPSSSLGTQTYICNVNHKPSNTKVDKKVE | |
| | | PKSCDKTHTCPPCPAPELLGGPSVFLFPPKPKDTLMISRTPEVTCVVVDV | |
| | | SHEDPEVKFNWYVDGVEVHNAKTKPREEQYNSTYRVVSVLTVLHQDWLNG | |
| | | KEYKCKVSNKALPAPIEKTISKAKGQPREPQVYTLPPSREEMTKNQVSLS | |
| | | CAVKGFYPSDIAVEWESNGQPENNYKTTPPVLDSDGSFFLVSKLTVDKSR | |
| | | WQQGNVFSCSVMHEALHNHYTQKSLSLSPGK | |
| | Cetuximab VK- | DILLTQSPVILSVSPGERVSFSCRASQSIGTNIHWYQQRTNGSPRLLIKY | 143 |
| | linker-AB002 VK- | ASESISGIPSRFSGSGSGTDFTLSINSVESEDIADYYCQQNNNWPTTFGA | |
| | Cκ | GTKLELKRTVAAPSVFIFPPQIVLTQSPAIMSASPGEKVTMTCRASSSVS | |
| | | YMNWYQQKSGTSPKRWIYDTSKVASGVPYRFSGSGSGTSYSLTISSMEAE | |
| | | DAATYYCQQWSSNPLTFGSGTKLEINRTVAAPSVFIFPPSDEQLKSGTAS | |
| | | VVCLLNNFYPREAKVQWKVDNALQSGNSQESVTEQDSKDSTYSLSSTLTL | |
| | | SKADYEKHKVYACEVTHQGLSSPVTKSFNRGEC | |
| TMH5 | AB765 VH-linker- | EVQLVESGGGLVQPGGSLRLSCAASGFAFSSYDMSWVRQAPGKGLEWVSY | 144 |
| | MH2k-hinge-CH2- | ISGGGFTYYPDTVKGRFTISRDNSKNTLYLQMNSLRAEDTAVYYCARQGA | |
| | CH3(knobs) | NWELVYWGQGTLVTVSSASTELPPKVSVFVPPRDGFFGNPRKSKLWCQAT | |
| | | GFSPRQIQVSWLREGKQVGSGVTTDQVQAEAKESGPTTYKVTSTLTIKES | |
| | | DWLGQSMFTCRVDHRGLTFQQNASSMCDKTHTCPPCPAPELLGGPSVFLF | |
| | | PPKPKDTLMISRTPEVTCVVVDVSHEDPEVKFNWYVDGVEVHNAKTKPRE | |
| | | EQYNSTYRVVSVLTVLHQDWLNGKEYKCKVSNKALPAPIEKTISKAKGQP | |
| | | REPQVYTLPPSREEMTKNQVSLWCLVKGFYPSDIAVEWESNGQPENNYKT | |
| | | TPPVLDSDGSFFLYSKLTVDKSRWQQGNVFSCSVMHEALHNHYTQKSLSL | |
| | | SPGK | |
| | AB765 VK-linker- | EIVLTQSPATLSLSPGERATLSCRASQSISDFLHWYQQKPGQAPRLLIKY | 145 |
| | MH2h | ASQSISGIPARFSGSGSGTDFTLTISSLEPEDFAVYYCQNGHNFPPTFGG | |
| | | GTKVEIKRTAELPPKVSVFVPPRDGFFGNPRKSKLACSATGFSPRQIQVS | |
| | | WLREGKQVGSGVTTDQVQAEAKESGPTTYKVASTLTIKESDWLGQSMFTC | |
| | | RVDHRGLTFQQNASSMC | |
| | Cetuximab VH- | QVQLKQSGPGLVQPSQSLSITCTVSGFSLTNYGVHWVRQSPGKGLEWLGV | 146 |
| | linker-AB002 VH- | IWSGGNTDYNTPFTSRLSINKDNSKSQVFFKMNSLQSNDTAIYYCARALT | |
| | CH1-hinge-CH2- | YYDYEFAYWGQGTLVTVSAASTKGPSVFPLAPQVQLQQSGAELARPGASV | |
| | CH3(holes) | KMSCKASGYTFTRYTMHWVKQRPGQGLEWIGYINPSRGYTNYNQKFKDKA | |
| | | TLTTDKSSSTAYMQLSSLTSEDSAVYYCARYYDDHYCLDYWGQGTTLTVS | |
| | | SASTKGPSVFPLAPSSKSTSGGTAALGCLVKDYFPEPVTVSWNSGALTSG | |
| | | VHTFPAVLQSSGLYSLSSVVTVPSSSLGTQTYICNVNHKPSNTKVDKKVE | |
| | | PKSCDKTHTCPPCPAPELLGGPSVFLFPPKPKDTLMISRTPEVTCVVVDV | |
| | | SHEDPEVKFNWYVDGVEVHNAKTKPREEQYNSTYRVVSVLTVLHQDWLNG | |
| | | KEYKCKVSNKALPAPIEKTISKAKGQPREPQVYTLPPSREEMTKNQVSLS | |
| | | CAVKGFYPSDIAVEWESNGQPENNYKTTPPVLDSDGSFFLVSKLTVDKSR | |
| | | WQQGNVFSCSVMHEALHNHYTQKSLSLSPGK | |
| | Cetuximab VK- | DILLTQSPVILSVSPGERVSFSCRASQSIGTNIHWYQQRTNGSPRLLIKY | 147 |
| | linker-AB002 VK- | ASESISGIPSRFSGSGSGTDFTLSINSVESEDIADYYCQQNNNWPTTFGA | |
| | Cκ | GTKLELKRTVAAPSVFIFPPQIVLTQSPAIMSASPGEKVTMTCRASSSVS | |
| | | YMNWYQQKSGTSPKRWIYDTSKVASGVPYRFSGSGSGTSYSLTISSMEAE | |
| | | DAATYYCQQWSSNPLTFGSGTKLEINRTVAAPSVFIFPPSDEQLKSGTAS | |
| | | VVCLLNNFYPREAKVQWKVDNALQSGNSQESVTEQDSKDSTYSLSSTLTL | |
| | | SKADYEKHKVYACEVTHQGLSSPVTKSFNRGEC | |
| PLY11 | TS2/18 VH-CH1- | EVQLVESGGGLVMPGGSLKLSCAASGFAFSSYDMSWVRQTPEKRLEWVAY | 148 |
| | hinge-CH2- | ISGGGFTYYPDTVKGRFTLSRDNAKNTLYLQMSSLKSEDTAMYYCARQGA | |
| | CH3(knobs) | NWELVYWGQGTLVTVSAASTKGPSVFPLAPSSKSTSGGTAALGCLVKDYF | |
| | | PEPVTVSWNSGALTSGVHTFPAVLQSSGLYSLSSVVTVPSSSLGTQTYIC | |
| | | NVNHKPSNTKVDKKVEPKSCDKTHTCPPCPAPELLGGPSVFLFPPKPKDT | |
| | | LMISRTPEVTCVVVDVSHEDPEVKFNWYVDGVEVHNAKTKPREEQYNSTY | |
| | | RVVSVLTVLHQDWLNGKEYKCKVSNKALPAPIEKTISKAKGQPREPQVYT | |
| | | LPPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGK | |
| | TS2/18 VK-CK | DIVMTQSPATLSVTPGDRVFLSCRASQSISDFLHWYQQKSHESPRLLIKY | 149 |
| | | ASQSISGIPSRFSGSGSGSDFTLSINSVEPEDVGVYFCQNGHNFPPTFGG | |
| | | GTKLEIKRTVAAPSVFIFPPSDEQLKSGTASVVCLLNNFYPREAKVQWKV | |
| | | DNALQSGNSQESVTEQDSKDSTYSLSSTLTLSKADYEKHKVYACEVTHQG | |
| | | LSSPVTKSFNRGEC | |
| | Cetuximab VH- | QVQLKQSGPGLVQPSQSLSITCTVSGFSLTNYGVHWVRQSPGKGLEWLGV | 150 |
| | linker-AB002 VH- | IWSGGNTDYNTPFTSRLSINKDNSKSQVFFKMNSLQSNDTAIYYCARALT | |
| | CH1-hinge-CH2- | YYDYEFAYWGQGTLVTVSAASTKGPSVFPLAPQVQLQQSGAELARPGASV | |
| | CH3(holes) | KMSCKASGYTFTRYTMHWVKQRPGQGLEWIGYINPSRGYTNYNQKFKDKA | |
| | | TLTTDKSSSTAYMQLSSLTSEDSAVYYCARYYDDHYCLDYWGQGTTLTVS | |
| | | SASTKGPSVFPLAPSSKSTSGGTAALGCLVKDYFPEPVTVSWNSGALTSG | |
| | | VHTFPAVLQSSGLYSLSSVVTVPSSSLGTQTYICNVNHKPSNTKVDKKVE | |
| | | PKSCDKTHTCPPCPAPELLGGPSVFLFPPKPKDTLMISRTPEVTCVVVDV | |
| | | SHEDPEVKFNWYVDGVEVHNAKTKPREEQYNSTYRVVSVLTVLHQDWLNG | |
| | | KEYKCKVSNKALPAPIEKTISKAKGQPREPQVYTLPPSREEMTKNQVSLS | |
| | | CAVKGFYPSDIAVEWESNGQPENNYKTTPPVLDSDGSFFLVSKLTVDKSR | |
| | | WQQGNVFSCSVMHEALHNHYTQKSLSLSPGK | |
| | Cetuximab VK- | DILLTQSPVILSVSPGERVSFSCRASQSIGTNIHWYQQRTNGSPRLLIKY | 151 |
| | linker-AB002 VK- | ASESISGIPSRFSGSGSGTDFTLSINSVESEDIADYYCQQNNNWPTTFGA | |
| | Cκ | GTKLELKRTVAAPSVFIFPPQIVLTQSPAIMSASPGEKVTMTCRASSSVS | |
| | | YMNWYQQKSGTSPKRWIYDTSKVASGVPYRFSGSGSGTSYSLTISSMEAE | |
| | | DAATYYCQQWSSNPLTFGSGTKLEINRTVAAPSVFIFPPSDEQLKSGTAS | |
| | | VVCLLNNFYPREAKVQWKVDNALQSGNSQESVTEQDSKDSTYSLSSTLTL | |
| | | SKADYEKHKVYACEVTHQGLSSPVTKSFNRGEC | |
| TS2/18 halfbody | TS2/18 VH-CH1- | EVQLVESGGGLVMPGGSLKLSCAASGFAFSSYDMSWVRQTPEKRLEWVAY | 152 |
| | hinge-CH2-CH3 | ISGGGFTYYPDTVKGRFTLSRDNAKNTLYLQMSSLKSEDTAMYYCARQGA | |
| | (halfbody) | NWELVYWGQGTLVTVSAASTKGPSVFPLAPSSKSTSGGTAALGCLVKDYF | |
| | | PEPVTVSWNSGALTSGVHTFPAVLQSSGLYSLSSVVTVPSSSLGTQTYIC | |
| | | NVNHKPSNTKVDKKVEPKSCDKTHTSPPSPAPELLGGPSVFLFPPKPKDT | |
| | | LMISRTPEVTCVVVDVSHEDPEVKFNWYVDGVEVHNAKTKPREEQYNSTY | |
| | | RVVSVLTVLHQDWLNGKEYKCKVSNKALPAPIEKTISKAEGQPREPQVYT | |
| | | LPPSREEMTKNQVSLTCLVKGFYPSDIAVEWESNGQPENNYKTTPPVLDS | |
| | | DGSFRLYSKLTVDKSRWQQGNVFSCSVMHEALHNHYTQKSLSLSPGK | |
| | T32/18 VK-CK | DIVMTQSPATLSVTPGDRVFLSCRASQSISDFLHWYQQKSHESPRLLIKY | 153 |
| | | ASQSISGIPSRFSGSGSGSDFTLSINSVEPEDVGVYFCQNGHNFPPTFGG | |
| | | GTKLEIKRTVAAPSVFIFPPSDEQLKSGTASVVCLLNNFYPREAKVQWKV | |
| | | DNALQSGNSQESVTEQDSKDSTYSLSSTLTLSKADYEKHKVYACEVTHQG | |
| | | LSSPVTKSFNRGEC | |
| DVD860 halfbody | Cetuximab VH- | QVQLKQSGPGLVQPSQSLSITCTVSGFSLTNYGVHWVRQSPGKGLEWLGV | 154 |
| | linker-AB002 VH- | IWSGGNTDYNTPFTSRLSINKDNSKSQVFFKMNSLQSNDTAIYYCARALT | |
| | CH1-hinge-CH2-CH3 | YYDYEFAYWGQGTLVTVSAASTKGPSVFPLAPQVQLQQSGAELARPGASV | |
| | (halfbody) | KMSCKASGYTFTRYTMHWVKQRPGQGLEWIGYINPSRGYTNYNQKFKDKA | |
| | | TLTTDKSSSTAYMQLSSLTSEDSAVYYCARYYDDHYCLDYWGQGTTLTVS | |
| | | SASTKGPSVFPLAPSSKSTSGGTAALGCLVKDYFPEPVTVSWNSGALTSG | |
| | | VHTFPAVLQSSGLYSLSSVVTVPSSSLGTQTYICNVNHKPSNTKVDKKVE | |
| | | PKSCDKTHTSPPSPAPELLGGPSVFLFPPKPKDTLMISRTPEVTCVVVDV | |
| | | SHEDPEVKFNWYVDGVEVHNAKTKPREEQYNSTYRVVSVLTVLHQDWLNG | |
| | | KEYKCKVSNKALPAPIEKTISKAKGQPREPQVYTLPPSREEMTKNQVSLT | |
| | | CLVKGFYPSDIAVEWESNGQPENNYKTTPPVLDSDGSFRLYSKLTVDKSR | |
| | | WQQGNVFSCSVMHEALHNHYTQKSLSLSPGK | |
| | Cetuximab VK- | DILLTQSPVILSVSPGERVSFSCRASQSIGTNIHWYQQRTNGSPRLLIKY | 155 |
| | linker-AB002 VK- | ASESISGIPSRFSGSGSGTDFTLSINSVESEDIADYYCQQNNNWPTTFGA | |
| | CK | GTKLELKRTVAAPSVFIFPPQIVLTQSPAIMSASPGEKVTMTCRASSSVS | |
| | | YMNWYQQKSGTSPKRWIYDTSKVASGVPYRFSGSGSGTSYSLTISSMEAE | |
| | | DAATYYCQQWSSNPLTFGSGTKLEINRTVAAPSVFIFPPSDEQLKSGTAS | |
| | | VVCLLNNFYPREAKVQWKVDNALQSGNSQESVTEQDSKDSTYSLSSTLTL | |
| | | SKADYEKHKVYACEVTHQGLSSPVTKSFNRGEC | |
| TMH6 | AB426 VH-linker- | QVQLVESGGGVVQPGRSLRLDCKASGITFSNSGMHWVRQAPGKGLEWVAV | 156 |
| | MH2-hinge- | IWYDGSKRYYADSVKGRFTISRDNSKNTLFLQMNSLRAEDTAVYYCATND | |
| | CH2(LALA)- | DYWGQGTLVTVSSASTELPPKVSVFVPPRDGFFGNPRKSKLICQATGFSP | |
| | CH3(knobs) | RQIQVSWLREGKQVGSGVTTDQVQAEAKESGPTTYKVTSTLTIKESDWLG | |
| | | QSMFTCRVDHRGLTFQQNASSMCDKTHTCPPCPAPEAAGGPSVFLFPPKP | |
| | | KDTLMISRTPEVTCVVVDVSHEDPEVKFNWYVDGVEVHNAKTKPREEQYN | |
| | | STYRVVSVLTVLHQDWLNGKEYKCKVSNKALPAPIEKTISKAKGQPREPQ | |
| | | VYTLPPSREEMTKNQVSLWCLVKGFYPSDIAVEWESNGQPENNYKTTPPV | |
| | | LDSDGSFFLYSKLTVDKSRWQQGNVFSCSVMHEALHNHYTQKSLSLSPGK | |
| | AB426 VL-linker- | EIVLTQSPATLSLSPGERATLSCRASQSVSSYLAWYQQKPGQAPRLLIYD | 157 |
| | MH2 | ASNRATGIPARFSGSGSGTDFTLTISSLEPEDFAVYYCQQSSNWPRTFGQ | |
| | | GTKVEIKRTAELPPKVSVFVPPRDGFFGNPRKSKLICQATGFSPRQIQVS | |
| | | WLREGKQVGSGVTTDQVQAEAKESGPTTYKVTSTLTIKESDWLGQSMFTC | |
| | | RVDHRGLTFQQNASSMC | |
| | Cetuximab.2 VH- | QVQLKQSGPGLVQPSQSLSITCTVSGFSLTNYGVHWVRQSPGKGLEWLGV | 158 |
| | linker-AB002.2 | IWSGGNTDYNTPFTSRLSINKDNSKSQVFFKMNSLQSEDTAIYYCARALT | |
| | VH-CH1-hinge- | YYDYEFAYWGQGTLVTVSAASTKGPSVFPLAPQVQLQQSGAELARPGASV | |
| | CH2(LALA)- | KMSCKASGYTFTRYTMHWVKQRPGQGLEWIGYINPSRGYTNYNQKFKDKA | |
| | CH3(holes) | TLTTDKSSSTAYMQLSSLTSEDSAVYYCARYYDDHYSLDYWGQGTTLTVS | |
| | | SASTKGPSVFPLAPSSKSTSGGTAALGCLVKDYFPEPVTVSWNSGALTSG | |
| | | VHTFPAVLQSSGLYSLSSVVTVPSSSLGTQTYICNVNHKPSNTKVDKKVE | |
| | | PKSCDKTHTCPPCPAPEAAGGPSVFLFPPKPKDTLMISRTPEVTCVVVDV | |
| | | SHEDPEVKFNWYVDGVEVHNAKTKPREEQYNSTYRVVSVLTVLHQDWLNG | |
| | | KEYKCKVSNKALPAPIEKTISKAKGQPREPQVYTLPPSREEMTKNQVSLS | |
| | | CAVKGFYPSDIAVEWESNGQPENNYKTTPPVLDSDGSFFLVSKLTVDKSR | |
| | | WQQGNVFSCSVMHEALHNHYTQKSLSLSPGK | |
| | Cetuximab.2 VK- | DILLTQSPVILSVSPGERVSFSCRASQSIGTNIHWYQQRTSGSPRLLIKY | 159 |
| | linker-AB002.2 | ASESISGIPSRFSGSGSGTDFTLSINSVESEDIADYYCQQNNNWPTTFGA | |
| | VK-CK | GTKLELKRTVAAPSVFIFPPQIVLTQSPAIMSASPGEKVTMTCRASSSVS | |
| | | YMNWYQQKSGTSPKRWIYDTSKVASGVPYRFSGSGSGTSYSLTISSMEAE | |
| | | DAATYYCQQWSSNPLTFGSGTKLEIKRTVAAPSVFIFPPSDEQLKSGTAS | |
| | | VVCLLNNFYPREAKVQWKVDNALQSGNSQESVTEQDSKDSTYSLSSTLTL | |
| | | SKADYEKHKVYACEVTHQGLSSPVTKSFNRGEC | |
| TMH7 | AB426 VH-linker- | QVQLVESGGGVVQPGRSLRLDCKASGITFSNSGMHWVRQAPGKGLEWVAV | 160 |
| | MH2n-hinge- | IWYDGSKRYYADSVKGRFTISRDNSKNTLFLQMNSLRAEDTAVYYCATND | |
| | CH2(LALA)- | DYWGQGTLVTVSSASTELPPKVSVFVPPRDGFFGNPRKSELICEATGFSP | |
| | CH3(knobs) | RQIQVSWLREGKQVGSGVTTDQVQAEAKESGPTTYDVTSTLTIKESDWLG | |
| | | QSMFTCRVDHRGLTFQQNASSMCDKTHTCPPCPAPEAAGGPSVFLFPPKP | |
| | | KDTLMISRTPEVTCVVVDVSHEDPEVKFNWYVDGVEVHNAKTKPREEQYN | |
| | | STYRVVSVLTVLHQDWLNGKEYKCKVSNKALPAPIEKTISKAKGQPREPQ | |
| | | VYTLPPSREEMTKNQVSLWCLVKGFYPSDIAVEWESNGQPENNYKTTPPV | |
| | | LDSDGSFFLYSKLTVDKSRWQQGNVFSCSVMHEALHNHYTQKSLSLSPGK | |
| | AB426 VL-linker- | EIVLTQSPATLSLSPGERATLSCRASQSVSSYLAWYQQKPGQAPRLLIYD | 161 |
| | MH2p | ASNRATGIPARFSGSGSGTDFTLTISSLEPEDFAVYYCQQSSNWPRTFGQ | |
| | | GTKVEIKRTAELPPKVSVFVPPRDGFFGNPRKSKLICKATGFSPRQIQVS | |
| | | WLREGKQVGSGVTTKQVQAEAKESGPTTYKVTSTLTIKESDWLGQSMFTC | |
| | | RVDHRGLTFQQNASSMC | |
| | Cetuximab.2 VH- | QVQLKQSGPGLVQPSQSLSITCTVSGFSLTNYGVHWVRQSPGKGLEWLGV | 162 |
| | linker-AB002.2 | IWSGGNTDYNTPFTSRLSINKDNSKSQVFFKMNSLQSEDTAIYYCARALT | |
| | VH-CH1-hinge- | YYDYEFAYWGQGTLVTVSAASTKGPSVFPLAPQVQLQQSGAELARPGASV | |
| | CH2(LALA)- | KMSCKASGYTFTRYTMHWVKQRPGQGLEWIGYINPSRGYTNYNQKFKDKA | |
| | CH3(holes) | TLTTDKSSSTAYMQLSSLTSEDSAVYYCARYYDDHYSLDYWGQGTTLTVS | |
| | | SASTKGPSVFPLAPSSKSTSGGTAALGCLVKDYFPEPVTVSWNSGALTSG | |
| | | VHTFPAVLQSSGLYSLSSVVTVPSSSLGTQTYICNVNHKPSNTKVDKKVE | |
| | | PKSCDKTHTCPPCPAPEAAGGPSVFLFPPKPKDTLMISRTPEVTCVVVDV | |
| | | SHEDPEVKFNWYVDGVEVHNAKTKPREEQYNSTYRVVSVLTVLHQDWLNG | |
| | | KEYKCKVSNKALPAPIEKTISKAKGQPREPQVYTLPPSREEMTKNQVSLS | |
| | | CAVKGFYPSDIAVEWESNGQPENNYKTTPPVLDSDGSFFLVSKLTVDKSR | |
| | | WQQGNVFSCSVMHEALHNHYTQKSLSLSPGK | |
| | Cetuximab.2 VK- | DILLTQSPVILSVSPGERVSFSCRASQSIGTNIHWYQQRTSGSPRLLIKY | 163 |
| | linker-AB002.2 | ASESISGIPSRFSGSGSGTDFTLSINSVESEDIADYYCQQNNNWPTTFGA | |
| | VK-CK | GTKLELKRTVAAPSVFIFPPQIVLTQSPAIMSASPGEKVTMTCRASSSVS | |
| | | YMNWYQQKSGTSPKRWIYDTSKVASGVPYRFSGSGSGTSYSLTISSMEAE | |
| | | DAATYYCQQWSSNPLTFGSGTKLEIKRTVAAPSVFIFPPSDEQLKSGTAS | |
| | | VVCLLNNFYPREAKVQWKVDNALQSGNSQESVTEQDSKDSTYSLSSTLTL | |
| | | SKADYEKHKVYACEVTHQGLSSPVTKSFNRGEC | |
| TMH8 | AB426 VH-linker- | QVQLVESGGGVVQPGRSLRLDCKASGITFSNSGMHWVRQAPGKGLEWVAV | 164 |
| | MH2p-hinge- | IWYDGSKRYYADSVKGRFTISRDNSKNTLFLQMNSLRAEDTAVYYCATND | |
| | CH2(LALA)- | DYWGQGTLVTVSSASTELPPKVSVFVPPRDGFFGNPRKSKLICKATGFSP | |
| | CH3(knobs) | RQIQVSWLREGKQVGSGVTTKQVQAEAKESGPTTYKVTSTLTIKESDWLG | |
| | | QSMFTCRVDHRGLTFQQNASSMCDKTHTCPPCPAPEAAGGPSVFLFPPKP | |
| | | KDTLMISRTPEVTCVVVDVSHEDPEVKFNWYVDGVEVHNAKTKPREEQYN | |
| | | STYRVVSVLTVLHQDWLNGKEYKCKVSNKALPAPIEKTISKAKGQPREPQ | |
| | | VYTLPPSREEMTKNQVSLWCLVKGFYPSDIAVEWESNGQPENNYKTTPPV | |
| | | LDSDGSFFLYSKLTVDKSRWQQGNVFSCSVMHEALHNHYTQKSLSLSPGK | |
| | AB426 VL-linker- | EIVLTQSPATLSLSPGERATLSCRASQSVSSYLAWYQQKPGQAPRLLIYD | 165 |
| | MH2n | ASNRATGIPARFSGSGSGTDFTLTISSLEPEDFAVYYCQQSSNWPRTFGQ | |
| | | GTKVEIKRTAELPPKVSVFVPPRDGFFGNPRKSELICEATGFSPRQIQVS | |
| | | WLREGKQVGSGVTTDQVQAEAKESGPTTYDVTSTLTIKESDWLGQSMFTC | |
| | | RVDHRGLTFQQNASSMC | |
| | Cetuximab.2 VH- | QVQLKQSGPGLVQPSQSLSITCTVSGFSLTNYGVHWVRQSPGKGLEWLGV | 166 |
| | linker-CD3.2 VH- | IWSGGNTDYNTPFTSRLSINKDNSKSQVFFKMNSLQSEDTAIYYCARALT | |
| | CH1-hinge- | YYDYEFAYWGQGTLVTVSAASTKGPSVFPLAPQVQLQQSGAELARPGASV | |
| | CH2(LALA)- | KMSCKASGYTFTRYTMHWVKQRPGQGLEWIGYINPSRGYTNYNQKFKDKA | |
| | CH3(holes) | TLTTDKSSSTAYMQLSSLTSEDSAVYYCARYYDDHYSLDYWGQGTTLTVS | |
| | | SASTKGPSVFPLAPSSKSTSGGTAALGCLVKDYFPEPVTVSWNSGALTSG | |
| | | VHTFPAVLQSSGLYSLSSVVTVPSSSLGTQTYICNVNHKPSNTKVDKKVE | |
| | | PKSCDKTHTCPPCPAPEGPSVFLFPPKPKDTLMISRTPEVTCVVVDV | |
| | | SHEDPEVKFNWYVDGVEVHNAKTKPREEQYNSTYRVVSVLTVLHQDWLNG | |
| | | KEYKCKVSNKALPAPIEKTISKAKGQPREPQVYTLPPSREEMTKNQVSLS | |
| | | CAVKGFYPSDIAVEWESNGQPENNYKTTPPVLDSDGSFFLVSKLTVDKSR | |
| | | WQQGNVFSCSVMHEALHNHYTQKSLSLSPGK | |
| | Cetuximab.2 VK- | DILLTQSPVILSVSPGERVSFSCRASQSIGTNIHWYQQRTSGSPRLLIKY | 167 |
| | linker-AB002.2 | ASESISGIPSRFSGSGSGTDFTLSINSVESEDIADYYCQQNNNWPTTFGA | |
| | VK-CK | GTKLELKRTVAAPSVFIFPPQIVLTQSPAIMSASPGEKVTMTCRASSSVS | |
| | | YMNWYQQKSGTSPKRWIYDTSKVASGVPYRFSGSGSGTSYSLTISSMEAE | |
| | | DAATYYCQQWSSNPLTFGSGTKLEIKRTVAAPSVFIFPPSDEQLKSGTAS | |
| | | VVCLLNNFYPREAKVQWKVDNALQSGNSQESVTEQDSKDSTYSLSSTLTL | |
| | | SKADYEKHKVYACEVTHQGLSSPVTKSFNRGEC | |
| TMH9 | AB426 VH-linker- | QVQLVESGGGVVQPGRSLRLDCKASGITFSNSGMHWVRQAPGKGLEWVAV | 168 |
| | MH2h-hinge- | IWYDGSKRYYADSVKGRFTISRDNSKNTLFLQMNSLRAEDTAVYYCATND | |
| | CH2(LALA)- | DYWGQGTLVTVSSASTELPPKVSVFVPPRDGFFGNPRKSKLACSATGFSP | |
| | CH3(knobs) | RQIQVSWLREGKQVGSGVTTDQVQAEAKESGPTTYKVASTLTIKESDWLG | |
| | | QSMFTCRVDHRGLTFQQNASSMCDKTHTCPPCPAPEAAGGPSVFLFPPKP | |
| | | KDTLMISRTPEVTCVVVDVSHEDPEVKFNWYVDGVEVHNAKTKPREEQYN | |
| | | STYRVVSVLTVLHQDWLNGKEYKCKVSNKALPAPIEKTISKAKGQPREPQ | |
| | | VYTLPPSREEMTKNQVSLWCLVKGFYPSDIAVEWESNGQPENNYKTTPPV | |
| | | LDSDGSFFLYSKLTVDKSRWQQGNVFSCSVMHEALHNHYTQKSLSLSPGK | |
| | AB426 VL-linker- | EIVLTQSPATLSLSPGERATLSCRASQSVSSYLAWYQQKPGQAPRLLIYD | 169 |
| | MH2k | ASNRATGIPARFSGSGSGTDFTLTISSLEPEDFAVYYCQQSSNWPRTFGQ | |
| | | GTKVEIKRTAELPPKVSVFVPPRDGFFGNPRKSKLWCQATGFSPRQIQVS | |
| | | WLREGKQVGSGVTTDQVQAEAKESGPTTYKVTSTLTIKESDWLGQSMFTC | |
| | | RVDHRGLTFQQNASSMC | |
| | Cetuximab.2 VH- | QVQLKQSGPGLVQPSQSLSITCTVSGFSLTNYGVHWVRQSPGKGLEWLGV | 170 |
| | linker-AB002.2 | IWSGGNTDYNTPFTSRLSINKDNSKSQVFFKMNSLQSEDTAIYYCARALT | |
| | VH-CH1-hinge- | YYDYEFAYWGQGTLVTVSAASTKGPSVFPLAPQVQLQQSGAELARPGASV | |
| | CH2(LALA)- | KMSCKASGYTFTRYTMHWVKQRPGQGLEWIGYINPSRGYTNYNQKFKDKA | |
| | CH3(holes) | TLTTDKSSSTAYMQLSSLTSEDSAVYYCARYYDDHYSLDYWGQGTTLTVS | |
| | | SASTKGPSVFPLAPSSKSTSGGTAALGCLVKDYFPEPVTVSWNSGALTSG | |
| | | VHTFPAVLQSSGLYSLSSVVTVPSSSLGTQTYICNVNHKPSNTKVDKKVE | |
| | | PKSCDKTHTCPPCPAPEAAGGPSVFLFPPKPKDTLMISRTPEVTCVVVDV | |
| | | SHEDPEVKFNWYVDGVEVHNAKTKPREEQYNSTYRVVSVLTVLHQDWLNG | |
| | | KEYKCKVSNKALPAPIEKTISKAKGQPREPQVYTLPPSREEMTKNQVSLS | |
| | | CAVKGFYPSDIAVEWESNGQPENNYKTTPPVLDSDGSFFLVSKLTVDKSR | |
| | | WQQGNVFSCSVMHEALHNHYTQKSLSLSPGK | |
| | Cetuximab.2 VK- | DILLTQSPVILSVSPGERVSFSCRASQSIGTNIHWYQQRTSGSPRLLIKY | 171 |
| | linker-AB002.2 | ASESISGIPSRFSGSGSGTDFTLSINSVESEDIADYYCQQNNNWPTTFGA | |
| | VK-CK | GTKLELKRTVAAPSVFIFPPQIVLTQSPAIMSASPGEKVTMTCRASSSVS | |
| | | YMNWYQQKSGTSPKRWIYDTSKVASGVPYRFSGSGSGTSYSLTISSMEAE | |
| | | DAATYYCQQWSSNPLTFGSGTKLEIKRTVAAPSVFIFPPSDEQLKSGTAS | |
| | | VVCLLNNFYPREAKVQWKVDNALQSGNSQESVTEQDSKDSTYSLSSTLTL | |
| | | SKADYEKHKVYACEVTHQGLSSPVTKSFNRGEC | |
| TMH-1.0 | AB426 VH-linker- | QVQLVESGGGVVQPGRSLRLDCKASGITFSNSGMHWVRQAPGKGLEWVAV | 172 |
| | MH2k-hinge- | IWYDGSKRYYADSVKGRFTISRDNSKNTLFLQMNSLRAEDTAVYYCATND | |
| | CH2(LALA)- | DYWGQGTLVTVSSASTELPPKVSVFVPPRDGFFGNPRKSKLWCQATGFSP | |
| | CH3(knobs) | RQIQVSWLREGKQVGSGVTTDQVQAEAKESGPTTYKVTSTLTIKESDWLG | |
| | | QSMFTCRVDHRGLTFQQNASSMCDKTHTCPPCPAPEAAGGPSVFLFPPKP | |
| | | KDTLMISRTPEVTCVVVDVSHEDPEVKFNWYVDGVEVHNAKTKPREEQYN | |
| | | STYRVVSVLTVLHQDWLNGKEYKCKVSNKALPAPIEKTISKAKGQPREPQ | |
| | | VYTLPPSREEMTKNQVSLWCLVKGFYPSDIAVEWESNGQPENNYKTTPPV | |
| | | LDSDGSFFLYSKLTVDKSRWQQGNVFSCSVMHEALHNHYTQKSLSLSPGK | |
| | AB426 VL-linker- | EIVLTQSPATLSLSPGERATLSCRASQSVSSYLAWYQQKPGQAPRLLIYD | 173 |
| | MH2h | ASNRATGIPARFSGSGSGTDFTLTISSLEPEDFAVYYCQQSSNWPRTFGQ | |
| | | GTKVEIKRTAELPPKVSVFVPPRDGFFGNPRKSKLACSATGFSPRQIQVS | |
| | | WLREGKQVGSGVTTDQVQAEAKESGPTTYKVASTLTIKESDWLGQSMFTC | |
| | | RVDHRGLTFQQNASSMC | |
| | Cetuximab.2 VH- | QVQLKQSGPGLVQPSQSLSITCTVSGFSLTNYGVHWVRQSPGKGLEWLGV | 174 |
| | linker-AB002.2 | IWSGGNTDYNTPFTSRLSINKDNSKSQVFFKMNSLQSEDTAIYYCARALT | |
| | VH-CH1-hinge- | YYDYEFAYWGQGTLVTVSAASTKGPSVFPLAPQVQLQQSGAELARPGASV | |
| | CH2(LALA)- | KMSCKASGYTFTRYTMHWVKQRPGQGLEWIGYINPSRGYTNYNQKFKDKA | |
| | CH3(holes) | TLTTDKSSSTAYMQLSSLTSEDSAVYYCARYYDDHYSLDYWGQGTTLTVS | |
| | | SASTKGPSVFPLAPSSKSTSGGTAALGCLVKDYFPEPVTVSWNSGALTSG | |
| | | VHTFPAVLQSSGLYSLSSVVTVPSSSLGTQTYICNVNHKPSNTKVDKKVE | |
| | | PKSCDKTHTCPPCPAPEAAGGPSVFLFPPKPKDTLMISRTPEVTCVVVDV | |
| | | SHEDPEVKFNWYVDGVEVHNAKTKPREEQYNSTYRVVSVLTVLHQDWLNG | |
| | | KEYKCKVSNKALPAPIEKTISKAKGQPREPQVYTLPPSREEMTKNQVSLS | |
| | | CAVKGFYPSDIAVEWESNGQPENNYKTTPPVLDSDGSFFLVSKLTVDKSR | |
| | | WQQGNVFSCSVMHEALHNHYTQKSLSLSPGK | |
| | Cetuximab.2 VK- | DILLTQSPVILSVSPGERVSFSCRASQSIGTNIHWYQQRTSGSPRLLIKY | 175 |
| | linker-AB002.2 | ASESISGIPSRFSGSGSGTDFTLSINSVESEDIADYYCQQNNNWPTTFGA | |
| | VK-CK | GTKLELKRTVAAPSVFIFPPQIVLTQSPAIMSASPGEKVTMTCRASSSVS | |
| | | YMNWYQQKSGTSPKRWIYDTSKVASGVPYRFSGSGSGTSYSLTISSMEAE | |
| | | DAATYYCQQWSSNPLTFGSGTKLEIKRTVAAPSVFIFPPSDEQLKSGTAS | |
| | | VVCLLNNFYPREAKVQWKVDNALQSGNSQESVTEQDSKDSTYSLSSTLTL | |
| | | SKADYEKHKVYACEVTHQGLSSPVTKSFNRGEC | |
| TMH-1.1 | YW243 VH-linker- | EVQLVESGGGLVQPGGSLRLSCAASGFTFSDSWIHWVRQAPGKGLEWVAW | 176 |
| | MH2-hinge- | ISPYGGSTYYADSVKGRFTISADTSKNTAYLQMNSLRAEDTAVYYCARRH | |
| | CH2(LALA)-CH3 | YPGGFDYWGQGTLVTVSAASTELPPKVSVFVPPRDGFFGNPRKSKLICQA | |
| | (knobs) | TGFSPRQIQVSWLREGKQVGSGVTTDQVQAEAKESGPTTYKVTSTLTIKE | |
| | | SDWLGQSMFTCRVDHRGLTFQQNASSMCDKTHTCPPCPAPEAAGGPSVFL | |
| | | FPPKPKDTLMISRTPEVTCVVVDVSHEDPEVKFNWYVDGVEVHNAKTKPR | |
| | | EEQYNSTYRVVSVLTVLHQDWLNGKEYKCKVSNKALPAPIEKTISKAKGQ | |
| | | PREPQVYTLPPSREEMTKNQVSLWCLVKGFYPSDIAVEWESNGQPENNYK | |
| | | TTPPVLDSDGSFFLYSKLTVDKSRWQQGNVFSCSVMHEALHNHYTQKSLS | |
| | | LSPGK | |
| | YW243 VK-linker- | DIQMTQSPSSLSASVGDRVTITCRASQDVSTAVAWYQQKPGKAPKLLIYS | 177 |
| | MH2 | ASFLYSGVPSRFSGSGSGTDFTLTISSLQPEDFATYYCQQYLYHPATFGQ | |
| | | GTKVEIKRTAELPPKVSVFVPPRDGFFGNPRKSKLICQATGFSPRQIQVS | |
| | | WLREGKQVGSGVTTDQVQAEAKESGPTTYKVTSTLTIKESDWLGQSMFTC | |
| | | RVDHRGLTFQQNASSMC | |
| | Cetuximab.2 VH- | QVQLKQSGPGLVQPSQSLSITCTVSGFSLTNYGVHWVRQSPGKGLEWLGV | 178 |
| | linker-AB002.2 | IWSGGNTDYNTPFTSRLSINKDNSKSQVFFKMNSLQSEDTAIYYCARALT | |
| | VH-CH1-hinge- | YYDYEFAYWGQGTLVTVSAASTKGPSVFPLAPQVQLQQSGAELARPGASV | |
| | CH2(LALA)- | KMSCKASGYTFTRYTMHWVKQRPGQGLEWIGYINPSRGYTNYNQKFKDKA | |
| | CH3(holes) | TLTTDKSSSTAYMQLSSLTSEDSAVYYCARYYDDHYSLDYWGQGTTLTVS | |
| | | SASTKGPSVFPLAPSSKSTSGGTAALGCLVKDYFPEPVTVSWNSGALTSG | |
| | | VHTFPAVLQSSGLYSLSSVVTVPSSSLGTQTYICNVNHKPSNTKVDKKVE | |
| | | PKSCDKTHTCPPCPAPEAAGGPSVFLFPPKPKDTLMISRTPEVTCVVVDV | |
| | | SHEDPEVKFNWYVDGVEVHNAKTKPREEQYNSTYRVVSVLTVLHQDWLNG | |
| | | KEYKCKVSNKALPAPIEKTISKAKGQPREPQVYTLPPSREEMTKNQVSLS | |
| | | CAVKGFYPSDIAVEWESNGQPENNYKTTPPVLDSDGSFFLVSKLTVDKSR | |
| | | WQQGNVFSCSVMHEALHNHYTQKSLSLSPGK | |
| | Cetuximab.2 VK- | DILLTQSPVILSVSPGERVSFSCRASQSIGTNIHWYQQRTSGSPRLLIKY | 179 |
| | linker-AB002.2 | ASESISGIPSRFSGSGSGTDFTLSINSVESEDIADYYCQQNNNWPTTFGA | |
| | VK-CK | GTKLELKRTVAAPSVFIFPPQIVLTQSPAIMSASPGEKVTMTCRASSSVS | |
| | | YMNWYQQKSGTSPKRWIYDTSKVASGVPYRFSGSGSGTSYSLTISSMEAE | |
| | | DAATYYCQQWSSNPLTFGSGTKLEIKRTVAAPSVFIFPPSDEQLKSGTAS | |
| | | VVCLLNNFYPREAKVQWKVDNALQSGNSQESVTEQDSKDSTYSLSSTLTL | |
| | | SKADYEKHKVYACEVTHQGLSSPVTKSFNRGEC | |
| TMH12 | YW243 VH-linker- | EVQLVESGGGLVQPGGSLRLSCAASGFTFSDSWIHWVRQAPGKGLEWVAW | 180 |
| | MH2n-hinge- | ISPYGGSTYYADSVKGRFTISADTSKNTAYLQMNSLRAEDTAVYYCARRH | |
| | CH2(LALA)-CH3 | YPGGFDYWGQGTLVTVSAASTELPPKVSVFVPPRDGFFGNPRKSELICEA | |
| | (knobs) | TGFSPRQIQVSWLREGKQVGSGVTTDQVQAEAKESGPTTYDVTSTLTIKE | |
| | | SDWLGQSMFTCRVDHRGLTFQQNASSMCDKTHTCPPCPAPEAAGGPSVFL | |
| | | FPPKPKDTLMISRTPEVTCVVVDVSHEDPEVKFNWYVDGVEVHNAKTKPR | |
| | | EEQYNSTYRVVSVLTVLHQDWLNGKEYKCKVSNKALPAPIEKTISKAKGQ | |
| | | PREPQVYTLPPSREEMTKNQVSLWCLVKGFYPSDIAVEWESNGQPENNYK | |
| | | TTPPVLDSDGSFFLYSKLTVDKSRWQQGNVFSCSVMHEALHNHYTQKSLS | |
| | | LSPGK | |
| | YW243 VK-linker- | DIQMTQSPSSLSASVGDRVTITCRASQDVSTAVAWYQQKPGKAPKLLIYS | 181 |
| | MH2p | ASFLYSGVPSRFSGSGSGTDFTLTISSLQPEDFATYYCQQYLYHPATFGQ | |
| | | GTKVEIKRTAELPPKVSVFVPPRDGFFGNPRKSKLICKATGFSPRQIQVS | |
| | | WLREGKQVGSGVTTKQVQAEAKESGPTTYKVTSTLTIKESDWLGQSMFTC | |
| | | RVDHRGLTFQQNASSMC | |
| | Cetuximab.2 VH- | QVQLKQSGPGLVQPSQSLSITCTVSGFSLTNYGVHWVRQSPGKGLEWLGV | 182 |
| | linker-AB002.2 | IWSGGNTDYNTPFTSRLSINKDNSKSQVFFKMNSLQSEDTAIYYCARALT | |
| | VH-CH1-hinge- | YYDYEFAYWGQGTLVTVSAASTKGPSVFPLAPQVQLQQSGAELARPGASV | |
| | CH2(LALA)- | KMSCKASGYTFTRYTMHWVKQRPGQGLEWIGYINPSRGYTNYNQKFKDKA | |
| | CH3(holes) | TLTTDKSSSTAYMQLSSLTSEDSAVYYCARYYDDHYSLDYWGQGTTLTVS | |
| | | SASTKGPSVFPLAPSSKSTSGGTAALGCLVKDYFPEPVTVSWNSGALTSG | |
| | | VHTFPAVLQSSGLYSLSSVVTVPSSSLGTQTYICNVNHKPSNTKVDKKVE | |
| | | PKSCDKTHTCPPCPAPEAAGGPSVFLFPPKPKDTLMISRTPEVTCVVVDV | |
| | | SHEDPEVKFNWYVDGVEVHNAKTKPREEQYNSTYRVVSVLTVLHQDWLNG | |
| | | KEYKCKVSNKALPAPIEKTISKAKGQPREPQVYTLPPSREEMTKNQVSLS | |
| | | CAVKGFYPSDIAVEWESNGQPENNYKTTPPVLDSDGSFFLVSKLTVDKSR | |
| | | WQQGNVFSCSVMHEALHNHYTQKSLSLSPGK | |
| | Cetuximab.2 VK- | DILLTQSPVILSVSPGERVSFSCRASQSIGTNIHWYQQRTSGSPRLLIKY | 183 |
| | linker-AB002.2 | ASESISGIPSRFSGSGSGTDFTLSINSVESEDIADYYCQQNNNWPTTFGA | |
| | VK-CK | GTKLELKRTVAAPSVFIFPPQIVLTQSPAIMSASPGEKVTMTCRASSSVS | |
| | | YMNWYQQKSGTSPKRWIYDTSKVASGVPYRFSGSGSGTSYSLTISSMEAE | |
| | | DAATYYCQQWSSNPLTFGSGTKLEIKRTVAAPSVFIFPPSDEQLKSGTAS | |
| | | VVCLLNNFYPREAKVQWKVDNALQSGNSQESVTEQDSKDSTYSLSSTLTL | |
| | | SKADYEKHKVYACEVTHQGLSSPVTKSFNRGEC | |
| TMH13 | YW243 VH-linker- | EVQLVESGGGLVQPGGSLRLSCAASGFTFSDSWIHWVRQAPGKGLEWVAW | 184 |
| | MH2p-hinge- | ISPYGGSTYYADSVKGRFTISADTSKNTAYLQMNSLRAEDTAVYYCARRH | |
| | CH2(LALA)-CH3 | YPGGFDYWGQGTLVTVSAASTELPPKVSVFVPPRDGFFGNPRKSKLICKA | |
| | (knobs) | TGFSPRQIQVSWLREGKQVGSGVTTKQVQAEAKESGPTTYKVTSTLTIKE | |
| | | SDWLGQSMFTCRVDHRGLTFQQNASSMCDKTHTCPPCPAPEAAGGPSVFL | |
| | | FPPKPKDTLMISRTPEVTCVVVDVSHEDPEVKFNWYVDGVEVHNAKTKPR | |
| | | EEQYNSTYRVVSVLTVLHQDWLNGKEYKCKVSNKALPAPIEKTISKAKGQ | |
| | | PREPQVYTLPPSREEMTKNQVSLWCLVKGFYPSDIAVEWESNGQPENNYK | |
| | | TTPPVLDSDGSFFLYSKLTVDKSRWQQGNVFSCSVMHEALHNHYTQKSLS | |
| | | LSPGK | |
| | YW243 VK-linker- | DIQMTQSPSSLSASVGDRVTITCRASQDVSTAVAWYQQKPGKAPKLLIYS | 185 |
| | MH2n | ASFLYSGVPSRFSGSGSGTDFTLTISSLQPEDFATYYCQQYLYHPATFGQ | |
| | | GTKVEIKRTAELPPKVSVFVPPRDGFFGNPRKSELICEATGFSPRQIQVS | |
| | | WLREGKQVGSGVTTDQVQAEAKESGPTTYDVTSTLTIKESDWLGQSMFTC | |
| | | RVDHRGLTFQQNASSMC | |
| | Cetuximab.2 VH- | QVQLKQSGPGLVQPSQSLSITCTVSGFSLTNYGVHWVRQSPGKGLEWLGV | 186 |
| | linker-AB002.2 | IWSGGNTDYNTPFTSRLSINKDNSKSQVFFKMNSLQSEDTAIYYCARALT | |
| | VH-CH1-hinge- | YYDYEFAYWGQGTLVTVSAASTKGPSVFPLAPQVQLQQSGAELARPGASV | |
| | CH2(LALA)- | KMSCKASGYTFTRYTMHWVKQRPGQGLEWIGYINPSRGYTNYNQKFKDKA | |
| | CH3(holes) | TLTTDKSSSTAYMQLSSLTSEDSAVYYCARYYDDHYSLDYWGQGTTLTVS | |
| | | SASTKGPSVFPLAPSSKSTSGGTAALGCLVKDYFPEPVTVSWNSGALTSG | |
| | | VHTFPAVLQSSGLYSLSSVVTVPSSSLGTQTYICNVNHKPSNTKVDKKVE | |
| | | PKSCDKTHTCPPCPAPEAAGGPSVFLFPPKPKDTLMISRTPEVTCVVVDV | |
| | | SHEDPEVKFNWYVDGVEVHNAKTKPREEQYNSTYRVVSVLTVLHQDWLNG | |
| | | KEYKCKVSNKALPAPIEKTISKAKGQPREPQVYTLPPSREEMTKNQVSLS | |
| | | CAVKGFYPSDIAVEWESNGQPENNYKTTPPVLDSDGSFFLVSKLTVDKSR | |
| | | WQQGNVFSCSVMHEALHNHYTQKSLSLSPGK | |
| | Cetuximab.2 VK- | DILLTQSPVILSVSPGERVSFSCRASQSIGTNIHWYQQRTSGSPRLLIKY | 187 |
| | linker-AB002.2 | ASESISGIPSRFSGSGSGTDFTLSINSVESEDIADYYCQQNNNWPTTFGA | |
| | VK-CK | GTKLELKRTVAAPSVFIFPPQIVLTQSPAIMSASPGEKVTMTCRASSSVS | |
| | | YMNWYQQKSGTSPKRWIYDTSKVASGVPYRFSGSGSGTSYSLTISSMEAE | |
| | | DAATYYCQQWSSNPLTFGSGTKLEIKRTVAAPSVFIFPPSDEQLKSGTAS | |
| | | VVCLLNNFYPREAKVQWKVDNALQSGNSQESVTEQDSKDSTYSLSSTLTL | |
| | | SKADYEKHKVYACEVTHQGLSSPVTKSFNRGEC | |
| TMH14 | YW243 VH-linker- | EVQLVESGGGLVQPGGSLRLSCAASGFTFSDSWIHWVRQAPGKGLEWVAW | 188 |
| | MH2h-hinge- | ISPYGGSTYYADSVKGRFTISADTSKNTAYLQMNSLRAEDTAVYYCARRH | |
| | CH2(LALA)-CH3 | YPGGFDYWGQGTLVTVSAASTELPPKVSVFVPPRDGFFGNPRKSKLACSA | |
| | (knobs) | TGFSPRQIQVSWLREGKQVGSGVTTDQVQAEAKESGPTTYKVASTLTIKE | |
| | | SDWLGQSMFTCRVDHRGLTFQQNASSMCDKTHTCPPCPAPEAAGGPSVFL | |
| | | FPPKPKDTLMISRTPEVTCVVVDVSHEDPEVKFNWYVDGVEVHNAKTKPR | |
| | | EEQYNSTYRVVSVLTVLHQDWLNGKEYKCKVSNKALPAPIEKTISKAKGQ | |
| | | PREPQVYTLPPSREEMTKNQVSLWCLVKGFYPSDIAVEWESNGQPENNYK | |
| | | TTPPVLDSDGSFFLYSKLTVDKSRWQQGNVFSCSVMHEALHNHYTQKSLS | |
| | | LSPGK | |
| | YW243 VK-linker- | DIQMTQSPSSLSASVGDRVTITCRASQDVSTAVAWYQQKPGKAPKLLIYS | 189 |
| | MH2k | ASFLYSGVPSRFSGSGSGTDFTLTISSLQPEDFATYYCQQYLYHPATFGQ | |
| | | GTKVEIKRTAELPPKVSVFVPPRDGFFGNPRKSKLWCQATGFSPRQIQVS | |
| | | WLREGKQVGSGVTTDQVQAEAKESGPTTYKVTSTLTIKESDWLGQSMFTC | |
| | | RVDHRGLTFQQNASSMC | |
| | Cetuximab.2 VH- | QVQLKQSGPGLVQPSQSLSITCTVSGFSLTNYGVHWVRQSPGKGLEWLGV | 190 |
| | linker-AB002.2 | IWSGGNTDYNTPFTSRLSINKDNSKSQVFFKMNSLQSEDTAIYYCARALT | |
| | VH-CH1-hinge- | YYDYEFAYWGQGTLVTVSAASTKGPSVFPLAPQVQLQQSGAELARPGASV | |
| | CH2(LALA)- | KMSCKASGYTFTRYTMHWVKQRPGQGLEWIGYINPSRGYTNYNQKFKDKA | |
| | CH3(holes) | TLTTDKSSSTAYMQLSSLTSEDSAVYYCARYYDDHYSLDYWGQGTTLTVS | |
| | | SASTKGPSVFPLAPSSKSTSGGTAALGCLVKDYFPEPVTVSWNSGALTSG | |
| | | VHTFPAVLQSSGLYSLSSVVTVPSSSLGTQTYICNVNHKPSNTKVDKKVE | |
| | | PKSCDKTHTCPPCPAPEAAGGPSVFLFPPKPKDTLMISRTPEVTCVVVDV | |
| | | SHEDPEVKFNWYVDGVEVHNAKTKPREEQYNSTYRVVSVLTVLHQDWLNG | |
| | | KEYKCKVSNKALPAPIEKTISKAKGQPREPQVYTLPPSREEMTKNQVSLS | |
| | | CAVKGFYPSDIAVEWESNGQPENNYKTTPPVLDSDGSFFLVSKLTVDKSR | |
| | | WQQGNVFSCSVMHEALHNHYTQKSLSLSPGK | |
| | Cetuximab.2 VK- | DILLTQSPVILSVSPGERVSFSCRASQSIGTNIHWYQQRTSGSPRLLIKY | 191 |
| | linker-AB002.2 | ASESISGIPSRFSGSGSGTDFTLSINSVESEDIADYYCQQNNNWPTTFGA | |
| | VK-CK | GTKLELKRTVAAPSVFIFPPQIVLTQSPAIMSASPGEKVTMTCRASSSVS | |
| | | YMNWYQQKSGTSPKRWIYDTSKVASGVPYRFSGSGSGTSYSLTISSMEAE | |
| | | DAATYYCQQWSSNPLTFGSGTKLEIKRTVAAPSVFIFPPSDEQLKSGTAS | |
| | | VVCLLNNFYPREAKVQWKVDNALQSGNSQESVTEQDSKDSTYSLSSTLTL | |
| | | SKADYEKHKVYACEVTHQGLSSPVTKSFNRGEC | |
| TMH15 | YW243 VH-linker- | EVQLVESGGGLVQPGGSLRLSCAASGFTFSDSWIHWVRQAPGKGLEWVAW | 192 |
| | MH2k-hinge- | ISPYGGSTYYADSVKGRFTISADTSKNTAYLQMNSLRAEDTAVYYCARRH | |
| | CH2(LALA)- | YPGGFDYWGQGTLVTVSAASTELPPKVSVFVPPRDGFFGNPRKSKLWCQA | |
| | CH3(knobs) | TGFSPRQIQVSWLREGKQVGSGVTTDQVQAEAKESGPTTYKVTSTLTIKE | |
| | | SDWLGQSMFTCRVDHRGLTFQQNASSMCDKTHTCPPCPAPEAAGGPSVFL | |
| | | FPPKPKDTLMISRTPEVTCVVVDVSHEDPEVKFNWYVDGVEVHNAKTKPR | |
| | | EEQYNSTYRVVSVLTVLHQDWLNGKEYKCKVSNKALPAPIEKTISKAKGQ | |
| | | PREPQVYTLPPSREEMTKNQVSLWCLVKGFYPSDIAVEWESNGQPENNYK | |
| | | TTPPVLDSDGSFFLYSKLTVDKSRWQQGNVFSCSVMHEALHNHYTQKSLS | |
| | | LSPGK | |
| | YW243 VK-linker- | DIQMTQSPSSLSASVGDRVTITCRASQDVSTAVAWYQQKPGKAPKLLIYS | 193 |
| | MH2h | ASFLYSGVPSRFSGSGSGTDFTLTISSLQPEDFATYYCQQYLYHPATFGQ | |
| | | GTKVEIKRTAELPPKVSVFVPPRDGFFGNPRKSKLACSATGFSPRQIQVS | |
| | | WLREGKQVGSGVTTDQVQAEAKESGPTTYKVASTLTIKESDWLGQSMFTC | |
| | | RVDHRGLTFQQNASSMC | |
| | Cetuximab.2 VH- | QVQLKQSGPGLVQPSQSLSITCTVSGFSLTNYGVHWVRQSPGKGLEWLGV | 194 |
| | linker-AB002.2 | IWSGGNTDYNTPFTSRLSINKDNSKSQVFFKMNSLQSEDTAIYYCARALT | |
| | VH-CH1-hinge- | YYDYEFAYWGQGTLVTVSAASTKGPSVFPLAPQVQLQQSGAELARPGASV | |
| | CH2(LALA)- | KMSCKASGYTFTRYTMHWVKQRPGQGLEWIGYINPSRGYTNYNQKFKDKA | |
| | CH3(holes) | TLTTDKSSSTAYMQLSSLTSEDSAVYYCARYYDDHYSLDYWGQGTTLTVS | |
| | | SASTKGPSVFPLAPSSKSTSGGTAALGCLVKDYFPEPVTVSWNSGALTSG | |
| | | VHTFPAVLQSSGLYSLSSVVTVPSSSLGTQTYICNVNHKPSNTKVDKKVE | |
| | | PKSCDKTHTCPPCPAPEAAGGPSVFLFPPKPKDTLMISRTPEVTCVVVDV | |
| | | SHEDPEVKFNWYVDGVEVHNAKTKPREEQYNSTYRVVSVLTVLHQDWLNG | |
| | | KEYKCKVSNKALPAPIEKTISKAKGQPREPQVYTLPPSREEMTKNQVSLS | |
| | | CAVKGFYPSDIAVEWESNGQPENNYKTTPPVLDSDGSFFLVSKLTVDKSR | |
| | | WQQGNVFSCSVMHEALHNHYTQKSLSLSPGK | |
| | Cetuximab.2 VK- | DILLTQSPVILSVSPGERVSFSCRASQSIGTNIHWYQQRTSGSPRLLIKY | 195 |
| | linker-AB002.2 | ASESISGIPSRFSGSGSGTDFTLSINSVESEDIADYYCQQNNNWPTTFGA | |
| | VK-CK | GTKLELKRTVAAPSVFIFPPQIVLTQSPAIMSASPGEKVTMTCRASSSVS | |
| | | YMNWYQQKSGTSPKRWIYDTSKVASGVPYRFSGSGSGTSYSLTISSMEAE | |
| | | DAATYYCQQWSSNPLTFGSGTKLEIKRTVAAPSVFIFPPSDEQLKSGTAS | |
| | | VVCLLNNFYPREAKVQWKVDNALQSGNSQESVTEQDSKDSTYSLSSTLTL | |
| | | SKADYEKHKVYACEVTHQGLSSPVTKSFNRGEC | |
| TMH16 | YW243 VH-linker- | EVQLVESGGGLVQPGGSLRLSCAASGFTFSDSWIHWVRQAPGKGLEWVAW | 196 |
| | MH2-hinge- | ISPYGGSTYYADSVKGRFTISADTSKNTAYLQMNSLRAEDTAVYYCARRH | |
| | CH2(LALA)-CH3 | YPGGFDYWGQGTLVTVSAASTELPPKVSVFVPPRDGFFGNPRKSKLICQA | |
| | (knobs) | TGFSPRQIQVSWLREGKQVGSGVTTDQVQAEAKESGPTTYKVTSTLTIKE | |
| | | SDWLGQSMFTCRVDHRGLTFQQNASSMCDKTHTCPPCPAPEAAGGPSVFL | |
| | | FPPKPKDTLMISRTPEVTCVVVDVSHEDPEVKFNWYVDGVEVHNAKTKPR | |
| | | EEQYNSTYRVVSVLTVLHQDWLNGKEYKCKVSNKALPAPIEKTISKAKGQ | |
| | | PREPQVYTLPPSREEMTKNQVSLWCLVKGFYPSDIAVEWESNGQPENNYK | |
| | | TTPPVLDSDGSFFLYSKLTVDKSRWQQGNVFSCSVMHEALHNHYTQKSLS | |
| | | LSPGK | |
| | YW243 VK-linker- | DIQMTQSPSSLSASVGDRVTITCRASQDVSTAVAWYQQKPGKAPKLLIYS | 197 |
| | MH2 | ASFLYSGVPSRFSGSGSGTDFTLTISSLQPEDFATYYCQQYLYHPATFGQ | |
| | | GTKVEIKRTAELPPKVSVFVPPRDGFFGNPRKSKLICQATGFSPRQIQVS | |
| | | WLREGKQVGSGVTTDQVQAEAKESGPTTYKVTSTLTIKESDWLGQSMFTC | |
| | | RVDHRGLTFQQNASSMC | |
| | Cetuximab VH- | QVQLKQSGPGLVQPSQSLSITCTVSGFSLTNYGVHWVRQSPGKGLEWLGV | 198 |
| | linker-AB002 VH- | IWSGGNTDYNTPFTSRLSINKDNSKSQVFFKMNSLQSNDTAIYYCARALT | |
| | CH1-hinge- | YYDYEFAYWGQGTLVTVSAASTKGPSVFPLAPQVQLQQSGAELARPGASV | |
| | CH2(LALA)- | KMSCKASGYTFTRYTMHWVKQRPGQGLEWIGYINPSRGYTNYNQKFKDKA | |
| | CH3(holes) | TLTTDKSSSTAYMQLSSLTSEDSAVYYCARYYDDHYCLDYWGQGTTLTVS | |
| | | SASTKGPSVFPLAPSSKSTSGGTAALGCLVKDYFPEPVTVSWNSGALTSG | |
| | | VHTFPAVLQSSGLYSLSSVVTVPSSSLGTQTYICNVNHKPSNTKVDKKVE | |
| | | PKSCDKTHTCPPCPAPEAAGGPSVFLFPPKPKDTLMISRTPEVTCVVVDV | |
| | | SHEDPEVKFNWYVDGVEVHNAKTKPREEQYNSTYRVVSVLTVLHQDWLNG | |
| | | KEYKCKVSNKALPAPIEKTISKAKGQPREPQVYTLPPSREEMTKNQVSLS | |
| | | CAVKGFYPSDIAVEWESNGQPENNYKTTPPVLDSDGSFFLVSKLTVDKSR | |
| | | WQQGNVFSCSVMHEALHNHYTQKSLSLSPGK | |
| | Cetuximab VK- | DILLTQSPVILSVSPGERVSFSCRASQSIGTNIHWYQQRTNGSPRLLIKY | 199 |
| | linker-AB002 VK- | ASESISGIPSRFSGSGSGTDFTLSINSVESEDIADYYCQQNNNWPTTFGA | |
| | CK | GTKLELKRTVAAPSVFIFPPQIVLTQSPAIMSASPGEKVTMTCRASSSVS | |
| | | YMNWYQQKSGTSPKRWIYDTSKYASDVPYRFSGSGSGTSYSLTISSMEAE | |
| | | DAATYYCQQWSSNPLTFGSGTKLEINRTVAAPSVFIFPPSDEQLKSGTAS | |
| | | VVCLLNNFYPREAKVQWKVDNALQSGNSQESVTEQDSKDSTYSLSSTLTL | |
| | | SKADYEKHKVYACEVTHQGLSSPVTKSFNRGEC | |
| TMH17 | YW243 VH-linker- | EVQLVESGGGLVQPGGSLRLSCAASGFTFSDSWIHWVRQAPGKGLEWVAW | 200 |
| | MH2n-hinge- | ISPYGGSTYYADSVKGRFTISADTSKNTAYLQMNSLRAEDTAVYYCARRH | |
| | CH2(LALA)-CH3 | YPGGFDYWGQGTLVTVSAASTELPPKVSVFVPPRDGFFGNPRKSELICEA | |
| | (knobs) | TGFSPRQIQVSWLREGKQVGSGVTTDQVQAEAKESGPTTYDVTSTLTIKE | |
| | | SDWLGQSMFTCRVDHRGLTFQQNASSMCDKTHTCPPCPAPEAAGGPSVFL | |
| | | FPPKPKDTLMISRTPEVTCVVVDVSHEDPEVKFNWYVDGVEVHNAKTKPR | |
| | | EEQYNSTYRVVSVLTVLHQDWLNGKEYKCKVSNKALPAPIEKTISKAKGQ | |
| | | PREPQVYTLPPSREEMTKNQVSLWCLVKGFYPSDIAVEWESNGQPENNYK | |
| | | TTPPVLDSDGSFFLYSKLTVDKSRWQQGNVFSCSVMHEALHNHYTQKSLS | |
| | | LSPGK | |
| | YW243 VK-linker- | DIQMTQSPSSLSASVGDRVTITCRASQDVSTAVAWYQQKPGKAPKLLIYS | 201 |
| | MH2p | ASFLYSGVPSRFSGSGSGTDFTLTISSLQPEDFATYYCQQYLYHPATFGQ | |
| | | GTKVEIKRTAELPPKVSVFVPPRDGFFGNPRKSKLICKATGFSPRQIQVS | |
| | | WLREGKQVGSGVTTKQVQAEAKESGPTTYKVTSTLTIKESDWLGQSMFTC | |
| | | RVDHRGLTFQQNASSMC | |
| | Cetuximab VH- | QVQLKQSGPGLVQPSQSLSITCTVSGFSLTNYGVHWVRQSPGKGLEWLGV | 202 |
| | linker-AB002 VH- | IWSGGNTDYNTPFTSRLSINKDNSKSQVFFKMNSLQSNDTAIYYCARALT | |
| | CH1-hinge- | YYDYEFAYWGQGTLVTVSAASTKGPSVFPLAPQVQLQQSGAELARPGASV | |
| | CH2(LALA)- | KMSCKASGYTFTRYTMHWVKQRPGQGLEWIGYINPSRGYTNYNQKFKDKA | |
| | CH3(holes) | TLTTDKSSSTAYMQLSSLTSEDSAVYYCARYYDDHYCLDYWGQGTTLTVS | |
| | | SASTKGPSVFPLAPSSKSTSGGTAALGCLVKDYFPEPVTVSWNSGALTSG | |
| | | VHTFPAVLQSSGLYSLSSVVTVPSSSLGTQTYICNVNHKPSNTKVDKKVE | |
| | | PKSCDKTHTCPPCPAPEAAGGPSVFLFPPKPKDTLMISRTPEVTCVVVDV | |
| | | SHEDPEVKFNWYVDGVEVHNAKTKPREEQYNSTYRVVSVLTVLHQDWLNG | |
| | | KEYKCKVSNKALPAPIEKTISKAKGQPREPQVYTLPPSREEMTKNQVSLS | |
| | | CAVKGFYPSDIAVEWESNGQPENNYKTTPPVLDSDGSFFLVSKLTVDKSR | |
| | | WQQGNVFSCSVMHEALHNHYTQKSLSLSPGK | |
| | Cetuximab VK- | DILLTQSPVILSVSPGERVSFSCRASQSIGTNIHWYQQRTNGSPRLLIKY | 203 |
| | linker-AB002 VK- | ASESISGIPSRFSGSGSGTDFTLSINSVESEDIADYYCQQNNNWPTTFGA | |
| | CK | GTKLELKRTVAAPSVFIFPPQIVLTQSPAIMSASPGEKVTMTCRASSSVS | |
| | | YMNWYQQKSGTSPKRWIYDTSKVASGVPYRFSGSGSGTSYSLTISSMEAE | |
| | | DAATYYCQQWSSNPLTFGSGTKLEINRTVAAPSVFIFPPSDEQLKSGTAS | |
| | | VVCLLNNFYPPEAKVQWKVDNALQSGNSQESVTEQDSKDSTYSLSSTLTL | |
| | | SKADYEKHKVYACEVTHQGLSSPVTKSFNRGEC | |
| TMH18 | YW243 VH-linker- | EVQLVESGGGLVQPGGSLRLSCAASGFTFSDSWIHWVRQAPGKGLEWVAW | 204 |
| | MH2p-hinge- | ISPYGGSTYYADSVKGRFTISADTSKNTAYLQMNSLRAEDTAVYYCARRH | |
| | CH2(LALA)-CH3 | YPGGFDYWGQGTLVTVSAASTELPPKVSVFVPPRDGFFGNPRKSKLICKA | |
| | (knobs) | TGFSPRQIQVSWLREGKQVGSGVTTKQVQAEAKESGPTTYKVTSTLTIKE | |
| | | SDWLGQSMFTCRVDHRGLTFQQNASSMCDKTHTCPPCPAPEAAGGPSVFL | |
| | | FPPKPKDTLMISRTPEVTCVVVDVSHEDPEVKFNWYVDGVEVHNAKTKPR | |
| | | EEQYNSTYRVVSVLTVLHQDWLNGKEYKCKVSNKALPAPIEKTISKAKGQ | |
| | | PREPQVYTLPPSREEMTKNQVSLWCLVKGFYPSDIAVEWESNGQPENNYK | |
| | | TTPPVLDSDGSFFLYSKLTVDKSRWQQGNVFSCSVMHEALHNHYTQKSLS | |
| | | LSPGK | |
| | YW243 VK-linker- | DIQMTQSPSSLSASVGDRVTITCRASQDVSTAVAWYQQKPGKAPKLLIYS | 205 |
| | MH2n | ASFLYSGVPSRFSGSGSGTDFTLTISSLQPEDFATYYCQQYLYHPATFGQ | |
| | | GTKVEIKRTAELPPKVSVFVPPRDGFFGNPRKSELICEATGFSPRQIQVS | |
| | | WLREGKQVGSGVTTDQVQAEAKESGPTTYDVTSTLTIKESDWLGQSMFTC | |
| | | RVDHRGLTFQQNASSMC | |
| | Cetuximab VH- | QVQLKQSGPGLVQPSQSLSITCTVSGFSLTNYGVHWVRQSPGKGLEWLGV | 206 |
| | linker-AB002 VH- | IWSGGNTDYNTPFTSRLSINKDNSKSQVFFKMNSLQSNDTAIYYCARALT | |
| | CH1-hinge- | YYDYEFAYWGQGTLVTVSAASTKGPSVFPLAPQVQLQQSGAELARPGASV | |
| | CH2(LALA)- | KMSCKASGYTFTRYTMHWVKQRPGQGLEWIGYINPSRGYTNYNQKFKDKA | |
| | CH3(holes) | TLTTDKSSSTAYMQLSSLTSEDSAVYYCARYYDDHYCLDYWGQGTTLTVS | |
| | | SASTKGPSVFPLAPSSKSTSGGTAALGCLVKDYFPEPVTVSWNSGALTSG | |
| | | VHTFPAVLQSSGLYSLSSVVTVPSSSLGTQTYICNVNHKPSNTKVDKKVE | |
| | | PKSCDKTHTCPPCPAPEAAGGPSVFLFPPKPKDTLMISRTPEVTCVVVDV | |
| | | SHEDPEVKFNWYVDGVEVHNAKTKPREEQYNSTYRVVSVLTVLHQDWLNG | |
| | | KEYKCKVSNKALPAPIEKTISKAKGQPREPQVYTLPPSREEMTKNQVSLS | |
| | | CAVKGFYPSDIAVEWESNGQPENNYKTTPPVLDSDGSFFLVSKLTVDKSR | |
| | | WQQGNVFSCSVMHEALHNHYTQKSLSLSPGK | |
| | Cetuximab VK- | DILLTQSPVILSVSPGERVSFSCRASQSIGTNIHWYQQRTNGSPRLLIKY | 207 |
| | linker-AB002 VK- | ASESISGIPSRFSGSGSGTDFTLSINSVESEDIADYYCQQNNNWPTTFGA | |
| | CK | GTKLELKRTVAAPSVFIFPPQIVLTQSPAIMSASPGEKVTMTCRASSSVS | |
| | | YMNWYQQKSGTSPKRWIYDTSKVASGVPYRFSGSGSGTSYSLTISSMEAE | |
| | | DAATYYCQQWSSNPLTFGSGTKLEINRTVAAPSVFIFPPSDEQLKSGTAS | |
| | | VVCLLNNFYPREAKVQWKVDNALQSGNSQESVTEQDSKDSTYSLSSTLTL | |
| | | SKADYEKHKVYACEVTHQGLSSPVTKSFNRGEC | |
| TMH19 | YW243 VH-linker- | EVQLVESGGGLVQPGGSLRLSCAASGFTFSDSWIHWVRQAPGKGLEWVAW | 208 |
| | MH2h-hinge- | ISPYGGSTYYADSVKGRFTISADTSKNTAYLQMNSLRAEDTAVYYCARRH | |
| | CH2(LALA)-CH3 | YPGGFDYWGQGTLVTVSAASTELPPKVSVFVPPRDGFFGNPRKSKLACSA | |
| | (knobs) | TGFSPRQIQVSWLREGKQVGSGVTTDQVQAEAKESGPTTYKVASTLTIKE | |
| | | SDWLGQSMFTCRVDHRGLTFQQNASSMCDKTHTCPPCPAPEAAGGPSVFL | |
| | | FPPKPKDTLMISRTPEVTCVVVDVSHEDPEVKFNWYVDGVEVHNAKTKPR | |
| | | EEQYNSTYRVVSVLTVLHQDWLNGKEYKCKVSNKALPAPIEKTISKAKGQ | |
| | | PREPQVYTLPPSREEMTKNQVSLWCLVKGFYPSDIAVEWESNGQPENNYK | |
| | | TTPPVLDSDGSFFLYSKLTVDKSRWQQGNVFSCSVMHEALHNHYTQKSLS | |
| | | LSPGK | |
| | YW243 VK-linker- | DIQMTQSPSSLSASVGDRVTITCRASQDVSTAVAWYQQKPGKAPKLLIYS | 209 |
| | MH2k | ASFLYSGVPSRFSGSGSGTDFTLTISSLQPEDFATYYCQQYLYHPATFGQ | |
| | | GTKVEIKRTAELPPKVSVFVPPRDGFFGNPRKSKLWCQATGFSPRQIQVS | |
| | | WLREGKQVGSGVTTDQVQAEAKESGPTTYKVTSTLTIKESDWLGQSMFTC | |
| | | RVDHRGLTFQQNASSMC | |
| | Cetuximab VH- | QVQLKQSGPGLVQPSQSLSITCTVSGFSLTNYGVHWVRQSPGKGLEWLGV | 210 |
| | linker-AB002 VH- | IWSGGNTDYNTPFTSRLSINKDNSKSQVFFKMNSLQSNDTAIYYCARALT | |
| | CH1-hinge- | YYDYEFAYWGQGTLVTVSAASTKGPSVFPLAPQVQLQQSGAELARPGASV | |
| | CH2(LALA)- | KMSCKASGYTFTRYTMHWVKQRPGQGLEWIGYINPSRGYTNYNQKFKDKA | |
| | CH3(holes) | TLTTDKSSSTAYMQLSSLTSEDSAVYYCARYYDDHYCLDYWGQGTTLTVS | |
| | | SASTKGPSVFPLAPSSKSTSGGTAALGCLVKDYFPEPVTVSWNSGALTSG | |
| | | VHTFPAVLQSSGLYSLSSVVTVPSSSLGTQTYICNVNHKPSNTKVDKKVE | |
| | | PKSCDKTHTCPPCPAPEAAGGPSVFLFPPKPKDTLMISRTPEVTCVVVDV | |
| | | SHEDPEVKFNWYVDGVEVHNAKTKPREEQYNSTYRVVSVLTVLHQDWLNG | |
| | | KEYKCKVSNKALPAPIEKTISKAKGQPREPQVYTLPPSREEMTKNQVSLS | |
| | | CAVKGFYPSDIAVEWESNGQPENNYKTTPPVLDSDGSFFLVSKLTVDKSR | |
| | | WQQGNVFSCSVMHEALHNHYTQKSLSLSPGK | |
| | Cetuximab VK- | DILLTQSPVILSVSPGERVSFSCRASQSIGTNIHWYQQRTNGSPRLLIKY | 211 |
| | linker-AB002 VK- | ASESISGIPSRFSGSGSGTDFTLSINSVESEDIADYYCQQNNNWPTTFGA | |
| | CK | GTKLELKRTVAAPSVFIFPPQIVLTQSPAIMSASPGEKVTMTCRASSSVS | |
| | | YMNNYQQKSGTSPKPWIYDTSKVASGVPYRFSGSGSGTSYELTISSMEAE | |
| | | DAATYYCQQWSSNPLTFGSGTKLEINRTVAAPSVFIFPPSDEQLKSGTAS | |
| | | VVCLLNNFYPREAKVQWKVDNALQSGNSQESVTEQDSKDSTYSLSSTLTL | |
| | | SKADYEKHKVYACEVTHQGLSSPVTKSFNRGEC | |
| TMH20 | YW243 VH-linker- | EVQLVESGGGLVQPGGSLRLSCAASGFTFSDSWIHWVRQAPGKGLEWVAW | 212 |
| | MH2k-hinge- | ISPYGGSTYYADSVKGRFTISADTSKNTAYLQMNSLRAEDTAVYYCARRH | |
| | CH2(LALA)- | YPGGFDYWGQGTLVTVSAASTELPPKVSVFVPPRDGFFGNPRKSKLWCQA | |
| | CH3(knobs) | TGFSPRQIQVSWLREGKQVGSGVTTDQVQAEAKESGPTTYKVTSTLTIKE | |
| | | SDWLGQSMFTCRVDHRGLTFQQNASSMCDKTHTCPPCPAPEPAGGPSVFL | |
| | | FPPKPKDTLMISRTPEVTCVVVDVSHEDPEVKFNWYVDGVEVHNAKTKPR | |
| | | EEQYNSTYRVVSVLTVLHQDWLNGKEYKCKVSNKALPAPIEKTISKAKGQ | |
| | | PREPQVYTLPPSREEMTKNQVSLWCLVKGFYPSDIAVEWESNGQPENNYK | |
| | | TTPPVLDSDGSFFLYSKLTVDKSRWQQGNVFSCSVMHEALHNHYTQKSLS | |
| | | LSPGK | |
| | YW243 VK-linker- | DIQMTQSPSSLSASVGDRVTITCRASQDVSTAVAWYQQKPGKAPKLLIYS | 213 |
| | MH2h | ASFLYSGVPSRFSGSGSGTDFTLTISSLQPEDFATYYCQQYLYHPATFGQ | |
| | | GTKVEIKRTAELPPKVSVFVPPRDGFFGNPRKSKLACSATGFSPRQIQVS | |
| | | WLREGKQVGSGVTTDQVQAEAKESGPTTYKVASTLTIKESDWLGQSMFTC | |
| | | RVDHRGLTFQQNASSMC | |
| | Cetuximab VH- | QVQLKQSGPGLVQPSQSLSITCTVSGFSLTNYGVHWVRQSPGKGLEWLGV | 214 |
| | linker-AB002 VH- | IWSGGNTDYNTPFTSRLSINKDNSKSQVFFKMNSLQSNDTAIYYCARALT | |
| | CH1-hinge- | YYDYEFAYWGQGTLVTVSAASTKGPSVFPLAPQVQLQQSGAELARPGASV | |
| | CH2(LALA)- | KMSCKASGYTFTRYTMHWVKQRPGQGLEWIGYINPSRGYTNYNQKFKDKA | |
| | CH3(holes) | TLTTDKSSSTAYMQLSSLTSEDSAVYYCARYYDDHYCLDYWGQGTTLTVS | |
| | | SASTKGPSVFPLAPSSKSTSGGTAALGCLVKDYFPEPVTVSWNSGALTSG | |
| | | VHTFPAVLQSSGLYSLSSVVTVPSSSLGTQTYICNVNHKPSNTKVDKKVE | |
| | | PKSCDKTHTCPPCPAPEAAGGPSVFLFPPKPKDTLMISRTPEVTCVVVDV | |
| | | SHEDPEVKFNWYVDGVEVHNAKTKPREEQYNSTYRVVSVLTVLHQDWLNG | |
| | | KEYKCKVSNKALPAPIEKTISKAKGQPREPQVYTLPPSREEMTKNQVSLS | |
| | | CAVKGFYPSDIAVEWESNGQPENNYKTTPPVLDSDGSFFLVSKLTVDKSR | |
| | | WQQGNVFSCSVMHEALHNHYTQKSLSLSPGK | |
| | Cetuximab VK- | DILLTQSPVILSVSPGERVSFSCRASQSIGTNIHWYQQRTNGSPRLLIKY | 215 |
| | linker-AB002 VK- | ASESISGIPSRFSGSGSGTDFTLSINSVESEDIADYYCQQNNNWPTTFGA | |
| | CK | GTKLELKRTVAAPSVFIFPPQIVLTQSPAIMSASPGEKVTMTCRASSSVS | |
| | | YMNWYQQKSGTSPKRWTYDTSKVASGVPYRFSGSGSGTSYSLTISSMEAE | |
| | | DAATYYCQQWSSNPLTFGSGTKLEINRTVAAPSVFIFPPSDEQLKSGTAS | |
| | | VVCLLNNFYPREAKVQWKVDNALQSGNSQESVTEQDSKDSTYSLSSTLTL | |
| | | SKADYEKHKVYACEVTHQGLSSPVTKSFNRGEC | |
| TMH21 | AB426 VH-linker- | QVQLVESGGGVVQPGRSLRLDCKASGITFSNSGMHWVRQAPGKGLEWVAV | 216 |
| | MH2-hinge- | IWYDGSKRYYADSVKGRFTISRDNSKNTLFLQMNSLRAEDTAVYYCATND | |
| | CH2(LALA)- | DYWGQGTLVTVSSASTELPPKVSVFVPPRDGFFGNPRKSKLICQATGFSP | |
| | CH3(knobs) | RQIQVSWLREGKQVGSGVTTDQVQAEAKESGPTTYKVTSTLTIKESDWLG | |
| | | QSMFTCRVDHRGLTFQQNASSMCDKTHTCPPCPAPEAAGGPSVFLFPPKP | |
| | | KDTLMISRTPEVTCVVVDVSHEDPEVKFNWYVDGVEVHNAKTKPREEQYN | |
| | | STYRVVSVLTVLHQDWLNGKEYKCKVSNKALPAPIEKTISKAKGQPREPQ | |
| | | VYTLPPSREEMTKNQVSLWCLVKGFYPSDIAVEWESNGQPENNYKTTPPV | |
| | | LDSDGSFFLYSKLTVDKSRWQQGNVFSCSVMHEALHNHYTQKSLSLSPGK | |
| | AB426 VK-linker- | EIVLTQSPATLSLSPGERATLSCRASQSVSSYLAWYQQKPGQAPRLLIYD | 217 |
| | MH2 | ASNRATGIPARFSGSGSGTDFTLTISSLEPEDFAVYYCQQSSNWPRTFGQ | |
| | | GTKVEIKRTAELPPKVSVFVPPRDGFFGNPRKSKLICQATGFSPRQIQVS | |
| | | WLREGKQVGSGVTTDQVQAEAKESGPTTYKVTSTLTIKESDWLGQSMFTC | |
| | | RVDHRGLTFQQNASSMC | |
| | Cetuximab VH- | QVQLKQSGPGLVQPSQSLSITCTVSGFSLTNYGVHWVRQSPGKGLEWLGV | 218 |
| | linker-AB002 VH- | IWSGGNTDYNTPFTSRLSINKDNSKSQVFFKMNSLQSNDTAIYYCARALT | |
| | CH1-hinge- | YYDYEFAYWGQGTLVTVSAASTKGPSVFPLAPQVQLQQSGAELARPGASV | |
| | CH2(LALA)- | KMSCKASGYTFTRYTMHWVKQRPGQGLEWIGYINPSRGYTNYNQKFKDKA | |
| | CH3(holes) | TLTTDKSSSTAYMQLSSLTSEDSAVYYCARYYDDHYCLDYWGQGTTLTVS | |
| | | SASTKGPSVFPLAPSSKSTSGGTAALGCLVKDYFPEPVTVSWNSGALTSG | |
| | | VHTFPAVLQSSGLYSLSSVVTVPSSSLGTQTYICNVNHKPSNTKVDKKVE | |
| | | PKSCDKTHTCPPCPAPEAAGGPSVFLFPPKPKDTLMISRTPEVTCVVVDV | |
| | | SHEDPEVKFNWYVDGVEVHNAKTKPREEQYNSTYRVVSVLTVLHQDWLNG | |
| | | KEYKCKVSNKALPAPIEKTISKAKGQPREPQVYTLPPSREEMTKNQVSLS | |
| | | CAVKGFYPSDIAVEWESNGQPENNYKTTPPVLDSDGSFFLVSKLTVDKSR | |
| | | WQQGNVFSCSVMHEALHNHYTQKSLSLSPGK | |
| | Cetuximab VK- | DILLTQSPVILSVSPGERVSFSCRASQSIGTNIHWYQQRTNGSPRLLIKY | 219 |
| | linker-AB002 VK- | ASESISGIPSRFSGSGSGTDFTLSINSVESEDIADYYCQQNNNWPTTFGA | |
| | CK | GTKLELKRTVAAPSVFIFPPQIVLTQSPAIMSASPGEKVTMTCRASSSVS | |
| | | YMNWYQQKSGTSPKRWIYDTSKVASGVPYRFSGSGSGTSYSLTISSMEAE | |
| | | DAATYYCQQWSSNPLTFGSGTKLEINRTVAAPSVFIFPPSDEQLKSGTAS | |
| | | VVCLLNNFYPREAKVQWKVDNALQSGNSQESVTEQDSKDSTYSLSSTLTL | |
| | | SKADYEKHKVYACEVTHQGLSSPVTKSFNRGEC | |
| TMH22 | AB426 VH-linker- | QVQLVESGGGVVQPGRSLRLDCKASGITFSNSGMHWVRQAPGKGLEWVAV | 220 |
| | MH2n-hinge- | IWYDGSKRYYADSVKGRFTISRDNSKNTLFLQMNSLRAEDTAVYYCATND | |
| | CH2(LALA)- | DYWGQGTLVTVSSASTELPPKVSVFVPPRDGFFGNPRKSELICEATGFSP | |
| | CH3(knobs) | RQIQVSWLREGKQVGSGVTTDQVQAEAKESGPTTYDVTSTLTIKESDWLG | |
| | | QSMFTCRVDHRGLTFQQNASSMCDKTHTCPPCPAPEAAGGPSVFLFPPKP | |
| | | KDTLMISRTPEVTCVVVDVSHEDPEVKFNWYVDGVEVHNAKTKPREEQYN | |
| | | STYRVVSVLTVLHQDWLNGKEYKCKVSNKALPAPIEKTISKAKGQPREPQ | |
| | | VYTLPPSREEMTKNQVSLWCLVKGFYPSDIAVEWESNGQPENNYKTTPPV | |
| | | LDSDGSFFLYSKLTVDKSRWQQGNVFSCSVMHEALHNHYTQKSLSLSPGK | |
| | AB426 VK-linker- | EIVLTQSPATLSLSPGERATLSCRASQSVSSYLAWYQQKPGQAPRLLIYD | 221 |
| | MH2p | ASNRATGIPARFSGSGSGTDFTLTISSLEPEDFAVYYCQQSSNWPRTFGQ | |
| | | GTKVEIKRTAELPPKVSVFVPPRDGFFGNPRKSKLICKATGFSPRQIQVS | |
| | | WLREGKQVGSGVTTKQVQAEAKESGPTTYKVTSTLTIKESDWLGQSMFTC | |
| | | RVDHRGLTFQQNASSMC | |
| | Cetuximab VH- | QVQLKQSGPGLVQPSQSLSITCTVSGFSLTNYGVHWVRQSPGKGLEWLGV | 222 |
| | linker-AB002 VH- | IWSGGNTDYNTPFTSRLSINKDNSKSQVFFKMNSLQSNDTAIYYCARALT | |
| | CH1-hinge- | YYDYEFAYWGQGTLVTVSAASTKGPSVFPLAPQVQLQQSGAELARPGASV | |
| | CH2(LALA)- | KMSCKASGYTFTRYTMHWVKQRPGQGLEWIGYINPSRGYTNYNQKFKDKA | |
| | CH3(holes) | TLTTDKSSSTAYMQLSSLTSEDSAVYYCARYYDDHYCLDYWGQGTTLTVS | |
| | | SASTKGPSVFPLAPSSKSTSGGTAALGCLVKDYFPEPVTVSWNSGALTSG | |
| | | VHTFPAVLQSSGLYSLSSVVTVPSSSLGTQTYICNVNHKPSNTKVDKKVE | |
| | | PKSCDKTHTCPPCPAPEAAGGPSVFLFPPKPKDTLMISRTPEVTCVVVDV | |
| | | SHEDPEVKFNWYVDGVEVHNAKTKPREEQYNSTYRVVSVLTVLHQDWLNG | |
| | | KEYKCKVSNKALPAPIEKTISKAKGQPREPQVYTLPPSREEMTKNQVSLS | |
| | | CAVKGFYPSDIAVEWESNGQPENNYKTTPPVLDSDGSFFLVSKLTVDKSR | |
| | | WQQGNVFSCSVMHEALHNHYTQKSLSLSPGK | |
| | Cetuximab VK- | DILLTQSPVILSVSPGERVSFSCRASQSIGTNIHWYQQRTNGSPRLLIKY | 223 |
| | linker-AB002 VK- | ASESISGIPSRFSGSGSGTDFTLSINSVESEDIADYYCQQNNNWPTTFGA | |
| | CK | GTKLELKRTVAAPSVFIFPPQIVLTQSPAIMSASPGEKVTMTCRASSSVS | |
| | | YMNWYQQKSGTSPKRWIYDTSKVASGVPYRFSGSGSGTSYSLTISSMEAE | |
| | | DAATYYCQQWSSNPLTFGSGTKLEINRTVAAPSVFIFPPSDEQLKSGTAS | |
| | | VVCLLNNFYPREAKVQWKVDNALQSGNSQESVTEQDSKDSTYSLSSTLTL | |
| | | SKADYEKHKVYACEVTHQGLSSPVTKSFNRGEC | |
| TMH23 | AB426 VH-linker- | QVQLVESGGGVVQPGRSLRLDCKASGITFSNSGMHWVRQAPGKGLEWVAV | 224 |
| | MH2p-hinge- | IWYDGSKRYYADSVKGRFTISRDNSKNTLFLQMNSLRAEDTAVYYCATND | |
| | CH2(LALA)- | DYWGQGTLVTVSSASTELPPKVSVFVPPRDGFFGNPRKSKLICKATGFSP | |
| | CH3(knobs) | RQIQVSWLREGKQVGSGVTTKQVQAEAKESGPTTYKVTSTLTIKESDWLG | |
| | | QSMFTCRVDHRGLTFQQNASSMCDKTHTCPPCPAPEAAGGPSVFLFPPKP | |
| | | KDTLMISRTPEVTCVVVDVSHEDPEVKFNWYVDGVEVHNAKTKPREEQYN | |
| | | STYRVVSVLTVLHQDWLNGKEYKCKVSNKALPAPIEKTISKAKGQPREPQ | |
| | | VYTLPPSREEMTKNQVSLWCLVKGFYPSDIAVEWESNGQPENNYKTTPPV | |
| | | LDSDGSFFLYSKLTVDKSRWQQGNVFSCSVMHEALHNHYTQKSLSLSPGK | |
| | AB426 VK-linker- | EIVLTQSPATLSLSPGERATLSCRASQSVSSYLAWYQQKPGQAPRLLIYD | 225 |
| | MH2n | ASNRATGIPARFSGSGSGTDFTLTISSLEPEDFAVYYCQQSSNWPRTFGQ | |
| | | GTKVEIKRTAELPPKVSVFVPPRDGFFGNPRKSELICEATGFSPRQIQVS | |
| | | WLREGKQVGSGVTTDQVQAEAKESGPTTYDVTSTLTIKESDWLGQSMFTC | |
| | | RVDHRGLTFQQNASSMC | |
| | Cetuximab VH- | QVQLKQSGPGLVQPSQSLSITCTVSGFSLTNYGVHWVRQSPGKGLEWLGV | 226 |
| | linker-AB002 VH- | IWSGGNTDYNTPFTSRLSINKDNSKSQVFFKMNSLQSNDTAIYYCARALT | |
| | CH1-hinge- | YYDYEFAYWGQGTLVTVSAASTKGPSVFPLAPQVQLQQSGAELARPGASV | |
| | CH2(LALA)- | KMSCKASGYTFTRYTMHWVKQRPGQGLEWIGYINPSRGYTNYNQKFKDKA | |
| | CH3(holes) | TLTTDKSSSTAYMQLSSLTSEDSAVYYCARYYDDHYCLDYWGQGTTLTVS | |
| | | SASTKGPSVFPLAPSSKSTSGGTAALGCLVKDYFPEPVTVSWNSGALTSG | |
| | | VHTFPAVLQSSGLYSLSSVVTVPSSSLGTQTYICNVNHKPSNTKVDKKVE | |
| | | PKSCDKTHTCPPCPAPEAAGGPSVFLFPPKPKDTLMISRTPEVTCVVVDV | |
| | | SHEDPEVKFNWYVDGVEVHNAKTKPREEQYNSTYRVVSVLTVLHQDWLNG | |
| | | KEYKCKVSNKALPAPIEKTISKAKGQPREPQVYTLPPSREEMTKNQVSLS | |
| | | CAVKGFYPSDIAVEWESNGQPENNYKTTPPVLDSDGSFFLVSKLTVDKSR | |
| | | WQQGNVFSCSVMHEALHNHYTQKSLSLSPGK | |
| | Cetuximab VK- | DILLTQSPVILSVSPGERVSFSCRASQSIGTNIHWYQQRTNGSPRLLIKY | 227 |
| | linker-AB002 VK- | ASESISGIPSRFSGSGSGTDFTLSINSVESEDIADYYCQQNNNQPTTFGA | |
| | CK | GTKLELKRTVAAPSVFIFPPQIVLTQSPAIMSASPGEKVTMTCRASSSVS | |
| | | YMNWYQQKSGTSPKRWIYDTSKVASGVPYRFSGSGSGTSYSLTISSMEAE | |
| | | DAATYYCQQWSSNPLTFGSGTKLEINRTVAAPSVFIFPPSDEQLKSGTAS | |
| | | VVCLLNNFYPREAKVQWKVDNALQSGNSQESVTEQDSKDSTYSLSSTLTL | |
| | | SKADYEKHKVYACEVTHQGLSSPVTKSFNRGEC | |
| TMH24 | AB426 VH-linker- | QVQLVESGGGVVQPGRSLRLDCKASGITFSNSGMHWVRQAPGKGLEWVAV | 228 |
| | MH2h-hinge- | IWYDGSKRYYADSVKGRFTISRDNSKNTLFLQMNSLRAEDTAVYYCATND | |
| | CH2(LALA)- | DYWGQGTLVTVSSASTELPPKVSVFVPPRDGFFGNPRKSKLACSATGFSP | |
| | CH3(knobs) | RQIQVSWLREGKQVGSGVTTDQVQAEAKESGPTTYKVASTLTIKESDWLG | |
| | | QSMFTCRVDHRGLTFQQNASSMCDKTHTCPPCPAPEAAGGPSVFLFPPKP | |
| | | KDTLMISRTPEVTCVVVDVSHEDPEVKFNWYVDGVEVHNAKTKPREEQYN | |
| | | STYRVVSVLTVLHQDWLNGKEYKCKVSNKALPAPIEKTISKAKGQPREPQ | |
| | | VYTLPPSREEMTKNQVSLWCLVKGFYPSDIAVEWESNGQPENNYKTTPPV | |
| | | LDSDGSFFLYSKLTVDKSRWQQGNVFSCSVMHEALHNHYTQKSLSLSPGK | |
| | AB426 VK-linker- | EIVLTQSPATLSLSPGERATLSCRASQSVSSYLAWYQQKPGQAPRLLIYD | 229 |
| | MH2k | ASNRATGIPARFSGSGSGTDFTLTISSLEPEDFAVYYCQQSSNWPRTFGQ | |
| | | GTKVEIKRTAELPPKVSVFVPPRDGFFGNPRKSKLWCQATGFSPRQIQVS | |
| | | WLREGKQVGSGVTTDQVQAEAKESGPTTYKVTSTLTIKESDWLGQSMFTC | |
| | | RVDHRGLTFQQNASSMC | |
| | Cetuximab VH- | QVQLKQSGPGLVQPSQSLSITCTVSGFSLTNYGVHWVRQSPGKGLEWLGV | 230 |
| | linker-AB002 VH- | IWSGGNTDYNTPFTSRLSINKDNSKSQVFFKMNSLQSNDTAIYYCARALT | |
| | CH1-hinge- | YYDYEFAYWGQGTLVTVSAASTKGPSVFPLAPQVQLQQSGAELARPGASV | |
| | CH2(LALA)- | KMSCKASGYTFTRYTMHWVKQRPGQGLEWIGYINPSRGYTNYNQKFKDKA | |
| | CH3(holes) | TLTTDKSSSTAYMQLSSLTSEDSAVYYCARYYDDHYCLDYWGQGTTLTVS | |
| | | SASTKGPSVFPLAPSSKSTSGGTAALGCLVKDYFPEPVTVSWNSGALTSG | |
| | | VHTFPAVLQSSGLYSLSSVVTVPSSSLGTQTYICNVNHKPSNTKVDKKVE | |
| | | PKSCDKTHTCPPCPAPEAAGGPSVFLFPPKPKDTLMISRTPEVTCVVVDV | |
| | | SHEDPEVKFNWYVDGVEVHNAKTKPREEQYNSTYRVVSVLTVLHQDWLNG | |
| | | KEYKCKVSNKALPAPIEKTISKAKGQPREPQVYTLPPSREEMTKNQVSLS | |
| | | CAVKGFYPSDIAVEWESNGQPENNYKTTPPVLDSDGSFFLVSKLTVDKSR | |
| | | WQQGNVFSCSVMHEALHNHYTQKSLSLSPGK | |
| | Cetuximab VK- | DILLTQSPVILSVSPGERVSFSCRASQSIGTNIHWYQQRTNGSPRLLIKY | 231 |
| | linker-AB002 VK- | ASESISGIPSRFSGSGSGTDFTLSINSVESEDIADYYCQQNNNWPTTFGA | |
| | CK | GTKLELKRTVAAPSVFIFPPQIVLTQSPAIMSASPGEKVTMTCRASSSVS | |
| | | YMNWYQQKSGTSPKRWIYDTSKVASGVPYRFSGSGSGTSYSLTISSMEAE | |
| | | DAATYYCQQWSSNPLTFGSGTKLEINRTVAAPSVFIFPPSDEQLKSGTAS | |
| | | VVCLLNNFYPREAKVQWKVDNALQSGNSQESVTEQDSKDSTYSLSSTLTL | |
| | | SKADYEKHKVYACEVTHQGLSSPVTKSFNRGEC | |
| TMH25 | AB426 VH-linker- | QVQLVESGGGVVQPGRSLRLDCKASGITFSNSGMHWVRQAPGKGLEWVAV | 232 |
| | MH2k-hinge- | IWYDGSKRYYADSVKGRFTISRDNSKNTLFLQMNSLRAEDTAVYYCATND | |
| | CH2(LALA)- | DYWGQGTLVTVSSASTELPPKVSVFVPPRDGFFGNPRKSKLWCQATGFSP | |
| | CH3(knobs) | RQIQVSWLREGKQVGSGVTTDQVQAEAKESGPTTYKVTSTLTIKESDWLG | |
| | | QSMFTCRVDHRGLTFQQNASSMCDKTHTCPPCPAPEAAGGPSVFLFPPKP | |
| | | KDTLMISRTPEVTCVVVDVSHEDPEVKFNWYVDGVEVHNAKTKPREEQYN | |
| | | STYRVVSVLTVLHQDWLNGKEYKCKVSNKALPAPIEKTISKAKGQPREPQ | |
| | | VYTLPPSREEMTKNQVSLWCLVKGFYPSDIAVEWESNGQPENNYKTTPPV | |
| | | LDSDGSFFLYSKLTVDKSRWQQGNVFSCSVMHEALHNHYTQKSLSLSPGK | |
| | AB426 VK-linker- | EIVLTQSPATLSLSPGERATLSCRASQSVSSYLAWYQQKPGQAPRLLIYD | 233 |
| | MH2h | ASNRATGIPARFSGSGSGTDFTLTISSLEPEDFAVYYCQQSSNWPRTFGQ | |
| | | GTKVEIKRTAELPPKVSVFVPPRDGFFGNPRKSKLACSATGFSPRQIQVS | |
| | | WLREGKQVGSGVTTDQVQAEAKESGPTTYKVASTLTIKESDWLGQSMFTC | |
| | | RVDHRGLTFQQNASSMC | |
| | Cetuximab VH- | QVQLKQSGPGLVQPSQSLSITCTVSGFSLTNYGVHWVRQSPGKGLEWLGV | 234 |
| | linker-AB002 VH- | IWSGGNTDYNTPFTSRLSINKDNSKSQVFFKMNSLQSNDTAIYYCARALT | |
| | CH1-hinge- | YYDYEFAYWGQGTLVTVSAASTKGPSVFPLAPQVQLQQSGAELARPGASV | |
| | CH2(LALA)- | KMSCKASGYTFTRYTMHWVKQRPGQGLEWIGYINPSRGYTNYNQKFKDKA | |
| | CH3(holes) | TLTTDKSSSTAYMQLSSLTSEDSAVYYCARYYDDHYCLDYWGQGTTLTVS | |
| | | SASTKGPSVFPLAPSSKSTSGGTAALGCLVKDYFPEPVTVSWNSGALTSG | |
| | | VHTFPAVLQSSGLYSLSSVVTVPSSSLGTQTYICNVNHKPSNTKVDKKVE | |
| | | PKSCDKTHTCPPCPAPEAAGGPSVFLFPPKPKDTLMISRTPEVTCVVVDV | |
| | | SHEDPEVKFNWYVDGVEVHNAKTKPREEQYNSTYRVVSVLTVLHQDWLNG | |
| | | KEYKCKVSNKALPAPIEKTISKAKGQPREPQVYTLPPSREEMTKNQVSLS | |
| | | CAVKGFYPSDIAVEWESNGQPENNYKTTPPVLDSDGSFFLVSKLTVDKSR | |
| | | WQQGNVFSCSVMHEALHNHYTQKSLSLSPGK | |
| | Cetuximab VK- | DILLTQSPVILSVSPGERVSFSCRASQSIGTNIHWYQQRTNGSPRLLIKY | 235 |
| | linker-AB002 VK- | ASESISGIPSRFSGSGSGTDFTLSINSVESEDIADYYCQQNNNWPTTFGA | |
| | CK | GTKLELKRTVAAPSVFIFPPQIVLTQSPAIMSASPGEKVTMTCRASSSVS | |
| | | YMNWYQQKSGTSPKRWIYDTSKVASGVPYRFSGSGSGTSYSLTISSMEAE | |
| | | DAATYYCQQWSSNPLTFGSGTKLEINRTVAAPSVFIFPPSDEQLKSGTAS | |
| | | VVCLLNNFYPREAKVQWKVDNALQSGNSQESVTEQDSKDSTYSLSSTLTL | |
| | | SKADYEKHKVYACEVTHQGLSSPVTKSFNRGEC | |
| KIH4 | AB426 VH-CH1- | QVQLVESGGGVVQPGRSLRLDCKASGITFSNSGMHWVRQAPGKGLEWVAV | 236 |
| | hinge-CH2(LALA)- | IWYDGSKRYYADSVKGRFTISRDNSKNTLFLQMNSLRAEDTAVYYCATND | |
| | CH3(knobs) | DYWGQGTLVTVSSASTKGPSVFPLAPSSKSTSGGTAALGCLVKDYFPEPV | |
| | | TVSWNSGALTSGVHTFPAVLQSSGLYSLSSVVTVPSSSLGTQTYICNVNH | |
| | | KPSNTKVDKKVEPKSCDKTHTCPPCPAPEAAGGPSVFLFPPKPKDTLMIS | |
| | | RTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVYTLPPS | |
| | | REEMTKNQVSLWCLVKGFYPSDIAVEWESNGQPENNYKTTPPVLDSDGSF | |
| | | FLYSKLTVDKSRWQQGNVFSCSVMHEALHNHYTQKSLSLSPGK | |
| | AB426 VL-CK | EIVLTQSPATLSLSPGERATLSCRASQSVSSYLAWYQQKPGQAPRLLIYD | 237 |
| | | ASNRATGIPARFSGSGSGTDFTLTISSLEPEDFAVYYCQQSSNWPRTFGQ | |
| | | GTKVEIKRTVAAPSVFIFPPSDEQLKSGTASVVCLLNNFYPREAKVQWKV | |
| | | DNALQSGNSQESVTEQDSKDSTYSLSSTLTLSKADYEKHKVYACEVTHQG | |
| | | LSSPVTKSFNRGEC | |
| | Cetuximab.2 VH- | QVQLKQSGPGLVQPSQSLSITCTVSGFSLTNYGVHWVRQSPGKGLEWLGV | 238 |
| | linker-AB002.2 | IWSGGNTDYNTPFTSRLSINKDNSKSQVFFKMNSLQSEDTAIYYCARALT | |
| | VH-CH1-hinge- | YYDYEFAYWGQGTLVTVSAASTKGPSVFPLAPQVQLQQSGAELARPGASV | |
| | CH2(LALA)- | KMSCKASGYTFTRYTMHWVKQRPGQGLEWIGYINPSRGYTNYNQKFKDKA | |
| | CH3(holes) | TLTTDKSSSTAYMQLSSLTSEDSAVYYCARYYDDHYSLDYWGQGTTLTVS | |
| | | SASTKGPSVFPLAPSSKSTSGGTAALGCLVKDYFPEPVTVSWNSGALTSG | |
| | | VHTFPAVLQSSGLYSLSSVVTVPSSSLGTQTYICNVNHKPSNTKVDKKVE | |
| | | PKSCDKTHTCPPCPAPEAAGGPSVFLFPPKPKDTLMISRTPEVTCVVVDV | |
| | | SHEDPEVKFNWYVDGVEVHNAKTKPREEQYNSTYRVVSVLTVLHQDWLNG | |
| | | KEYKCKVSNKALPAPIEKTISKAKGQPREPQVYTLPPSREEMTKNQVSLS | |
| | | CAVKGFYPSDIAVEWESNGQPENNYKTTPPVLDSDGSFFLVSKLTVDKSR | |
| | | WQQGNVFSCSVMHEALHNHYTQKSLSLSPGK | |
| | Cetuximab.2 VK- | DILLTQSPVILSVSPGERVSFSCRASQSIGTNIHWYQQRTSGSPRLLIKY | 239 |
| | linker-AB002.2 | ASESISGIPSRFSGSGSGTDFTLSINSVESEDIADYYCQQNNNWPTTFGA | |
| | VK-CK | GTKLELKRTVAAPSVFIFPPQIVLTQSPAIMSASPGEKVTMTCRASSSVS | |
| | | YMNWYQQKSGTSPKRWIYDTSKVASGVPYRFSGSGSGTSYSLTISSMEAE | |
| | | DAATYYCQQWSSNPLTFGSGTKLEIKRTVAAPSVFIFPPSDEQLKSGTAS | |
| | | VVCLLNNFYPREAKVQWKVDNALQSGNSQESVTEQDSKDSTYSLSSTLTL | |
| | | SKADYEKHKVYACEVTHQGLSSPVTKSFNRGEC | |
| KIH5 | YW243 VH-CH1- | EVQLVESGGGLVQPGGSLRLSCAASGFTFSDSWIHWVRQAPGKGLEWVAW | 240 |
| | hinge-CH2(LALA)- | ISPYGGSTYYADSVKGRFTISADTSKNTAYLQMNSLRAEDTAVYYCARRH | |
| | CH3 (knobs) | YPGGFDYWGQGTLVTVSAASTKGPSVFPLAPSSKSTSGGTAALGCLVKDY | |
| | | FPEPVTVSWNSGALTSGVHTFPAVLQSSGLYSLSSVVTVPSSSLGTQTYI | |
| | | CNVNHKPSNTKVDKKVEPKSCDKTHTCPPCPAPEAAGGPSVFLFPPKPKD | |
| | | TIMISRTPEVTCVVDVSHEDPEVKFNWYVDGVEVHNAKTKPREEQYNST | |
| | | YRVVSVLTVLHQDWLNGKEYKCKVSNKALPAPIEKTISKAKGQPREPQVY | |
| | | TLPPSREEMTKNQVSLWCLVKGFYPSDIAVEWESNGQPENNYKTTPPVLD | |
| | | SDGSFFLYSKLTVDKSRWQQGNVFSCSVMHEALHNHYTQKSLSLSPGK | |
| | YW243 VK-CK | DIQMTQSPSSLSASVGDRVTITCRASQDVSTAVAWYQQKPGKAPKLLIYS | 241 |
| | | ASFLYSGVPSRFSGSGSGTDFTLTISSLQPEDFATYYCQQYLYHPATFGQ | |
| | | GTKVEIKRTVAAPSVFIFPPSDEQLKSGTASVVCLLNNFYPREAKVQWKV | |
| | | DNALQSGNSQESVTEQDSKDSTYSLSSTLTLSKADYEKHKVYACEVTHQG | |
| | | LSSPVTKSFNRGEC | |
| | Cetuximab.2 VH- | QVQLKQSGPGLVQPSQSLSITCTVSGFSLTNYGVHWVRQSPGKGLEWLGV | 242 |
| | linker-AB002.2 | IWSGGNTDYNTPFTSRLSINKDNSKSQVFFKMNSLQSEDTAIYYCARALT | |
| | VH-CH1-hinge- | YYDYEFAYWGQGTLVTVSAASTKGPSVFPLAPQVQLQQSGAELARPGASV | |
| | CH2(LALA)- | KMSCKASGYTFTRYTMHWVKQRPGQGLEWIGYINPSRGYTNYNQKFKDKA | |
| | CH3(holes) | TLTTDKSSSTAYMQLSSLTSEDSAVYYCARYYDDHYSLDYWGQGTTLTVS | |
| | | SASTKGPSVFPLAPSSKSTSGGTAALGCLVKDYFPEPVTVSWNSGALTSG | |
| | | VHTFPAVLQSSGLYSLSSVVTVPSSSLGTQTYICNVNHKPSNTKVDKKVE | |
| | | PKSCDKTHTCPPCPAPEAAGGPSVFLFPPKPKDTLMISRTPEVTCVVVDV | |
| | | SHEDPEVKFNWYVDGVEVHNAKTKPREEQYNSTYRVVSVLTVLHQDWLNG | |
| | | KEYKCKVSNKALPAPIEKTISKAKGQPREPQVYTLPPSREEMTKNQVSLS | |
| | | CAVKGFYPSDIAVEWESNGQPENNYKTTPPVLDSDGSFFLVSKLTVDKSR | |
| | | WQQGNVFSCSVMHEALHNHYTQKSLSLSPGK | |
| | Cetuximab.2 VK- | DILLTQSPVILSVSPGERVSFSCRASQSIGTNIHWYQQRTSGSPRLLIKY | 243 |
| | linker-AB002.2 | ASESISGIPSRFSGSGSGTDFTLSINSVESEDIADYYCQQNNNWPTTFGA | |
| | VK-CK | GTKLELKRTVAAPSVFIFPPQIVLTQSPAIMSASPGEKVTMTCRASSSVS | |
| | | YMNWYQQKSGTSPKRWIYDTSKVASGVPYRFSGSGSGTSYSLTISSMEAE | |
| | | DAATYYCQQWSSNPLTFGSGTKLEIKRTVAAPSVFIFPPSDEQLKSGTAS | |
| | | VVCLLNNFYPREAKVQWKVDNALQSGNSQESVTEQDSKDSTYSLSSTLTL | |
| | | SKADYEKHKVYACEVTHQGLSSPVTKSFNRGEC | |
| KIH6 | YW243 VH-CH1- | EVQLVESGGGLVQPGGSLRLSCAASGFTFSDSWIHWVRQAPGKGLEWVAW | 244 |
| | hinge-CH2(LALA)- | ISPYGGSTYYADSVKGRFTISADTSKNTAYLQMNSLRAEDTAVYYCARRH | |
| | CH3 (knobs) | YPGGFDYWGQGTLVTVSAASTKGPSVFPLAPSSKSTSGGTAALGCLVKDY | |
| | | FPEPVTVSWNSGALTSGVHTFPAVLQSSGLYSLSSVVTVPSSSLGTQTYI | |
| | | CNVNHKPSNTKVDKKVEPKSCDKTHTCPPCPAPEAAGGPSVFLFPPKPKD | |
| | | TIMISRTPEVTCVVDVSHEDPEVKFNWYVDGVEVHNAKTKPREEQYNST | |
| | | YRVVSVLTVLHQDWLNGKEYKCKVSNKALPAPIEKTISKAKGQPREPQVY | |
| | | TLPPSREEMTKNQVSLWCLVKGFYPSDIAVEWESNGQPENNYKTTPPVLD | |
| | | SDGSFFLYSKLTVDKSRWQQGNVFSCSVMHEALHNHYTQKSLSLSPGK | |
| | YW243 VK-CK | DIQMTQSPSSLSASVGDRVTITCRASQDVSTAVAWYQQKPGKAPKLLIYS | 245 |
| | | ASFLYSGVPSRFSGSGSGTDFTLTISSLQPEDFATYYCQQYLYHPATFGQ | |
| | | GTKVEIKRTVAAPSVFIFPPSDEQLKSGTASVVCLLNNFYPREAKVQWKV | |
| | | DNALQSGNSQESVTEQDSKDSTYSLSSTLTLSKADYEKHKVYACEVTHQG | |
| | | LSSPVTKSFNRGEC | |
| | Cetuximab VH- | QVQLKQSGPGLVQPSQSLSITCTVSGFSLTNYGVHWVRQSPGKGLEWLGV | 246 |
| | linker-AB002 VH- | IWSGGNTDYNTPFTSRLSINKDNSKSQVFFKMNSLQSNDTAIYYCARALT | |
| | CH1-hinge- | YYDYEFAYWGQGTLVTVSAASTKGPSVFPLAPQVQLQQSGAELARPGASV | |
| | CH2(LALA)- | KMSCKASGYTFTRYTMHWVKQRPGQGLEWIGYINPSRGYTNYNQKFKDKA | |
| | CH3(holes) | TLTTDKSSSTAYMQLSSLTSEDSAVYYCARYYDDHYCLDYWGQGTTLTVS | |
| | | SASTKGPSVFPLAPSSKSTSGGTAALGCLVKDYFPEPVTVSWNSGALTSG | |
| | | VHTFPAVLQSSGLYSLSSVVTVPSSSLGTQTYICNVNHKPSNTKVDKKVE | |
| | | PKSCDKTHTCPPCPAPEAAGGPSVFLFPPKPKDTLMISRTPEVTCVVVDV | |
| | | SHEDPEVKFNWYVDGVEVHNAKTKPREEQYNSTYRVVSVLTVLHQDWLNG | |
| | | KEYKCKVSNKALPAPIEKTISKAKGQPREPQVYTLPPSREEMTKNQVSLS | |
| | | CAVKGFYPSDIAVEWESNGQPENNYKTTPPVLDSDGSFFLVSKLTVDKSR | |
| | | WQQGNVFSCSVMHEALHNHYTQKSLSLSPGK | |
| | Cetuximab VK- | DILLTQSPVILSVSPGERVSFSCRASQSIGTNIHWYQQRTNGSPRLLIKY | 247 |
| | linker-AB002 VK- | ASESISGIPSRFSGSGSGTDFTLSINSVESEDIADYYCQQNNNWPTTFGA | |
| | CK | GTKLELKRTVAAPSVFIFPPQIVLTQSPAIMSASPGEKVTMTCRASSSVS | |
| | | YMNWYQQKSGTSPKRWIYDTSKVASGVPYRFSGSGSGTSYSLTISSMEAE | |
| | | DAATYYCQQWSSNPLTFGSGTKLEINRTVAAPSVFIFPPSDEQLKSGTAS | |
| | | VVCLLNNFYPREAKVQWKVDNALQSGNSQESVTEQDSKDSTYSLSSTLTL | |
| | | SKADYEKHKVYACEVTHQGLSSPVTKSFNRGEC | |
| KIH7 | AB426 VH-CH1- | QVQLVESGGGVVQPGRSLRLDCKASGITFSNSGMHWVRQAPGKGLEWVAV | 248 |
| | hinge-CH2(LALA)- | IWYDGSKRYYADSVKGRFTISRDNSKNTLFLQMNSLRAEDTAVYYCATND | |
| | CH3(knobs) | DYWGQGTLVTVSSASTKGPSVFPLAPSSKSTSGGTAALGCLVKDYFPEPV | |
| | | TVSWNSGALTSGVHTFPAVLQSSGLYSLSSVVTVPSSSLGTQTYICNVNH | |
| | | KPSNTKVDKKVEPKSCDKTHTCPPCPAPEAAGGPSVFLFPPKPKDTLMIS | |
| | | RTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVYTLPPS | |
| | | REEMTKNQVSLWCLVKGFYPSDIAVEWESNGQPENNYKTTPPVLDSDGSF | |
| | | FLYSKLTVDKSRWQQGNVFSCSVMHEALHNHYTQKSLSLSPGK | |
| | AB426 VK-CK | EIVLTQSPATLSLSPGERATLSCRASQSVSSYLAWYQQKPGQAPRLLIYD | 249 |
| | | ASNRATGIPARFSGSGSGTDFTLTISSLEPEDFAVYYCQQSSNWPRTFGQ | |
| | | GTKVEIKRTVAAPSVFIFPPSDEQLKSGTASVVCLLNNFYPREAKVQWKV | |
| | | DNALQSGNSQESVTEQDSKDSTYSLSSTLTLSKADYEKHKVYACEVTHQG | |
| | | LSSPVTKSFNRGEC | |
| | Cetuximab VH- | QVQLKQSGPGLVQPSQSLSITCTVSGFSLTNYGVHWVRQSPGKGLEWLGV | 250 |
| | linker-AB002 VH- | IWSGGNTDYNTPFTSRLSINKDNSKSQVFFKMNSLQSNDTAIYYCARALT | |
| | CH1-hinge- | YYDYEFAYWGQGTLVTVSAASTKGPSVFPLAPQVQLQQSGAELARPGASV | |
| | CH2(LALA)- | KMSCKASGYTFTRYTMHWVKQRPGQGLEWIGYINPSRGYTNYNQKFKDKA | |
| | CH3(holes) | TLTTDKSSSTAYMQLSSLTSEDSAVYYCARYYDDHYCLDYWGQGTTLTVS | |
| | | SASTKGPSVFPLAPSSKSTSGGTAALGCLVKDYFPEPVTVSWNSGALTSG | |
| | | VHTFPAVLQSSGLYSLSSVVTVPSSSLGTQTYICNVNHKPSNTKVDKKVE | |
| | | PKSCDKTHTCPPCPAPEAAGGPSVFLFPPKPKDTLMISRTPEVTCVVVDV | |
| | | SHEDPEVKFNWYVDGVEVHNAKTKPREEQYNSTYRVVSVLTVLHQDWLNG | |
| | | KEYKCKVSNKALPAPIEKTISKAKGQPREPQVYTLPPSREEMTKNQVSLS | |
| | | CAVKGFYPSDIAVEWESNGQPENNYKTTPPVLDSDGSFFLVSKLTVDKSR | |
| | | WQQGNVFSCSVMHEALHNHYTQKSLSLSPGK | |
| | Cetuximab VK- | DILLTQSPVILSVSPGERVSFSCRASQSIGTNIHWYQQRTNGSPRLLIKY | 251 |
| | linker-AB002 VK- | ASESISGIPSRFSGSGSGTDFTLSINSVESEDIADYYCQQNNNWPTTFGA | |
| | CK | GTKLELKRTVAAPSVFIFPPQIVLTQSPAIMSASPGEKVTMTCRASSSVS | |
| | | YMNWYQQKSGTSPKRWIYDTSKVASGVPYRFSGSGSGTSYSLTISSMEAE | |
| | | DAATYYCQQWSSNPLTFGSGTKLEINRTVAAPSVFIFPPSDEQLKSGTAS | |
| | | VVCLLNNFYPREAKVQWKVDNALQSGNSQESVTEQDSKDSTYSLSSTLTL | |
| | | SKADYEKHKVYACEVTHQGLSSPVTKSFNRGEC | |
| DVD889 [hu | DVD889 heavy | EVQLLESGGDLVRPGGSLRLSCAASGFSFSRYGMSWVRQAPGKGLDWVAH | 252 |
| IgG1/k] | chain | ISASAGATYYADSVKGRFTISRDNSKNTLFLQMNNLRADDTAIYYCAKGG | |
| | | KQWLIPWFDPWGQGTLVTVSSASTKGPEVQLLESGGDLVRPGGSLRLSCA | |
| | | ASGFSFSRYGMSWVRQAPGKGLDWVAHISASAGATYYADSVKGRFTISRD | |
| | | NSKNTLFLQMNNLRADDTAIYYCAKGGKQWLIPWFDPWGQGTLVTVSSAS | |
| | | TKGPSVFPLAPSSKSTSGGTAALGCLVKDYFPEPVTVSWNSGALTSGVHT | |
| | | FPAVLQSSGLYSLSSVVTVPSSSLGTQTYICNVNHKPSNTKVDKKVEPKS | |
| | | CDKTHTCPPCPAPELLGGPSVFLFPPKPKDTLMISRTPEVTCVVVDVSHE | |
| | | DPEVKFNWYVDGVEVHNAKTKPREEQYNSTYRVVSVLTVLHQDWLNGKEY | |
| | | KCKVSNKALPAPIEKTISKAKGQPREPQVYTLPPSREEMTKNQVSLTCLV | |
| | | KGFYPSDIAVEWESNGQPENNYKTTPPVLDSDGSFFLYSKLTVDKSRWQQ | |
| | | GNVFSCSVMHEALHNHYTQKSLSLSPGK | |
| | DVD889 light | DIQMTQSPSSVSASVGDRVTIACRASQDISDRLAWYQQKPGKVPKVLIYG | 253 |
| | chain | ASSLQSGVPSRFSGSGSGTDFTLTINSLQPEDFATYYCQQANSFPLTFGG | |
| | | GTKVEMKRTVAAPDIQMTQSPSSVSASVGDRVTIACRASQDISDRLAWYQ | |
| | | QKPGKVPKVLIYGASSLQSGVPSRFSGSGSGTDFTLTINSLQPEDFATYY | |
| | | CQQANSFPLTFGGGTKVEMKRTVAAPSVFIFPPSDEQLKSGTASVVCLLN | |
| | | NFYPREAKVQWKVDNALQSGNSQESVTEQDSKDSTYSLSSTLTLSKADYE | |
| | | KHKVYACEVTHQGLSSPVTKSFNRGEC | |

All cloning was completed using homologous recombination and transformation in DH5a cells. All trispecific molecules were expressed in HEK293 cells and purified with MabSelect SuRe beads. Table 17 summarizes the expression yield of each trispecific molecule listed in Table 16.

TABLE 17
Expression Yield Of The Trispecific Molecules

| | Octet Titer | Yield | SEC |
|---|---|---|---|
| Antibody ID | (mg/L) | (mg/L) | Monomer % |
| TMH1 | 14.25 | 12.1 | 96.49 |
| TMH2 | 11.55 | 11 | 69.6 |
| TMH3 | 2.4 | 1.8 | 77.26 |
| TMH4 | 1.15 | 0.8 | 76.52 |
| TMH5 | 7.05 | 6.8 | 57.97 |
| TMH6 | 1.0 | 0.5 | 75.09 |
| TMH7 | 1.2 | 0.84 | 86.74 |
| TMH8 | 1.5 | 1.06 | 81.98 |
| TMH9 | 0.6 | 0.3 | 47.82 |
| TMH10 | 1.6 | 1.46 | 81.45 |
| KIH4 | 5.6 | 2.38 | |
| TMH11 | 4.8 | 5.94 | 56.06 |
| TMH12 | 2.7 | 4.3 | 62.49 |
| TMH13 | 3.4 | 5.02 | 67.15 |
| TMH14 | 1.1 | 1.38 | 38.41 |
| TMH15 | 1.1 | 1.3 | 30.69 |
| KIH5 | | 30.34 | 47.13 |
| TMH16 | 22.3 | 19.6 | 73.65 |
| TMH17 | 10 | 10.2 | 74.4 |
| TMH18 | 35.9 | 27.2 | 69.6 |
| TMH19 | 5.6 | 6 | 84.1 |
| TMH20 | 1.4 | 1.3 | 62.62 |
| KIH6 | 56.3 | 34.6 | 46.65 |
| TMH21 | 1.6 | 1.46 | 69.89 |
| TMH22 | 0.6 | 0.5 | 56.92 |
| TMH23 | 1 | 0.8 | 52.78 |
| TMH24 | 1.2 | 1.36 | 66.59 |
| TMH25 | 1 | 1.06 | 71.64 |
| KIH7 | 11.8 | 8 | 61.08 |

### Example 11.2: FACS Binding Assay for Trispecific Molecules Containing MH2 Domains

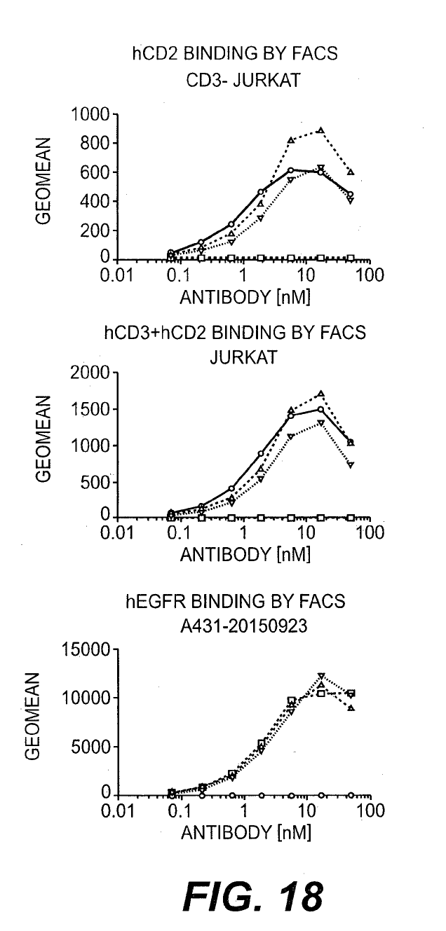

Trispecific molecule TMH1, containing MH2 domains, was tested in a FACS binding assay to confirm that it retained binding affinity to all the three targets (CD2, CD3, and EGFR). The PLY11 knobs-into-holes binding protein, the TS2/18 anti-CD2 halfbody, and the DVD860 anti-EGFR/CD3 halfbody were also tested for comparison. CD3 negative Jurkat cells were used for testing CD2 binding. Regular Jurkat cells were used to test CD2 and CD3 binding. A431 cells were used for testing EGFR binding. Cells were incubated with different concentrations of antibodies for 30 minutes and then incubated with fluorescence-conjugated secondary antibodies for another 30 minutes. Cells were then analyzed by flow cytometry and data was analyzed using FlowJo software. As shown in FIG. 18, TMH1 maintained its binding affinity to all the three targets.

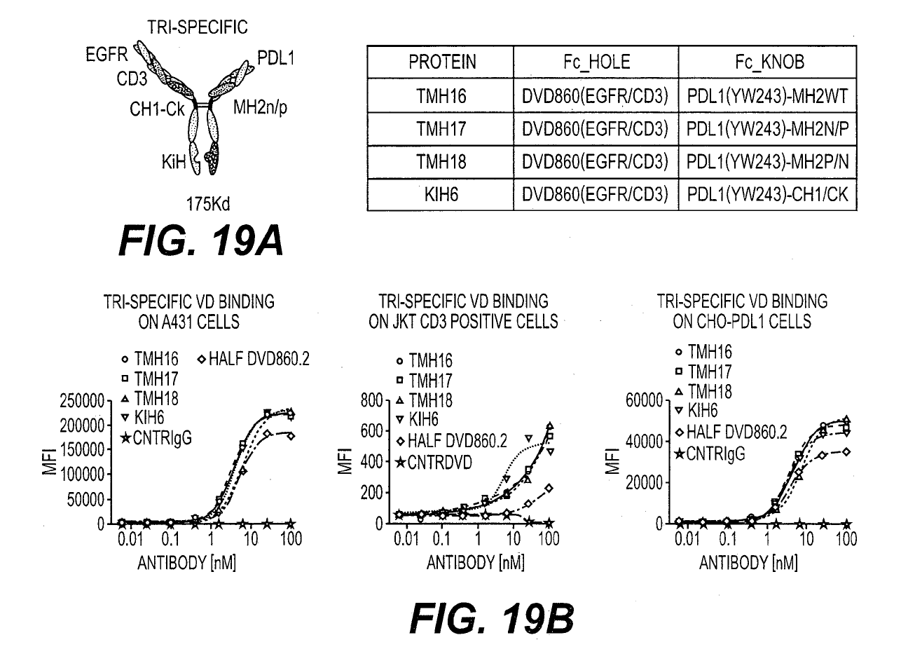

Trispecific molecules TMH16-18 were tested in a FACS binding assay to confirm that they retained binding affinity to all three targets (PDL1, CD3, and EGFR). The KIH6 knobs-into-holes binding protein, the anti-PDL1 YW243 halfbody and anti-EGFR/CD3 DVD860.2 halfbody were also tested for comparison. A431 cells were used for testing EGFR binding. Jurkat CD3 positive cells were used for testing CD3 binding. CHO-PDL1 cells were used for testing PDL1 binding. Cells were incubated with different concentrations of antibodies for 30 minutes and then incubated with incubated with fluorescence-conjugated secondary antibodies for another 30 minutes. Cells were then analyzed by flow cytometry and data was analyzed using FlowJo software. As shown in FIG. 19B, TMH16-18 maintained their binding affinity to all the three targets. DVD889 [hu IgG1/k], which binds Tetanus toxoid, was used as a negative control.

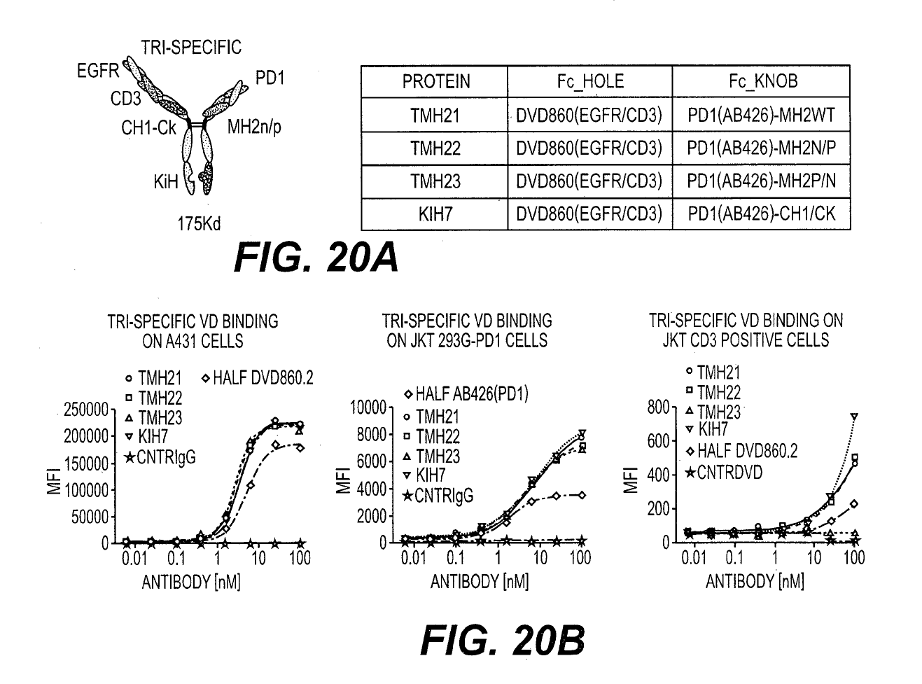

Trispecific molecules TMH21-23 were tested in a FACS binding assay to confirm that they retained binding affinity to all three targets (PD1, CD3, and EGFR). The KIH7 knobs-into-holes binding protein, the anti-PD1 AB426 halfbody and anti-EGFR/CD3 DVD860.2 halfbody were also tested for comparison. A431 cells were used for testing EGFR binding. Jurkat CD3 positive cells were used for testing CD3 binding. 293G-PD1 cells were used for testing PD1 binding. Cells were incubated with different concentrations of antibodies for 30 minutes and then incubated with incubated with fluorescence-conjugated secondary antibodies for another 30 minutes. Cells were then analyzed by flow cytometry and data was analyzed using FlowJo software. As shown in FIG. 20B, TMH21-23 maintained their binding affinity to all the three targets. DVD889 [hu IgG1/k] was used as a negative control.

### Example 12: Generation of Tetraspecific Molecules with Wild Type MH2 Homodimer or Engineered MH2a/MH2b Heterodimer

Table 18 summarizes ten exemplary combinations that can be used to build tetraspecific molecules using MH2/MH2, MH2p/MH2n, or MH2k/MH2h domains in a knobs-into-holes format. In each, VH1 and VL1 are the variable domains taken from one parental antibody, VH2 and VL2 are variable domains from a second parental antibody, VH3 and VL3 are variable domains from a third parental antibody, and VH4 and VL4 are variable domains from a fourth parental antibody. Each tetraspecific molecule is generated with four chains: 2 heavy chains (chain 1 and chain 3) and 2 light chains (chain 2 and chain 4).

TABLE 18
Combinations of 4 Chains That Generate Tetraspecific
Molecules Using MH2/MH2, MH2p/MH2n, Or MH2k/MH2h
Dimers In A Knobs-Into-Holes Format

| | Trispecific IgG Combination | | | |
|---|---|---|---|---|
| | Chain 1 | Chain 2 | Chain 3 | Chain 4 |
| 1 | VH1-linker- | VL1-linker- | VH3-linker- | VL3-linker- |
| | VH2-linker- | VL2-linker- | VH4-CH1-hinge- | VL4-Cκ (λ) |
| | MH2-hinge- | MH2 | CH2—CH3 | |
| | CH2—CH3 | | (holes) | |
| | (knobs) | | | |
| 2 | VH1-linker- | VL1-linker- | VH3-linker- | VL3-linker- |
| | VH2-linker- | VL2-linker- | VH4-CH1-hinge- | VL4-Cκ (λ) |
| | MH2p-hinge- | MH2n | CH2—CH3 | |
| | CH2—CH3 | | (holes) | |
| | (knobs) | | | |
| 3 | VH1-linker- | VL1-linker- | VH3-linker- | VL3-linker- |
| | VH2-linker- | VL2-linker- | VH4-CH1-hinge- | VL4-Cκ (λ) |
| | MH2n-hinge- | MH2p | CH2—CH3 | |
| | CH2—CH3 | | (holes) | |
| | (knobs) | | | |
| 4 | VH1-linker- | VL1-linker- | VH3-linker- | VL3-linker- |
| | VH2-linker- | VL2-linker- | VH4-CH1-hinge- | VL4-Cκ (λ) |
| | MH2k-hinge- | MH2h | CH2—CH3 | |
| | CH2—CH3 | | (holes) | |
| | (knobs) | | | |
| 5 | VH1-linker- | VL1-linker- | VH3-linker- | VL3-linker- |
| | VH2-linker- | VL2-linker- | VH4-CH1-hinge- | VL4-Cκ (λ) |
| | MH2h-hinge- | MH2k | CH2—CH3 | |
| | CH2—CH3 | | (holes) | |
| | (knobs) | | | |
| 6 | VH1-linker- | VL1-linker- | VH3-linker- | VL3-linker- |
| | VH2-linker- | VL2-linker- | VH4-CH1-hinge- | VL4-Cκ (λ) |
| | MH2-hinge- | MH2 | CH2—CH3 | |
| | CH2—CH3 | | (knobs) | |
| | (holes) | | | |
| 7 | VH1-linker- | VL1-linker- | VH3-linker- | VL3-linker- |
| | VH2-linker- | VL2-linker- | VH4-CH1-hinge- | VL4-Cκ (λ) |
| | MH2p-hinge- | MH2n | CH2—CH3 | |
| | CH2—CH3 | | (knobs) | |
| | (holes) | | | |
| 8 | VH1-linker- | VL1-linker- | VH3-linker- | VL3-linker- |
| | VH2-linker- | VL2-linker- | VH4-CH1-hinge- | VL4-Cκ (λ) |
| | MH2n-hinge- | MH2p | CH2—CH3 | |
| | CH2—CH3 | | (knobs) | |
| | (holes) | | | |
| 9 | VH1-linker- | VL1-linker- | VH3-linker- | VL3-linker- |
| | VH2-linker- | VH2-linker- | VH4-CH1-hinge- | VL4-Cκ (λ) |
| | MH2k-hinge- | MH2h | CH2—CH3 | |
| | CH2—CH3 | | (knobs) | |
| | (holes) | | | |
| 10 | VH1-linkner- | VL1-linker- | VH3-linker- | VL3-linker- |
| | VH2-linker- | MH2k | VH4-CH1-hinge- | VL4-Cκ (λ) |
| | MH2h-hinge- | | CH2—CH3 | |
| | CH2—CH3 | | (knobs) | |
| | (holes) | | | |

Eight Tetraspecific molecules were generated based on chain combination 3 listed in Table 18 with the binding to 4-1BB, CD2, EGFR and CD3. The arrangements of variable domains in each tetra-specific molecule are summarized in the Table 19. All cloning was completed using homologous recombination and transformation in DH5a cells. All tetraspecific molecules were expressed in HEK293 cells and purified with MabSelect SuRe beads. The expression yield of each molecule is summarized in Table 19. The variable domain sequences used to generate the tetraspecific molecules are listed in Table 20. Table 21 summarizes the sequences of the four chains in each of the tetraspecific molecules that were generated and tested.

TABLE 19
Tetraspecific Molecules Containing MH2 Domains

| | Knob heavy/light | | | Hole heavy/light | | | OCTET | yield | |
|---|---|---|---|---|---|---|---|---|---|
| Tetraspecific | VH1/VL1 | linker | VH4/VL2 | VH3/VL3 | linker | VH4/VL4 | (ug/ml) | (mg/L) | SEC % |
| PLY13 | 4-1BB | LL | CD2 | EGFR | LL | CD3 | 0.5 | 0.378 | 93.63 |
| | (AB430) | | (AB765) | (Cetuximab) | | (AB002) | | | |
| PLY14 | 4-1BB | LS | CD2 | EGFR | LL | CD3 | 1.1 | 0.972 | 89.9 |
| | (AB430) | | (AB765) | (Cetuximab) | | (AB002) | | | |
| PLY15 | 4-1BB | SL | CD2 | EGFR | LL | CD3 | 0.6 | 0.567 | 94.92 |
| | (AB430) | | (AB765) | (Cetuximab) | | (AB002) | | | |
| PLY16 | 4-1BB | SS | CD2 | EGFR | LL | CD3 | 0.8 | 0.378 | 87.47 |
| | (AB430) | | (AB765) | (Cetuximab) | | (AB002) | | | |
| PLY17 | CD2 | LL | 4-1BB | EGFR | LL | CD3 | 0.5 | 0.441 | 88.27 |
| | (AB765) | | (AB430) | (Cetuximab) | | (AB002) | | | |
| PLY18 | CD2 | LS | 4-1BB | EGFR | LL | CD3 | 0.7 | 0.621 | 85.52 |
| | (AB765) | | (AB430) | (Cetuximab) | | (AB002) | | | |
| PLY19 | CD2 | SL | 4-1BB | EGFR | LL | CD3 | 0.6 | 0.486 | 94.53 |
| | (AB765) | | (AB430) | (Cetuximab) | | (AB002) | | | |
| PLY20 | CD2 | SS | 4-1BB | EGFR | LL | CD3 | 0.9 | 0.702 | 93.29 |
| | (AB765) | | (AB430) | (Cetuximab) | | (AB002) | | | |

TABLE 20
Antibody Variable Domains Used To Build IgG-Like Molecules Containing
MH2 Domains

| Antibody | | Variable Domain Sequence | SEQ ID |
|---|---|---|---|
| Target | Domain | 1234567890123456769012345678901234567890 | NO. |
| CD3 | CD3 VH | QVQLQQSGAELARPGASVKMSCKASGYTFTRYTMHWVKQR | 254 |
| (AB002) | | PGQGLEWIGYINPSRGYTNYNQKFKDKATLTTDKSSSTAY | |
| | | MQLSSLTSEDSAVYYCARYYDDHYCLDYWGQGTTLTVSS | |
| | CD3 VK | QIVLTQSPAIMSASPGEKVTMTCRASSSVSYMNWYQQKSG | 255 |
| | | TSPKRWIYDTSKVASGVPYRFSGSGSGTSYSLTISSMEAE | |
| | | DAATYYCQWSSNPLTFGSGTKLEIN | |
| EGFR | Cetuximab | QVQLKQSGPGLVQPSQSLSITCTVSGFSLTNYGNHWVRQS | 256 |
| (Cetuximab) | VH | PGKGLEWLGVIWSGGNTDYNTPFTSRLSINKDNSKSQVFF | |
| | | KMNSLQSNDTAIYYCARALTYYDYEFAYWGQGTLVTVSA | |
| | Cetuximab | DILLTQSPVILSVSPGERVSFSCRASQSIGTNIHWYQQRT | 257 |
| | VK | NGSPRLLIKYASESISGIPSRFSGSGSGTDFTLSINSVES | |
| | | EDIADYYCQNNNWPTTFGAGTKLELK | |
| 4-1BB | AB430 VH | QVQLQQWGAGLLKPSETLSLTCAVYGGSFSGYYWSWIRQS | 258 |
| (AB430) | | PEKGLEWIGEINHGGYVTYNPSLESRVTISVDTSKNQFSL | |
| | | KLSSVTAADTAVYYCARDYGPGNYDWYFDLWGRGTLVTVS | |
| | | S | |
| | AB430 VK | EIVLTQSPATLSLSPGERATLSCRASQSVSSYLAWYQQKP | 259 |
| | | GQAPRLLIYDASNRATGIPARFSGSGSGTDFTLTISSLEP | |
| | | EDFAVYYCQQRSNWPPALTFGGGTKVEIK | |
| CD2 | AB765 VH | EVQLVESGGGLVQPGGSLRLSCAASGFAFSSYDMSWVRQA | 260 |
| (AB765) | | PGKGLEWVSYISGGGFTYYPDTVKGRFTISRDNSKNTLYL | |
| | | QMNSLRAEDTAVYYCARQGANWELVYWGQGTLVTVSS | |
| | | EIVLTQSPATLSLSPGERATLSCRASQSISDFLHWYQQKP | 261 |
| | AB765 VK | GQAPRLLIKYASQSISGIPARFSGSGSGTDFTLTISSLEP | |
| | | EDFAVYYCQNGHNFPPTFGGGTKVEIK | |

TABLE 21
Sequences Of Generated Tetraspecific Molecules

| Tetra- | | Sequence | SEQ |
|---|---|---|---|
| specific | Chain | 1234567890123456789012345678901234567890 | NO: |
| PLY13 | AB430VH-L-AB765VH- | QVQLQQWGAGLLKPSETLSLTCAVYGGSFSGYYWSWIRQS | 262 |
| | linker-MH2n-hinge- | PEKGLEWIGEINHGGYVTYNPSLESRVTISVDTSKNQFSL | |
| | CH2-CH3 (knobs) | KLSSVTAADTAVYYCARDYGPGNYDWYFDLWGRGTLVTVS | |
| | | SASTKGPSVFPLAPEVQLVESGGGLVQPGGSLRLSCAASG | |
| | | FAFSSYDMSWVRQAPGKGLEWVSYISGGGFTYYPDTVKGR | |
| | | FTISRDNSKNTLYLQMNSLRAEDTAVYYCARQGANWELVY | |
| | | WGQGTLVTVSSASTELPPKVSVFVPPRDGFFGNPRKSELI | |
| | | CEATGFSPRQIQVSWLREGKQVGSGVTTDQVQAEAKESGP | |
| | | TTYDVTSTLTIKESDWLGQSMFTCRVDHRGLTFQQNASSM | |
| | | CDKTHTCPPCPAPELLGGPSVFLFPPKPKDTLMISRTPEV | |
| | | TCVVVDVSHEDPEVKFNWYVDGVEVHNAKTKPREEQYNST | |
| | | YRVVSVLTVLHQDWLNGKEYKCKVSNKALPAPIEKTISKA | |
| | | KGQPREPQVYTLPPSREEMTKNQVSLWCLVKGFYPSDIAV | |
| | | EWESNGQPENNYKTTPPVLDSDGSFFLYSKLTVDKSRWQQ | |
| | | GNVFSCSVMHEALHNHYTQKSLSLSPGK | |
| | AB430VL-L- | EIVLTQSPATLSLSPGERATLSCRASQSVSSYLAWYQQKP | 263 |
| | AB765VL-linker- | GQAPRLLIYDASNRATGIPARFSGSGSGTDFTLTISSLEP | |
| | MH2p | EDFAVYYCQQRSNWPPALTFGGGTKVEIKRTVAAPSVFIF | |
| | | PPEIVLTQSPATLSLSPGERATLSCRASQSISDFLHWYQQ | |
| | | KPGQAPRLLIKYASQSISGIPARFSGSGSGTDFTLTISSL | |
| | | EPEDFAVYYCQNGHNFPPTFGGGTKVEIKRTAELPPKVSV | |
| | | FVPPRDGFFGNPRKSKLICKATGFSPRQIQVSWLREGKQV | |
| | | GSGVTTKQVQAEAKESGPTTYKVTSTLTIKESDWLGQSMF | |
| | | TCRVDHRGLTFQQNASSMC | |
| | Cetuximab VH- | QVQLKQSGPGLVQPSQSLSITCTVSGFSLTNYGVHWVRQS | 264 |
| | linker-CD3VH- | PGKGLEWLGVIWSGGNTDYNTPFTSRLSINKDNSKSQVFF | |
| | linker-AB002 VH- | KMNSLQSNDTAIYYCARALTYYDYEFAYWGQGTLVTVSAA | |
| | CH1-hinge-CH2- | STKGPSVFPLAPQVQLQQSGAELARPGASVKMSCKASGYT | |
| | CH3 (holes) | FTRYTMHWVKQRPGQGLEWIGYINPSRGYTNYNQKFKDKA | |
| | | TLTTDKSSSTAYMQLSSLTSEDSAVYYCARYYDDHYCLDY | |
| | | WGQGTTLTVSSASTKGPSVFPLAPSSKSTSGGTAALGCLV | |
| | | KDYFPEPVTVSWNSGALTSGVHTFPAVLQSSGLYSLSSVV | |
| | | TVPSSSLGTQTYICNVNHKPSNTKVDKKVEPKSCDKTHTC | |
| | | PPCPAPELLGGPSVFLFPPKPKDTLMISRTPEVTCVVVDV | |
| | | SHEDPEVKFNWYVDGVEVHNAKTKPREEQYNSTYRVVSVL | |
| | | TVLHQDWLNGKEYKCKVSNKALPAPIEKTISKAKGQPREP | |
| | | QVYTLPPSREEMTKNQVSLSCAVKGFYPSDIAVEWESNGQ | |
| | | PENNYKTTPPVLDSDGSFFLVSKLTVDKSRWQQGNVFSCS | |
| | | VMHEALHNHYTQKSLSLSPGK | |
| | Cetuximab VK- | DILLTQSPVILSVSPGERVSFSCRASQSIGTNIHWYQQRT | 265 |
| | linker-CD3VK- | NGSPRLLIKYASESISGIPSRFSGSGSGTDFTLSINSVES | |
| | linker-AB002 VK- | EDIADYYCQQNNNWPTTFGAGTKLELKRTVAAPSVFIFPP | |
| | Ck | QIVLTQSPAIMSASPGEKVTMTCRASSSVSYMNWYQQKSG | |
| | | TSPKRWIYDTSKVASGVPYRFSGSGSGTSYSLTISSMEAE | |
| | | DAATYYCQQWSSNPLTFGSGTKLEINRTVAAPSVFIFPPS | |
| | | DEQLKSGTASVVCLLNNFYPREAKVQWKVDNALQSGNSQE | |
| | | SVTEQDSKDSTYSLSSTLTLSKADYEKHKVYACEVTHQGL | |
| | | SSPVTKSFNRGEC | |
| PLY14 | AB430VH-L- | QVQLQQWGAGLLKPSETLSLTCAVYGGSFSGYYWSWIRQS | 266 |
| | AB765VH-linker- | PEKGLEWIGEINHGGYVTYNPSLESRVTISVDTSKNQFSL | |
| | MH2n-hinge-CH2- | KLSSVTAADTAVYYCARDYGPGNYDWYFDLWGRGTLVTVS | |
| | CH3 (knobs) | aASTKGPSVFPLAPEVQLVESGGGLVQPGGSLRLSCAASG | |
| | | FAFSSYDMSWVRQAPGKGLEWVSYISGGGFTYYPDTVKGR | |
| | | FTISRDNSKNTLYLQMNSLRAEDTAVYYCARQGANWELVY | |
| | | WGQGTLVTVSSASTELPPKVSVFVPPRDGFFGNPRKSELI | |
| | | CEATGFSPRQIQVSWLREGKQVGSGVTTDQVQAEAKESGP | |
| | | TTYDVTSTLTIKESDWLGQSMFTCRVDHRGLTFQQNASSM | |
| | | CDKTHTCPPCPAPELLGGPSVFLFPPKPKDTLMISRTPEV | |
| | | TCVVVDVSHEDPEVKFNWYVDGVEVHNAKTKPREEQYNST | |
| | | YRVVSVLTVLHQDWLNGKEYKCKVSNKALPAPIEKTISKA | |
| | | KGQPREPQVYTLPPSREEMTKNQVSLWCLVKGFYPSDIAV | |
| | | EWESNGQPENNYKTTPPVLDSDGSFFLYSKLTVDKSRWQQ | |
| | | GNVFSCSVMHEALHNHYTQKSLSLSPGK | |
| | AB430VL-S- | EIVLTQSPATLSLSPGERATLSCRASQSVSSYLAWYQQKP | 267 |
| | AB765VL-linker- | GQAPRLLIYDASNRATGIPARFSGSGSGTDFTLTISSLEP | |
| | MH2p | EDFAVYYCQQRSNWPPALTFGGGTKVEIKRTVAAPEIVLT | |
| | | QSPATLSLSPGERATLSCRASQSISDFLHWYQQKPGQAPR | |
| | | LLIKYASQSISGIPARFSGSGSGTDFTLTISSLEPEDFAV | |
| | | YYCQNGHNFPPTFGGGTKVEIKRTAELPPKVSVFVPPRDG | |
| | | FFGNPRKSKLICKATGFSPRQIQVSWLREGKQVGSGVTTK | |
| | | QVQAEAKESGPTTYKVTSTLTIKESDWLGQSMFTCRVDHR | |
| | | GLTFQQNASSMC | |
| | Cetuximab VH- | QVQLKQSGPGLVQPSQSLSITCTVSGFSLTNYGVHWVRQS | 268 |
| | linker-CD3VH- | PGKGLEWLGVIWSGGNTDYNTPFTSRLSINKDNSKSQVFF | |
| | linker-AB002 VH- | KMNSLQSNDTAIYYCARALTYYDYEFAYWGQGTLVTVSAA | |
| | CH1-hinge-CH2- | STKGPSVFPLAPQVQLQQSGAELARPGASVKMSCKASGYT | |
| | CH3 (holes) | FTRYTMHWVKQRPGQGLEWIGYINPSRGYTNYNQKFKDKA | |
| | | TLTTDKSSSTAYMQLSSLTSEDSAVYYCARYYDDHYCLDY | |
| | | WGQGTTLTVSSASTKGPSVFPLAPSSKSTSGGTAALGCLV | |
| | | KDYFPEPVTVSWNSGALTSGVHTFPAVLQSSGLYSLSSVV | |
| | | TVPSSSLGTQTYICNVNHKPSNTKVDKKVEPKSCDKTHTC | |
| | | PPCPAPELLGGPSVFLFPPKPKDTLMISRTPEVTCVVVDV | |
| | | SHEDPEVKFNWYVDGVEVHNAKTKPREEQYNSTYRVVSVL | |
| | | TVLHQDWLNGKEYKCKVSNKALPAPIEKTISKAKGQPREP | |
| | | QVYTLPPSREEMTKNQVSLSCAVKGFYPSDIAVEWESNGQ | |
| | | PENNYKTTPPVLDSDGSFFLVSKLTVDKSRWQQGNVFSCS | |
| | | VMHEALHNHYTQKSLSLSPGK | |
| | Cetuximab VK- | DILLTQSPVILSVSPGERVSFSCRASQSIGTNIHWYQQRT | 269 |
| | linker-CD3VK- | NGSPRLLIKYASESISGIPSRFSGSGSGTDFTLSINSVES | |
| | linker-AB002 VK- | EDIADYYCQQNNNWPTTFGAGTKLELKRTVAAPSVFIFPP | |
| | Ck | QIVLTQSPAIMSASPGEKVTMTCRASSSVSYMNWYQQKSG | |
| | | TSPKRWIYDTSKVASGVPYRFSGSGSGTSYSLTISSMEAE | |
| | | DAATYYCQQWSSNPLTFGSGTKLEINRTVAAPSVFIFPPS | |
| | | DEQLKSGTASVVCLLNNFYPREAKVQWKVDNALQSGNSQE | |
| | | SVTEQDSKDSTYSLSSTLTLSKADYEKHKVYACEVTHQGL | |
| | | SSPVTKSFNRGEC | |
| PLY15 | AB430VH-S- | QVQLQQWGAGLLKPSETLSLTCAVYGGSFSGYYWSWIRQS | 270 |
| | AB765VH-linker- | PEKGLEWIGEINHGGYVTYNPSLESRVTISVDTSKNQFSL | |
| | MH2n-hinge-CH2- | KLSSVTAADTAVYYCARDYGPGNYDWYFDLWGRGTLVTVS | |
| | CH3 (knobs) | aASTKGPEVQLVESGGGLVQPGGSLRLSCAASGFAFSSYD | |
| | | MSWVRQAPGKGLEWVSYISGGGFTYYPDTVKGRFTISRDN | |
| | | SKNTLYLQMNSLRAEDTAVYYCARQGANWELVYWGQGTLV | |
| | | TVSSASTELPPKVSVFVPPRDGFFGNPRKSELICEATGFS | |
| | | PRQIQVSWLREGKQVGSGVTTDQVQAEAKESGPTTYDVTS | |
| | | TLTIKESDWLGQSMFTCRVDHRGLTFQQNASSMCDKTHTC | |
| | | PPCPAPELLGGPSVFLFPPKPKDTLMISRTPEVTCVVVDV | |
| | | SHEDPEVKFNWYVDGVEVHNAKTKPREEQYNSTYRVVSVL | |
| | | TVLHQDWLNGKEYKCKVSNKALPAPIEKTISKAKGQPREP | |
| | | QVYTLPPSREEMTKNQVSLWCLVKGFYPSDIAVEWESNGQ | |
| | | PENNYKTTPPVLDSDGSFFLYSKLTVDKSRWQQGNVFSCS | |
| | | VMHEALHNHYTQKSLSLSPGK | |
| | AB430VL-L- | EIVLTQSPATLSLSPGERATLSCRASQSVSSYLAWYQQKP | 271 |
| | AB765VL-linker- | GQAPRLLIYDASNRATGIPARFSGSGSGTDFTLTISSLEP | |
| | MH2p | EDFAVYYCQQRSNWPPALTFGGGTKVEIKRTVAAPSVFIF | |
| | | PPEIVLTQSPATLSLSPGERATLSCRASQSISDFLHWYQQ | |
| | | KPGQAPRLLIKYASQSISGIPARFSGSGSGTDFTLTISSL | |
| | | EPEDFAVYYCQNGHNFPPTFGGGTKVEIKRTAELPPKVSV | |
| | | EVPPRDGFFGNPRKSKLICKATGFSPRQIQVSWLREGKQV | |
| | | GSGVTTKQVQAEAKESGPTTYKVTSTLTIKESDWLGQSMF | |
| | | TCRVDHRGLTFQQNASSMC | |
| | Cetuximab VH- | QVQLKQSGPGLVQPSQSLSITCTVSGFSLTNYGVHWVRQS | 272 |
| | linker-CD3VH- | PGKGLEWLGVIWSGGNTDYNTPFTSRLSINKDNSKSQVFF | |
| | linker-AB002 VH- | KMNSLQSNDTAIYYCARALTYYDYEFAYWGQGTLVTVSAA | |
| | CH1-hinge-CH2- | STKGPSVFPLAPQVQLQQSGAELARPGASVKMSCKASGYT | |
| | CH3 (holes) | FTRYTMHWVKQRPGQGLEWIGYINPSRGYTNYNQKFKDKA | |
| | | TLTTDKSSSTAYMQLSSLTSEDSAVYYCARYYDDHYCLDY | |
| | | WGQGTTLTVSSASTKGPSVFPLAPSSKSTSGGTAALGCLV | |
| | | KDYFPEPVTVSWNSGALTSGVHTFPAVLQSSGLYSLSSVV | |
| | | TVPSSSLGTQTYICNVNHKPSNTKVDKKVEPKSCDKTHTC | |
| | | PPCPAPELLGGPSVFLFPPKPKDTLMISRTPEVTCVVVDV | |
| | | SHEDPEVKFNWYVDGVEVHNAKTKPREEQYNSTYRVVSVL | |
| | | TVLHQDWLNGKEYKCKVSNKALPAPIEKTISKAKGQPREP | |
| | | QVYTLPPSREEMTKNQVSLSCAVKGFYPSDIAVEWESNGQ | |
| | | PENNYKTTPPVLDSDGSFFLVSKLTVDKSRWQQGNVFSCS | |
| | | VMHEALHNHYTQKSLSLSPGK | |
| | Cetuximab VK- | DILLTQSPVILSVSPGERVSFSCRASQSIGTNIHWYQQRT | 273 |
| | linker-CD3VK- | NGSPRLLIKYASESISGIPSRFSGSGSGTDFTLSINSVES | |
| | linker-AB002 VK- | EDIADYYCQQNNNWPTTFGAGTKLELKRTVAAPSVFIFPP | |
| | Ck | QIVLTQSPAIMSASPGEKVTMTCRASSSVSYMNWYQQKSG | |
| | | TSPKRWIYDTSKVASGVPYRFSGSGSGTSYSLTISSMEAE | |
| | | DAATYYCQQWSSNPLTFGSGTKLEINRTVAAPSVFIFPPS | |
| | | VDEQLKSGTASVVCLLNNFYPREAKVQWKVDNALQSGNSQE | |
| | | SVTEQDSKDSTYSLSSTLTLSKADYEKHKVYACEVTHQGL | |
| | | SSPVTKSFNRGEC | |
| PLY16 | AB430VH-S- | QVQLQQWGAGLLKPSETLSLTCAVYGGSFSGYYWSWIRQS | 274 |
| | AB765VH-linker- | PEKGLEWIGEINHGGYVTYNPSLESRVTISVDTSKNQFSL | |
| | MH2n-hinge-CH2- | KLSSVTAADTAVYYCARDYGPGNYDWYFDLWGRGTLVTVS | |
| | CH3 (knobs) | SASTKGPEVQLVESGGGLVQPGGSLRLSCAASGFAFSSYD | |
| | | MSWVRQAPGKGLEWVSYISGGGFTYYPDTVKGRFTISRDN | |
| | | SKNTLYLQMNSLRAEDTAVYYCARQGANWELVYWGQGTLV | |
| | | TVSSASTELPPKVSVFVPPRDGFFGNPRKSELICEATGFS | |
| | | PRQIQVSWLREGKQVGSGVTTDQVQAEAKESGPTTYDVTS | |
| | | TLTIKESDWLGQSMFTCRVDHRGLTFQQNASSMCDKTHTC | |
| | | PPCPAPELLGGPSVFLFPPKPKDTLMISRTPEVTCVVVDV | |
| | | SHEDPEVKFNWYVDGVEVHNAKTKPREEQYNSTYRVVSVL | |
| | | TVLHQDWLNGKEYKCKVSNKALPAPIEKTISKAKGQPREP | |
| | | QVYTLPPSREEMTKNQVSLWCLVKGFYPSDIAVEWESNGQ | |
| | | PENNYKTTPPVLDSDGSFFLYSKLTVDKSRWQQGNVFSCS | |
| | | VMHEALHNHYTQKSLSLSPGK | |
| | AB430VL-S- | EIVLTQSPATLSLSPGERATLSCRASQSVSSYLAWYQQKP | 275 |
| | AB765VL-linker- | GQAPRLLIYDASNRATGIPARFSGSGSGTDFTLTISSLEP | |
| | MH2p | EDFAVYYCQQRSNWPPALTFGGGTKVEIKRTVAAPEIVLT | |
| | | QSPATLSLSPGERATLSCRASQSISDFLHWYQQKPGQAPR | |
| | | LLIKYASQSISGIPARFSGSGSGTDFTLTISSLEPEDFAV | |
| | | YYCQNGHNFPPTFGGGTKVEIKRTAELPPKVSVFVPPRDG | |
| | | FFGNPRKSKLICKATGFSPRQIQVSWLREGKQVGSGVTTK | |
| | | QVQAEAKESGPTTYKVTSTLTIKESDWLGQSMFTCRVDHR | |
| | | GLTFQQNASSMC | |
| | Cetuximab VH- | QVQLKQSGPGLVQPSQSLSITCTVSGFSLTNYGVHWVRQS | 276 |
| | linker-CD3VH- | PGKGLEWLGVIWSGGNTDYNTPFTSRLSINKDNSKSQVFF | |
| | linker-AB002 VH- | KMNSLQSNDTAIYYCARALTYYDYEFAYWGQGTLVTVSAA | |
| | CH1-hinge-CH2- | STKGPSVFPLAPQVQLQQSGAELARPGASVKMSCKASGYT | |
| | CH3 (holes) | FTRYTMHWVKQRPGQGLEWIGYINPSRGYTNYNQKFKDKA | |
| | | TLTTDKSSSTAYMQLSSLTSEDSAVYYCARYYDDHYCLDY | |
| | | WGQGTTLTVSSASTKGPSVFPLAPSSKSTSGGTAALGCLV | |
| | | KDYFPEPVTVSWNSGALTSGVHTFPAVLQSSGLYSLSSVV | |
| | | TVPSSSLGTQTYICNVNHKPSNTKVDKKVEPKSCDKTHTC | |
| | | PPCPAPELLGGPSVFLFPPKPKDTLMISRTPEVTCVVVDV | |
| | | SHEDPEVKFNWYVDGVEVHNAKTKPREEQYNSTYRVVSVL | |
| | | TVLHQDWLNGKEYKCKVSNKALPAPIEKTISKAKGQPREP | |
| | | QVYTLPPSREEMTKNQVSLSCAVKGFYPSDIAVEWESNGQ | |
| | | PENNYKTTPPVLDSDGSFFLVSKLTVDKSRWQQGNVFSCS | |
| | | VMHEALHNHYTQKSLSLSPGK | |
| | Cetuximab VK- | DILLTQSPVILSVSPGERVSFSCRASQSIGTNIHWYQQRT | 277 |
| | linker-CD3VK- | NGSPRLLIKYASESISGIPSRFSGSGSGTDFTLSINSVES | |
| | linker-AB002 VK- | EDIADYYCQQNNNWPTTFGAGTKLELKRTVAAPSVFIFPP | |
| | Ck | QIVLTQSPAIMSASPGEKVTMTCRASSSVSYMNWYQQKSG | |
| | | TSPKRWIYDTSKVASGVPYRFSGSGSGTSYSLTISSMEAE | |
| | | DAATYYCQQWSSNPLTFGSGTKLEINRTVAAPSVFIFPPS | |
| | | DEQLKSGTASVVCLLNNFYPREAKVQWKVDNALQSGNSQE | |
| | | SVTEQDSKDSTYSLSSTLTLSKADYEKHKVYACEVTHQGL | |
| | | SSPVTKSFNRGEC | |
| PLY17 | AB765VH-L- | EVQLVESGGGLVQPGGSLRLSCAASGFAFSSYDMSWVRQA | 278 |
| | AB430VH-linker- | PGKGLEWVSYISGGGFTYYPDTVKGRFTISRDNSKNTLYL | |
| | MH2n-hinge-CH2- | QMNSLRAEDTAVYYCARQGANWELVYWGQGTLVTVSSAST | |
| | CH3 (knobs) | KGPSVFPLAPQVQLQQWGAGLLKPSETLSLTCAVYGGSFS | |
| | | GYYWSWIRQSPEKGLEWIGEINHGGYVTYNPSLESRVTIS | |
| | | VDTSKNQFSLKLSSVTAADTAVYYCARDYGPGNYDWYFDL | |
| | | WGRGTLVTVSSASTELPPKVSVFVPPRDGFFGNPRKSELI | |
| | | CEATGFSPRQIQVSWLREGKQVGSGVTTDQVQAEAKESGP | |
| | | TTYDVTSTLTIKESDWLGQSMFTCRVDHRGLTFQQNASSM | |
| | | CDKTHTCPPCPAPELLGGPSVFLFPPKPKDTLMISRTPEV | |
| | | TCVVVDVSHEDPEVKFNWYVDGVEVHNAKTKPREEQYNST | |
| | | YRVVSVLTVLHQDWLNGKEYKCKVSNKALPAPIEKTISKA | |
| | | KGQPREPQVYTLPPSREEMTKNQVSLWCLVKGFYPSDIAV | |
| | | EWESNGQPENNYKTTPPVLDSDGSFFLYSKLTVDKSRWQQ | |
| | | GNVFSCSVMHEALHNHYTQKSLSLSPGK | |
| | AB765VL-L- | EIVLTQSPATLSLSPGERATLSCRASQSISDFLHWYQQKP | 279 |
| | AB430VL-linker- | GQAPRLLIKYASQSISGIPARFSGSGSGTDFTLTISSLEP | |
| | MH2p | EDFAVYYCQNGHNFPPTFGGGTKVEIKRTVAAPSVFIFPP | |
| | | EIVLTQSPATLSLSPGERATLSCRASQSVSSYLAWYQQKP | |
| | | GQAPRLLIYDASNRATGIPARFSGSGSGTDFTLTISSLEP | |
| | | EDFAVYYCQQRSNWPPALTFGGGTKVEIKRTAELPPKVSV | |
| | | FVPPRDGFFGNPRKSKLICKATGFSPRQIQVSWLREGKQV | |
| | | GSGVTTKQVQAEAKESGPTTYKVTSTLTIKESDWLGQSMF | |
| | | TCRVDHRGLTFQQNASSMC | |
| | Cetuximab VH- | QVQLKQSGPGLVQPSQSLSITCTVSGFSLTNYGVHWVRQS | 280 |
| | linker-CD3VH- | PGKGLEWLGVIWSGGNTDYNTPFTSRLSINKDNSKSQVFF | |
| | linker-AB002 VH- | KMNSLQSNDTAIYYCARALTYYDYEFAYWGQGTLVTVSAA | |
| | CH1-hinge-CH2- | STKGPSVFPLAPQVQLQQSGAELARPGASVKMSCKASGYT | |
| | CH3 (holes) | FTRYTMHWVKQRPGQGLEWIGYINPSRGYTNYNQKFKDKA | |
| | | TLTTDKSSSTAYMQLSSLTSEDSAVYYCARYYDDHYCLDY | |
| | | WGQGTTLTVSSASTKGPSVFPLAPSSKSTSGGTAALGCLV | |
| | | KDYFPEPVTVSWNSGALTSGVHTFPAVLQSSGLYSLSSVV | |
| | | TVPSSSLGTQTYICNVNHKPSNTKVDKKVEPKSCDKTHTC | |
| | | PPCPAPELLGGPSVFLFPPKPKDTLMISRTPEVTCVVVDV | |
| | | SHEDPEVKFNWYVDGVEVHNAKTKPREEQYNSTYRVVSVL | |
| | | TVLHQDWLNGKEYKCKVSNKALPAPIEKTISKAKGQPREP | |
| | | QVYTLPPSREEMTKNQVSLSCAVKGFYPSDIAVEWESNGQ | |
| | | PENNYKTTPPVLDSDGSFFLVSKLTVDKSRWQQGNVFSCS | |
| | | VMHEALHNHYTQKSLSLSPGK | |
| | Cetuximab VK- | DILLTQSPVILSVSPGERVSFSCRASQSIGTNIHWYQQRT | 281 |
| | linker-CD3VK- | NGSPRLLIKYASESISGIPSRFSGSGSGTDFTLSINSVES | |
| | linker-AB002 VK- | EDIADYYCQQNNNWPTTFGAGTKLELKRTVAAPSVFIFPP | |
| | Ck | QIVLTQSPAIMSASPGEKVTMTCRASSSVSYMNWYQQKSG | |
| | | TSPKRWIYDTSKVASGVPYRFSGSGSGTSYSLTISSMEAE | |
| | | DAATYYCQQWSSNPLTFGSGTKLEINRTVAAPSVFIFPPS | |
| | | DEQLKSGTASVVCLLNNFYPREAKVQWKVDNALQSGNSQE | |
| | | SVTEQDSKDSTYSLSSTLTLSKADYEKHKVYACEVTHQGL | |
| | | SSPVTKSFNRGEC | |
| PLY18 | AB765VH-L- | EVQLVESGGGLVQPGGSLRLSCAASGFAFSSYDMSWVRQA | 282 |
| | AB430VH-linker- | PGKGLEWVSYISGGGFTYYPDTVKGRFTISRDNSKNTLYL | |
| | MH2n-hinge-CH2- | QMNSLRAEDTAVYYCARQGANWELVYWGQGTLVTVSSAST | |
| | CH3 (knobs) | KGPSVFPLAPQVQLQQWGAGLLKPSETLSLTCAVYGGSFS | |
| | | GYYWSWIRQSPEKGLEWIGEINHGGYVTYNPSLESRVTIS | |
| | | VDTSKNQFSLKLSSVTAADTAVYYCARDYGPGNYDWYFDL | |
| | | WGRGTLVTVSSASTELPPKVSVFVPPRDGFFGNPRKSELI | |
| | | CEATGFSPRQIQVSWLREGKQVGSGVTTDQVQAEAKESGP | |
| | | TTYDVTSTLTIKESDWLGQSMFTCRVDHRGLTFQQNASSM | |
| | | CDKTHTCPPCPAPELLGGPSVFLFPPKPKDTLMISRTPEV | |
| | | TCVVVDVSHEDPEVKFNWYVDGVEVHNAKTKPREEQYNST | |
| | | YRVVSVLTVLHQDWLNGKEYKCKVSNKALPAPIEKTISKA | |
| | | KGQPREPQVYTLPPSREEMTKNQVSLWCLVKGFYPSDIAV | |
| | | EWESNGQPENNYKTTPPVLDSDGSFFLYSKLTVDKSRWQQ | |
| | | GNVFSCSVMHEALHNHYTQKSLSLSPGK | |
| | AB765VL-S- | EIVLTQSPATLSLSPGERATLSCRASQSISDFLHWYQQKP | 283 |
| | AB430VL-linker- | GQAPRLLIKYASQSISGIPARFSGSGSGTDFTLTISSLEP | |
| | MH2p | EDFAVYYCQNGHNFPPTFGGGTKVEIKRTVAAPEIVLTQS | |
| | | PATLSLSPGERATLSCRASQSVSSYLAWYQQKPGQAPRLL | |
| | | IYDASNRATGIPARFSGSGSGTDFTLTISSLEPEDFAVYY | |
| | | CQQRSNWPPALTFGGGTKVEIKRTAELPPKVSVFVPPRDG | |
| | | FFGNPRKSKLICKATGFSPRQIQVSWLREGKQVGSGVTTK | |
| | | QVQAEAKESGPTTYKVTSTLTIKESDWLGQSMFTCRVDHR | |
| | | GLTFQQNASSMC | |
| | Cetuximab VH- | QVQLKQSGPGLVQPSQSLSITCTVSGFSLTNYGVHWVRQS | 284 |
| | linker-CD3VH- | PGKGLEWLGVIWSGGNTDYNTPFTSRLSINKDNSKSQVFF | |
| | linker-AB002 VH- | KMNSLQSNDTAIYYCARALTYYDYEFAYWGQGTLVTVSAA | |
| | CH1-hinge-CH2- | STKGPSVFPLAPQVQLQQSGAELARPGASVKMSCKASGYT | |
| | CH3 (holes) | FTRYTMHWVKQRPGQGLEWIGYINPSRGYTNYNQKFKDKA | |
| | | TLTTDKSSSTAYMQLSSLTSEDSAVYYCARYYDDHYCLDY | |
| | | WGQGTTLTVSSASTKGPSVFPLAPSSKSTSGGTAALGCLV | |
| | | KDYFPEPVTVSWNSGALTSGVHTFPAVLQSSGLYSLSSVV | |
| | | TVPSSSLGTQTYICNVNHKPSNTKVDKKVEPKSCDKTHTC | |
| | | PPCPAPELLGGPSVFLFPPKPKDTLMISRTPEVTCVVVDV | |
| | | SHEDPEVKFNWYVDGVEVHNAKTKPREEQYNSTYRVVSVL | |
| | | TVLHQDWLNGKEYKCKVSNKALPAPIEKTISKAKGQPREP | |
| | | QVYTLPPSREEMTKNQVSLSCAVKGFYPSDIAVEWESNGQ | |
| | | PENNYKTTPPVLDSDGSFFLVSKLTVDKSRWQQGNVFSCS | |
| | | VMHEALHNHYTQKSLSLSPGK | |
| | Cetuximab VK- | DILLTQSPVILSVSPGERVSFSCRASQSIGTNIHWYQQRT | 285 |
| | linker-CD3VK- | NGSPRLLIKYASESISGIPSRFSGSGSGTDFTLSINSVES | |
| | linker-AB002 VK- | EDIADYYCQQNNNWPTTFGAGTKLELKRTVAAPSVFIFPP | |
| | Ck | QIVLTQSPAIMSASPGEKVTMTCRASSSVSYMNWYQQKSG | |
| | | TSPKRWIYDTSKVASGVPYRFSGSGSGTSYSLTISSMEAE | |
| | | DAATYYCQQWSSNPLTFGSGTKLEINRTVAAPSVFIFPPS | |
| | | DEQLKSGTASVVCLLNNFYPREAKVQWKVDNALQSGNSQE | |
| | | SVTEQDSKDSTYSLSSTLTLSKADYEKHKVYACEVTHQGL | |
| | | SSPVTKSFNRGEC | |
| PLY19 | AB765VH-S- | EVQLVESGGGLVQPGGSLRLSCAASGFAFSSYDMSWVRQA | 286 |
| | AB430VH-linker- | PGKGLEWVSYISGGGFTYYPDTVKGRFTISRDNSKNTLYL | |
| | MH2n-hinge-CH2- | QMNSLRAEDTAVYYCARQGANWELVYWGQGTLVTVSSAST | |
| | CH3 (knobs) | KGPQVQLQQWGAGLLKPSETLSLTCAVYGGSFSGYYWSWI | |
| | | RQSPEKGLEWIGEINHGGYVTYNPSLESRVTISVDTSKNQ | |
| | | FSLKLSSVTAADTAVYYCARDYGPGNYDWYFDLWGRGTLV | |
| | | TVSSASTELPPKVSVFVPPRDGFFGNPRKSELICEATGFS | |
| | | PRQIQVSWLREGKQVGSGVTTDQVQAEAKESGPTTYDVTS | |
| | | TLTIKESDWLGQSMFTCRVDHRGLTFQQNASSMCDKTHTC | |
| | | PPCPAPELLGGPSVFLFPPKPKDTLMISRTPEVTCVVVDV | |
| | | SHEDPEVKFNWYVDGVEVHNAKTKPREEQYNSTYRVVSVL | |
| | | TVLHQDWLNGKEYKCKVSNKALPAPIEKTISKAKGQPREP | |
| | | QVYTLPPSREEMTKNQVSLWCLVKGFYPSDIAVEWESNGQ | |
| | | PENNYKTTPPVLDSDGSFFLYSKLTVDKSRWQQGNVFSCS | |
| | | VMHEALHNHYTQKSLSLSPGK | |
| | AB765VL-L- | EIVLTQSPATLSLSPGERATLSCRASQSISDFLHWYQQKP | 287 |
| | AB430VL-linker- | GQAPRLLIKYASQSISGIPARFSGSGSGTDFTLTISSLEP | |
| | MH2p | EDFAVYYCQNGHNFPPTFGGGTKVEIKRTVAAPSVFIFPP | |
| | | EIVLTQSPATLSLSPGERATLSCRASQSVSSYLAWYQQKP | |
| | | GQAPRLLIYDASNRATGIPARFSGSGSGTDFTLTISSLEP | |
| | | EDFAVYYCQQRSNWPPALTFGGGTKVEIKRTAELPPKVSV | |
| | | EVPPRDGFFGNPRKSKLICKATGFSPRQIQVSWLREGKQV | |
| | | GSGVTTKQVQAEAKESGPTTYKVTSTLTIKESDWLGQSMF | |
| | | TCRVDHRGLTFQQNASSMC | |
| | Cetuximab VH- | QVQLKQSGPGLVQPSQSLSITCTVSGFSLTNYGVHWVRQS | 288 |
| | linker-CD3VH- | PGKGLEWLGVIWSGGNTDYNTPFTSRLSINKDNSKSQVFF | |
| | linker-AB002 VH- | KMNSLQSNDTAIYYCARALTYYDYEFAYWGQGTLVTVSAA | |
| | CH1-hinge-CH2- | STKGPSVFPLAPQVQLQQSGAELARPGASVKMSCKASGYT | |
| | CH3 (holes) | FTRYTMHWVKQRPGQGLEWIGYINPSRGYTNYNQKFKDKA | |
| | | TLTTDKSSSTAYMQLSSLTSEDSAVYYCARYYDDHYCLDY | |
| | | WGQGTTLTVSSASTKGPSVFPLAPSSKSTSGGTAALGCLV | |
| | | KDYFPEPVTVSWNSGALTSGVHTFPAVLQSSGLYSLSSVV | |
| | | TVPSSSLGTQTYICNVNHKPSNTKVDKKVEPKSCDKTHTC | |
| | | PPCPAPELLGGPSVFLFPPKPKDTLMISRTPEVTCVVVDV | |
| | | SHEDPEVKFNWYVDGVEVHNAKTKPREEQYNSTYRVVSVL | |
| | | TVLHQDWLNGKEYKCKVSNKALPAPIEKTISKAKGQPREP | |
| | | QVYTLPPSREEMTKNQVSLSCAVKGFYPSDIAVEWESNGQ | |
| | | PENNYKTTPPVLDSDGSFFLVSKLTVDKSRWQQGNVFSCS | |
| | | VMHEALHNHYTQKSLSLSPGK | |
| | Cetuximab VK- | DILLTQSPVILSVSPGERVSFSCRASQSIGTNIHWYQQRT | 289 |
| | linker-CD3VK- | NGSPRLLIKYASESISGIPSRFSGSGSGTDFTLSINSVES | |
| | linker-AB002 VK- | EDIADYYCQQNNNWPTTFGAGTKLELKRTVAAPSVFIFPP | |
| | Ck | QIVLTQSPAIMSASPGEKVTMTCRASSSVSYMNWYQQKSG | |
| | | TSPKRWIYDTSKVASGVPYRFSGSGSGTSYSLTISSMEAE | |
| | | DAATYYCQQWSSNPLTFGSGTKLEINRTVAAPSVFIFPPS | |
| | | DEQLKSGTASVVCLLNNFYPREAKVQWKVDNALQSGNSQE | |
| | | SVTEQDSKDSTYSLSSTLTLSKADYEKHKVYACEVTHQGL | |
| | | SSPVTKSFNRGEC | |
| PLY20 | AB765VH-S- | EVQLVESGGGLVQPGGSLRLSCAASGFAFSSYDMSWVRQA | 290 |
| | AB430VH-linker- | PGKGLEWVSYISGGGFTYYPDTVKGRFTISRDNSKNTLYL | |
| | MH2n-hinge-CH2- | QMNSLRAEDTAVYYCARQGANWELVYWGQGTLVTVSSAST | |
| | CH3 (knobs) | KGPQVQLQQWGAGLLKPSETLSLTCAVYGGSFSGYYWSWI | |
| | | RQSPEKGLEWIGEINHGGYVTYNPSLESRVTISVDTSKNQ | |
| | | FSLKLSSVTAADTAVYYCARDYGPGNYDWYFDLWGRGTLV | |
| | | TVSSASTELPPKVSVFVPPRDGFFGNPRKSELICEATGFS | |
| | | PRQIQVSWLREGKQVGSGVTTDQVQAEAKESGPTTYDVTS | |
| | | TLTIKESDWLGQSMFTCRVDHRGLTFQQNASSMCDKTHTC | |
| | | PPCPAPELLGGPSVFLFPPKPKDTLMISRTPEVTCVVVDV | |
| | | SHEDPEVKFNWYVDGVEVHNAKTKPREEQYNSTYRVVSVL | |
| | | TVLHQDWLNGKEYKCKVSNKALPAPIEKTISKAKGQPREP | |
| | | QVYTLPPSREEMTKNQVSLWCLVKGFYPSDIAVEWESNGQ | |
| | | PENNYKTTPPVLDSDGSFFLYSKLTVDKSRWQQGNVFSCS | |
| | | VMHEALHNHYTQKSLSLSPGK | |
| | AB765VL-S- | EIVLTQSPATLSLSPGERATLSCRASQSISDFLHWYQQKP | 291 |
| | AB430VL-linker- | GQAPRLLIKYASQSISGIPARFSGSGSGTDFTLTISSLEP | |
| | MH2p | EDFAVYYCQNGHNFPPTFGGGTKVEIKRTVAAPEIVLTQS | |
| | | PATLSLSPGERATLSCRASQSVSSYLAWYQQKPGQAPRLL | |
| | | IYDASNRATGIPARFSGSGSGTDFTLTISSLEPEDFAVYY | |
| | | CQQRSNWPPALTFGGGTKVEIKRTAELPPKVSVFVPPRDG | |
| | | FFGNPRKSKLICKATGFSPRQIQVSWLREGKQVGSGVTTK | |
| | | QVQAEAKESGPTTYKVTSTLTIKESDWLGQSMFTCRVDHR | |
| | | GLTFQQNASSMC | |
| | Cetuximab VH- | QVQLKQSGPGLVQPSQSLSITCTVSGFSLTNYGVHWVRQS | 292 |
| | linker-CD3VH- | PGKGLEWLGVIWSGGNTDYNTPFTSRLSINKDNSKSQVFF | |
| | linker-AB002 VH- | KMNSLQSNDTAIYYCARALTYYDYEFAYWGQGTLVTVSAA | |
| | CH1-hinge-CH2- | STKGPSVFPLAPQVQLQQSGAELARPGASVKMSCKASGYT | |
| | CH3 (holes) | FTRYTMHWVKQRPGQGLEWIGYINPSRGYTNYNQKFKDKA | |
| | | TLTTDKSSSTAYMQLSSLTSEDSAVYYCARYYDDHYCLDY | |
| | | WGQGTTLTVSSASTKGPSVFPLAPSSKSTSGGTAALGCLV | |
| | | KDYFPEPVTVSWNSGALTSGVHTFPAVLQSSGLYSLSSVV | |
| | | TVPSSSLGTQTYICNVNHKPSNTKVDKKVEPKSCDKTHTC | |
| | | PPCPAPELLGGPSVFLFPPKPKDTLMISRTPEVTCVVVDV | |
| | | SHEDPEVKFNWYVDGVEVHNAKTKPREEQYNSTYRVVSVL | |
| | | TVLHQDWLNGKEYKCKVSNKALPAPIEKTISKAKGQPREP | |
| | | QVYTLPPSREEMTKNQVSLSCAVKGFYPSDIAVEWESNGQ | |
| | | PENNYKTTPPVLDSDGSFFLVSKLTVDKSRWQQGNVFSCS | |
| | | VMHEALHNHYTQKSLSLSPGK | |
| | Cetuximab VK- | DILLTQSPVILSVSPGERVSFSCRASQSIGTNIHWYQQRT | 293 |
| | linker-CD3VK- | NGSPRLLIKYASESISGIPSRFSGSGSGTDFTLSINSVES | |
| | linker-AB002 VK- | EDIADYYCQQNNNWPTTFGAGTKLELKRTVAAPSVFIFPP | |
| | Ck | QIVLTQSPAIMSASPGEKVTMTCRASSSVSYMNWYQQKSG | |
| | | TSPKRWIYDTSKVASGVPYRFSGSGSGTSYSLTISSMEAE | |
| | | DAATYYCQQWSSNPLTFGSGTKLEINRTVAAPSVFIFPPS | |
| | | DEQLKSGTASVVCLLNNFYPREAKVQWKVDNALQSGNSQE | |
| | | SVTEQDSKDSTYSLSSTLTLSKADYEKHKVYACEVTHQGL | |
| | | SSPVTKSFNRGEC | |

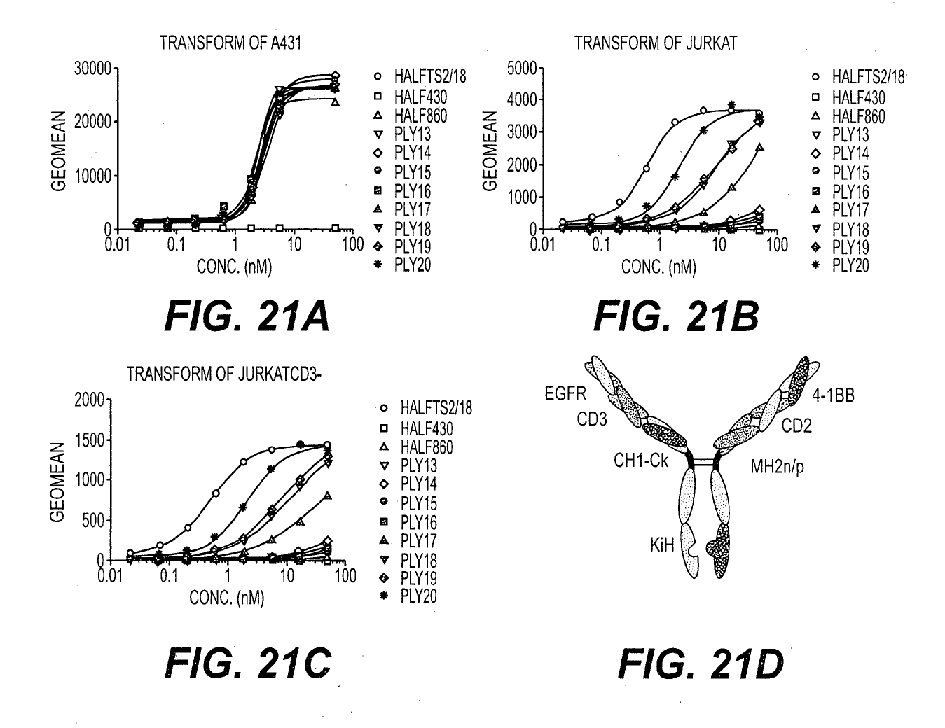

Tetraspecific molecules PLY13-20 were tested in a FACS binding assay to confirm that they retained binding affinity to 4-1BB, CD2, CD3, and EGFR. The anti-CD2 TS2/18 half body, anti-4-1BB AB430 halfbody, and anti-EGFR/CD3 DVD860 halfbody were also tested for comparison. A431 cells were used for testing EGFR binding. Jurkat CD3 positive cells were used for testing the combination binding to CD3, CD2 and 4-1BB. Jurkat CD3 negative cells were used for testing the combination binding to CD2 and 4-1BB. Cells were incubated with different concentrations of antibodies for 30 minutes and then incubated with incubated with fluorescence-conjugated secondary antibodies for another 30 minutes. Cells were then analyzed by flow cytometry and data was analyzed using FlowJo software. As shown in FIGS. 21A-C, Tetra-specific molecules maintained their binding affinity to all targets. The inner domain affinity was affected by the linker between the outer and inner VDs on each arm.

### Example 13: Generation of Duo-Fab-Ig Molecules with Wild Type MH2 Homodimer or Engineered MH2a/MH2b Heterodimer

As discussed in Example 7 and shown in FIG. 11, wild type MH2 homo-dimers or engineered MH2a/MH2b hetero-dimers may be used in DVD-Ig constructs to stabilize the outer variable domain and increase inner domain accessibility by using a single linker on the heavy or light chain. Duo-Fab-Ig is one of the formats listed in FIG. 11. Variable domains from one anti-STEAP1 antibody TPP3956 and one anti-PSMA antibody hPSMA17.1 were used to build Duo-Fab-Ig molecules. Two Duo-Fab-Ig molecules (NBDV001 and NBDV002) and one non-MH2 containing molecule (NBDV003) were generated. As shown in Table 22 and FIG. 22A, there are three chains in each molecules: one heavy chain containing MH2n after the first variable domain, one light chain containing MH2p with the variable domain pairing with the first variable domain on the heavy chain, and one light chain with the variable domain pairing with the second variable domain on the heavy chain incorporated in Ck. All cloning was completed using homologous recombination and transformation in DH5a cells. All Duo-Fab-Ig molecules were expressed in HEK293 cells and purified with MabSelect SuRe beads. The Duo-Fab-Ig molecules containing MH2 domains showed comparable expression levels and improved SEC monomer %, when compared to the expression level and SEC profile of the non-MH2 molecule NBDV003. The variable domain sequences used to generate the Duo-Fab-Ig molecules are listed in Table 23. Table 24 summarizes the sequences of the four chains in each of the tetraspecific molecules that were generated and tested.

TABLE 22
Duo-Fab-Ig Molecules Containing MH2 Domains

| | | | | Yield | |
|---|---|---|---|---|---|
| | Chain 1 | Chain 2 | Chain 3 | (mg/L) | SEC |
| NBDV001 | TPP3956VH-MH2n- | TPP3956VL-MH2p | hPSAM17.1VL-CK | 22 | 92.77 |
| | hPSMA17.1VH-CH1- | | | | |
| | hinge-CH2—CH3 | | | | |
| NBDV002 | TPP3956VL-MH2n- | TPP3956VH-MH2p | hPSAM17.1VL-CK | 13.6 | 94.6 |
| | hPSMA17.1VH-CH1- | | | | |
| | hinge-CH2—CH3 | | | | |
| NBDV003 | TPP3956VL-CK- | TPP3956VH-CH1 | hPSAM17.1VL-CK | 28 | 68.52 |
| | hPSMA17.1VH-CH1- | | | | |
| | hinge-CH2—CH3 | | | | |

TABLE 23
Antibody Variable Domains Used To Build IgG-Like Molecules Containing
MH2 Domains

| Antibody | | Variable Domain Sequence |
|---|---|---|
| Target | Domain | 12345678901234567890123456789012345678901234567890 |
| STEAP1 | STEAP1 | EVQLVESGGGVVQPGRSLRLSCVASGFPFNNYWMTWIRQAPGKGLEWIAS |
| (TPP3956) | (TPP3956) | ITITGGTTYYPGSVKGRFTISRDNSKSTLYLQMNSLRAEDTAVYYCTRER |
| | VH | YSTEYYPYYWYFDFWGQGTMVTVSS (SEQ ID NO: 294) |
| | STEAP1 | DIQMTQSPSSLSASVGDRVTITCRTSQDIGNYLRWFQQKPGKSPKLMIYD |
| | (TPP3956) | AINLAAGVPSRFSGSGSGTDYTLTISSLQPEDVATYYCLQHNEYPYTFGQ |
| | VK | GTKLEIK (SEQ ID NO: 295) |
| PSMA | PSMA | EVQLVQSGAEVKKPGSSVKVSCKASGYTFTDYYMNWVKQAPGKGLEWIGL |
| (hPSMA17. | (hPSMA17. | INPNSGGINYNQKFKVKATLTVDKSTSTAYMELSSLRSEDTAVYYCARRD |
| 1) | 1) VH | YGTSGDYWGQGTTVTVSS (SEQ ID NO: 296) |
| | PSMA | DIQMTQSPSSLSASVGDRVTITCHASQNINVWLSWYQQKPGKAPKLLIYK |
| | (hPSMA17. | ASNLHTGVPSRFSGSGSGTDFTLTISSLQPEDFATYYCQQGQSYPLTFGQ |
| | 1) VK | GTKLEIK (SEQ ID NO: 297) |

TABLE 24
Sequences Of Generated Duo-Fab-Ig Molecules

| Duo- | | Sequence | SEQ ID |
|---|---|---|---|
| Fab-Ig | Chain | 12345678901234567890123456789012345678901234567890 | NO: |
| NBDV001 | Chain 1 | EVQLVESGGGVVQPGRSLRLSCVASGFPFNNYWMTWIRQAPGKGLEWIAS | 298 |
| | | ITITGGTTYYPGSVKGRFTISRDNSKSTLYLQMNSLRAEDTAVYYCTRER | |
| | | YSTEYYPYYWYFDFWGQGTMVTVSSVIAELPPKVSVFVPPRDGFFGNPRK | |
| | | SELICEATGFSPRQIQVSWLREGKQVGSGVTTDQVQAEAKESGPTTYDVT | |
| | | STLTIKESDWLGQSMFTCRVDHRGLTFQQNASSMCGGEVQLVQSGAEVKK | |
| | | PGSSVKVSCKASGYTFTDYYMNWVKQAPGKGLEWIGLINPNSGGINYNQK | |
| | | FKVKATLTVDKSTSTAYMELSSLRSEDTAVYYCARRDYGTSGDYWGQGTT | |
| | | VTVSSASTKGPSVFPLAPSSKSTSGGTAALGCLVKDYFPEPVTVSWNSGA | |
| | | LTSGVHTFPAVLQSSGLYSLSSVVTVPSSSLGTQTYICNVNHKPSNTKVD | |
| | | KKVEPKSCDKTHTCPPCPAPEAAGGPSVFLFPPKPKDTLMISRTPEVTCV | |
| | | VVDVSHEDPEVKFNWYVDGVEVHNAKTKPREEQYNSTYRVVSVLTVLHQD | |
| | | WLNGKEYKCKVSNKALPAPIEKTISKAKGQPREPQVYTLPPSREEMTKNQ | |
| | | VSLTCLVKGFYPSDIAVEWESNGQPENNYKTTPPVLDSDGSFFLYSKLTV | |
| | | DKSRWQQGNVFSCSVMHEALHNHYTQKSLSLSPGK | |
| | Chain 2 | DIQMTQSPSSLSASVGDRVTITCRTSQDIGNYLRWFQQKPGKSPKLMIYD | 299 |
| | | AINLAAGVPSRFSGSGSGTDYTLTISSLQPEDVATYYCLQHNEYPYTFGQ | |
| | | GTKLEIKVIAELPPKVSVFVPPRDGFFGNPRKSKLICKATGFSPRQIQVS | |
| | | WLREGKQVGSGVTTKQVQAEAKESGPTTYKVTSTLTIKESDWLGQSMFTC | |
| | | RVDHRGLTFQQNASSMC | |
| | Chain 3 | DIQMTQSPSSLSASVGDRVTITCHASQNINVWLSWYQQKPGKAPKLLIYK | 300 |
| | | ASNLHTGVPSRFSGSGSGTDFTLTISSLQPEDFATYYCQQGQSYPLTFGQ | |
| | | VGTKLEIKRTVAAPSVFIFPPSDEQLKSGTASVVCLLNNFYPREAKVQWKV | |
| | | DNALQSGNSQESVTEQDSKDSTYSLSSTLTLSKADYEKHKVYACEVTHQG | |
| | | LSSPVTKSFNRGEC | |
| NBDV002 | Chain 1 | DIQMTQSPSSLSASVGDRVTITCRTSQDIGNYLRWFQQKPGKSPKLMIYD | 301 |
| | | AINLAAGVPSRFSGSGSGTDYTLTISSLQPEDVATYYCLQHNEYPYTFGQ | |
| | | GTKLEIKVIAELPPKVSVFVPPRDGFFGNPRKSELICEATGFSPRQIQVS | |
| | | WLREGKQVGSGVTTDQVQAEAKESGPTTYDVTSTLTIKESDWLGQSMFTC | |
| | | RVDHRGLTFQQNASSMCGGEVQLVQSGAEVKKPGSSVKVSCKASGYTFTD | |
| | | YYMNWVKQAPGKGLEWIGLINPNSGGINYNQKFKVKATLTVDKSTSTAYM | |
| | | ELSSLRSEDTAVYYCARRDYGTSGDYWGQGTTVTVSSASTKGPSVFPLAP | |
| | | SSKSTSGGTAALGCLVKDYFPEPVTVSWNSGALTSGVHTFPAVLQSSGLY | |
| | | SLSSVVTVPSSSLGTQTYICNVNHKPSNTKVDKKVEPKSCDKTHTCPPCP | |
| | | APEAAGGPSVFLFPPKPKDTLMISRTPEVTCVVVDVSHEDPEVKFNWYVD | |
| | | GVEVHNAKTKPREEQYNSTYRVVSVLTVLHQDWLNGKEYKCKVSNKALPA | |
| | | PIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHE | |
| | | ALHNHYTQKSLSLSPGK | |
| | Chain 2 | EVQLVESGGGVVQPGRSLRLSCVASGFPFNNYWMTWIRQAPGKGLEWIAS | 302 |
| | | ITITGGTTYYPGSVKGRFTISRDNSKSTLYLQMNSLRAEDTAVYYCTRER | |
| | | YSTEYYPYYWYFDFWGQGTMVTVSSVIAELPPKVSVFVPPRDGFFGNPRK | |
| | | SKLICKATGFSPRQIQVSWLREGKQVGSGVTTKQVQAEAKESGPTTYKVT | |
| | | STLTIKESDWLGQSMFTCRVDHRGLTFQQNASSMC | |
| | Chain 3 | DIQMTQSPSSLSASVGDRVTITCHASQNINVWLSWYQQKPGKAPKLLIYK | 303 |
| | | ASNLHTGVPSRFSGSGSGTDFTLTISSLQPEDFATYYCQQGQSYPLTFGQ | |
| | | GTKLEIKRTVAAPSVFIFPPSDEQLKSGTASVVCLLNNFYPREAKVQWKV | |
| | | DNALQSGNSQESVTEQDSKDSTYSLSSTLTLSKADYEKHKVYACEVTHQG | |
| | | LSSPVTKSFNRGEC | |
| NBDV003 | Chain 1 | DIQMTQSPSSLSASVGDRVTITCRTSQDIGNYLRWFQQKPGKSPKLMIYD | 304 |
| | | AINLAAGVPSRFSGSGSGTDYTLTISSLQPEDVATYYCLQHNEYPYTFGQ | |
| | | GTKLEIKRTVAAPSVFIFPPSDEQLKSGTASVVCLLNNFYPREAKVQWKV | |
| | | DNALQSGNSQESVTEQDSKDSTYSLSSTLTLSKADYEKHKVYACEVTHQG | |
| | | LSSPVTKSFNRGECGGEVQLVQSGAEVKKPGSSVKVSCKASGYTFTDYYM | |
| | | NWVKQAPGKGLEWIGLINPNSGGINYNQKFKVKATLTVDKSTSTAYMELS | |
| | | SLRSEDTAVYYCARRDYGTSGDYWGQGTTVTVSSASTKGPSVFPLAPSSK | |
| | | STSGGTAALGCLVKDYFPEPVTVSWNSGALTSGVHTFPAVLQSSGLYSLS | |
| | | SVVTVPSSSLGTQTYICNVNHKPSNTKVDKKVEPKSCDKTHTCPPCPAPE | |
| | | AAGGPSVFLFPPKPKDTLMISRTPEVTCVVVDVSHEDPEVKFNWYVDGVE | |
| | | VHNAKTKPREEQYNSTYRVVSVLTVLHQDWLNGKEYKCKVSNKALPAPIE | |
| | | KTISKAKGQPREPQVYTLPPSREEMTKNQVSLTCLVKGFYPSDIAVEWES | |
| | | NGQPENNYKTTPPVLDSDGSFFLYSKLTVDKSRWQQGNVFSCSVMHEALH | |
| | | NHYTQKSLSLSPGK | |
| | Chain 2 | EVQLVESGGGVVQPGRSLRLSCVASGFPFNNYWMTWIRQAPGKGLEWIAS | 305 |
| | | ITITGGTTYYPGSVKGRFTISRDNSKSTLYLQMNSLRAEDTAVYYCTRER | |
| | | YSTEYYPYYWYFDFWGQGTMVTVSSASTKGPSVFPLAPSSKSTSGGTAAL | |
| | | GCLVKDYFPEPVTVSWNSGALTSGVHTFPAVLQSSGLYSLSSVVTVPSSS | |
| | | LGTQTYICNVNHKPSNTKVDKKVEPKSC | |
| | Chain 3 | DIQMTQSPSSLSASVGDRVTITCHASQNINVWLSWYQQKPGKAPKLLIYK | 306 |
| | | ASNLHTGVPSRFSGSGSGTDFTLTISSLQPEDFATYYCQQGQSYPLTFGQ | |
| | | GTKLEIKRTVAAPSVFIFPPSDEQLKSGTASVVCLLNNFYPREAKVQWKV | |
| | | DNALQSGNSQESVTEQDSKDSTYSLSSTLTLSKADYEKHKVYACEVTHQG | |
| | | LSSPVTKSFNRGEC | |

Duo-Fab-Ig molecules were tested in a FACS binding assay to confirm that they retained binding affinity to STEAP1 and PSMA. The parental anti-STEAP1 antibody TPP3956 and anti-PSMA hPSMA17.1 were also tested for comparison. 293/PSMA cells were used for testing PSMA binding. 293/STEAP1 cells were used for testing STEAP1 binding. LnCap cells were used for testing the combination binding to PSMA/STEAP1. Cells were incubated with different concentrations of antibodies for 30 minutes and then incubated with incubated with fluorescence-conjugated secondary antibodies for another 30 minutes. Cells were then analyzed by flow cytometry and data was analyzed using FlowJo software. As shown in FIG. 22B, Duo-Fab-Igs maintained binding affinity to both targets comparable to their parental antibodies. The Duo-Fab-Igs showed enhanced binding on LnCap cells which has both PSMA and STEAP1 expressed on the cell surface. DVD889 [hu IgG1/k] was used as a negative control.

### Example 14: Analytical Methods and Techniques for Bispecific Molecules Characterizations

### Example 14.1: Size Exclusion Chromatography (SEC)

Bispecific molecules were separated on an SEC column based on protein dynamic size (Instrument: Dionex HPLC; Column: TOSOH, TSKgel G3000sw ×L, CN#08541; Buffer: 0.1M sodium phosphate buffer, 0.1 sodium sulfide, pH6.8; UV280: monitor proteins at UV 280 nm; Flow rate: 1.0 mL/minute, isocratic). The molecular weights of desired bispecific molecule BMH1, BMH2, BMH3, BMH4, and BMH5 were about 150 kDa. After protein A purification, the molecular profiles of BMH1, BMH2, BMH3, BMH4, and BMH5 were analyzed by SEC

### Example 14.2: Hydrophobic Interaction Chromatography (HIC)

Bispecific molecules were separated on an HIC column based on protein hydrophobicity (Instrument: Dionex HPLC; Column: TOSOH, TSKgel G3000sw xL, CN#08541; Buffer A: 1.8M ammonia sulfide, 20 mM phosphate buffer, pH7.2; Buffer B: 20 mM phosphate buffer, pH7.2; UV280: monitor proteins at UV 280 nm; Flow rate: 1.0 mL/minute; Gradient: 0% to 17% buffer B in 17 minutes, 100% buffer for 3 minutes, and back to 100% buffer A for 7 minutes).

### Example 14.3: Isoelectric Focusing (iCE3)

Bispecific molecules were separated on an HIC column based on isoelectric point (pI) and hydrodynamic charge (Instrument: ProteinSimple iCE3; Capillary: ProteinSimple, PN#101700; Chemicals: ProteinSimple: 0.5% Methyl Cellulose (PN#102505), iCE electrolyte kit (PN#102506), 1% Methyl Cellulose (PN#101876), Pharmalyte (PN#17-0456-01) and pI markers; Instrument conditions: focusing time: 8 minutes; UV280: monitor proteins at UV 280 nm).

### Example 14.4: Mass Spectrometry (MS)

Bispecific molecular weight and identification was determined by mass spec (MS) (Instrument: Agilent HPLC-TOF or HPLC-QTOF; Column: Vydac C4, CN#214MS5115, and CapTrap cartridge; Buffer A: 0.1% FA+0.01% TFA in H2O, buffer B: 0.1% FA+0.01% TFA in CAN; Flow rate: 50 μL/minute; Gradient: 5% buffer B for 5 minutes, 28% to 50% buffer B in 10 minutes, 50% to 95% buffer B in 10 minutes and back to 5% buffer B for 3 minutes for C4 column. 5% buffer B for 7 minutes, 100% buffer B for 7 minutes and back to 5% buffer B for 5 minutes for CapTrap cartridge; MS conditions: For reduced protein: gas temperature 350 C, drying gas 12 L/min, nebulizer 60 psg, fragmentor 350v, skimmer 75v, OCTI RF Vpp 750v, Vcap 5000v. For intact protein: gas temperature 300 C, drying gas 12 L/min, nebulizer 60 psg, fragmentor 350v, skimmer 85v, OCTI RF Vpp 750v, Vcap 5500v).

### Example 15: Generation of IgG-Like Molecules with MH2 Domains

Additional bivalent monospecific molecules with CH1/Cκ replaced by MH2n/MH2p can be constructed using variable domains known in the art. Table 25 summarizes exemplary variable domain sequences that can be used for constructing IgG-like molecules containing MH2 domains. Exemplary bivalent monospecific molecules comprising the variable domains listed in Table 25 are shown in Table 26. Table 26 also shows an exemplary halfbody that can be constructed by using the variable domains of cetuximab.

TABLE 25
Exemplary Antibody Variable Domains That Can Be Used To Build IgG-Like
Molecules Containing MH2 Domains

| Antibody | | Variable Domain Sequence | SEQ ID |
|---|---|---|---|
| Target | Domain | 1234567890123456789012345678901234567890 | NO: |
| EGFR | Cetuximab | QVQLKQSGPGLVQPSQSLSITCTVSGFSLTNYGVHWVRQS | 307 |
| (Cetuximab) | VH | PGKGLEWLGVIWSGGNTDYNTPFTSRLSINKDNSKSQVFF | |
| | | KMNSLQSNDTAIYYCARALTYYDYEFAYWGQGTLVTVSA | |
| | Cetuximab | DILLTQSPVILSVSPGERVSFSCRASQSIGTNIHWYQQRT | 308 |
| | VK | NGSPRLLIKYASESISGIPSRFSGSGSGTDFTLSINSVES | |
| | | EDIADYYCQQNNNWPTTFGAGTKLELK | |
| HER2 | Herceptin | EVQLVESGGGLVQPGGSLRLSCAASGFNIKDTYIHWVRQA | 309 |
| (Herceptin) | VH | PGKGLEWVARIYPTNGYTRYADSVKGRFTISADTSKNTAY | |
| | | LQMNSLRAEDTAVYYCSRWGGDGFYAMDYWGQGTLVTVSS | |
| | Herceptin | DIQMTQSPSSLSASVGDRVTITCRASQDVNTAVAWYQQKP | 310 |
| | VK | GKAPKLLIYSASFLYSGVPSRFSGSRSGTDFTLTISSLQP | |
| | | EDFATYYCQQHYTTPPTFGQGTKVEIK | |

TABLE 26
Sequences of Exemplary Bivalent Monospecific Molecules and Halfbody

| Mono- | | |
|---|---|---|
| Specific | | Sequence |
| Molecules | Chain | 12345678901234567890123456789012345678901234567890 |
| MMH1 | Herceptin VH- | EVQLVESGGGLVQPGGSLRLSCAASGFNIKDTYIHWVRQAPGKGLEWVAR |
| | linker-MH2- | IYPTNGYTRYADSVKGRFTISADTSKNTAYLQMNSLRAEDTAVYYCSRWG |
| | hinge-CH2-CH3 | GDGFYAMDYWGQGTLVTVSSASTELPPKVSVFVPPRDGFFGNPRKSKLIC |
| | | QATGFSPRQIQVSWLREGKQVGSGVTTDQVQAEAKESGPTTYKVTSTLTI |
| | | KESDWLGQSMFTCPVDHRGLTFQQNASSMCDKTHTCPPCPAPELLGGPSV |
| | | FLFPPKPKDTLMISRTPEVTCVVVDVSHEDPEVKFNWYVDGVEVENAKTK |
| | | PREEQYNSTYRVVSVLTVLHQDWLNGKEYKCKVSNKALPAPIEKTISKAK |
| | | GQPREPQVYTLPPSREEMTKNQVSLTCLVKGFYPSDIAVEWESNGQPENN |
| | | YKTTPPVLDSDGSFFLYSKLTVDKSRWQQGNVFSCSVMHEALHNHYTQKS |
| | | LSLSPGK (SEQ ID NO: 311) |
| | Herceptin VK- | DIQMTQSPSSLSASVGDRVTITCRASQDVNTAVAWYQQKPGKAPKLLIYS |
| | linker-MH2 | ASFLYSGVPSRFSGSRSGTDFTLTISSLQPEDHATYYCQQHYTTPPTFGQ |
| | | GTKVEIKRTAELPPKVSVFVPPRDGFFGNPRKSKLICQATGFSPRQIQVS |
| | | WLREGKQVGSGVTTDQVQAEAKESGPTTYKVTSTLTIKESDWLGQSMFTC |
| | | RVDHRGLTFQQNASSMC (SEQ ID NO: 312) |
| MMH2 | Herceptin VH- | EVQLVESGGGLVQPGGSLRLSCAASGFNIKDTYIHWVRQAPGKGLEWVAR |
| | linker-MH2p- | IYPTNGYTRYADSVKGRFTISADTSKNTAYLQMNSLRAEDTAVYYCSRWG |
| | hinge-CH2-CH3 | GDGFYAMDYWGQGTLVTVSSASTELPPKVSVFVPPPDGFFGNPRKSKLIC |
| | | KATGFSPRQIQVSWLREGKQVGSGVTTKQVQAEAKESGPTTYKVTSTLTI |
| | | KESDWLGQSMFTCRVDHRGLTFQQNASSMCDKTHTCPPCPAPELLGGPSV |
| | | FLFPPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTISKAK |
| | | GQPREPQVYTLPPSREEMTKNQVSLTCLVKGFYPSDIAVEWESNGQPENN |
| | | YKTTPPVLDSDGSFFLYSKLTVDKSRWQQGNVFSCSVMHEALHNHYTQKS |
| | | LSLSPGK (SEQ ID NO: 313) |
| | Herceptin- | DIQMTQSPSSLSASVGDRVTITCRASQDVNTAVAWYQQKPGKAPKLLIYS |
| | linker-MH2n | ASFLYSGVPSRFSGSPSGTDFTLTISSLQPEDFATYYCQQHYTTPPTFGQ |
| | | GTKVEIKPTAELPPKVSVFVPPRDGFFGNPRKSELICEATGFSPRQIQVS |
| | | WLREGKQVGSGVTTDQVQAEAKESGPTTYDVTSTLTIKESDWLGQSMFTC |
| | | RVDHRGLTFQQNASSMC (SEQ ID NO: 314) |
| MMH3 | Herceptin VH- | EVQLVESGGGLVQPGGSLRLSCAASGFNIKDTYIHWVRQAPGKGLEWVAR |
| | linker-MH2h- | IYPTNGYTRYADSVKGRFTISADTSKNTAYLQMNSLRAEDTAVYYCSRWG |
| | hinge-CH2-CH3 | GDGFYAMDYWGQGTLVTVSSASTELPPKVSVFVPPRDGFFGNPRKSKLAC |
| | | SATGFSPRQIQVSWLREGKQVGSGVTTDQVQAEAKESGPTTYKVASTLTI |
| | | KESDWLGQSMFTCRVDHRGLTFQQNASSMCDKTHTCPPCPAPELLGGPSV |
| | | FLFPPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTISKAK |
| | | GQPREPQVYTLPPSREEMTKNQVSLTCLVKGFYPSDIAVEWESNGQPENN |
| | | YKTTPPVLDSDGSFFLYSKLTVDKSRWQQGNVFSCSVMHEALHNHYTQKS |
| | | LSLSPGK (SEQ ID NO: 315) |
| | Herceptin VK- | DIQMTQSPSSLSASVGDRVTITCRASQDVNTAVAWYQQKPGKAPKLLIYS |
| | linker-MH2k | ASFLYSGVPSRFSGSRSGTDFTLTISSLQPEDFATYYCQQHYTTPPTFGQ |
| | | GTKVEIKRTAELPPKVSVFVPPRDGFFGNPRKSKLWCQATGFSPRQIQVS |
| | | WLREGKQVGSGVTTDQVQAEAKESGPTTYKVTSTLTIKESDWLGQSMFTC |
| | | RVDHRGLTFQQNASSMC (SEQ. ID NO: 316) |
| MMH4 | Herceptin VH- | EVQLVESGGGLVQPGGSLRLSCAASGFNIKDTYIHWVRQAPGKGLEWVAR |
| | linker-MH2n- | IYPTNGYTRYADSVKGRFTISADTSKNTAYLQMNSLRAEDTAVYYCSRWG |
| | hinqe-CH2-CH3 | GDGFYAMDYWGQGTLVTVSSASTELPPKVSVFVPPRDGFFGNPRKSELIC |
| | | EATGFSPRQIQVSWLREGKQVGSGVTTDQVQAEAKESGPTTYDVTSTLTI |
| | | KESDWLGQSMFTCRVDHRGLTFQQNASSMCDKTHTCPPCPAPELLGGPSV |
| | | FLFPPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTISKAK |
| | | GQPREPQVYTLPPSREEMTKNQVSLTCLVKGFYPSDIAVEWESNGQPENN |
| | | YKTTPPVLDSDGSFFLYSKLTVDKSRWQQGNVFSCSVMHEALHNHYTQKS |
| | | LSLSPGK (SEQ ID NO: 317) |
| | Herceptin VK | DIQMTQSPSSLSASVGDRVTITCRASQDVNTAVAWYQQKPGKAPKLLIYS |
| | linker-MH2p | ASFLYSGVPSRFSGSRSGTDFTLTISSLQPEDFATYYCQQHYTTPPTFGQ |
| | | GTKVEIKRTAELPPKVSVFVPPRDGFFGNPRKSKLICKATGFSPRQIQVS |
| | | WLREGKQVGSGVTTKQVQAEAKESGPTTYKVTSTLTIKESDWLGQSMFTC |
| | | RVDHRGLTFQQNASSMC (SEQ ID NO: 318) |
| MMH5 | Herceptin VH- | EVQLVESGGGLVQPGGSLRLSCAASGFNIKDTYIHWVPQAPGKGLENVAR |
| | linker-MH2k- | IYPTNGYTRYADSVKGRFTISADTSKNTAYLQMNSLRAEDTAVYYCSRWG |
| | hinge-CH2-CH3 | GDGFYAMDYWGQGTLVTVSSASTELPPKVSVFVPPRDGFFGNPRKSKLWC |
| | | QATGESPRQIQVSWLREGKQVGSGVTTDQVQAEAKESGPTTYKVTSTLTI |
| | | KESDWLGQSMFTCRVDHRGLTFQQNASSMCDKTHTCPPCPAPELLGGPSV |
| | | FLFPPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTISKAK |
| | | GQPREPQVYTLPPSREEMTKNQVSLTCLVKGFYPSDIAVEWESNGQPENN |
| | | YKTTPPVLDSDGSFFLYSKLTVDKSRWQQGNVFSCSVMHEALHNHYTQKS |
| | | LSLSPGK (SEQ ID NO: 319) |
| | Herceptin VH | DIQMTQSPSSLSASVGDRVTITCRASQDVNTAVAWYQQKPGKAPKLLIYS |
| | linker-MH2h | ASFLYSGVPSRFSGSRSGTDFTLTISSLQPEDFATYYCQQHYTTPPTFGQ |
| | | GTKVEIKRTAELPPKVSVFVPPRDGFFGNPRKSKLACSATGFSPRQIQVS |
| | | WLREGKQVGSGVTTDQVQAEAKESGPTTYKVASTLTIKESDWLGQSMFTC |
| | | RVDHRGLTFQQNASSMC (SEQ ID NO: 320) |
| Half- | Cetuximab-VH- | QVQLKQSGPGLVQPSQSLSITCTVSGFSLTNYGVHWVRQSPGKGLEWLGV |
| EGFR | CT1-hinge- | IWSGGNTDYNTPFTSRLSINKDNSKSQVFFKMNSLQSNDTAIYYCARALT |
| | CH2-CH3 | YYDYEFAYWGQGTLVTVSAASTKGPSVFPLAPSSKSTSGGTAALGCLVKD |
| | (halfbody) | YFPEPVTVSWNSGALTSGVHTFPAVLQSSGLYSLSSVVTVPSSSLGTQTY |
| | | ICNVNHKPSNTKVDKKVEPKSCDKTHTSPPSPAPELLGGPSVFLFPPKPK |
| | | DTLMISRTPEVTCVVVDVSHEDPEVKFNWYVDGVEVHNAKTKPREEQYNS |
| | | TYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVL |
| | | DSDGSFRLYSKLTVDKSPWQQGNVFSCSVMHEALHNHYTQKSLSLSPGK |
| | | (SEQ ID NO: 321) |
| | Cetuximab-VK- | DILLTQSPVILSVSPGERVSFSCRASQSIGTNIHWYQQRTNGSPRLLIKY |
| | CK | ASESISGIPSRFSGSGSGTDFTLSINSVESEDIADYYCQQNNNWPTTFGA |
| | | GTKLELKRTVAAPSVFIFPPSDEQLKSGTASVVCLLNNFYPREAKVQWKV |
| | | DNALQSGNSQESVTEQDSKDSTYSLSSTLTLSKADYEKHKVYACEVTHQG |
| | | LSSPVTKSFNRGEC (SEQ ID NO: 322) |

### INCORPORATION BY REFERENCE

The contents of all cited references (including literature references, patents, patent applications, and websites) that may be cited throughout this application are hereby expressly incorporated by reference in their entirety for any purpose, as are the references cited therein. Where a reference expressly or inherently contradicts anything in the present disclosure, the disclosure will control.

### EQUIVALENTS

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

1-115. (canceled)

116. A binding protein comprising a first heavy chain and a first light chain forming an antigen binding region and a constant region comprising a modified CH1 domain (CH1*) and a modified CL domain (CL*), wherein
a. the CH1* domain comprises an IgM CH2 domain, an IgE CH2 domain, or a variant thereof; and
b. the CL* domain comprises an IgM CH2 domain, an IgE CH2 domain, or a variant thereof,
and wherein the heavy chain and light chain interact at one or more interface between the CH1* and CL*.

117. The binding protein of claim 116, wherein the CH1* and CL* comprise variants of an IgM or IgE CH2 domain that have been modified to increase electrostatic or hydrophobic interactions at the one or more interface.

118. The binding protein of claim 116, wherein
a. the CH1* comprises a variant of an IgM or IgE CH2 domain, wherein the variant comprises at least one modified amino acid residue at the one or more interface, wherein the modified amino acid residue introduces a more positive or negative charge than the original residue being replaced; and
b. the CL* comprises a variant of an IgM or IgE CH2 domain, wherein the variant comprises at least one modified amino acid residue at the one or more interface, wherein the modified residue introduces a more negative charge than the original residue being replaced if the CH1* modified residue introduces a more positive charge, or wherein the CL* modified residue introduces a more positive charge if the CH1* modified residue introduces a more negative charge.

119. The binding protein of claim 116, wherein
a. the CH1* comprises a variant of an IgM or IgE CH2 domain, wherein the variant comprises at least one engineered protuberance at the one or more interface, the protuberance comprising at least one altered contact residue; and
b. the CL* comprises a variant of an IgM or IgE CH2 domain, wherein the variant comprises at least one engineered cavity at the one or more interface, the cavity comprising at least one altered contact residue; or
c. the CH1* comprises a variant of an IgM or IgE CH2 domain, wherein the variant comprises at least one engineered cavity at the one or more interface, the cavity comprising at least one altered contact residue; and
d. the CL* comprises a variant of an IgM or IgE CH2 domain, wherein the variant comprises at least one engineered protuberance at the one or more interface, the protuberance comprising at least one altered contact residue.

120. The binding protein of claim 119, further comprising at least one additional protuberance or cavity on the heavy chain, and at least one counterpart cavity or protuberance on the light chain, wherein the additional protuberance and cavity promote heavy chain and light chain pairing, and inhibit homodimer formation of two heavy chains or two light chains.

121. The binding protein of claim 117, wherein the IgM or IgE CH2 domain variants promote heavy chain and light chain heterodimer pairing, and inhibit homodimer pairing of two heavy chains or two light chains.

122. The binding protein of claim 117, wherein the IgM or IgE CH2 domain variant comprises a modification of a wild-type human IgM or IgE CH2 domain at one or more of amino acids D12, K20, I22, Q24, D81, K85.1, T86, and Q119.

123. The binding protein of claim 116, wherein the constant region comprises an IgG hinge region, and wherein the hinge region is further modified to remove at least one cysteine residue found in a wild-type IgG hinge region.

124. The binding protein of claim 116, wherein the IgM or IgE CH2 domain variant comprises a CH1, C kappa, or C lambda DE loop in place of a wild-type IgM or IgE CH2 DE loop.

125. The binding protein of claim 116, wherein the constant region prior to modification is a wild-type human IgG or fragment thereof, wherein the fragment lacks all or a part of an IgG CH3 domain.

126. The binding protein of claim 125, wherein the IgG constant region prior to modification is a human wild-type IgG1, IgG2a, IgG2b, IgG3, or IgG4 subtype.

127. The binding protein of claim 116, wherein the binding protein comprises a second heavy chain and a second light chain, wherein the second heavy chain and second light chain interact at one or more interface and form a second antigen binding region, wherein:
(a) the second heavy and light chains comprise a wild-type IgG heavy chain constant region and a wild-type IgG light chain constant region; or
(b) the first heavy chain comprises a modified CH3 domain, and the second heavy chain comprises a modified CH3 domain, and wherein the modified CH3 domains are preferably modified IgG CH3 domains, wherein the modifications promote pairing of the first and second heavy chains at one or more interface between the CH3 domains on the first and second heavy chains, and inhibit homodimer formation of two first heavy chains or two second heavy chains.

128. The binding protein of claim 116, wherein the binding protein is an antibody, a bispecific antibody, a dual variable domain immunoglobulin (DVD-Ig) binding protein, or a multispecific binding protein.

129. The binding protein of claim 116, wherein the IgM CH2 domain, IgE CH2 domain, or variant thereof is attached on the heavy chain and/or light chain via a linker.

130. The binding protein of claim 127, wherein the binding protein is a trispecific antibody comprising a second heavy chain and a second light chain, wherein:
(a) the second heavy chain comprises a second heavy chain variable domain (VH2) and a third heavy chain variable domain (VH3), and the second light chain comprises a second light chain variable domain (VL2) and a third light chain variable domain (VL3), which together form second and third binding sites for the same or different antigens; or
(b) the first heavy chain comprises a first heavy chain variable domain (VH1) and a second heavy chain variable domain (VH2), and the first light chain comprise a first light chain variable domain (VL1) and a second light chain variable domain (VL2), which together form first and second binding sites for the same or different antigens, and the second heavy chain comprises a third heavy chain variable domain (VH3), and the second light chain comprises a third light chain variable domain (VL3), which together form a third binding site for the same or a different antigen.

131. The binding protein of claim 127, wherein the binding protein is a tetraspecific antibody comprising a second heavy chain and a second light chain, wherein:
(a) wherein the first heavy chain comprises a first heavy chain variable domain (VH1) and a second heavy chain variable domain (VH2), and the first light chain comprise a first light chain variable domain (VL1) and a second light chain variable domain (VL2), which together form first and second binding sites for the same or different antigens; and
(b) wherein the second heavy chain comprises a third heavy chain variable domain (VH3) and a fourth heavy chain variable domain (VH4), and the second light chain comprises a third light chain variable domain (VL3) and a fourth light chain variable domain (VL4), which together form third and fourth binding sites for the same or different antigens.

132. The binding protein of claim 116, wherein the binding protein is capable of binding to one or more of CD3, EGFR, TNF, and a T-cell epitope.

133. A binding protein conjugate comprising the binding protein of claim 116, the binding protein conjugate further comprising an immunoadhesion molecule, an imaging agent, a therapeutic agent, or a cytotoxic agent.

134. An isolated nucleic acid encoding the binding protein of claim 116.

135. A vector comprising the isolated nucleic acid of claim 134.

136. A host cell comprising the vector of claim 135.

137. A method of producing a binding protein, comprising culturing the host cell of claim 136 in culture medium under conditions sufficient to produce the binding protein.

138. A pharmaceutical composition comprising the binding protein of claim 116, and a pharmaceutically acceptable carrier.

139. A method of treating a subject for a disease or a disorder by administering the binding protein of claim 116 to the subject.
